(19) United States
(12) Patent Application Publication
(10) Pub. No.: US 2026/0240938 A1
(43) Pub. Date: Aug. 20, 2026

(54) METHODS, COMPOSITIONS, AND RELATED USES FOR MEDICAL MANAGEMENT OF SUBJECTS WITH HEART FAILURE, IDIOPATHIC CARDIOMYOPATHY, DILATED CARDIOMYOPATHY, RESTRICTIVE CARDIOMYOPATHY, HYPERTROPHIC CARDIOMYOPATHY, OR HYPERTENSIVE CARDIOMYOPATHY

(71) Applicant: Stealth BioTherapeutics Inc., Needham, MA (US)

(72) Inventor: David Brown, Needham, MA (US)

(73) Assignee: Stealth BioTherapeutics Inc., (US)

(21) Appl. No.: 19/443,408

(22) Filed: Jan. 8, 2026

Related U.S. Application Data
(60) Provisional application No. 63/743,606, filed on Jan. 9, 2025.

(51) Int. Cl. A61K 38/07; A61K 31/401; A61K 31/403; A61K 31/41; A61K 31/4178; A61K 31/4184; A61K 31/4245; A61K 31/55; A61P 9/00

(52) U.S. Cl. CPC A61K 38/07; A61K 31/401; A61K 31/403; A61K 31/41; A61K 31/4178; A61K 31/4184; A61K 31/4245; A61K 31/55; A61P 9/00

(57) ABSTRACT

The disclosure provides compounds (e.g., peptides), compositions (e.g., formulations or medicaments), methods and related uses for the medical management of mammalian subjects diagnosed as having, or suspected of having, heart failure, idiopathic cardiomyopathy, dilated cardiomyopathy, restrictive cardiomyopathy, hypertrophic cardiomyopathy or hypertensive cardiomyopathy. The methods comprise administering to a subject an effective amount of a peptide, or mixture of peptides (alone or as prepared in a composition, formulation or medicament) of Formula A, or a pharmaceutically acceptable salt, hydrate, solvate, and/or tautomer thereof.

### CROSS-REFERENCE TO RELATED APPLICATIONS

This application claims the benefit of and priority to U.S. Provisional Patent Application No. 63/743,606, filed Jan. 9, 2025, the content of which is incorporated herein by reference in its entirety for any and all purposes.

### TECHNICAL FIELD

The present disclosure relates generally to compounds (e.g., peptides), compositions (e.g., medicaments or formulations), methods, and related uses for treating, preventing, inhibiting, ameliorating, or delaying the onset of heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM). The present technology relates to administering an effective amount of a peptide, mixture of peptides, or a composition, formulation or medicament comprising the peptide or mixture of peptides as described herein; or a pharmaceutically acceptable salt, hydrate, solvate, stereoisomer and/or tautomer of said peptide or peptides (including peptides in their carboxylate form), optionally in combination with an effective amount of an additional therapeutic agent such as an angiotensin-converting enzyme (ACE) inhibitor, an angiotensin receptor blocker (ARB), a beta blocker, a diuretic or a mineralocorticoid receptor antagonist.

### INTRODUCTION

The following description is provided to assist the understanding of the reader. None of the information provided or references cited is admitted as prior art to the compounds, compositions, methods and/or uses disclosed herein.

Heart failure (HF) is a condition that occurs when the heart is unable to pump enough oxygenated blood to supply the body's needs. When the heart is unable to meet the demands of the body, blood often backs up in the lungs, causing shortness of breath. Weakness, exercise intolerance and fatigue are other symptoms of heart failure. Heart failure is often associated with conditions that leave the heart too weak or stiff to properly fill the valves and therefore pump blood properly/efficiently throughout a subject's body. Heart failure can be categorized as: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. Heart failure can be categorized as congestive heart failure (CHF).

Idiopathic cardiomyopathy (ICM) is a cardiomyopathy associated with abnormalities of ventricular wall thickness, size of ventricular cavity, contraction, relaxation, conduction and/or rhythm and where there is no known/discernable cause. Symptoms include shortness of breath, fatigue, swelling in the legs and abnormal heart rhythms. Idiopathic cardiomyopathy can, inter alia, manifest itself as restrictive cardiomyopathy, dilated cardiomyopathy, or hypertrophic cardiomyopathy.

Dilated cardiomyopathy (DCM) is a condition whereby the heart's ventricle is enlarged as a result of stretching of the muscular wall, thereby becoming thinner and weaker. Although both the left ventricle and the right ventricle may become dilated, dilation usually occurs first in the left ventricle. This weaking of the heart structure results in a reduction in pumping efficiency and the delivery of oxygenated blood to the organs of the subject. Dilation of the heart ventricles often will eventually lead to heart failure.

Restrictive cardiomyopathy (RCM) is a condition whereby the heart muscle stiffens, thereby making it difficult for the chambers of the heart to fill properly with blood-ultimately reducing overall blood flow to the organs of the patient/subject.

Hypertrophic cardiomyopathy (HCM) is a condition whereby the heart muscle thickens, thereby making it harder for the heart to pump blood. Hypertrophic cardiomyopathy is often caused by a genetic mutation. Obstructive hypertrophic cardiomyopathy (a.k.a., hypertrophic obstructive cardiomyopathy (HOCM)) is the most common form of hypertrophic cardiomyopathy. Often HCM is caused by impaired growth of heart muscle whereby cells become layered instead of smooth, thereby leading to stiffness and scaring of the heart tissue. This stiffness and scaring of the heart tissue leads to a reduction in pumping efficiency and the delivery of oxygenated blood to the organs of the subject. Hypertrophy of the heart can eventually lead to heart failure.

Hypertensive cardiomyopathy (HTN-CM) refers to myriad changes in the left ventricle, left atrium and coronary arteries as a result of chronic high blood pressure. Often, the most noticeable effect of chronic hypertension on the heart is increase in left ventricle thickness (i.e., becomes hypertrophic). If left untreated, hypertensive cardiomyopathy can lead to the development of acute complications like acute coronary syndrome, decompensated heart failure, or sudden cardiac death. Hypertensive cardiomyopathy is predominantly a disease of impaired relaxation rather than impaired contractility. Therefore, patients are usually asymptomatic when resting but cannot produce appropriate cardiac output when exercising. Damage caused by hypertensive cardiomyopathy may eventually lead to heart failure.

Existing treatments for subjects with heart failure and/or cardiomyopathy depend on the type and severity of the heart damage. For example, different treatment regimens might be applied to treatment of hypertrophic cardiomyopathy as compared with dilated cardiomyopathy. Generally, treatments involve lifestyle changes, medications, surgical procedures and/or cardiac rehabilitation.

In summary, heart failure and/or cardiomyopathies are a global problem that effect millions of patients each year and are leading to the deaths of at least hundreds of thousands of patients in the United States each year. Although there are current treatments for heart failure, idiopathic cardiomyopathy, dilated cardiomyopathy, restrictive cardiomyopathy, hypertrophic cardiomyopathy and hypertensive cardiomyopathy, additional treatments and therapies that address deficiencies in, or that improve upon, the state of care would be beneficial to the patient population. The following discussion addresses some of those needs.

### SUMMARY

The present disclosure relates to peptides, compositions (i.e., formulations and medicaments) as well as related methods and uses pertaining to the treatment, prevention, inhibition, amelioration and/or delay the onset of heart failure, idiopathic cardiomyopathy, restrictive cardiomyopathy, dilated cardiomyopathy, hypertrophic cardiomyopathy, and/or hypertensive cardiomyopathy in a mammalian subject in need thereof. The peptides disclosed herein target, inter alia, mitochondrial dysfunction and are particularly useful in treating the cardiomyopathies described above because they are particularly effective at penetrating the heart tissue and thereby interacting with the mitochondria of the heart. In addition to exhibiting significant heart tissue penetration, these peptides generally appear to exhibit a significantly stronger effect (at identical concentrations) on mitochondria in the heart tissue as compared with other more well known molecules, such as elamipretide. Because these peptides can achieve efficacious dosing at lower concentrations than other drugs, these peptides are particularly well suited for addressing the above referenced cardiomyopathies, as well as various other signs, symptoms and conditions associated with the above referenced cardiomyopathies.

In one aspect, this disclosure pertains to a method for treating, preventing, inhibiting, ameliorating or delaying the onset of heart failure, idiopathic cardiomyopathy, dilated cardiomyopathy, or restrictive cardiomyopathy in a mammalian subject in need thereof, comprising administering to the subject a therapeutically effective amount of a peptide of Formula A, or a mixture of peptides of Formula A:

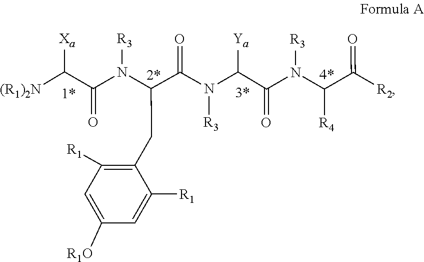

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

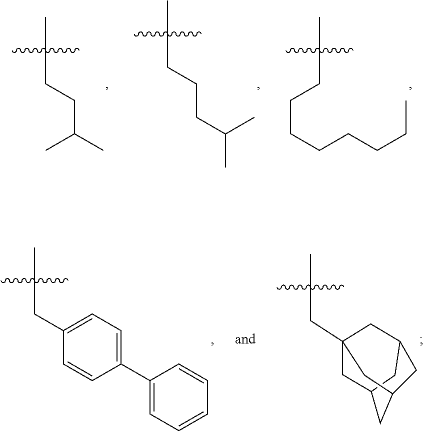

Xₐ is selected from

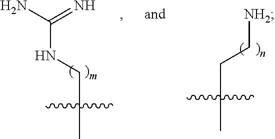

Yₐ is selected from

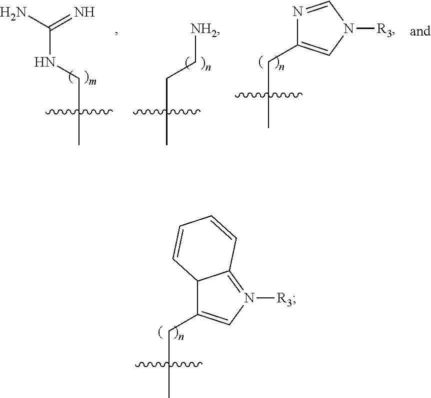

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments, the peptide or peptides of Formula A is/are a peptide/peptides of Formula A-1 or Formula A-2:

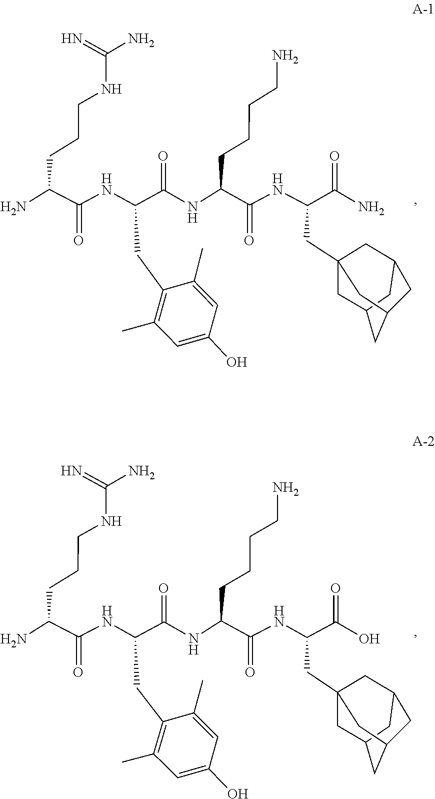 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide of Formula A is a peptide of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

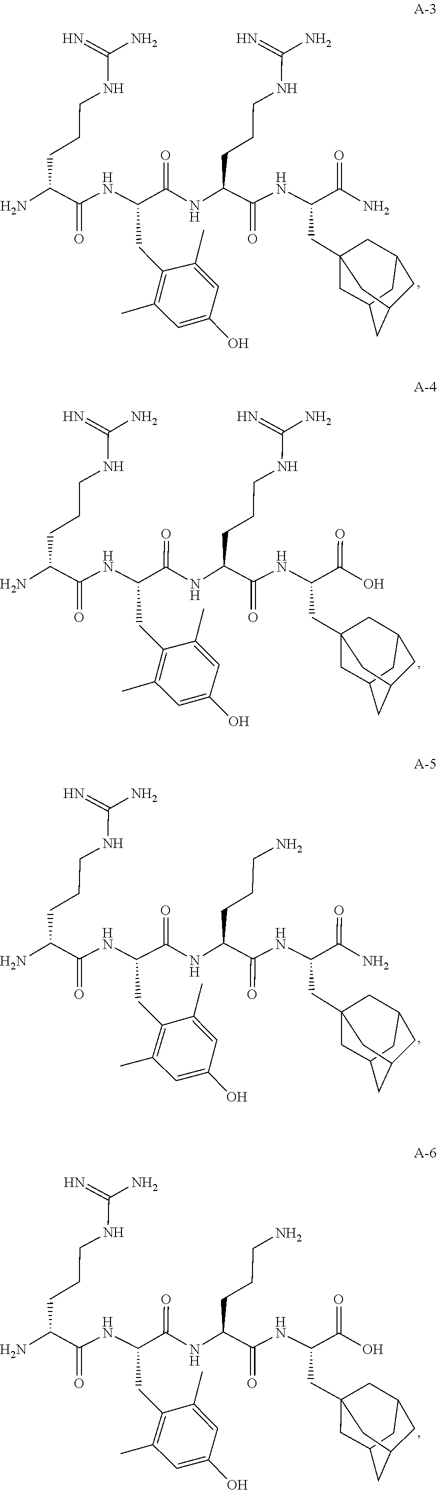 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

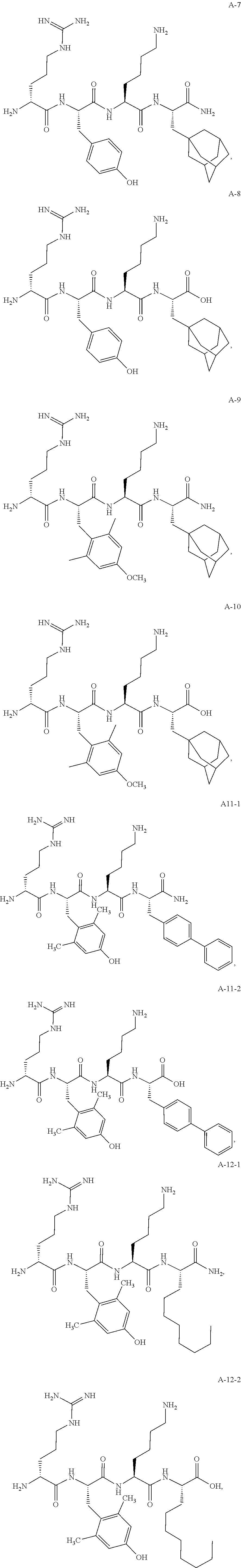

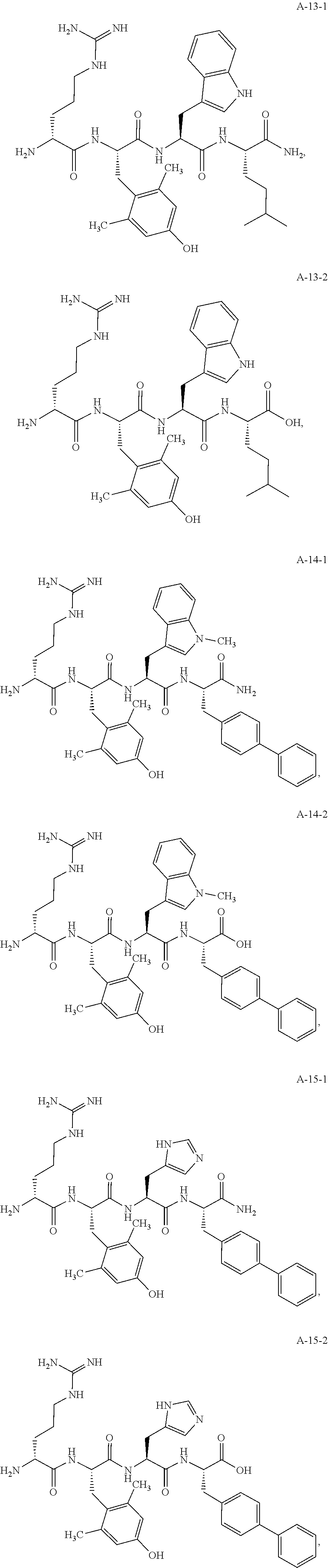

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the method, the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the method, the heart failure is congestive heart failure (CHF).

In some embodiments of the method, administration of the peptide or peptides; (i) ameliorates, (ii) delays the onset of, and/or (iii) delays the progression of one or more signs or symptoms of heart failure in the subject as compared with a control subject or group of control subjects to which the peptide or peptides was/were not administered. In some embodiments of the method, the signs or symptoms of heart failure include: (i) shortness of breath, (ii) ankle and/or foot swelling; (iii) leg swelling; (iv) fatigue, and/or (v) labored breathing when lying flat.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments, the method further comprises separately, sequentially, or simultaneously administering an additional therapeutic agent to the subject. In some embodiments, the additional therapeutic agent is an angiotensin-converting enzyme (ACE) inhibitor, an angiotensin receptor blocker (ARB), a beta blocker, a diuretic, a mineralocorticoid receptor antagonist or a calcium channel blocker (CCB). In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol and propranolol. In some embodiments of the method, the diuretic is bumetanide, furosemide or torsemide. In some embodiments of the method, the mineralocorticoid receptor antagonist is spironolactone or eplerenone. In some embodiments of the method, the calcium channel blocker is amlodipine (Norvasc®), diltiazem (Cardizem), felodipine, isradipine, nicardipine, nifedipine (Procardia®), nisoldipine (Sular®) or verapamil (Verelan®). In some embodiments of the method, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris-HCl salt.

In another aspect, this disclosure pertains to a composition or medicament for treating, preventing, inhibiting, ameliorating or delaying the onset of heart failure, idiopathic cardiomyopathy, dilated cardiomyopathy, or restrictive cardiomyopathy in a mammalian subject in need thereof, wherein the composition or medicament comprises a peptide of Formula A, or a mixture of peptides of Formula A:

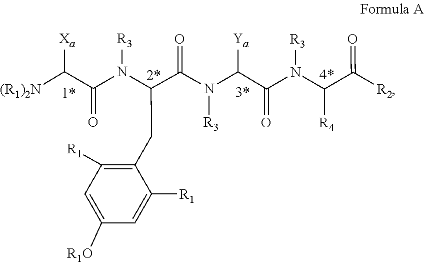

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

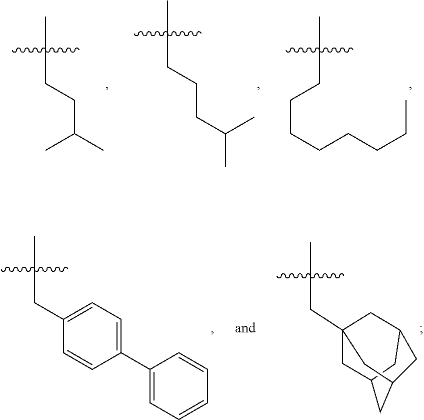

Xₐ is selected from

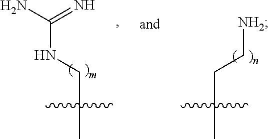

Yₐ is selected from

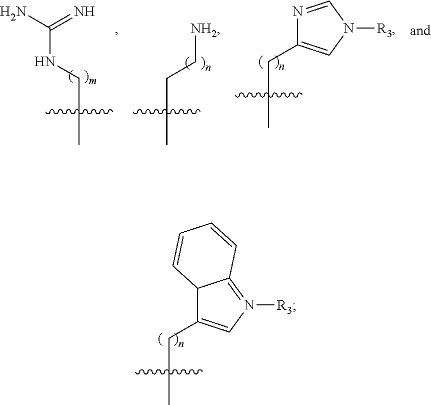

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments, the peptide or peptides of Formula A is/are a peptide/peptides of Formula A-1 or Formula A-2:

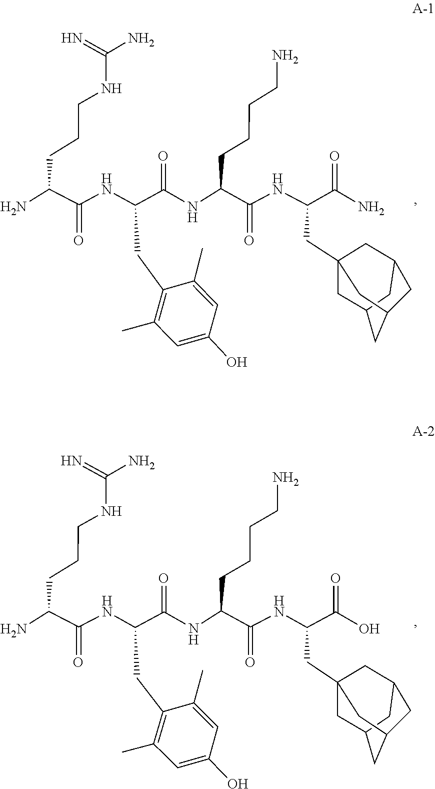 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

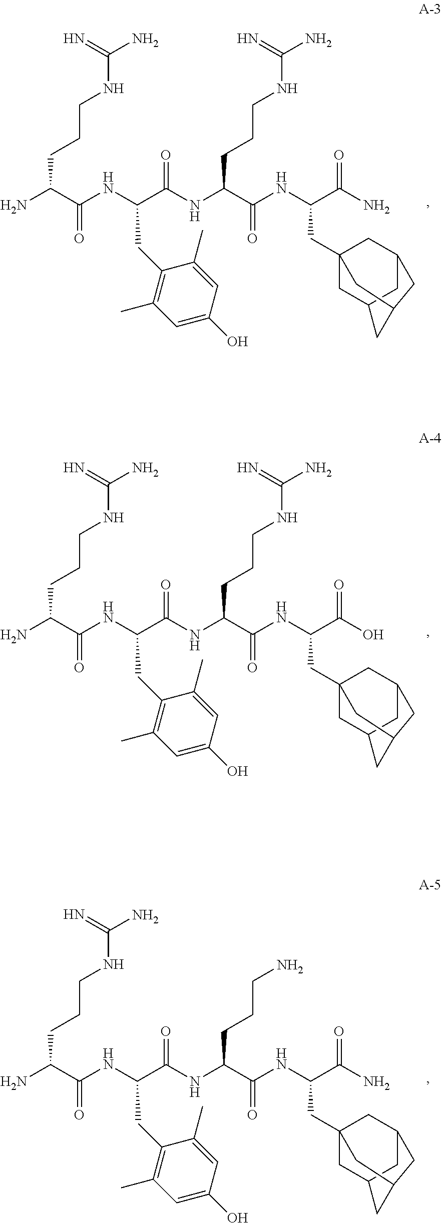 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

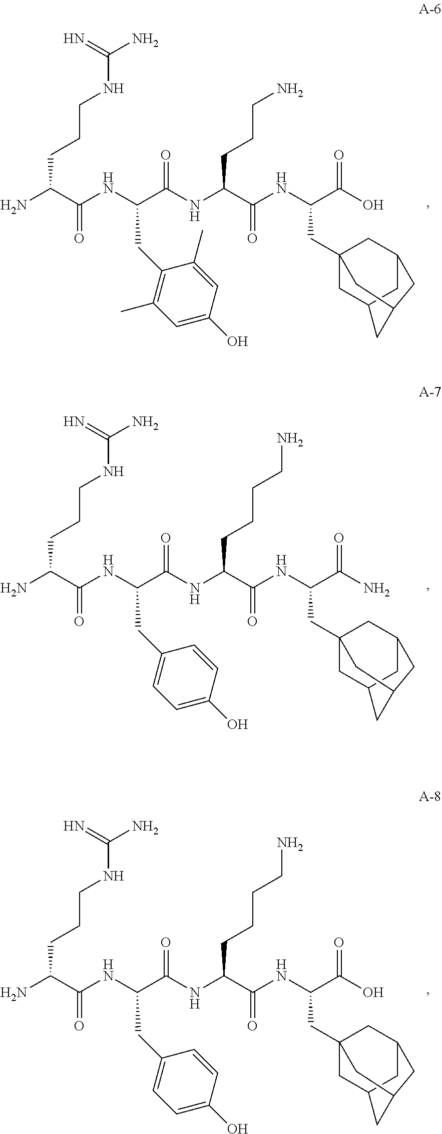 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

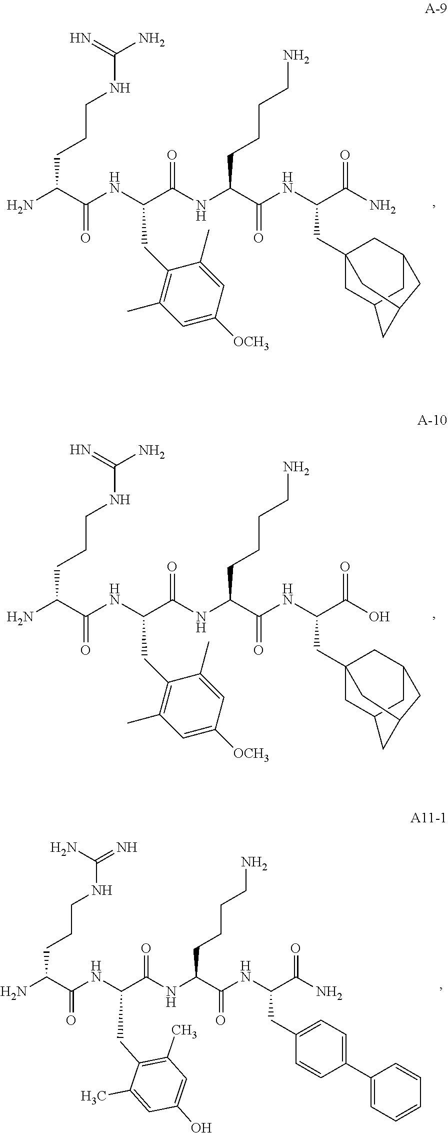 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

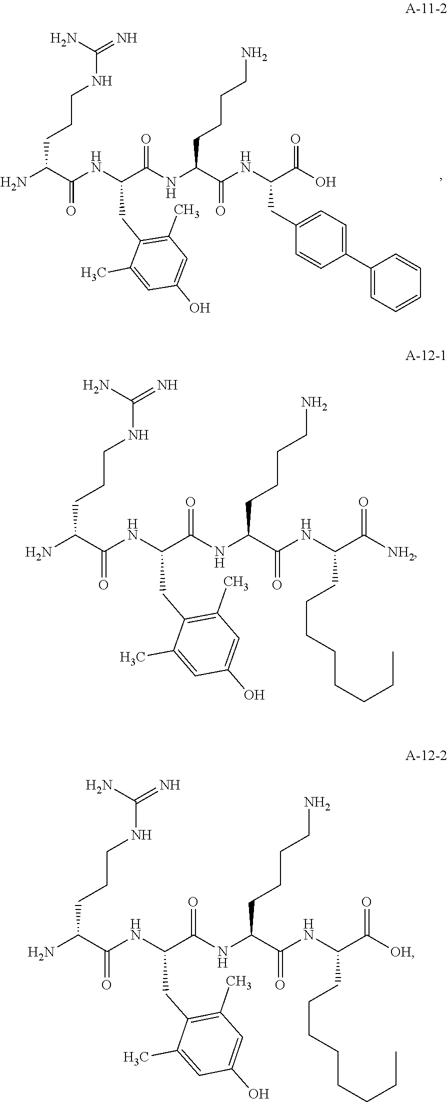 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

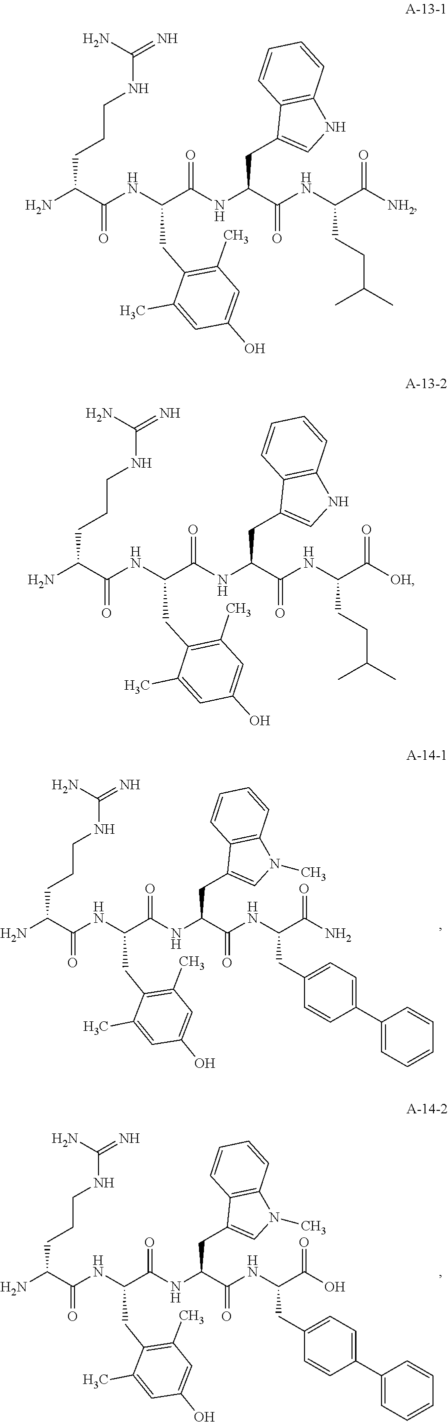 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

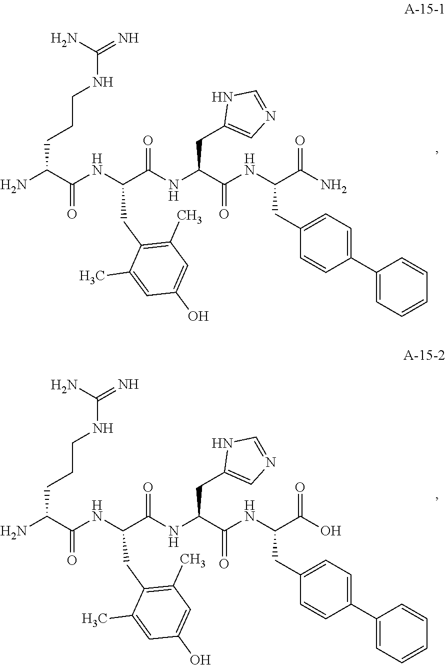 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the composition or medicament, the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the composition or medicament, the heart failure is congestive heart failure (CHF).

In some embodiments of the composition or medicament, administration of the composition or medicament to the subject; (i) ameliorates, (ii) delays the onset of, and/or (iii) delays the progression of one or more signs or symptoms of heart failure in the subject as compared with a control subject or group of control subjects to which the peptide or peptides was/were not administered. In some embodiments of the composition or medicament, the signs or symptoms of heart failure are: (i) shortness of breath, (ii) ankle and/or foot swelling; (iii) leg swelling; (iv) fatigue, and/or (v) labored breathing when lying flat.

In some embodiments of the composition or medicament, the composition or medicament comprising the peptide is administered daily for: (i) 12 weeks or more; (ii) 24 weeks or more; (iii) 48 weeks or more; (iv) 72 weeks or more; or (v) 96 weeks or more. In some embodiments of the method, the composition or medicament comprising the peptide or peptides is administered subcutaneously or intravenously. In some embodiments of the composition or medicament, the composition or medicament comprising the peptide or peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris-HCl salt.

In another aspect, this disclosure pertains to the use of a peptide, mixture of peptides, or a composition or medicament comprising the peptide or mixture of peptides, for treating, preventing, inhibiting, ameliorating or delaying the onset of heart failure, idiopathic cardiomyopathy, dilated cardiomyopathy, or restrictive cardiomyopathy in a mammalian subject in need thereof, wherein the peptide or peptides of the mixture is/are of Formula A:

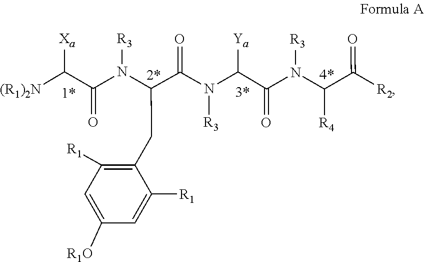

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

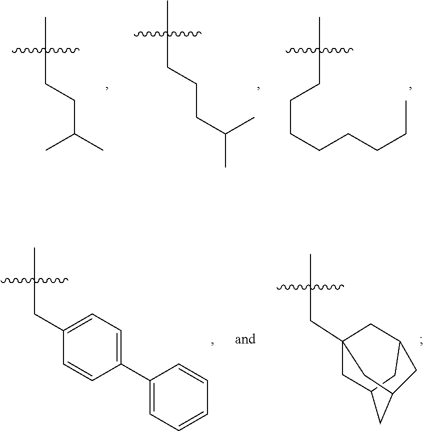

Xₐ is selected from

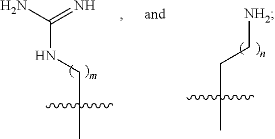

Yₐ is selected from

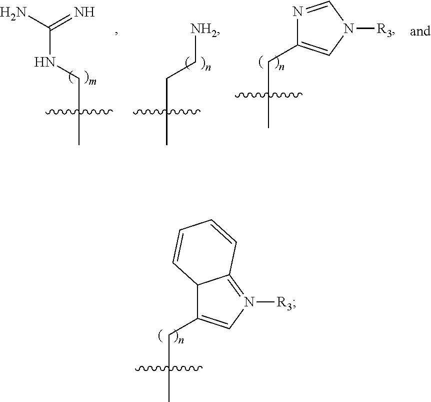

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom. In some embodiments of the use, the peptide or peptides of Formula A is/are a peptide/peptides of Formula A-1 or Formula A-2:

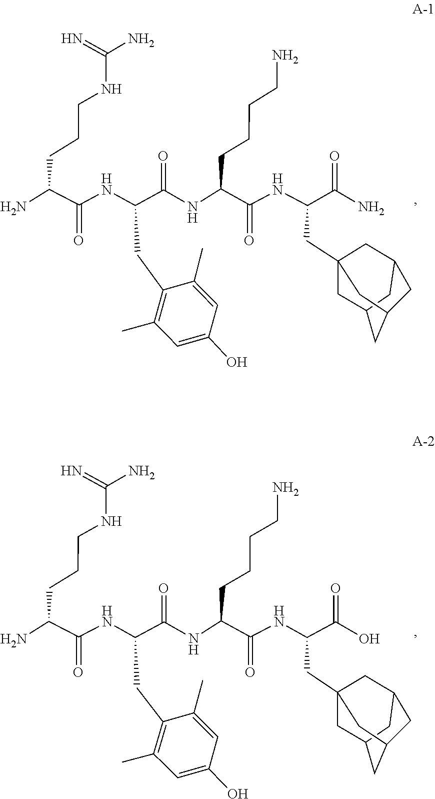 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the use, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

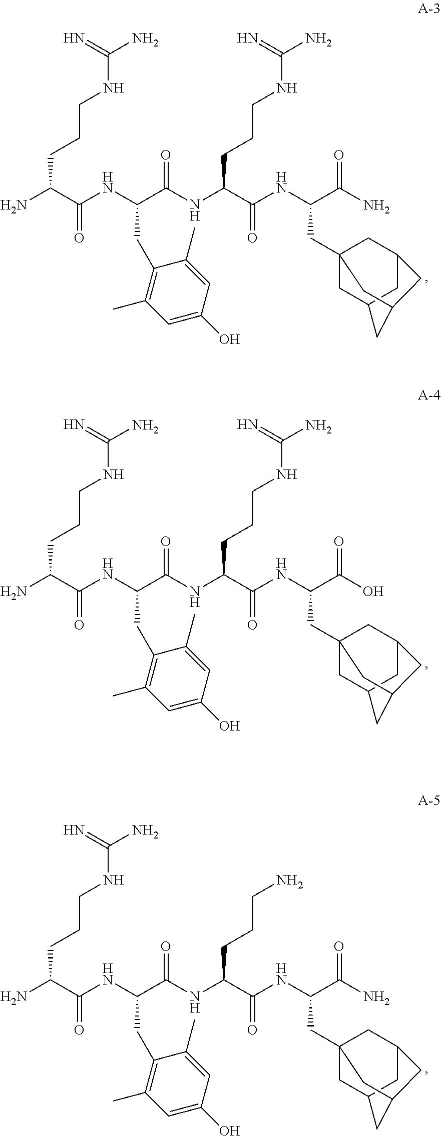 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

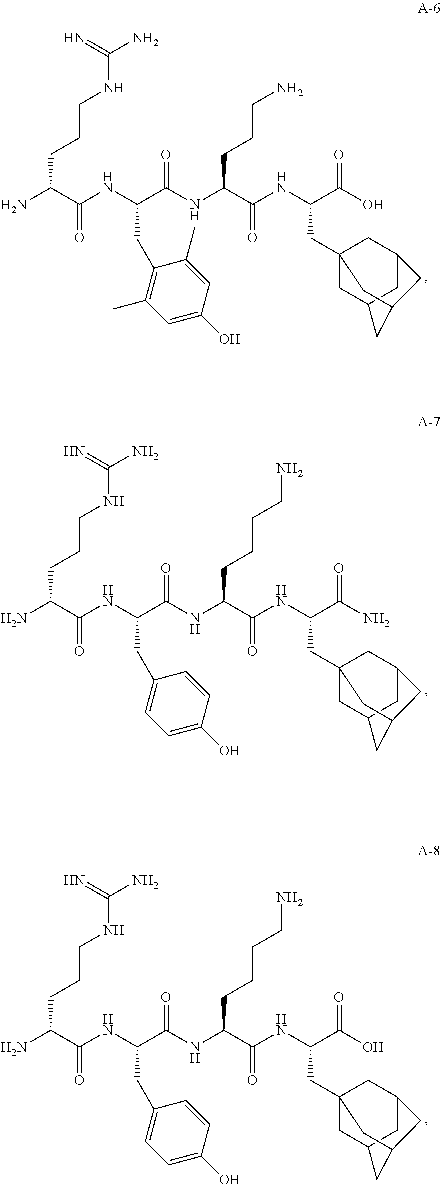 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

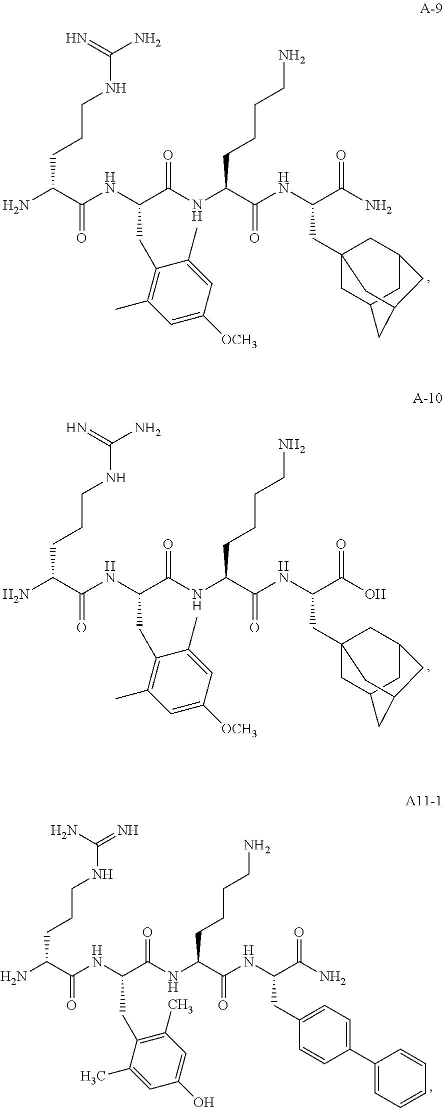 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

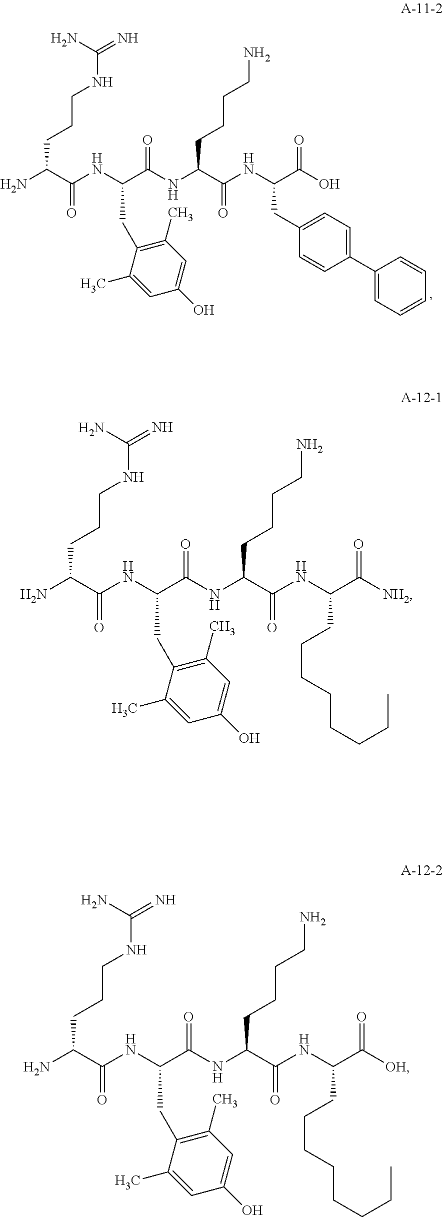 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

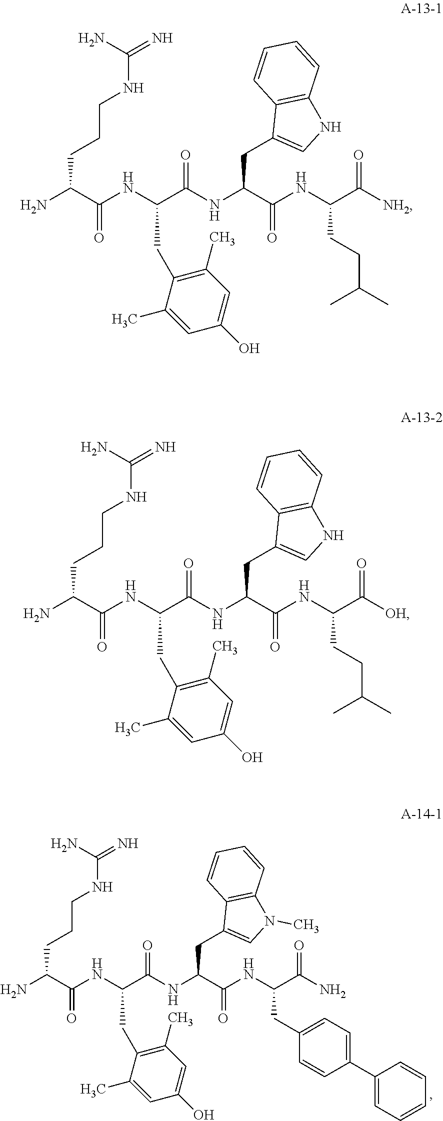 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

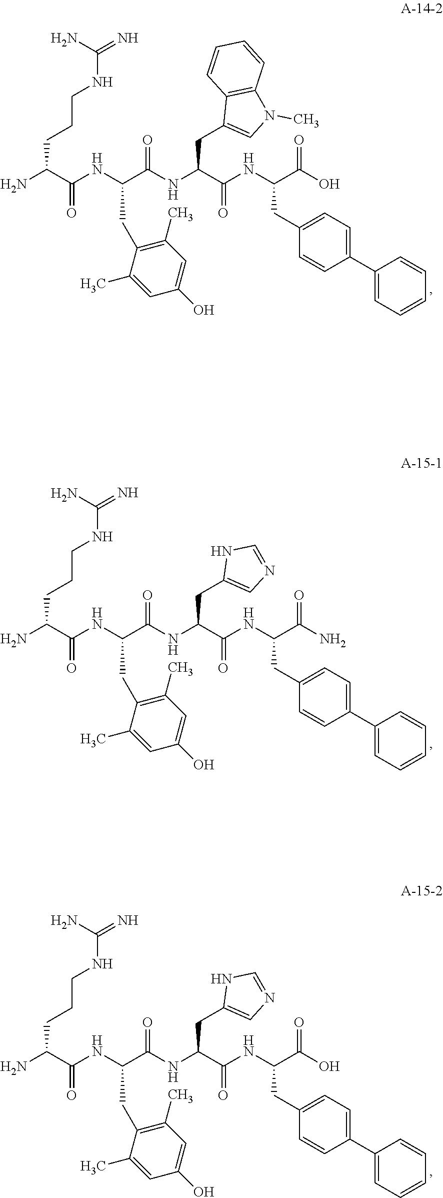 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the use, the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the use, the heart failure is congestive heart failure (CHF).

In some embodiments of the use, administration of the peptide, mixture of peptides, or the composition comprising the peptide or mixture of peptides to the subject; (i) ameliorates, (ii) delays the onset of, and/or (iii) delays the progression of one or more signs or symptoms of heart failure in the subject as compared with a control subject or group of control subjects to which the peptide or peptides was/were not administered. In some embodiments of the use, the signs or symptoms of heart failure are: (i) shortness of breath, (ii) ankle and/or foot swelling; (iii) leg swelling; (iv) fatigue, or (v) labored breathing when lying flat.

In some embodiments of the use, the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides is administered subcutaneously or intravenously. In some embodiments of the use, the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its/their tris-HCl salt.

In still another aspect, this disclosure pertains to a method for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, comprising administering to the subject a therapeutically effective amount of a peptide of Formula A, or a mixture of peptides of Formula A:

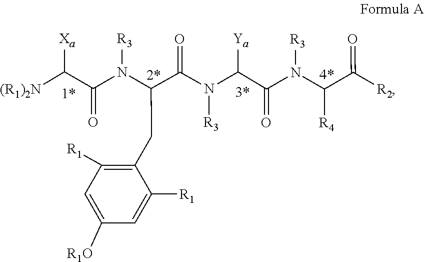

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

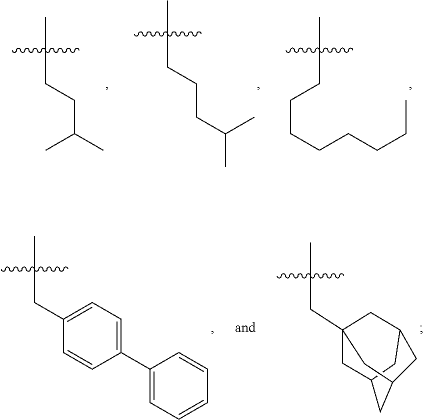

Xₐ is selected from

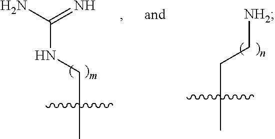

Yₐ is selected from

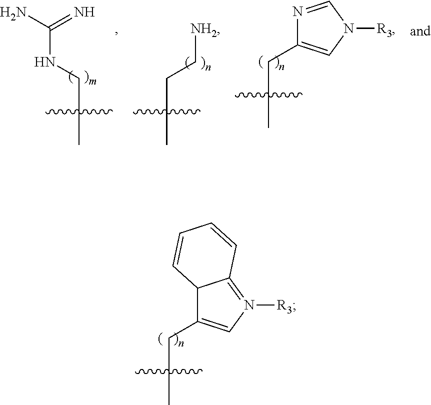

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments of the method, the peptide or peptides of Formula A is/are a peptide/peptides of Formula A-1 or Formula A-2:

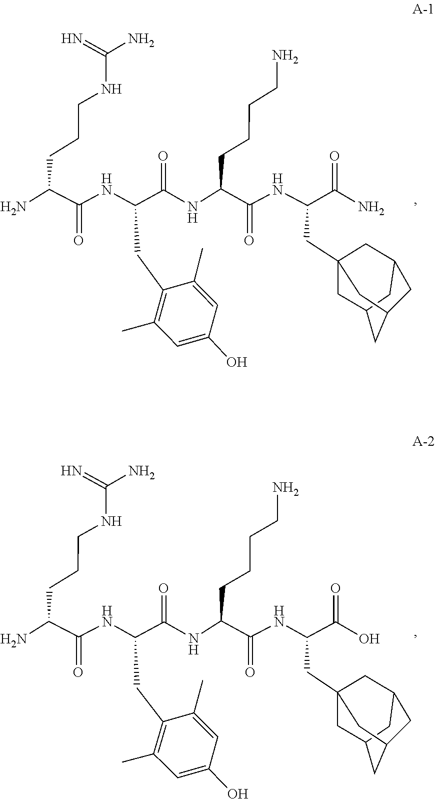 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

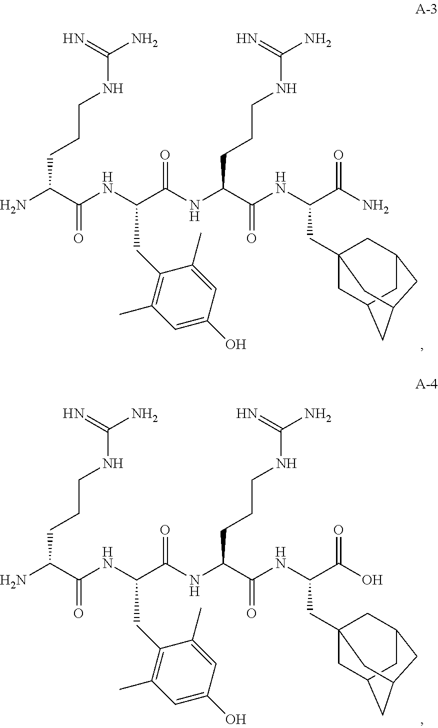 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

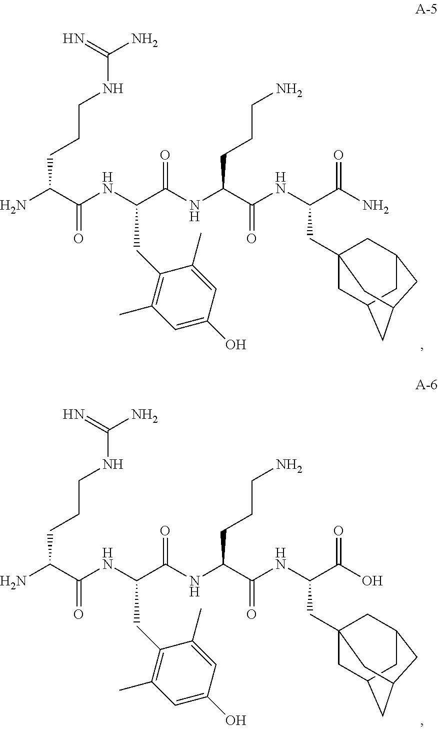 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

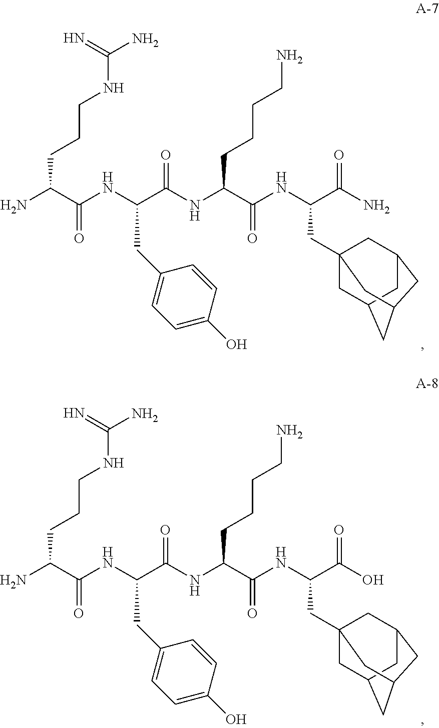 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

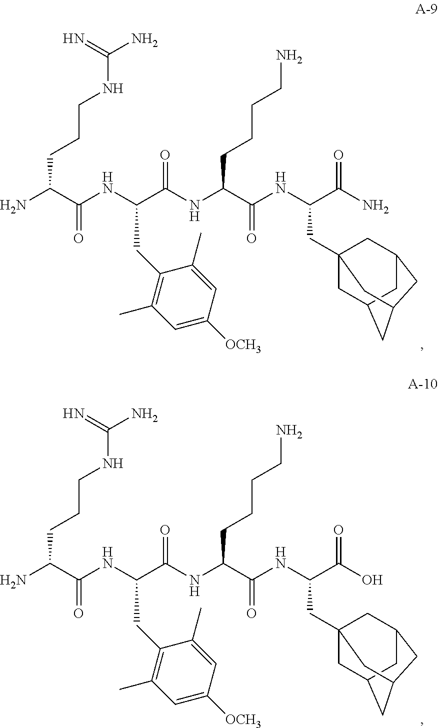 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

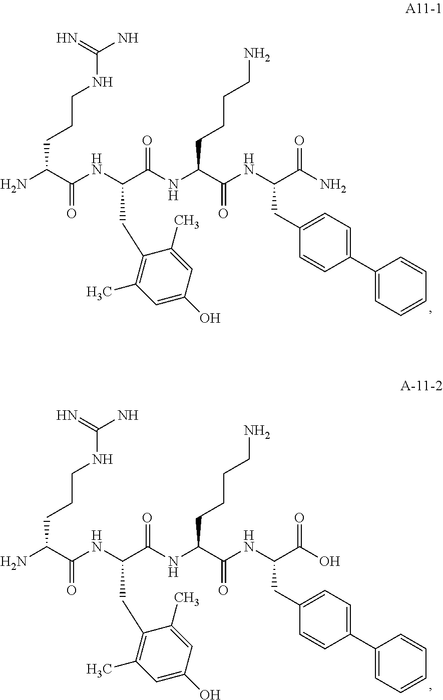 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

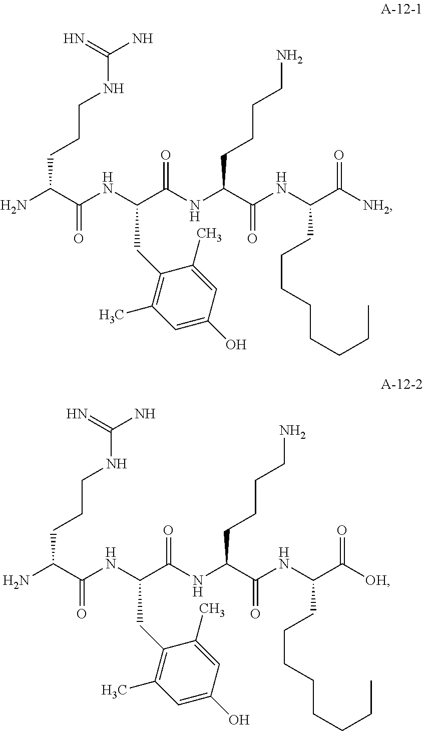 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

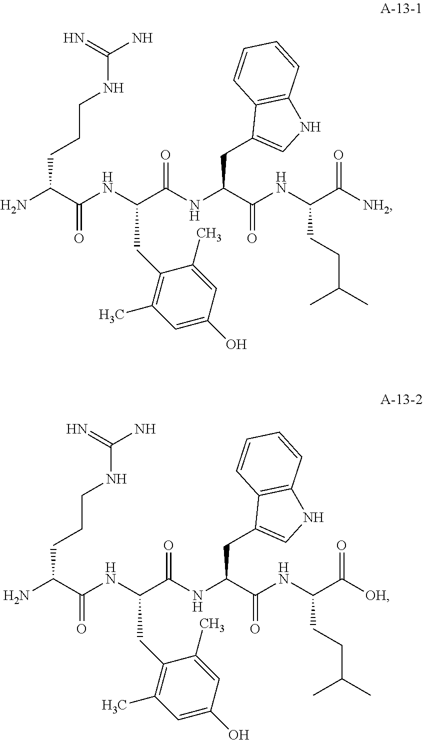 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

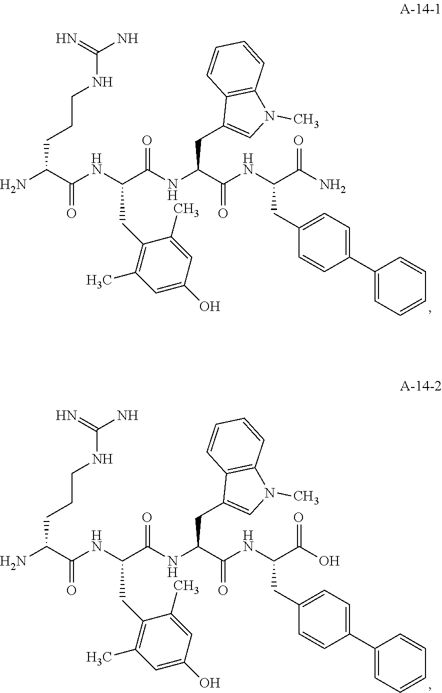 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

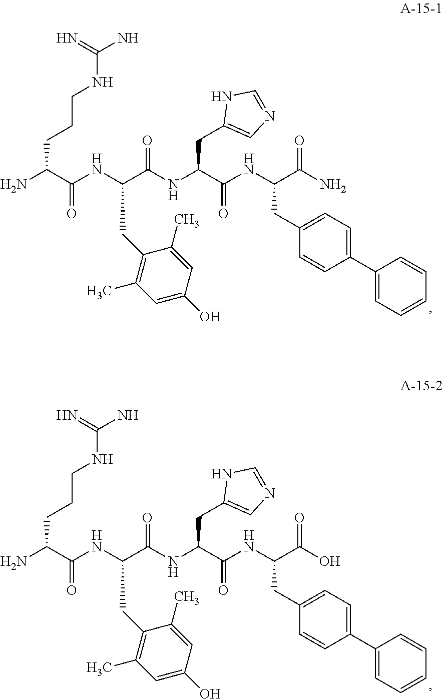 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments, the method further comprises separately, sequentially, or simultaneously administering an additional therapeutic agent to the subject. In some embodiments of the method, the additional therapeutic agent is a beta blocker, a diuretic, a mineralocorticoid receptor antagonist or a calcium channel blocker (CCB). In some embodiments of the method, the beta blocker is carvedilol, bisoprolol, metoprolol succinate, atenolol, esmolol, nebivolol and propranolol. In some embodiments, the diuretic is bumetanide, furosemide or torsemide. In some embodiments of the method, the mineralocorticoid receptor antagonist is spironolactone or eplerenone. In some embodiments of the method, the calcium channel blocker is amlodipine (Norvasc®), diltiazem (Cardizem), felodipine, isradipine, nicardipine, nifedipine (Procardia®), nisoldipine (Sular®) or verapamil (Verelan®). In some embodiments, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris-HCl salt.

In another aspect, this disclosure pertains to a composition or medicament for treating, preventing, inhibiting, ameliorating or delaying the onset of hypertrophic cardiomyopathy or hypertensive cardiomyopathy in a mammalian subject in need thereof, wherein the composition or medicament comprises a peptide or peptides of Formula A, or a mixture of peptides of Formula A:

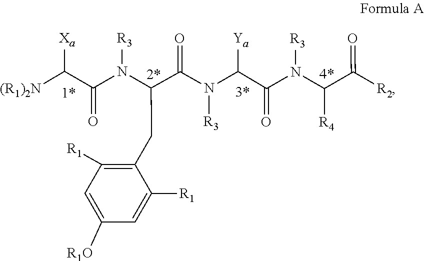

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

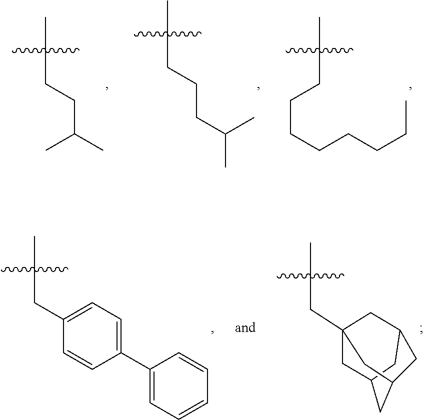

Xₐ is selected from

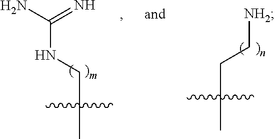

Yₐ is selected from

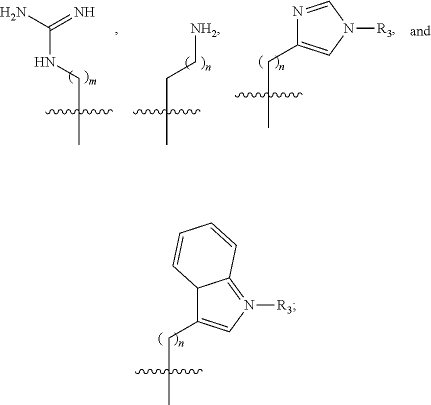

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom. In some embodiments, the peptide or peptides of Formula A is/are a peptide/peptides of Formula A-1 or Formula A-2:

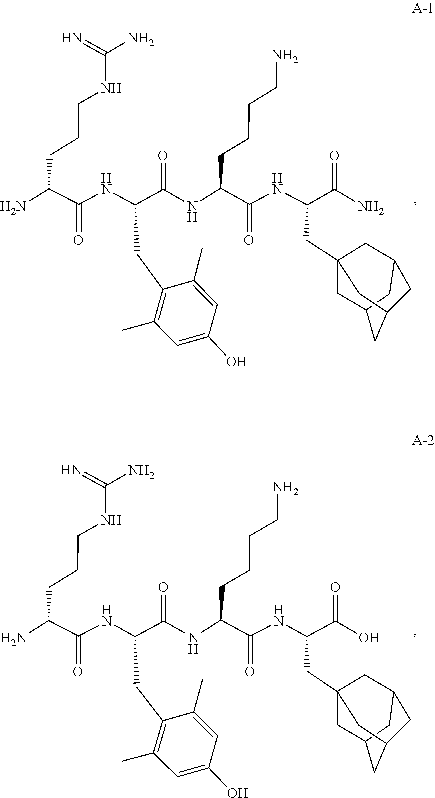 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

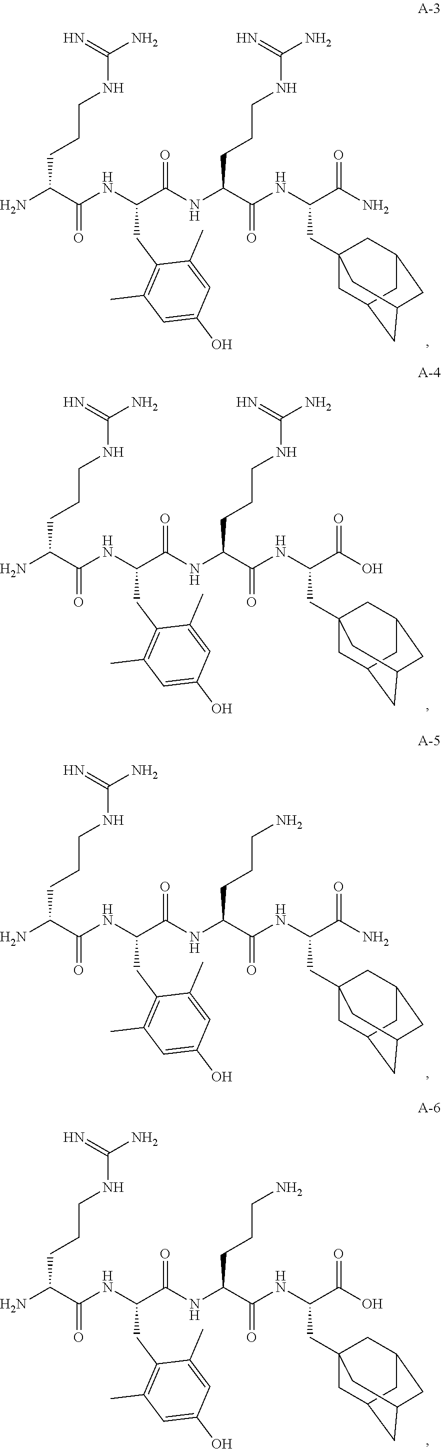 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

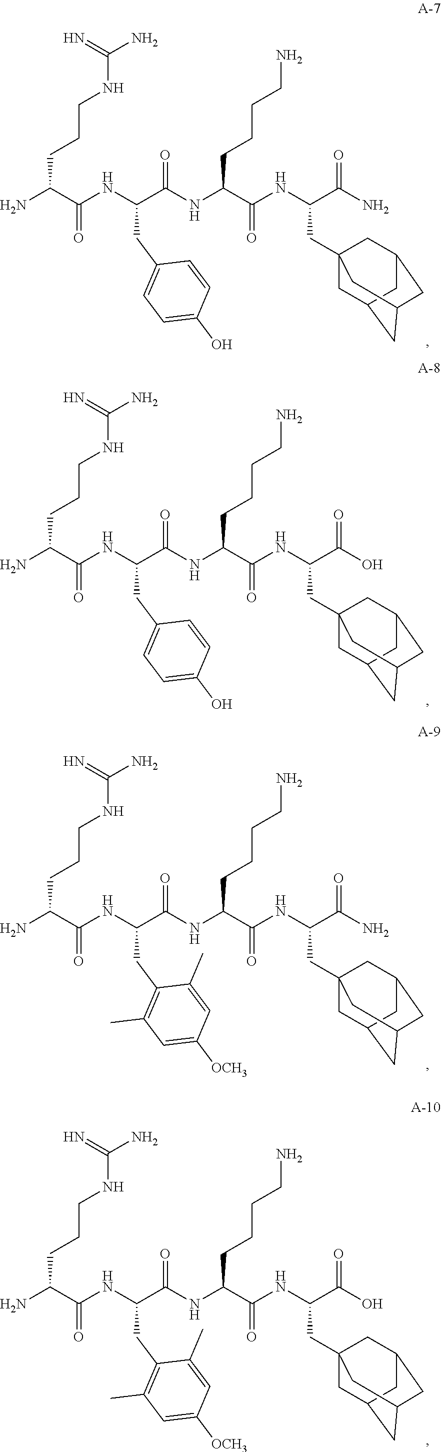 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

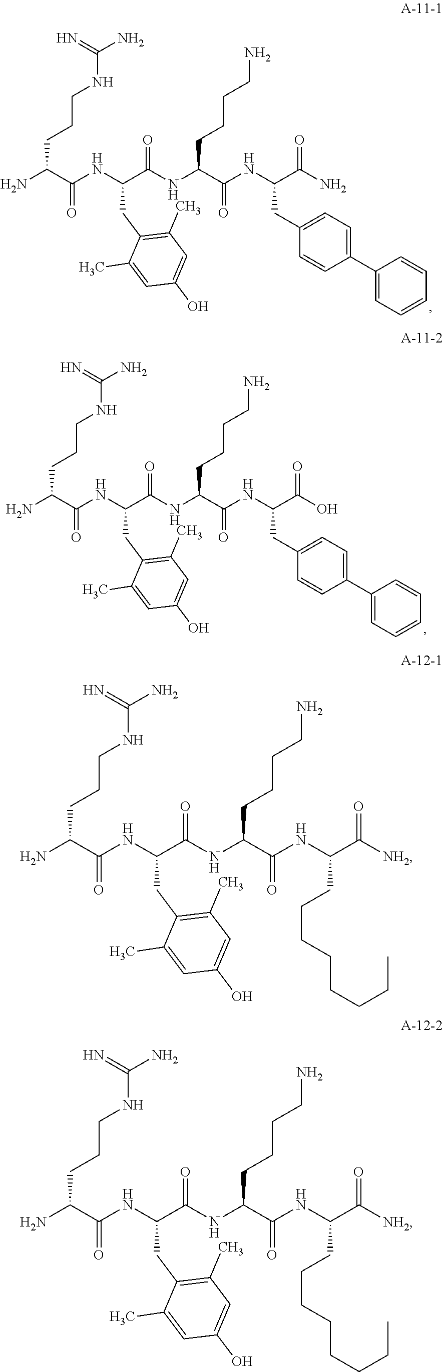 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

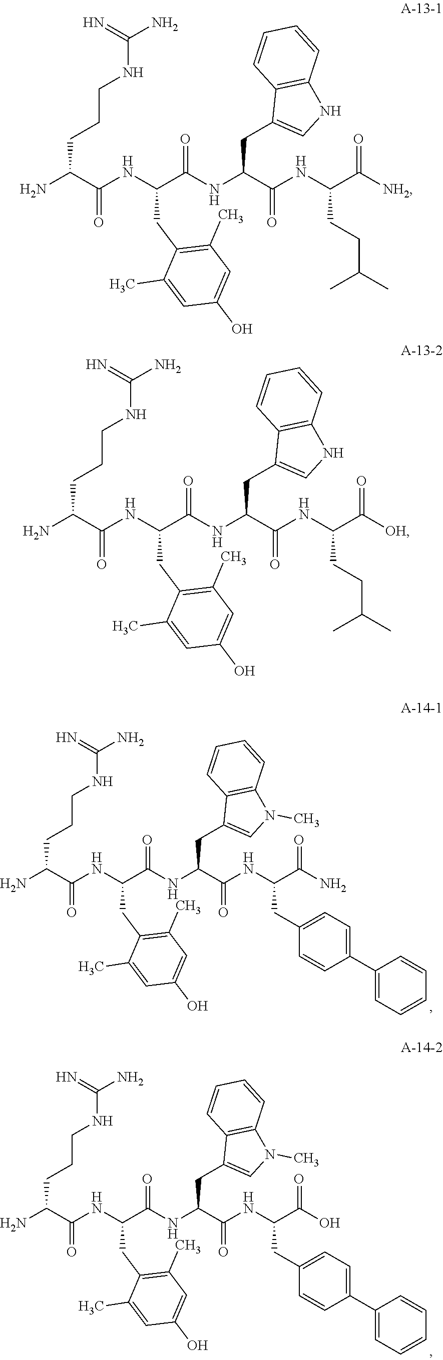 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

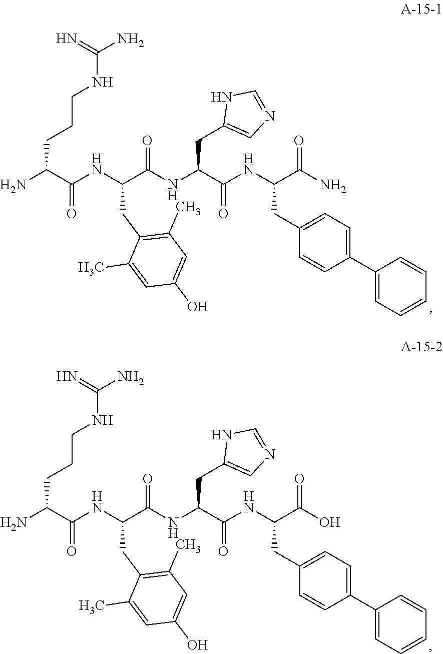 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the composition or medicament, the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament comprising the peptide or mixture of peptides is administered subcutaneously or intravenously. In some embodiments of the composition or medicament, the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris-HCl salt.

In another aspect, this disclosure relates to the use of a peptide, mixture of peptides, or a composition or medicament comprising the peptide or mixture of peptides for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, wherein the peptide or peptides of the mixture is/are of Formula A:

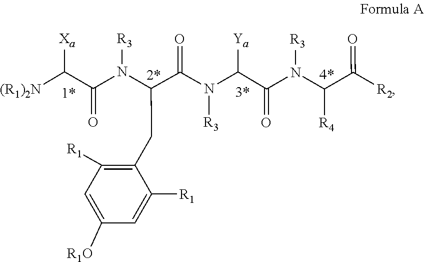

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

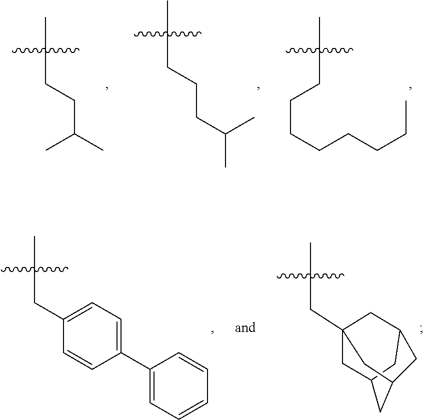

Xₐ is selected from

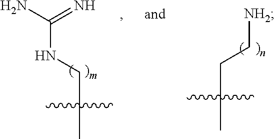

Yₐ is selected from

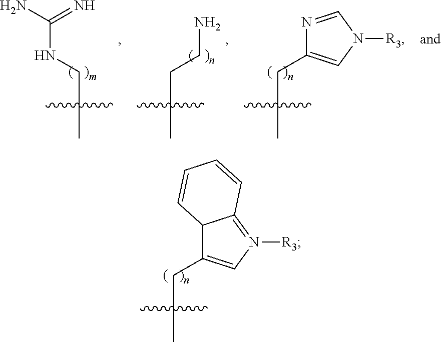

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom. In some embodiments of the use, the peptide or peptides of Formula A is/are a peptide/peptides of Formula A-1 or Formula A-2:

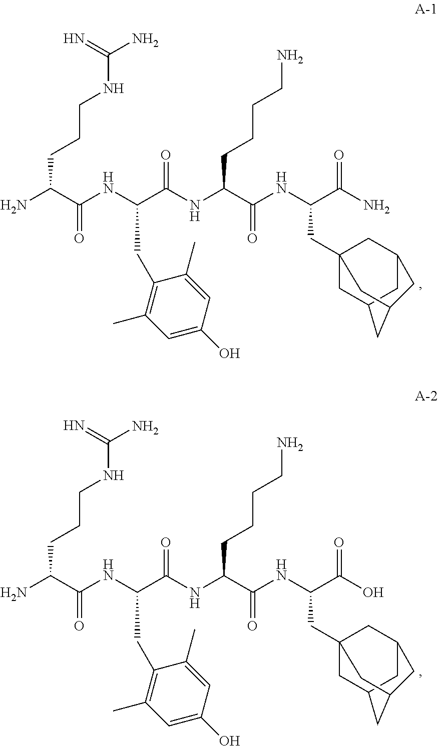 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the use, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

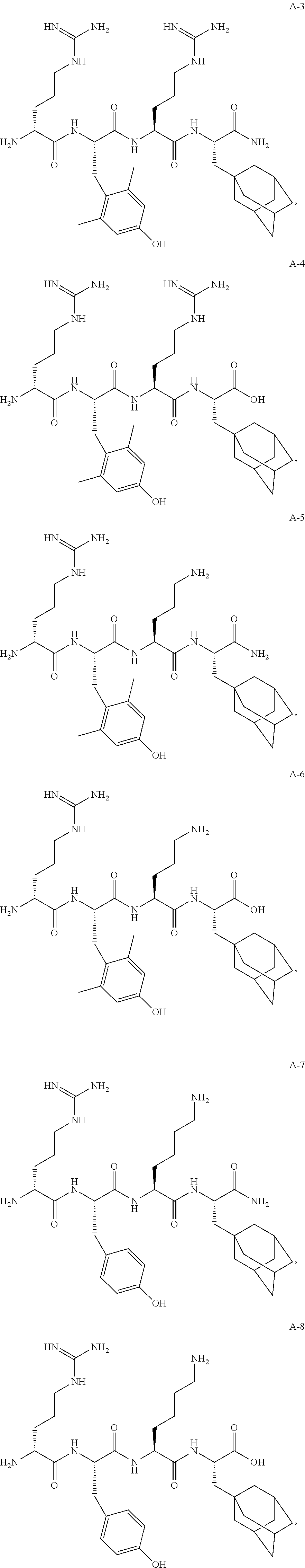

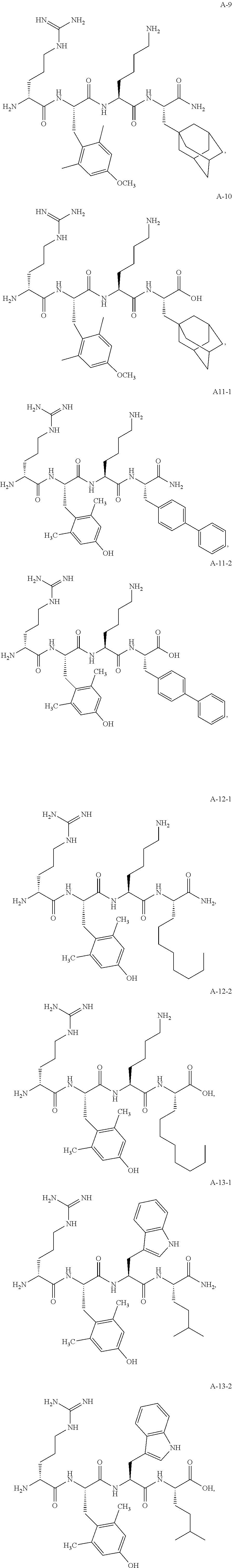

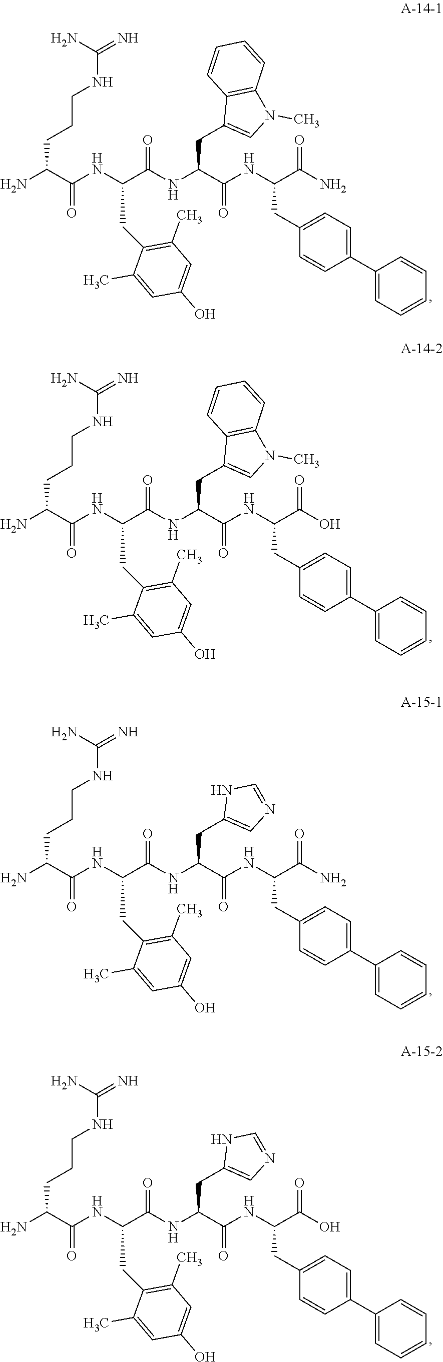 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the use, the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides is administered subcutaneously or intravenously. In some embodiments of the use, the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its/their tris-HCl salt.

In another aspect, this disclosure relates to a method for normalizing left ventricular ejection fraction, fractional shortening, and/or global longitudinal strain in a mammalian subject in need thereof, comprising administering to the subject a therapeutically effective amount of a peptide of Formula A, or a mixture of peptides of Formula A:

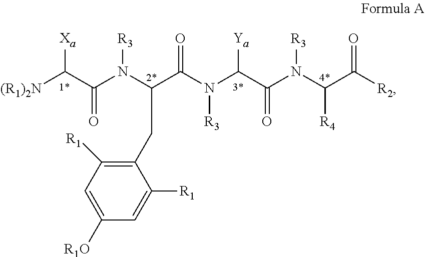

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

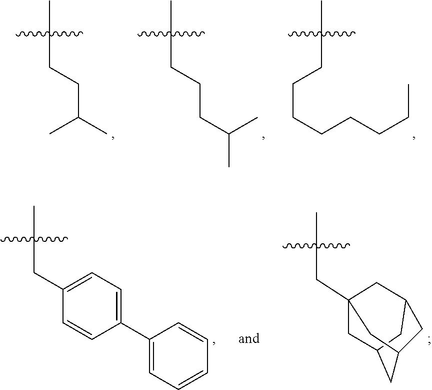

Xₐ is selected from

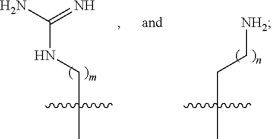

Yₐ is selected from

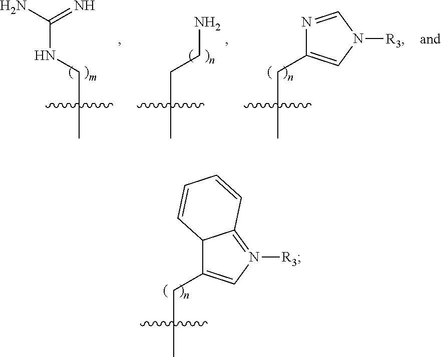

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom, wherein normalization of the left ventricular ejection fraction, fractional shortening, and/or global longitudinal strain occurs in the subject as compared with a control subject to control group of subjects not administered the peptide or peptides of Formula A. In some embodiments of the method, the peptide or peptides of Formula A is/are a peptide/peptides of Formula A-1 or Formula A-2:

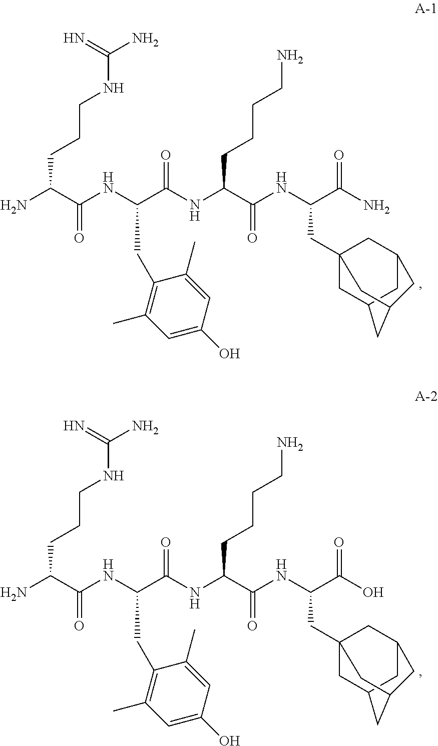 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

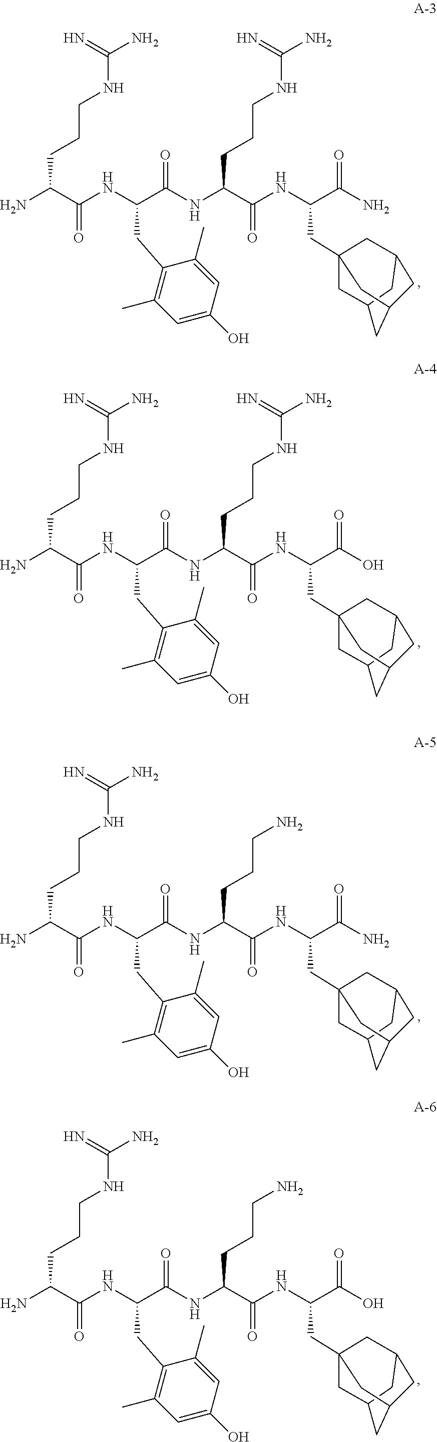 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

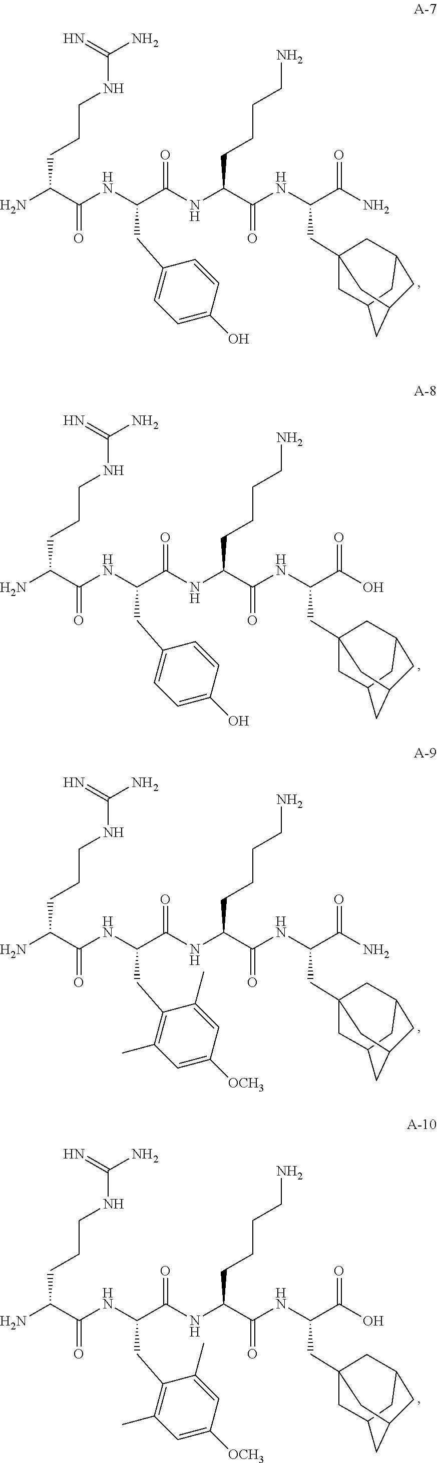 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

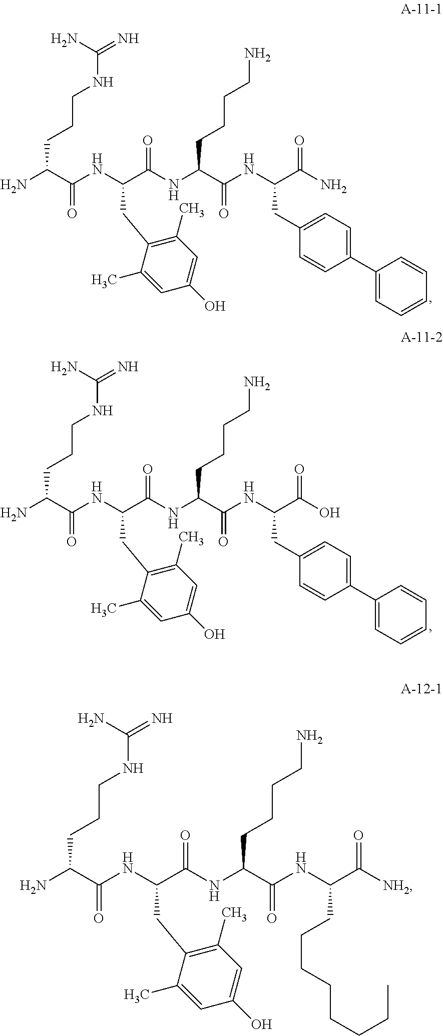 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

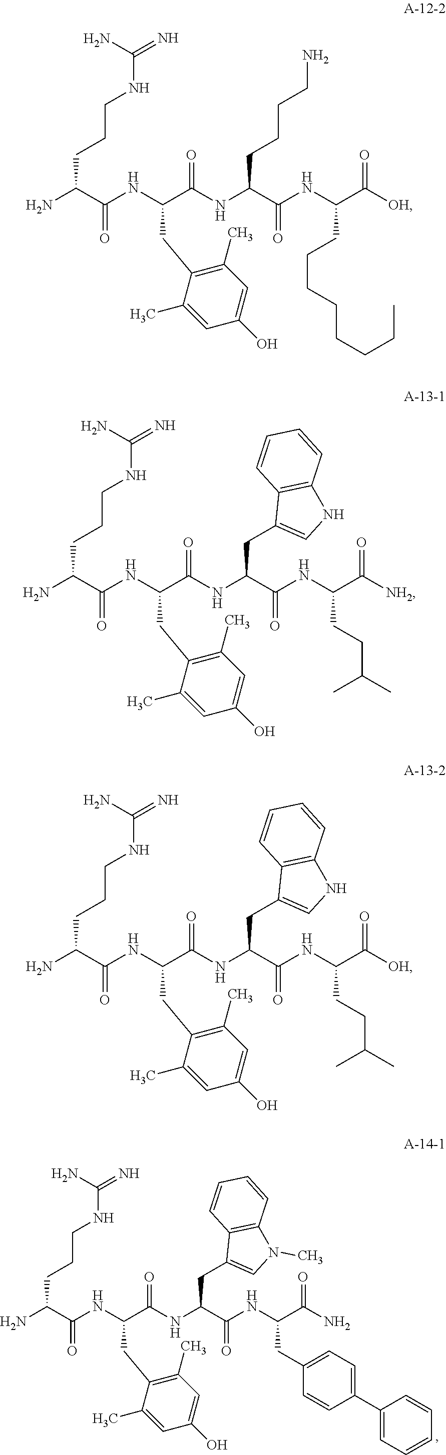 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(=O)O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

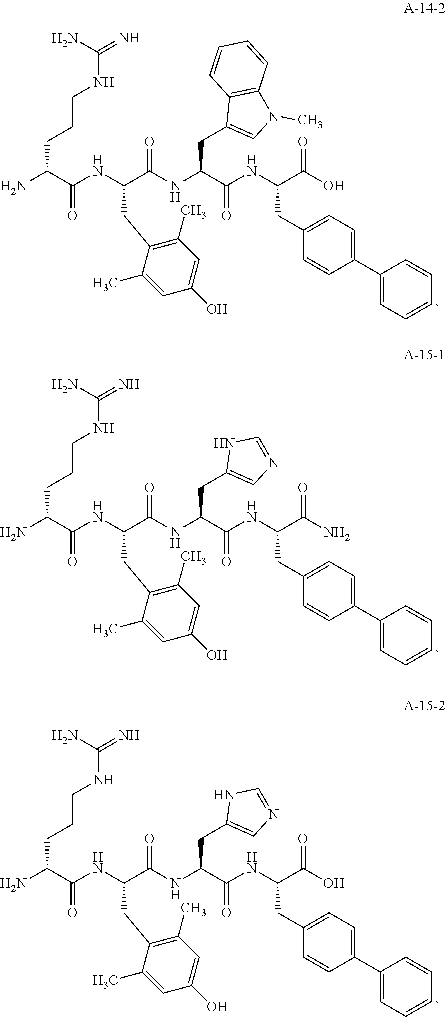 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments, the method further comprises separately, sequentially, or simultaneously administering an additional therapeutic agent to the subject. In some embodiments of the method, the additional therapeutic agent is a beta blocker, a diuretic, a mineralocorticoid receptor antagonist or a calcium channel blocker (CCB). In some embodiments of the method, the beta blocker is carvedilol, bisoprolol, metoprolol succinate, atenolol, esmolol, nebivolol and propranolol. In some embodiments, the diuretic is bumetanide, furosemide or torsemide. In some embodiments of the method, the mineralocorticoid receptor antagonist is spironolactone or eplerenone. In some embodiments of the method, the calcium channel blocker is amlodipine (Norvasc®), diltiazem (Cardizem), felodipine, isradipine, nicardipine, nifedipine (Procardia®), nisoldipine (Sular®) or verapamil (Verelan®). In some embodiments, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris-HCl salt.

In another aspect, this disclosure relates to a composition or medicament for normalizing left ventricular ejection fraction, fractional shortening, and/or global longitudinal strain in a mammalian subject in need thereof, wherein the composition or medicament comprises a peptide of Formula A, or a mixture of peptides of Formula A:

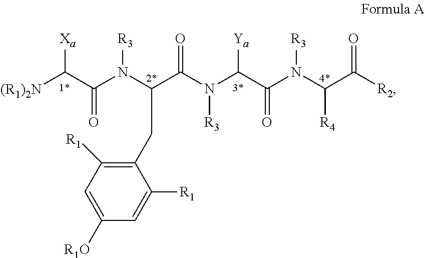

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

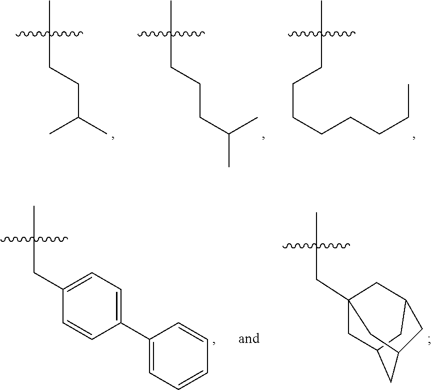

Xₐ is selected from

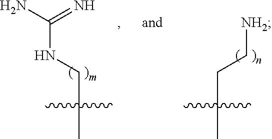

Yₐ is selected from

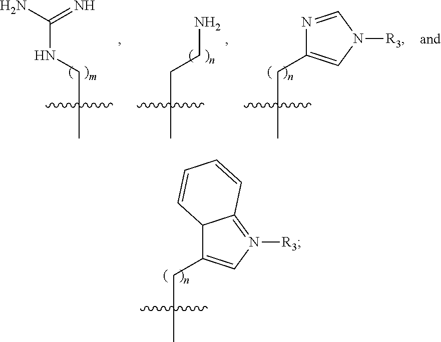

each m is 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, the peptide or peptides of Formula A is/are a peptide/peptides of Formula A-1 or Formula A-2:

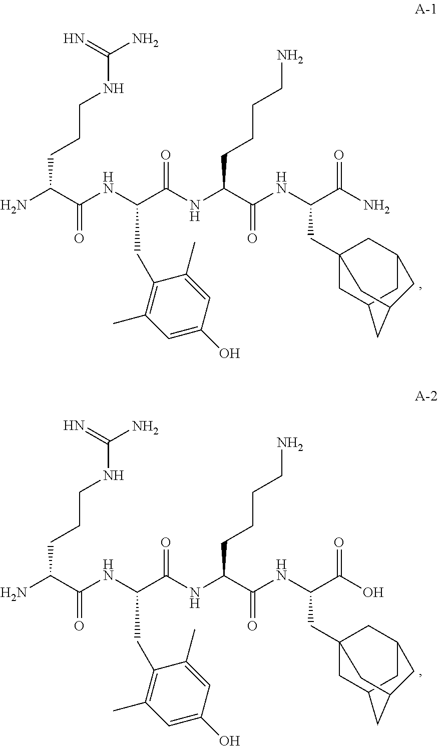 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

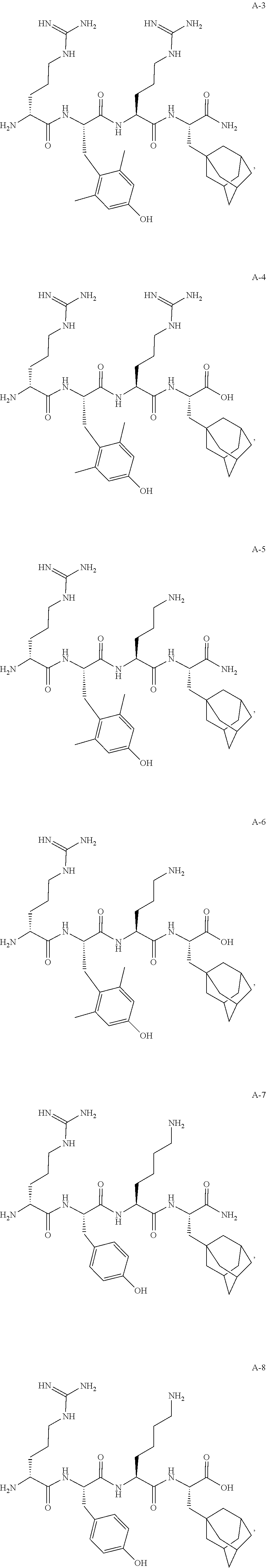

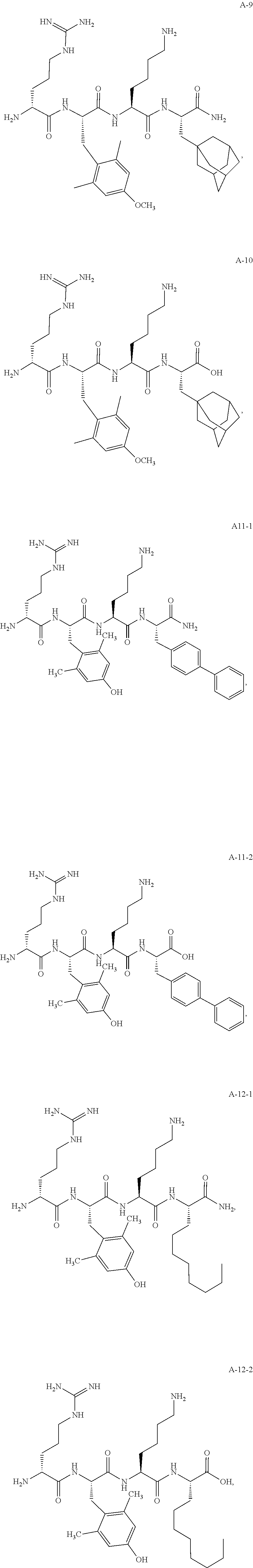

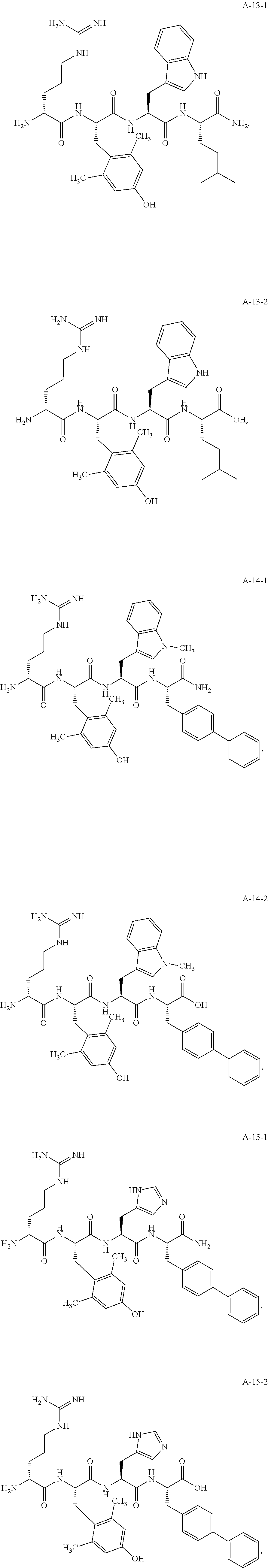

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the composition or medicament, the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament comprising the peptide or mixture of peptides is administered subcutaneously or intravenously. In some embodiments of the composition or medicament, the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris-HCl salt.

In another aspect, this disclosure relates to the use of a peptide, mixture of peptides or a composition or medicament comprising the peptide or the mixture of peptides for normalizing left ventricular ejection fraction, fractional shortening, and/or global longitudinal strain in a mammalian subject in need thereof, wherein the peptide or peptides of the mixture is/are of Formula A:

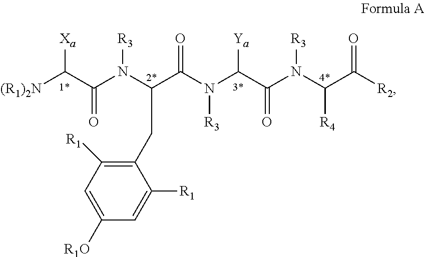

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

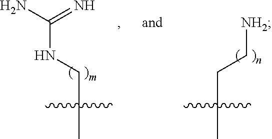

Xₐ is selected from

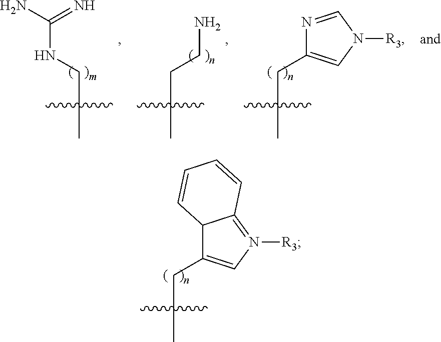

Yₐ is selected from

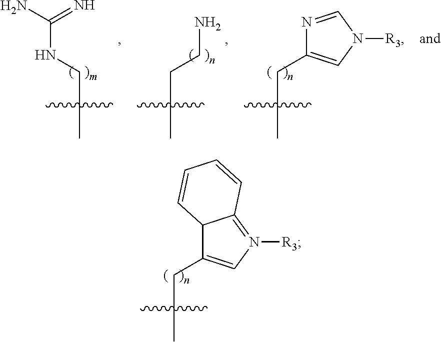

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide of Formula A is substituted with a deuterium or fluorine atom, wherein the normalization of the left ventricular ejection fraction, fractional shortening, and/or global longitudinal strain occurs in the subject as compared with a control subject to control group of subjects not administered the peptide or peptides of Formula A. In some embodiments of the use, the peptide or peptides of Formula A is/are a peptide/peptides of Formula A-1 or Formula A-2:

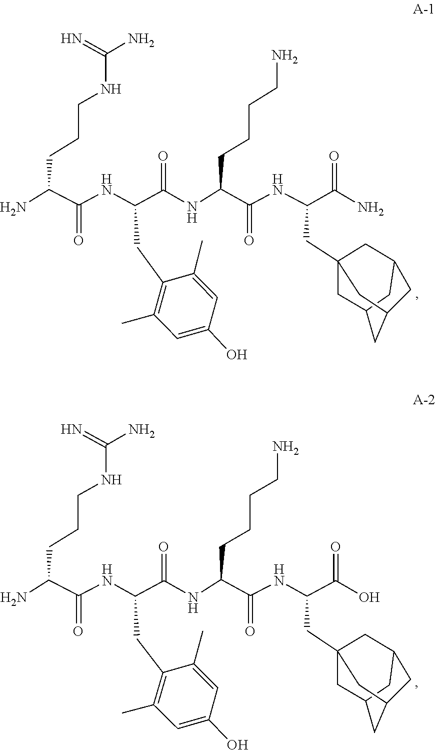 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the use, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

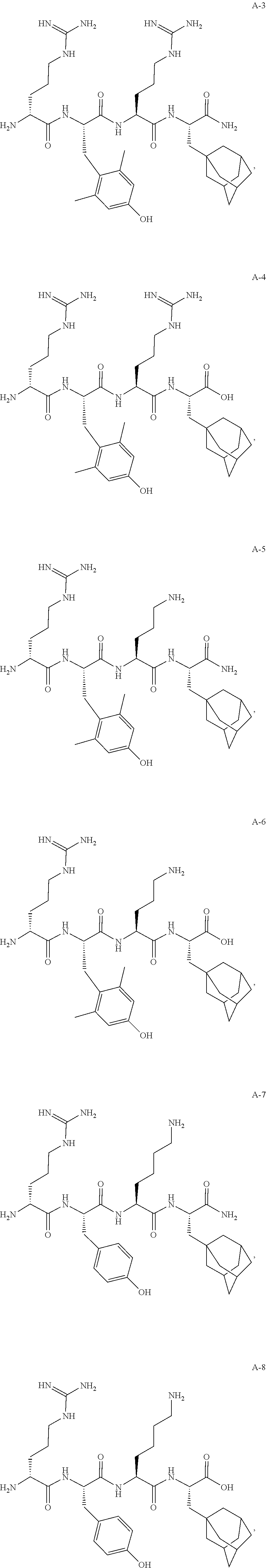

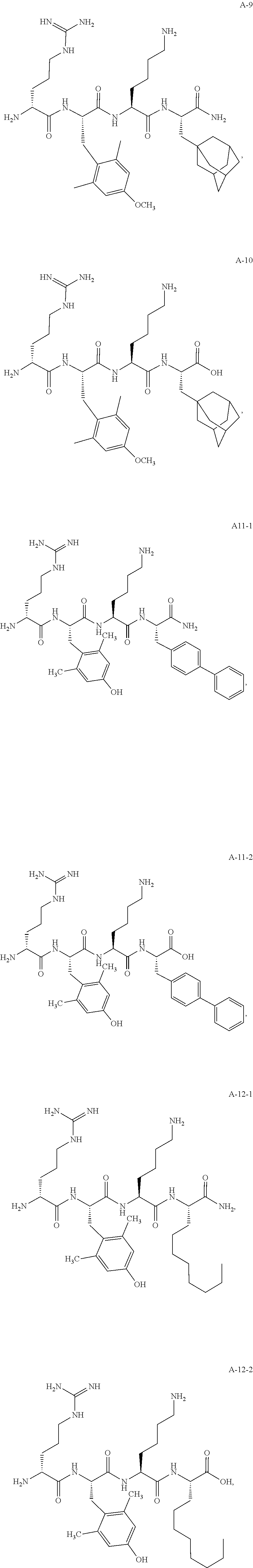

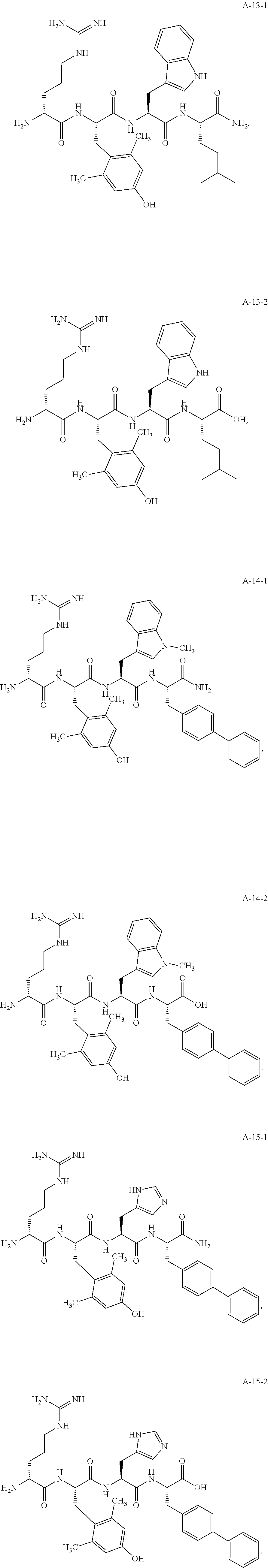

or a pharmaceutically acceptable salt, hydrate, solvate, stereoisomer and/or tautomer thereof. In some embodiments, one or more hydrogen atoms of the peptide or peptides of Formulas A are optionally substituted with a deuterium or fluorine atom.

In some embodiments of the use, the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides is administered subcutaneously or intravenously. In some embodiments of the use, the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its/their tris-HCl salt.

### BRIEF DESCRIPTION OF THE DRAWINGS

FIG. 1 is an illustration of a generic structure for peptides of Formula A and the structure for two specific examples of specific peptides (peptides of Formula A-1 and Formula A-2) within the scope of Formula A as defined herein.

FIG. 2 is an illustration of various salt forms of the peptide of Formula A-2.

FIG. 3 is an illustration of various salt forms of the peptide of Formula A-1.

FIG. 4 is a scheme for the synthesis of the peptide of Formula A-1.

FIG. 5A is a graphic presentation of the data for a time course examination of blood plasma concentrations of elamipretide (a.k.a., SS-31) and the peptide of Formula A-1 in C57/BL6 mice treated interperitoneally with a single 5 mg/kg dose of each compound as described in Example 1.

FIG. 5B is a graphic presentation of the data for a time course examination of the concentration of the peptide of Formula A-1 in: (i) blood plasma (same data as presented in FIG. 5A), (ii) heart tissue, and (iii) muscle tissue of C57/BL6 mice treated interperitoneally with a single 5 mg/kg dose as described in Example 1.

FIG. 6 is a bar graph presentation of data for the geometric mean tissue concentrations (ng/g) of elamipretide (also known as SBT-031) and the peptide of Formula A-1 in muscle tissue, heart tissue and liver tissue as described in Example 2.

FIG. 7A is a graphic presentation of data for O₂ flux of mitochondria collected from failing human hearts and treated with: (i) vehicle, (ii) 0.1 μM elamipretide (i.e., SS-31) or (iii) 0.1 μM peptide of Formula A-1 as described in Example 3.

FIG. 7B is a bar graphic presentation of data for mitochondrial superoxide production in tissue isolated from failing (explanted) human hearts treated with: (i) vehicle (water) control or (ii) elamipretide at 100 μM as described in Example 3.

FIG. 7C is a bar graphic presentation of data for mitochondrial superoxide production in tissue isolated from failing (explanted) human hearts treated with: (i) vehicle (water) control or (ii) the peptide of Formula A-1 at 100 nM as described in Example 3.

FIG. 7D is a graphic illustration of data for complex I+II respiration of failing human heart mitochondria treated with: (i) vehicle or (ii) the peptide of Formula A-1 at 10 nM as described in Example 3.

FIG. 7E is a graphic illustration of data for maximal respiration of failing human heart mitochondria treated with: (i) vehicle or (ii) the peptide of Formula A-1 at 10 nM as described in Example 3.

FIG. 7F is a graphic presentation of data for oxygen consumption rate of mitochondria collected from mouse heart put through a freeze/thaw cycles and then treated with either: (i) vehicle, (ii) elamipretide at 10 μM, (iii) the peptide of Formula A-1 at 100 nM, or (iv) the peptide of Formula A-1 at 1 μM as described in Example 4.

FIGS. 8A-8C are graphs showing cardiac function data of aged mice treated with the peptide of Formula A-1. Data is presented in bar graph form for the change in cardiac function, as measured by percentage of fractional shortening (FS) (FIG. 8A), ejection fraction (EF) (FIG. 8B), and global longitudinal strain (GS) (FIG. 8C), in aged mice after daily treatment with the peptide of Formula A-1 (3 mg/kg) for 8 weeks as described in Example 5.

FIG. 9A presents the structure of a peptide of Formula A-11-1 and accompanying graphic data presented for: (i) the white necrotic tissue/risk, and (ii) total risk assessed for mouse hearts treated with: (a) vehicle, (b) elamipretide, and (c) the peptide of Formula A-11-1; all subjected to the Langendorff procedure as described in Example 6.

FIG. 9B presents the structure of a peptide of Formula A-1 and accompanying graphic data presented for: (i) the white necrotic tissue/risk, and (ii) total risk assessed for mouse hearts treated with: (a) vehicle, (b) elamipretide, and (c) the peptide of Formula A-1; all subjected to the Langendorff procedure as described in Example 6.

FIG. 9C presents the structure of a peptide of Formula A-12-1 and accompanying graphic data presented for: (i) the white necrotic tissue/risk, and (ii) total risk assessed for mouse hearts treated with: (a) vehicle, (b) elamipretide, and (c) the peptide of Formula A-12-1; all subjected to the Langendorff procedure as described in Example 6.

FIG. 9D presents the structure of a peptide of Formula A-13-1 and accompanying graphic data presented for: (i) the white necrotic tissue/risk, and (ii) total risk assessed for mouse hearts treated with: (a) vehicle, (b) elamipretide, and (c) the peptide of Formula A-13-1; all subjected to the Langendorff procedure as described in Example 6.

FIG. 9E presents the structure of a peptide of Formula A-14-1 and accompanying graphic data presented for: (i) the white necrotic tissue/risk, and (ii) total risk assessed for mouse hearts treated with: (a) vehicle, (b) elamipretide, and (c) the peptide of Formula A-14-1; all subjected to the Langendorff procedure as described in Example 6.

FIG. 9F presents the structure of a peptide of Formula A-15-1 and accompanying graphic data presented for: (i) the white necrotic tissue/risk, and (ii) total risk assessed for mouse hearts treated with: (a) vehicle, (b) elamipretide, and (c) the peptide of Formula A-15-1; all subjected to the Langendorff procedure as described in Example 6.

FIG. 9G presents the structure of a peptide of Formula A-16 and accompanying graphic data presented for: (i) the white necrotic tissue/risk, and (ii) total risk assessed for mouse hearts treated with: (a) vehicle, (b) elamipretide, and (c) the peptide of Formula A-16; all subjected to the Langendorff procedure as described in Example 6.

### DEFINITIONS

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended claims, the singular forms “a,” “an,” and “the” include plural references unless the content clearly dictates otherwise. For example, reference to “a cell” includes a combination of two or more cells, and the like.

As used herein, the “administering” or the “administration” of an agent (e.g., a therapeutic agent, for example, a peptide or mixture of peptides, individually or as formulated) or drug to a subject refers to any route of introducing or delivering to a subject the peptide, mixture of peptides, formulation, medicament or other therapeutic agent(s) to perform its/their intended function. Administration can be carried out by any suitable route, such as oral administration. Administration can be carried out subcutaneously. Administration can be carried out intravenously. Administration can be carried out intraocularly. Administration can be carried out retro-orbitally. Administration can be carried out systemically. Administration may be carried out topically. Administration can be carried out intranasally. Administration can be carried out intraperitoneally. Administration can be carried out intradermally. Administration can be carried out ophthalmically. Administration can be carried out intrathecally. Administration can be carried out intracerebroventricularly. Administration can be carried out iontophoretically. Administration can be carried out transmucosally. Administration can be carried out intravitreally. Administration can be carried out intramuscularly. Administration includes self-administration, the administration by another or the administration by a device (e.g., a pump).

As used herein, to “ameliorate” or “ameliorating” a disease, disorder or condition refers to results that, in a statistical sample or specific subject, make the occurrence of the disease, disorder or condition (or a sign or symptom thereof) better or more tolerable in a subject administered a therapeutic agent(s) relative to a control sample, control subject or control group of subjects not administered the therapeutic agent(s).

As used herein, the term “amino acid” refers to naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein the term or phrase “carrier” and “pharmaceutically acceptable carrier” refers to a diluent, adjuvant, excipient, or vehicle with which a therapeutic agent (e.g., a peptide/mixture of peptides/compound/composition) is administered or formulated for administration. Non-limiting examples of such pharmaceutically acceptable carriers include liquids, such as water, saline, and oils; and solids, such as gum acacia, gelatin, starch paste, talc, keratin, colloidal silica, silica particles (nanoparticles or microparticles), urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “delaying the onset of” refers to, in a statistical sample, postponing, hindering, or causing a disease, disorder or condition (or a sign, symptom or condition thereof) to occur more slowly than normal in a sample or subject administered a therapeutic agent(s) relative to a control sample, control subject or control group of subjects not administered the therapeutic agent(s).

As used herein, the term “effective amount” refers to a quantity of therapeutic agent(s) (e.g., a peptide, mixture of peptides/compound/composition) sufficient to achieve a desired therapeutic and/or prophylactic effect, e.g., an amount that treats, inhibits, reduces, ameliorates, or delays the onset of heart failure, idiopathic cardiomyopathy, dilated cardiomyopathy, restrictive cardiomyopathy, hypertrophic cardiomyopathy, or hypertensive cardiomyopathy when “administered or “co-administered. In the context of therapeutic or prophylactic applications, in some embodiments, the amount of a composition administered to the subject will depend on the type and severity of the disease and on the characteristics of the individual, such as general health, age, sex, body weight, and tolerance to drugs. The skilled artisan will be able to determine appropriate dosages depending on these and other factors. The therapeutic agent(s) (e.g., a peptide/compound/composition) disclosed herein can be administered in an effective amount prior to the onset of one or more symptoms associated with heart failure, idiopathic cardiomyopathy, dilated cardiomyopathy, restrictive cardiomyopathy, hypertrophic cardiomyopathy, or hypertensive cardiomyopathy, or in response to a symptom that occurs in a subject suffering from heart failure, idiopathic cardiomyopathy, dilated cardiomyopathy, restrictive cardiomyopathy, hypertrophic cardiomyopathy, or hypertensive cardiomyopathy. The combination of peptide(s) and other therapeutic agent(s) disclosed herein can be administered simultaneously, sequentially, or by separate administration as would be appropriate, for example, based on accepted practice and their label as approved by a regulatory authority (e.g., the FDA).

In the methods described herein, therapeutic compounds (e.g., a peptide or mixture of peptides), or pharmaceutically acceptable salts, stereoisomers, mixtures of stereoisomers, tautomers, hydrates, and/or solvates thereof, may be administered to a subject having one or more signs, symptoms, or risk factors of heart failure, idiopathic cardiomyopathy, dilated cardiomyopathy, restrictive cardiomyopathy, hypertrophic cardiomyopathy, or hypertensive cardiomyopathy or in a patient diagnosed with or suspected of having any one or more of the previously described heart related conditions. For example, a “therapeutically effective amount” of a therapeutic compound(s) (e.g., a peptide or mixture of peptides) may include levels at which the presence, frequency, or severity of one or more signs, symptoms, or risk factors of heart failure, idiopathic cardiomyopathy, dilated cardiomyopathy, restrictive cardiomyopathy, hypertrophic cardiomyopathy, or hypertensive cardiomyopathy are inhibited, reduced or eliminated.

As used herein, the term “hydrate” refers to a therapeutic compound(s) (e.g., a peptide or mixture of peptides) which is associated with water. The number of the water molecules contained in a hydrate of a compound may be (or may not be) in a definite ratio to the number of the therapeutic compound(s) molecule(s) in the hydrate.

As used herein, the term “inhibit,” “inhibits,” or “inhibiting” refers to reduction in by an objectively measurable amount or degree of a disease, disorder or condition (or a sign, symptom or condition thereof) to occur more slowly than normal in a sample or subject administered a therapeutic agent(s) relative to a control sample, control subject or control group of subjects not administered the therapeutic agent(s). In some embodiments, to inhibit or inhibiting means reducing by at least 1-5 percent compared to a sample or subject administered a therapeutic agent(s) relative to a control sample, control subject or control group of subjects not administered the therapeutic agent(s). In various individual embodiments, inhibit or inhibiting means reducing by at least 2, 3, 4, 5, 6, 7, 8, 9, 10, 15, 20, 25, 30, 33, 40, 50, 60, 67, 70, 75, 80, 90, 95, or 99 percent compared to a sample or subject administered a therapeutic agent(s) relative to a control sample, control subject or control group of subjects not administered the therapeutic agent(s).

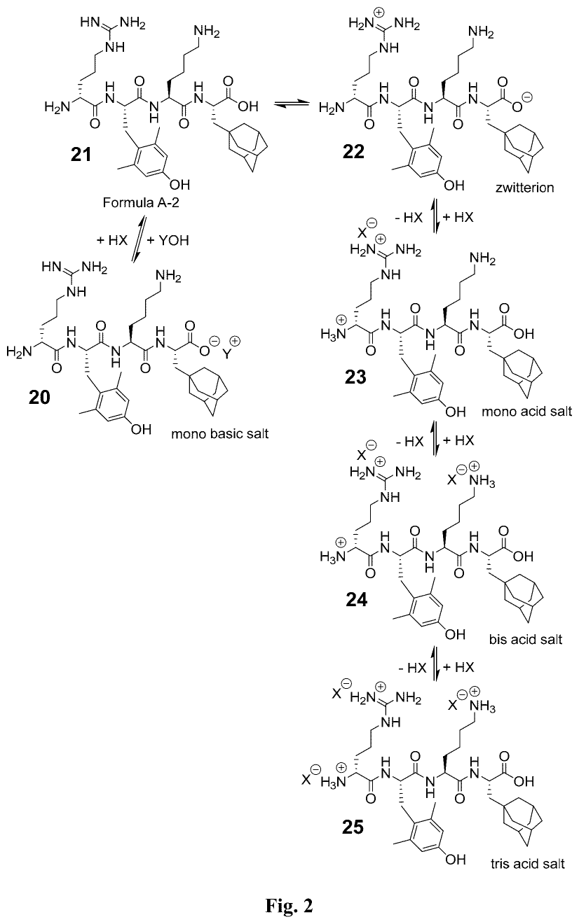

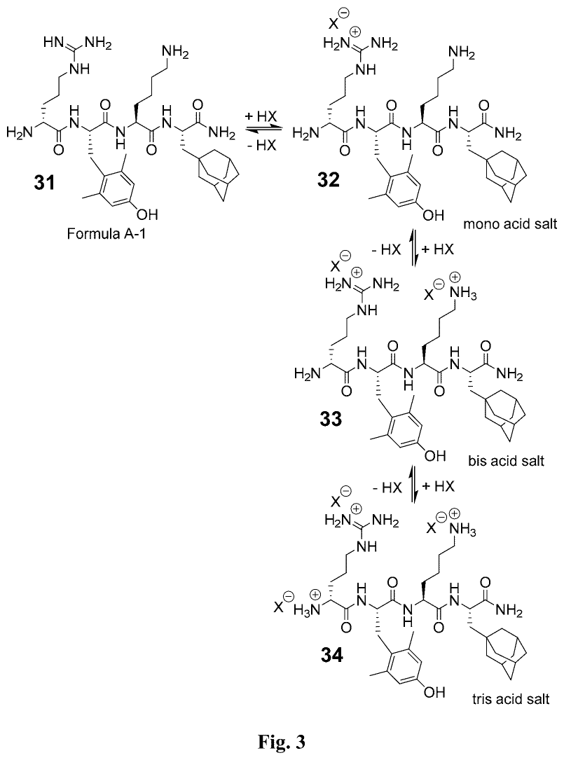

As used herein, the term “pharmaceutically acceptable salt” refers to a salt of a therapeutic agent (e.g., a peptide or mixture of peptides) that can be prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic and p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds of the present application may contain both basic and acidic functionalities that allow the compounds to be converted into either base or acid addition salts. These salts may be prepared by methods known to those skilled in the art. Other pharmaceutically acceptable carriers known to those of skill in the art are suitable for the present technology. In some embodiments, the compound is a zwitterion (an intramolecular salt). Exemplary salt forms of the peptide H-D-Arg-2′6′-Dmt-Lys-Phe-OH (A-2) are illustrated in FIG. 2. Exemplary salt forms of the peptide H-D-Arg-2′6′-Dmt-Lys-Phe-NH2 (A-1) are illustrated in FIG. 3.

As used herein the term “prevent,” “prevents,” “preventing,” or “prevention” refers to, in a statistical sample, reducing the occurrence of a disease state, disorder, symptom, or condition in a sample or subject administered a therapeutic agent(s) relative to a control sample, control subject or control group of subjects not administered the therapeutic agent(s).

As used herein the term “progression” refers to the process of moving gradually towards a more advanced state of a disease, disorder, or condition (or a sign or symptom thereof) in a subject.

As used herein the term “prophylactic” refers to an action intended to prevent a disease state, disorder, symptom, or condition from occurring.

As used herein, the term “separate” with respect to a therapeutic use refers to an administration of at least two active ingredients (i.e., at least two difference therapeutic agents) at the same time or at substantially the same time by different routes. The “active ingredients” can, for example, be a peptide or mixture of peptides as disclosed herein. In some embodiments, the active ingredients may be an additional therapeutic agent, for example, an angiotensin-converting enzyme (ACE) inhibitor, an angiotensin receptor blocker (ARB), a beta blocker, a diuretic, a mineralocorticoid receptor antagonist or a calcium channel blocker (CCB).

As used herein, the term “sequential” with respect to a therapeutic use refers to administration of at least two active ingredients (i.e., at least two difference therapeutic agents) at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this definition.

As used herein, the term “simultaneous” with respect to a therapeutic use refers to the administration of at least two active ingredients (i.e., at least two difference therapeutic agents) by the same or different route but at the same time or at substantially the same time.

As used herein, the term “solvate” refers to forms of a compound (e.g., a peptide or mixture of peptides) that are associated with a solvent, usually by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, the term “subject” and “patient” refers to a living animal. In various embodiments, a subject is a mammal. In some embodiments, a subject is a non-human mammal, including, without limitation, a mouse, rat, hamster, guinea pig, rabbit, sheep, goat, cat, dog, pig, minipig, horse, cow, or non-human primate. In some embodiments, the subject is a human. As used herein, the terms “subject” and “patient” are or can be used interchangeably.

It is also to be appreciated that the various modes of treatment or prevention of medical conditions as described herein, in some embodiments, are intended to mean “substantial,” which includes total but also less than total treatment or prevention, and wherein some biologically or medically relevant result is achieved.

As used herein the term “synergistic therapeutic effect” refers to a greater-than-additive therapeutic effect that is produced by a combination of at least two therapeutic agents, and which exceeds that which would otherwise result from the individual administration of the agents. For example, lower doses of two or more therapeutic agents may be used in treating heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM), resulting in increased therapeutic efficacy and decreased side-effects of one or both of the therapeutic agents. For example, the co-administration of a peptide or mixture of peptides as disclosed herein (e.g., a peptide of Formula A, A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 or A-15-2) may enhance the effectiveness of the administration of an additional therapeutic agent.

As used herein, the term “tautomer” refers to compounds (e.g., a peptide or mixture of peptides) that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the term “treat,” “treats,” “treating,” or “treatment” refers to therapeutic treatment, wherein the object is to reduce, alleviate or delay onset of the progression or advancement of, and/or reverse the progression of the targeted pathological disease, disorder or condition or its associated signs and/or symptoms in a sample or subject administered a therapeutic agent(s) relative to a control sample, control subject or control group of subjects not administered the therapeutic agent(s).

### DETAILED DESCRIPTION

The present disclosure relates generally to compounds (e.g., peptides), compositions (e.g., medicaments or formulations comprising the compounds and/or peptides), methods, and uses for treating, preventing, inhibiting, ameliorating, or delaying the onset of heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM), or the signs, symptoms or conditions associated therewith, in a mammalian subject in need thereof. The present disclosure further provides, in some embodiments, for compounds (e.g., peptides), compositions (e.g., medicaments or formulations), methods, and uses for treating, preventing, inhibiting, ameliorating, or delaying the onset of the signs or symptoms of heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM) in a subject. The present technology further relates to administering an effective amount of a peptide, mixture of peptides, or a composition, formulation or medicament comprising the peptide or mixture of peptides and optional additional therapeutic agent(s) as described herein; or a pharmaceutically acceptable salt, hydrate, solvate, stereoisomer and/or tautomer of said peptide or peptides (including peptides in their carboxylate form), wherein the additional therapeutic agent(s) can be an angiotensin-converting enzyme (ACE) inhibitor, an angiotensin receptor blocker (ARB), a beta blocker, a diuretic, a mineralocorticoid receptor antagonist, a calcium channel blocker, mavacamten (Camzyos®) or any two or more of the foregoing.

In some embodiments, the peptide, or peptides of a mixture, is/are a peptide/peptides of Formula A:

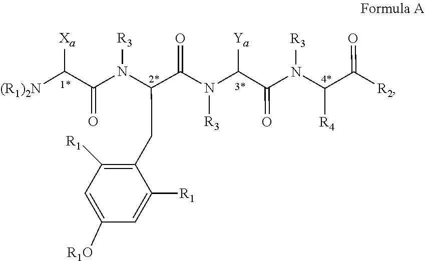

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

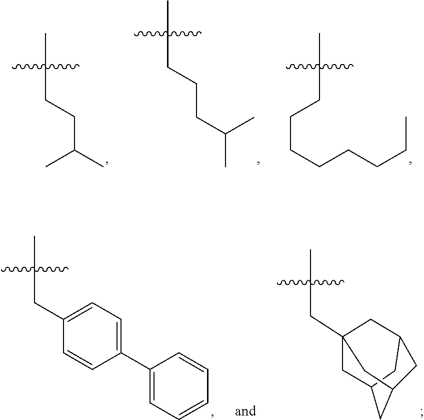

Xₐ is selected from

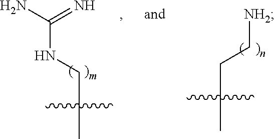

Yₐ is selected from

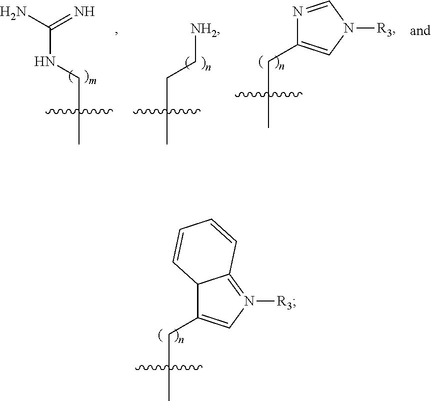

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom.

The peptide or a peptide within a mixture of peptides of Formula A can be of Formula A-1 (H-D-Arg-2,6-Dmt-Lys-ADM-NH₂, wherein Dmt is 2,6-dimethyltyrosine and ADM is adamantylalanine):

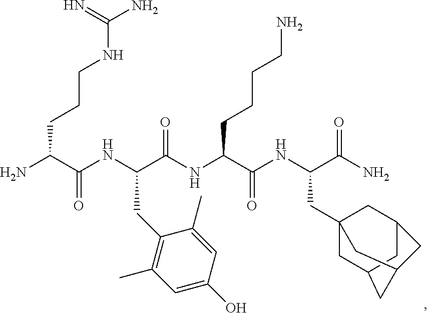 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O]

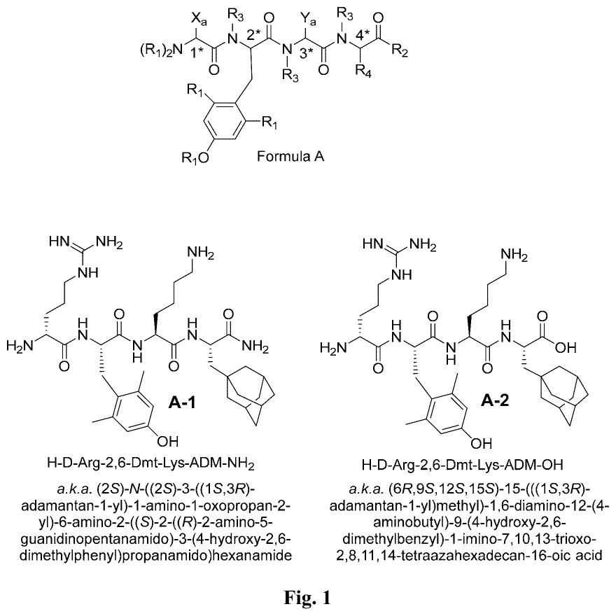

or a pharmaceutically acceptable salt, hydrate, solvate and/or tautomer thereof (See also FIG. 1 for names of this peptide). The peptide or a peptide within a mixture of peptides of Formula A can be of Formula A-2 (H-D-Arg-2,6-Dmt-Lys-ADM-OH):

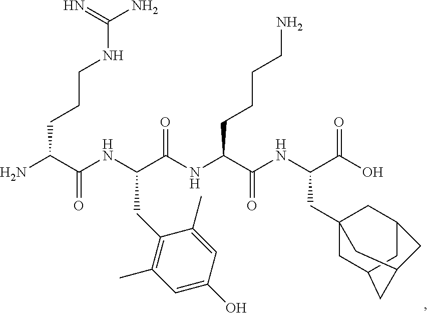 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate and/or tautomer thereof (See also FIG. 1 for names of this peptide). The peptide or a peptide within a mixture of peptides of Formula A can be a peptide of Formula A-3 (D-Arg-Dmt-Arg-AMD-NH₂):

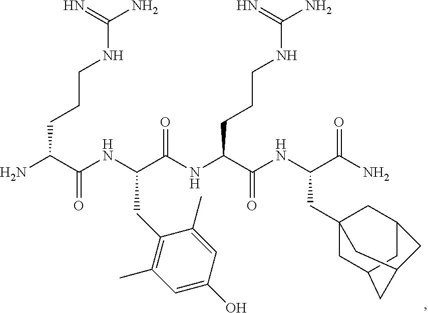 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-4 (D-Arg-Dmt-Arg-AMD-OH):

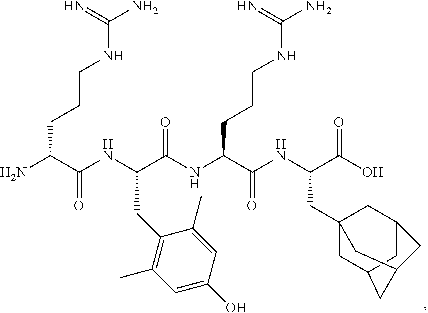 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-5 (D-Arg-Dmt-Orn-AMD-NH₂ wherein Orn is the amino acid ornithine):

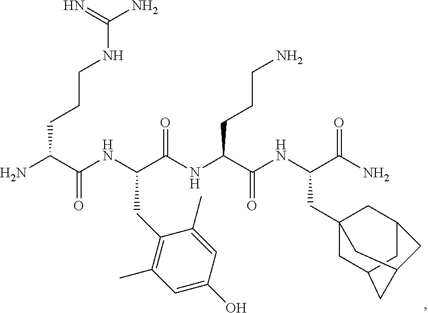 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-6 (D-Arg-Dmt-Orn-AMD-OH):

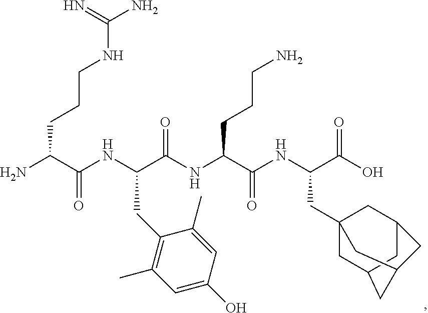 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-7 (D-Arg-Tyr-Lys-AMD-NH₂):

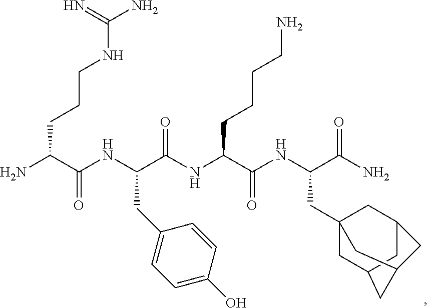 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O]

or a pharmaceutically acceptable salt, hydrate, solvate and/or tautomer thereof. The peptide or a peptide within a mixture of peptides of Formula A can be a peptide of Formula A-8 (D-Arg-Tyr-Lys-AMD-OH):

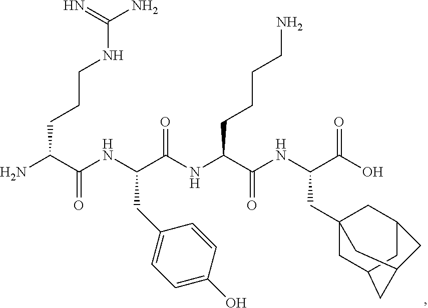 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

or a pharmaceutically acceptable salt, hydrate, solvate and/or tautomer thereof. The peptide or a peptide within a mixture of peptides of Formula A can be a peptide of Formula A-9 (D-Arg-4-Me-Dmt-Lys-AMD-NH₂, wherein 4-Me-Dmt is 4-methyl-2,6-dimethyltyrosine):

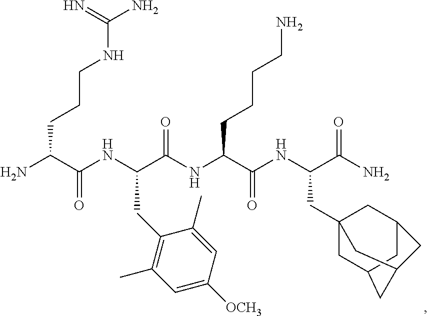 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1]

or a pharmaceutically acceptable salt, hydrate, solvate and/or tautomer thereof. The peptide or a peptide within a mixture of peptides of Formula A can be a peptide of Formula A-10 (D-Arg-4-Me-Dmt-Lys-AMD-OH):

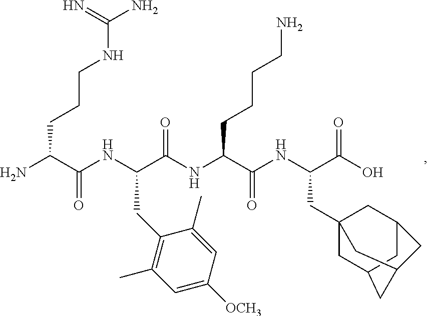 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

or a pharmaceutically acceptable salt, hydrate, solvate and/or tautomer thereof. The peptide or a peptide within a mixture of peptides of Formula A can be a peptide of Formula A-11-1 (D-Arg-Dmt-Lys-BPA-NH₂, wherein BPA is biphenylalanine):

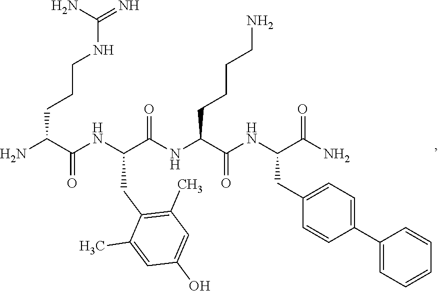 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

or a pharmaceutically acceptable salt, hydrate, solvate and/or tautomer thereof. The peptide or a peptide within a mixture of peptides of Formula A can be a peptide of Formula A-11-2 (D-Arg-Dmt-Lys-BPA-OH):

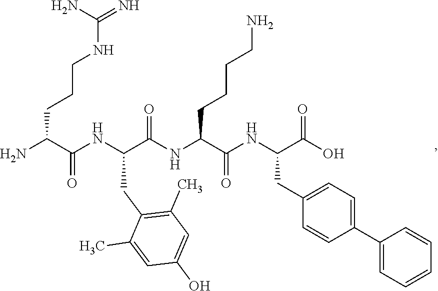 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

or a pharmaceutically acceptable salt, hydrate, solvate and/or tautomer thereof. The peptide or a peptide within a mixture of peptides of Formula A can be a peptide of Formula A-12-1 (D-Arg-Dmt-Lys-n-HA-NH₂, wherein n-HA is n-heptylalanine):

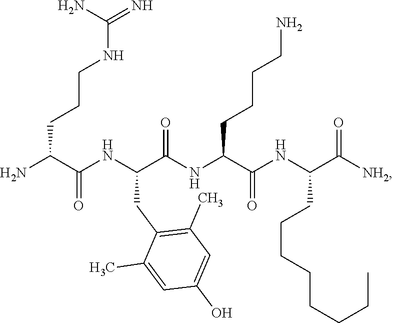 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

or a pharmaceutically acceptable salt, hydrate, solvate and/or tautomer thereof. The peptide or a peptide within a mixture of peptides of Formula A can be a peptide of Formula A-12-2 (D-Arg-Dmt-Lys-n-HA-OH):

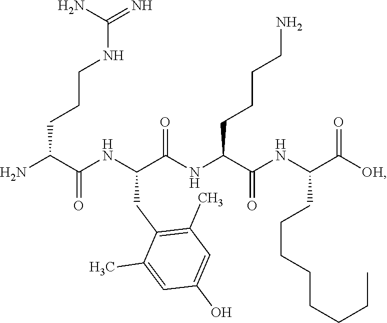 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

or a pharmaceutically acceptable salt, hydrate, solvate and/or tautomer thereof. The peptide or a peptide within a mixture of peptides of Formula A can be a peptide of Formula A-13-1 (D-Arg-Dmt-Trp-IBA-NH₂, wherein IBA is isobutylalanine):

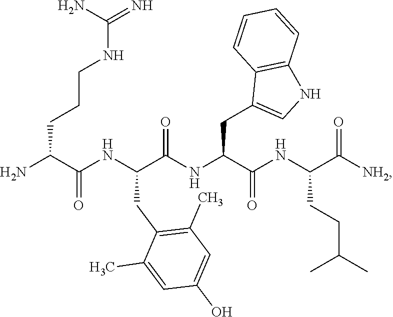 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O]

or a pharmaceutically acceptable salt, hydrate, solvate and/or tautomer thereof. The peptide or a peptide within a mixture of peptides of Formula A can be a peptide of Formula A-13-2 (D-Arg-Dmt-Trp-IBA-OH):

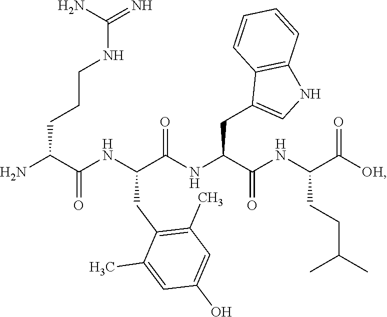 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

or a pharmaceutically acceptable salt, hydrate, solvate and/or tautomer thereof. The peptide or a peptide within a mixture of peptides of Formula A can be a peptide of Formula A-14-1 (D-Arg-Dmt-n-Me-Trp-BPA-NH₂, wherein n-Me-Trp is N-methyltryptophan):

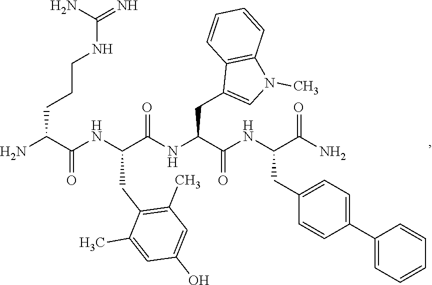 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

or a pharmaceutically acceptable salt, hydrate, solvate and/or tautomer thereof. The peptide or a peptide within a mixture of peptides of Formula A can be a peptide of Formula A-14-2 (D-Arg-Dmt-n-Me-Trp-BPA-OH):

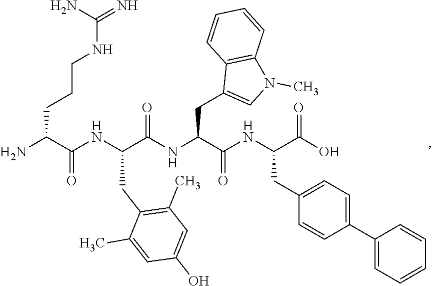 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

or a pharmaceutically acceptable salt, hydrate, solvate and/or tautomer thereof. The peptide or a peptide within a mixture of peptides of Formula A can be a peptide of Formula A-15-1 (D-Arg-Dmt-His-BPA-NH₂):

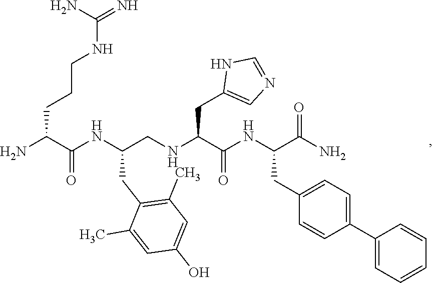 [SMILES: Cc1cc(O)cc(C)c1C[C@@H](CN[C@@H](Cc1cnc[nH]1)C(=O)N[C@@H](Cc1ccc(-c2ccccc2)cc1)C(N)=O)NC(=O)[C@H](N)CCCNC(=N)N]

or a pharmaceutically acceptable salt, hydrate, solvate and/or tautomer thereof. The peptide or a peptide within a mixture of peptides of Formula A can be a peptide of Formula A-15-2 (D-Arg-Dmt-His-BPA-OH):

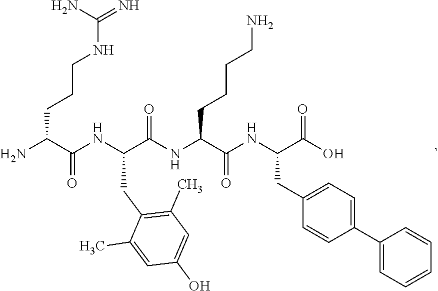 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

or a pharmaceutically acceptable salt, hydrate, solvate and/or tautomer thereof.

The peptide(s) can be administered individually or as a mixture comprising two or more of the peptides as defined herein. The peptide, or mixture of peptides, can be administered alone, in a formulation (e.g. medicament) or in combination with one or more other active ingredients. In some embodiments, the pharmaceutically acceptable salt can be selected from a hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate or trifluoroacetate salt. When formulated, in some embodiments, the composition, formulation or medicament can be prepared by formulating the tris-HCl salt of the peptide or peptides.

In some embodiments, mixtures of two or more of the above described peptides are used as a/the therapeutic agent. Such mixtures may be present intentionally (e.g., by mixing the peptides post synthesis) or fortuitously (e.g., by the hydrolysis of a C-terminal amide to a C-terminal carboxylic acid). Whenever reference is made herein to a peptide or mixture of peptides, it is implied and intended that each individual peptide of said peptide or mixture of peptides can exist as a free acid/base, in zwitterionic form or in any salt form, including in a pharmaceutically acceptable salt form (e.g., See: FIG. 2 and FIG. 3 for various salt forms of the peptides of Formula A-1 & A-2).

For example, FIG. 2 illustrates salt various forms that the peptide of Formula A-2 can take and FIG. 3 illustrates various salt forms that the peptide of Formula A-1 can take. With reference to FIG. 2, (20) illustrates a mono-basic salt form of the peptide of Formula A-2, wherein the C-terminal carboxylate has been ionized as its base-salt. As illustrated, the basic generic salt represented by YOH can ionize to produce Y+ and OH− and thereby ionize the peptide of Formula A-2 (21) to form (20). The generic basic salt represented by YOH could be, for example, sodium hydroxide (NaOH), potassium hydroxide (KOH) or lithium hydroxide (LiOH). The mono-basic salt form (20) can be protonated with acid (e.g., represented by HX wherein H⁺ is the proton and X-represents the counterion and, for example, is embodied by acids such as HCl, HBr or HI) to form the peptide of Formula A-2 (21). However, the peptide of Formula A-2 (21) can also be represented in zwitterionic form (22) resulting from the internal distribution of a proton between the carboxylate and one of the basic groups. The peptide of Formula A-2 ((21) or (22)) can be further protonated with a single equivalent of acid (i.e., HX) to thereby produce a mono-acid salt (23). The mono-acid salt (23) can be further acidified with another equivalent of acid (i.e., HX) to thereby produce a bis-acid salt (24). The bis-acid salt (24) can be further acidified with another equivalent of acid (i.e., HX) to thereby produce a (tri or) tris-acid salt (25). A similar analysis of FIG. 3 can be done to determine the various salt forms of the peptide of Formula A-3. In some embodiments, the peptide or peptides of the mixture of peptides is/are formulated from a mono-acid salt form. In some embodiments, the peptide or peptides of the mixture of peptides is/are formulated from a bis-acid salt form. In some embodiments, the peptide or peptides of the mixture of peptides is/are formulated from a tri-acid (a.k.a., a tris-acid) salt form.

One of skill in the art will appreciate that these transitions between the free-base and various salt forms are easily accomplished by using an appropriate amount of organic or inorganic acid or base. One of skill in the art will further appreciate that such transitions between salt forms are also applicable to any peptides represented by Formula A, including without limitation the peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1, A-15-2. The peptide or peptides may be formulated as a pharmaceutically acceptable salt as defined herein.

Certain compound(s)/peptide(s) disclosed in the present disclosure can exist in unsolvated forms as well as solvated forms, including hydrated forms. Solvated forms can exist, for example, because it is difficult or impossible to remove all the solvent from the peptide post synthesis. In general, the solvated forms are equivalent to unsolvated forms and are encompassed within the scope of the present disclosure.

Certain compound(s)/peptide(s) of the present disclosure may exist in crystalline form, multiple crystalline forms, amorphous forms, polymorphous forms or any combination of the foregoing. Certain compound(s)/peptide(s) of the present disclosure may exist in various tautomeric forms. Certain compound(s)/peptide(s) of the present disclosure may exist in various salt forms or mixtures of salt forms. In general, all physical forms of the compound(s)/peptide(s) disclosed herein are deemed equivalent for the uses contemplated by the present disclosure and are intended to be within the scope of the present disclosure.

Certain compound(s)/peptide(s) disclosed in the present disclosure can exist in various tautomeric forms. In general, all tautomeric forms of the peptides disclosed herein are equivalent with respect to their application to the embodiments of the present disclosure and deemed to be encompassed within the scope of the present disclosure.

### Chiral/Stereochemistry Considerations

Peptides/compounds described herein can comprise one or more asymmetric centers, and thus can exist in various isomeric forms, e.g., as enantiomers and/or diastereomers in relation to others of the peptides disclosed herein (i.e., stereoisomers). Chiral centers in illustrated structures (including the claims) may be identified herein by use of an asterisk (*). For example, the compounds described herein can be in the form of an individual enantiomer, diastereomer or geometric isomer, or can be in the form of a mixture of stereoisomers, including racemic mixtures and mixtures enriched in one or more stereoisomer, unless otherwise specifically illustrated as a particular stereoisomer. Stereoisomers can be isolated from mixtures by methods known to those skilled in the art, including chiral high-pressure liquid chromatography (HPLC) and the formation and crystallization of chiral salts. Alternatively, preferred stereoisomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure of the present application additionally encompasses compounds described herein as individual isomers (or stereoisomers) substantially free of other isomers (or stereoisomers), and alternatively in some embodiments, as mixtures of various isomers (or stereoisomers). Any of the compounds disclosed herein can be prepared as substantially pure stereoisomers (e.g., substantially pure stereoisomer) if desired.

As used herein, a pure enantiomeric compound is substantially free from other enantiomers or stereoisomers of the compound (i.e., in enantiomeric excess (ee) of the other stereoisomer); as purity is a relative term in the sense that it is exceedingly difficult to achieve 100% purity. In other words, an “S” form of the compound is substantially free from the “R” form of the compound and is, thus, in enantiomeric excess of the “R” form. With respect to common amino acids found in nature (which are more commonly described in terms of “D” and “L” enantiomer), it is to be understood that for a “D”-amino acid the configuration is “R” and for an “L”-amino acid, the configuration is “S”, with the known exception of L-cysteine which is R-cysteine and vice-versa. The measure of stereoisomeric purity is typically referred to using the designation “ee”, which refers to enantiomeric excess. The higher the “ee” value, the greater the stereochemical purity of the substance/compound. In some embodiments, ‘substantially free’ or ‘substantially pure’, refers to: (i) an aliquot of an “R” form of a peptide containing a specified chiral center contains less than 2% “S” form at that specified chiral center; or (ii) an aliquot of an “S” form of a peptide containing a specified chiral center contains less than 2% “R” form at that specified chiral center. In some embodiments, the term “enantiomerically pure” or “pure enantiomer” denotes that the compound or peptide comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the particularly identified enantiomer (e.g., as compared with the other enantiomer). Typically, when used as a therapeutic agent, the peptides disclosed herein will be substantially free of other stereoisomers (enantiomers or diastereoisomers) of the designated peptide. It is to be understood that when administered to a human subject, the peptide or peptides selected for use in methods, formulation of compositions or medicaments, or presented for uses will often be substantially free of enantiomeric or diastereomeric impurity peptides.

In certain embodiments, the weights are based upon total weight of all enantiomers or stereoisomers of the compound. When illustrated herein, a composition drawn in a particular stereoisomeric form would be considered to be in substantially pure form (i.e., substantially free of undesired enantiomeric or diastereomeric impurities) for administration to a subject when such level of stereoisomeric purity is approved for administration by a medicines regulating authority of a country for administration of such a therapeutic agent/composition for treating a particular indication or condition.

In the compositions provided herein, an enantiomerically pure compound (e.g., a peptide) can be present with other active or inactive ingredients. For example, a pharmaceutical composition comprising enantiomerically pure “R” form compound (i.e., the is substantially free of other stereoisomers) can comprise, for example, about 90% excipient and about 10% enantiomerically pure “R” form of the compound/peptide. In certain embodiments, the therapeutic agent (i.e., compound or peptide) can be formulated with little or no excipient or carrier.

### Peptide Synthesis:

The peptides may be synthesized by any of the methods well known in the art. The peptides can be prepared using solid-phase synthesis methodology. The peptides can be synthesized by using solution-phase methodology. Suitable methods for chemically synthesizing the peptides include, for example, those described in any of WO 2004/070054, and/or WO 2020/131283. For example, the tetrapeptide of Formula A-1 can be prepared by at 2+2 synthesis by preparing the partially protected dipeptides 41 and 42 (FIG. 4) followed by coupling the dipeptides to produce the protected tetrapeptide, followed by deprotection; essentially as described in WO2016/144905, Examples 5, wherein the phenylalanine amide is replaced with the appropriate adamantyl containing amino acid amide. Also see: WO2020/131283. In some embodiments, the peptides are C-terminal amides and in some embodiments the peptides are C-terminal carboxylic acids. The methodology used to prepare the peptide may be influenced by the desired C-terminal modification.

For example, the peptides disclosed herein can be prepared using any peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132; Stuart and Young in Solid Phase Peptide Synthesis, Second Edition, Pierce Chemical Company (1984), and in Methods Enzymol., 289, Academic Press, Inc., New York (1997)). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption. The peptides disclosed herein can be prepared as described in published WIPO/PCT application WO2022/131283 (Published on Jun. 25, 2020). The tetrapeptide of Formula A-1 was prepared using a solution phase methodology whereby two suitably protected dimers were coupled and then deprotected according to Scheme set forth in FIG. 4.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. Typically in solid phase synthesis, after coupling, a capping step is performed to cap (render unreactive) any unreacted amine groups. These steps (i.e., deprotection, coupling, and optionally capping) may be repeated to form the desired peptide chain. Once the desired peptide chain is complete, the peptide may be cleaved from the solid support and purified to the desired degree of purity.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z, cbz or Cbz), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl) isopropyloxycarbonyl, 2-(3,5-dimethoxyphenyl) isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methylsulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support. Further reference is also made to: Isidro-Llobet, A., Alvarez, M., Albericio, F., “Amino Acid-Protecting Groups”; Chem. Rev., 109:2455-2504 (2009) as a comprehensive review of protecting groups commonly used in peptide synthesis.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase or solution-phase peptide synthesis discussed herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), and its HCl salt (EDC·HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include (1-[Bis(dimethylamino)methylene]-1H-1,2,3-triazolo[4,5-b]pyridinium 3-oxide hexafluorophosphate (HATU) and (2-(1H-benzotriazol-1-yl)-1,1,3,3-tetramethyluronium hexafluorophosphate (HBTU), generally used in combination with an organic base such as N,N-diisopropylethylamine (DIEA) and a hindered pyridine-type base such as lutidine or collidine.

In some embodiments, the amino acids can be activated toward coupling by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996); and WO 2018/034901. Such methods of peptide synthesis may be used to produce the peptides disclosed herein either by solution-phase or solid-phase methodology.

### Discussion of Examples Supporting the Present Disclosure and Claims:

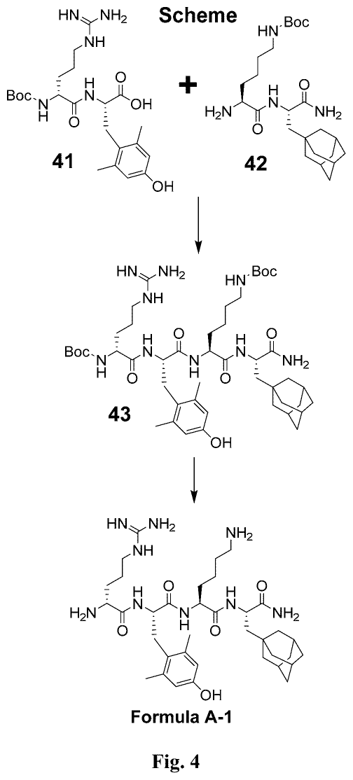

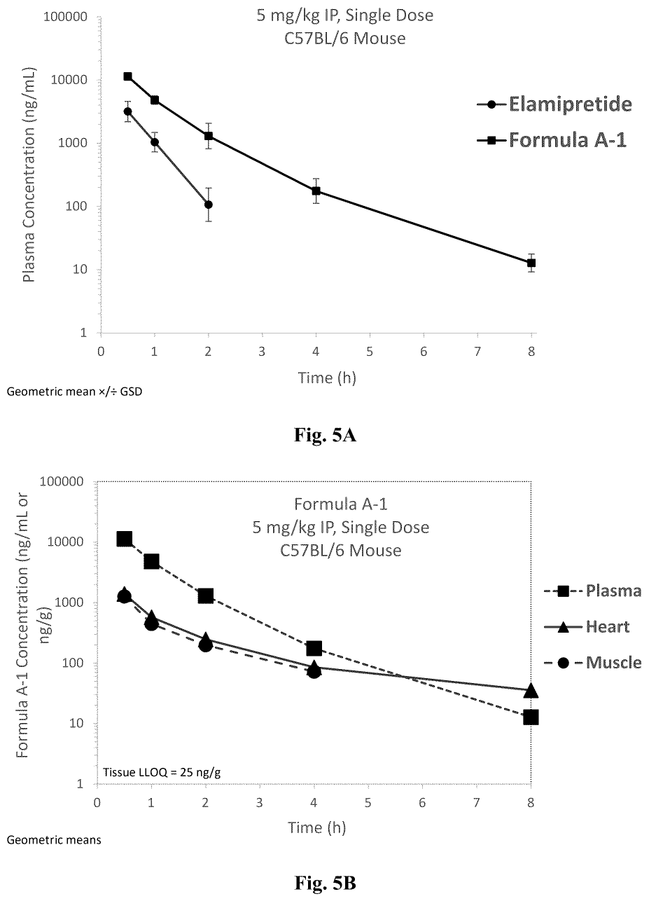

Example 1 provides a comparative assessment of the pharmacokinetics of elamipretide and the peptide of Formula A-1 based on the plasma concentration of the two peptides over time based on a single administered dose to mice at 5 mg/kg of each peptide intraperitoneally (IP). The data presented in FIG. 5A for this Example 1, demonstrates that at approximately 1 hour, the concentration of the peptide of Formula A-1 is 3-4 higher in the plasma and is still detectable in the plasma after 8 hours (whereas the elamipretide is not). This data suggests that the peptide of Formula A-1 is cleared more slowly (at least in this species of mouse) as compared with elamipretide and therefore may be available to produce a greater therapeutic effect when administered to a subject at the same dose as elamipretide.

In the data presented in FIG. 5B for this Example 1, the plasma concentration data from FIG. 5A is reproduced but also provided is the concentration data for the peptide of Formula A-1 in heart and muscle tissue over time. These data demonstrate that there is good uptake of the peptide of Formula A-1 in heart and muscle (albeit about 10× lower than in the plasma-initially), without any significant difference observed in uptake between the two tissues (i.e., muscle and heart). Whilst initially still about 10 times lower than the plasma concentration (but cleared more slowly from the tissues than from the plasma), the peptide of Formula A-1 provides reasonable penetration into muscle and heart tissue sufficient to potentially produce an efficacious drug effect. Eventually, the concentration of the peptide of Formula A-1 is present in the tissues (heart and muscle) is higher than that in the plasma. Furthermore, the data collected indicated that the peptide of Formula A-1 was well tolerated in these mice when dosed at this level.

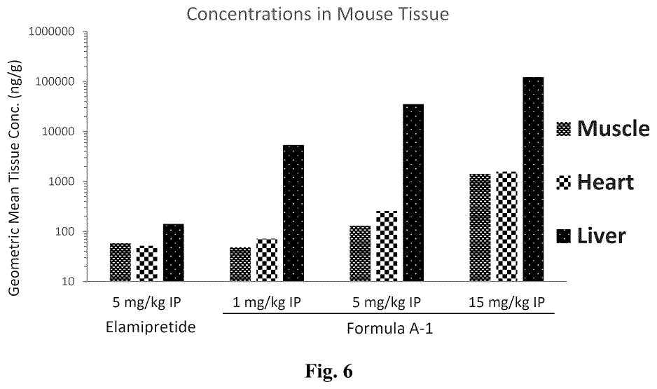

With reference to Example 2 and FIG. 6, data is presented in bar graph form for the concentrations of elamipretide and the peptide of Formula A-1 in muscle, heart and liver tissues where the elamipretide was administered at 5 mg/kg IP, and the peptide of Formula A-1 was administered at 1 mg/kg, 5 mg/kg or 15 mg/kg IP; in all cases repeated doses once per day over a five day period. As can be seen with reference to FIG. 6, the concentration of the peptide of Formula A-1 administered at 1 mg/kg is roughly equivalent to the concentration of elamipretide administered at 5 mg/kg in heart and muscle tissue, thereby substantially confirming the data presented in Example 1 regardless of the difference between single and repeated dosing (compare with FIGS. 5A and 5B). In comparison, the concentration of the peptide of Formula A-1 is much higher (approximately 30-fold) in the liver when administered at 1 mg/kg as compared with the concentration of elamipretide in the liver when administered at 5 mg/kg. With reference to FIG. 6, tissue uptake of the peptide of Formula A-1 increased in response to the amount dosed. Furthermore, the data collected indicated that elamipretide and the peptide of Formula A-1 was well tolerated in these mice at all dosing levels examined. Collectively, this data collected in this Example 2 provides a sound basis to expect that the peptide of Formula A-1 may perform at least equal to or substantially better than elamipretide in treating a condition involving a cardiomyopathy.

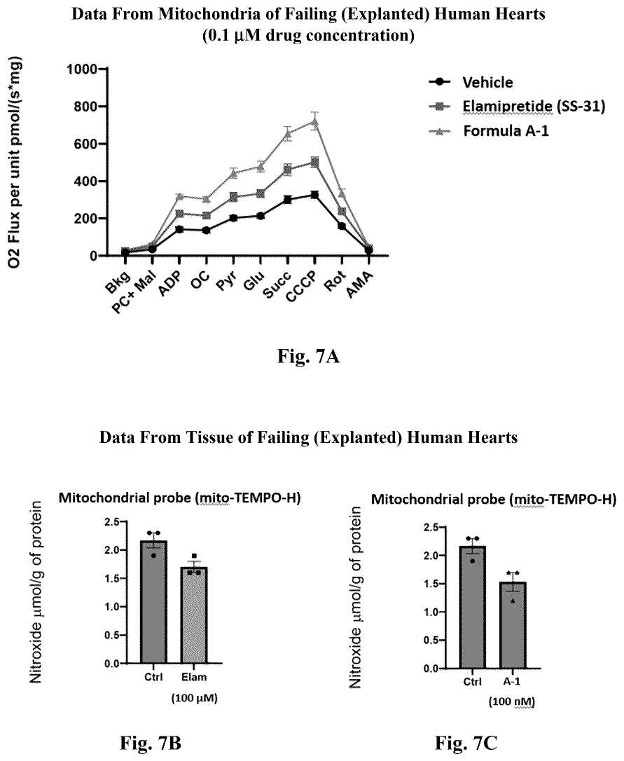

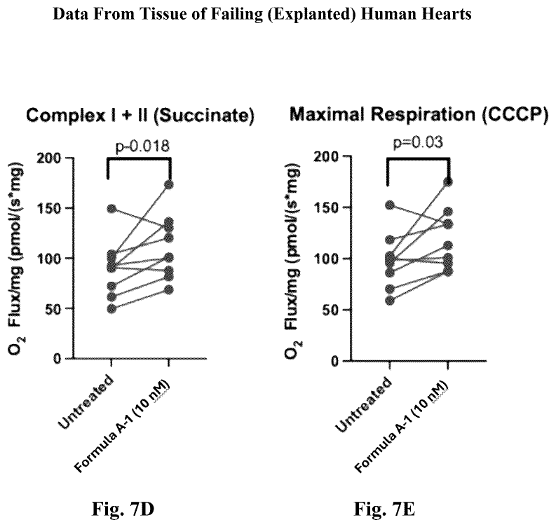

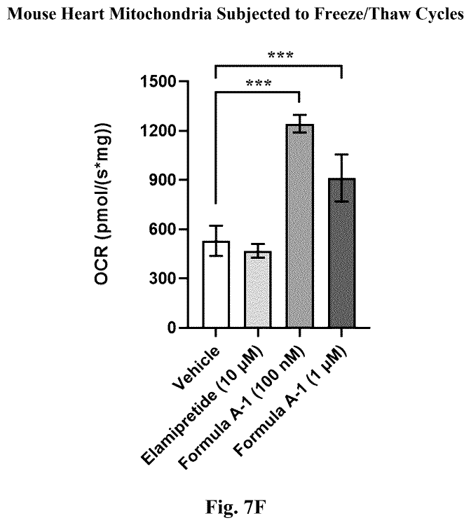

With reference to Example 3 and FIG. 7A, data is presented in line graph form for the O₂ flux of failing human heart mitochondria treated with vehicle (water), 0.1 μM elamipretide (SS-31), or 0.1 μM the peptide of Formula A-1 followed by the stepwise addition of 0.2 mM palmitoylcarnitine (PC), 1 mM malate (Mal), 4 mM ADP, 0.4 mM octonylcarnitine (OC), 5 mM pyruvate (Pyr), 10 mM glutamate (Glu), 10 mM succinate (Succ), 0.5 μM steps of CCCP (until maximum rate reached) followed by 2 μM rotenone (Rot) and 5 μM antimycin A (AMA). O₂ flux was measured by high resolution respirometry and rates were normalized using the amount of protein added in μg of homogenate. These data demonstrate that treatment with the peptide of Formula A-1 improves O₂ flux of failing human heart mitochondria with a higher potency than elamipretide (SS-31) relative to vehicle-treated control.

With reference to Example 3 and FIG. 7B, data is presented in bar graph form for the mitochondrial superoxide production by failing human hearts as measured by electron paramagnetic resonance (EPR) spectroscopy. These data demonstrate that treatment with 100 μM elamipretide reduced mitochondrial superoxide production by failing human hearts relative to vehicle-treated control.

With reference to Example 3 and FIG. 7C, data is presented in bar graph form for the mitochondrial superoxide production by failing human hearts as measured by electron paramagnetic resonance (EPR) spectroscopy. These data demonstrate that treatment with 100 nM (the concentration of the peptide of Formula A-1 (FIG. 7C) was three orders of magnitude lower than the concentration of elamipretide used to generate FIG. 7B) of the peptide of Formula A-1 reduced mitochondrial superoxide production by failing human hearts relative to vehicle-treated control.

With reference to Example 3 and FIG. 7D, data is presented in line graph form for O₂ flux of failing human heart mitochondria treated with vehicle (water) or 10 nM the peptide of Formula A-1 using succinate as a substrate. These data demonstrate that treatment with peptide of Formula A-1 improves oxygen consumption rate of complex I+II from the failing human heart.

With reference to Example 3 and FIG. 7E, data is presented in line graph form for O₂ flux of failing human heart mitochondria treated with vehicle (water) or 10 nM the peptide of Formula A-1 using the uncoupler CCCP as a substrate. These data demonstrate that treatment with peptide of Formula A-1 improves maximal respiration of mitochondria from the failing human heart.

With reference to Example 4 and FIG. 7F, data is presented in bar graph form the oxygen consumption rate (OCR) of failing mouse heart mitochondria following repeated freeze/thaw cycles and treatment with vehicle control, elamipretide (10 μM), or the peptide of Formula A-1 (100 nM or 1 μM). These data demonstrate that peptide of Formula A-1 (at orders of magnitude lower concentration as compared to elamipretide) improves oxygen consumption of failing mouse heart mitochondria relative to vehicle control, whereas treatment with elamipretide had no significant effect.

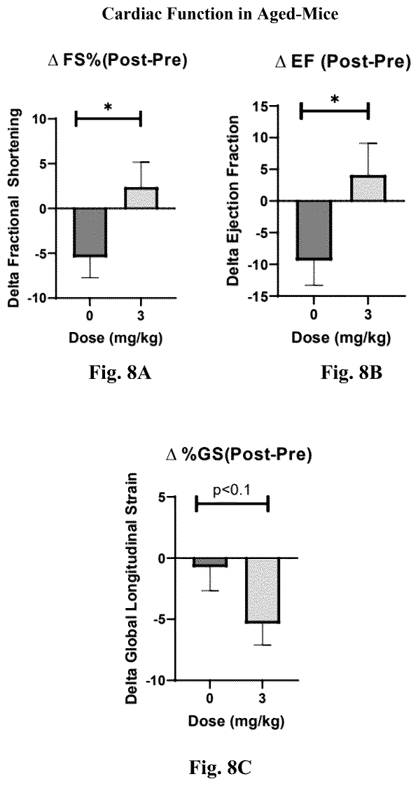

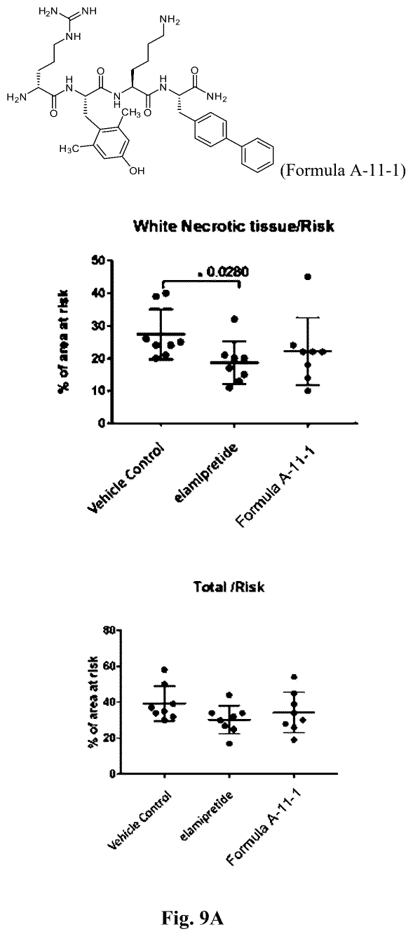

With reference to Example 5 and FIGS. 8A-8C, data is presented in bar graph form for the change in cardiac function, as measured by the change in the percentage of fractional shortening (FS), ejection fraction (EF), and global longitudinal strain (GS), in aged mice after daily treatment with the peptide of Formula A-1 (3 mg/kg) for 8 weeks. These data demonstrate that treatment of the mice with the peptide of Formula A-1 produces a statistically significant improvement in FS and EF, and exhibited a trend towards improvement of GS in aged mice.

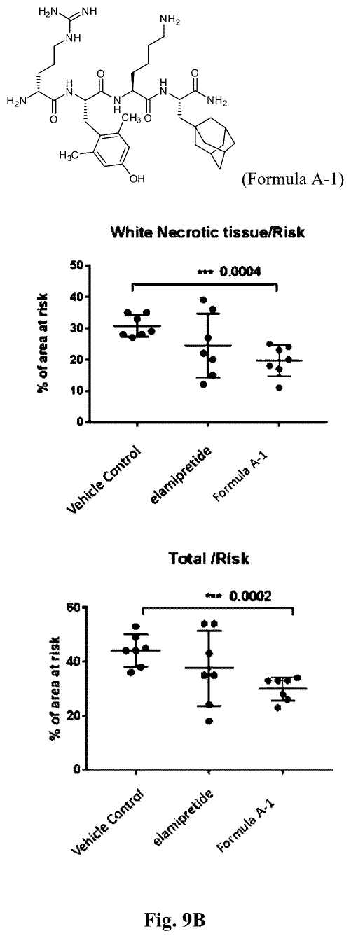

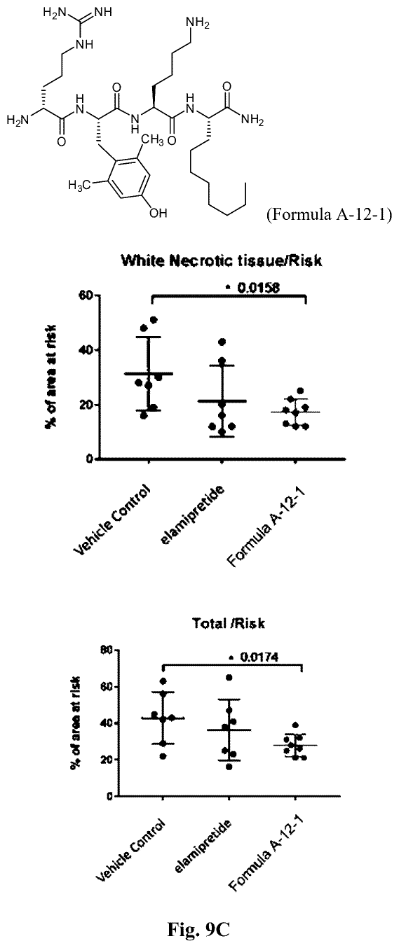

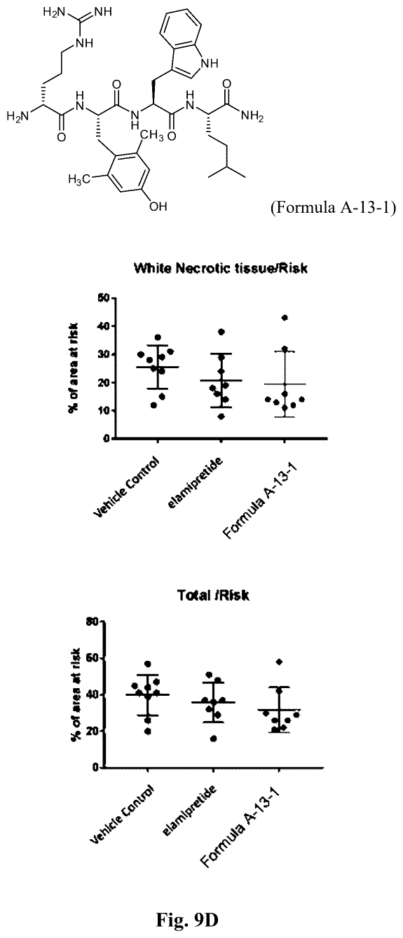

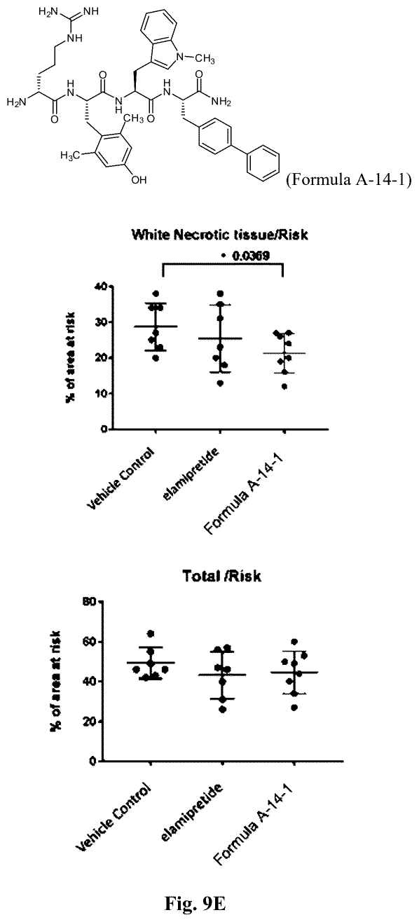

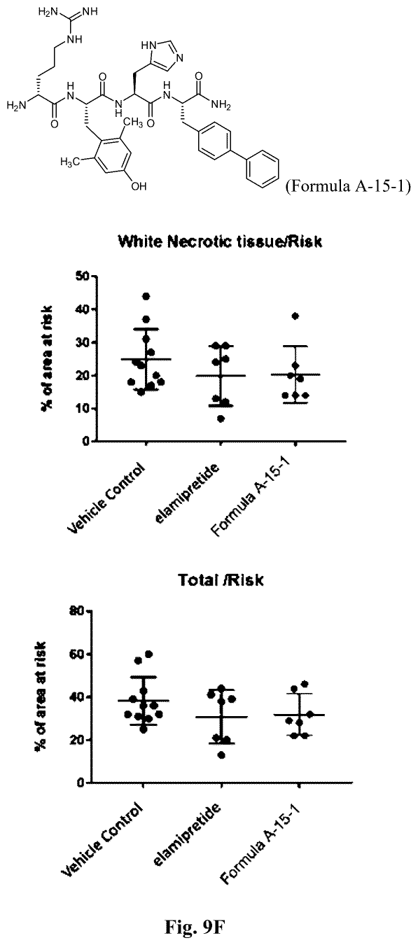

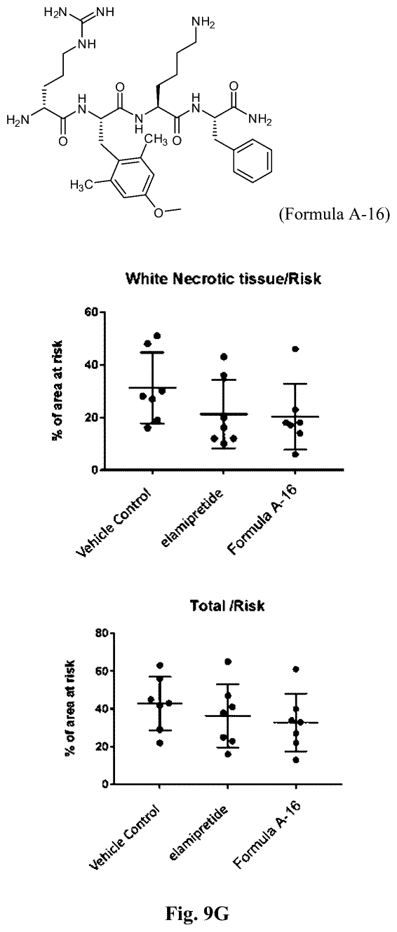

With reference to Example 6 and FIGS. 9A-9G, data is presented in bar graph form demonstrating the efficacy of peptides of Formulas A-1, A-11-1, A-12-1, A-13-1, A-14-1, A-15-1 and A-16 in exhibiting cardioprotective effects in a Langendorff study of induced ischemia-reperfusion injury (See Herr, D. J. et al. Induction and Assessment of Ischemia-reperfusion Injury in Langendorff-perfused Rat Hearts. J. Vis. Exp. (101), e52908, doi:10.3791/52908 (2015)). The modifications of the peptides used in Example 6 are representative of features/amino acids found in the peptide of Formula A. Generally, the peptides described in Example 6 all performed similar to or slightly better than did elamipretide in the ex-vivo Langendorff assay.

### Methods

As can be deduced from the Examples and Figs. presented, the peptide of Formula A (such and the peptide of Formula A-1) can be prepared and administered to mammalian subjects such that it is well tolerated and can be delivered in an efficacious dose to the heart and other muscles of the subject (FIGS. 5A, 5B, and 6). Indeed, the data suggests that the peptide of Formula A-1 may be superior in its ability to penetrate heart and muscle tissue as compared with a peptide (i.e., elamipretide) well-known for its potential in addressing cardiomyopathies (See: Daubert et al., Novel Mitochondria-Targeting Peptide in Heart Failure Treatment: A Randomized, Placebo-Controlled Trial of Elamipretide, Circ Heart Fail. 2017; 10: e001389, Gandhi et al., Cardiomyopathy in Duchenne Muscular Dystrophy and the Potential for Mitochondrial Therapeutics to Improve Treatment Response, Cells, 2024, 13, 1168 and Thompson et al., Long-term efficiency and safety of elamipretide in patients with Barth syndrome: 168-week open-label extension results of TAZPOWER, Genetics in Medicine (2024) 26, 101138). The data presented shows that the peptide of Formula A-1 appears to be able to improve mitochondrial function in dysfunctional human heart tissue (FIGS. 7A, 7B, 7C, 7D, and 7E) and in dysfunctional mouse heart tissue (FIG. 7F). The data presented also appears to show that the peptide of Formula A-1 may be able to normalize or improve cardiac function, including normalization of fractional shortening, ejection fraction, and global longitudinal strain, in aged-mice (FIGS. 8A, 8B, and 8C). The data in FIGS. 9A-9G demonstrates that variations of amino acids according to the peptide of Formula A appear to be similar to or somewhat superior to elamipretide, at least with respect to performance in the Langendorff assay-which assay is ex-vivo so it does not involve systemic administration of the peptide to a living mammal.

Therefore, in one aspect, the present disclosure provides methods for treating heart failure (HF), idiopathic cardiomyopathy (ICM), dilated cardiomyopathy (DCM), restrictive cardiomyopathy (RCM), hypertrophic cardiomyopathy (HCM), hypertensive cardiomyopathy (HTN-CM), or one or more of the signs or symptoms thereof, in a mammalian subject in need thereof, comprising administering to the subject a therapeutically effective amount of a peptide of Formula A, or a pharmaceutically acceptable salt, hydrate, solvate and/or tautomer thereof. In another aspect, the present disclosure provides methods for preventing heart failure (HF), idiopathic cardiomyopathy (ICM), dilated cardiomyopathy (DCM), restrictive cardiomyopathy (RCM), hypertrophic cardiomyopathy (HCM), hypertensive cardiomyopathy (HTN-CM) or one or more of the signs or symptoms thereof in a mammalian subject in need thereof, comprising administering to the subject a therapeutically effective amount of a peptide of Formula A, or a pharmaceutically acceptable salt, hydrate, solvate and/or tautomer thereof. In still one aspect, the present disclosure provides methods for inhibiting heart failure (HF), idiopathic cardiomyopathy (ICM), dilated cardiomyopathy (DCM), restrictive cardiomyopathy (RCM), hypertrophic cardiomyopathy (HCM), hypertensive cardiomyopathy (HTN-CM) or one or more of the signs or symptoms thereof in a mammalian subject in need thereof, comprising administering to the subject a therapeutically effective amount of a peptide of Formula A, or a pharmaceutically acceptable salt, hydrate, solvate and/or tautomer thereof. In yet one aspect, the present disclosure provides methods for ameliorating heart failure (HF), idiopathic cardiomyopathy (ICM), dilated cardiomyopathy (DCM), restrictive cardiomyopathy (RCM), hypertrophic cardiomyopathy (HCM), hypertensive cardiomyopathy (HTN-CM) or one or more of the signs or symptoms thereof in a mammalian subject in need thereof, comprising administering to the subject a therapeutically effective amount of a peptide of Formula A, or a pharmaceutically acceptable salt, hydrate, solvate and/or tautomer thereof. In yet one aspect, the present disclosure provides methods for delaying the onset of heart failure (HF), idiopathic cardiomyopathy (ICM), dilated cardiomyopathy (DCM), restrictive cardiomyopathy (RCM), hypertrophic cardiomyopathy (HCM), hypertensive cardiomyopathy (HTN-CM) or one or more of the signs or symptoms thereof in a mammalian subject in need thereof, comprising administering to the subject a therapeutically effective amount of a peptide of Formula A, or a pharmaceutically acceptable salt, hydrate, solvate and/or tautomer thereof.

Thus, in some embodiments, this disclosure pertains to a method for treating, preventing, inhibiting, ameliorating or delaying the onset of heart failure (HF), idiopathic cardiomyopathy (ICM), dilated cardiomyopathy (DCM), or restrictive cardiomyopathy (RCM) in a mammalian subject in need thereof, comprising administering to the subject a therapeutically effective amount of a peptide of Formula A, or a mixture of peptides of Formula A:

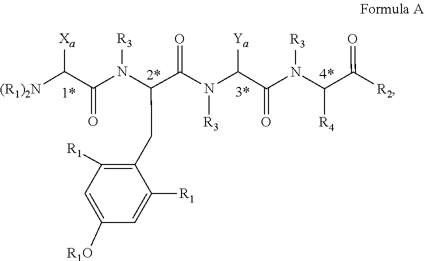

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

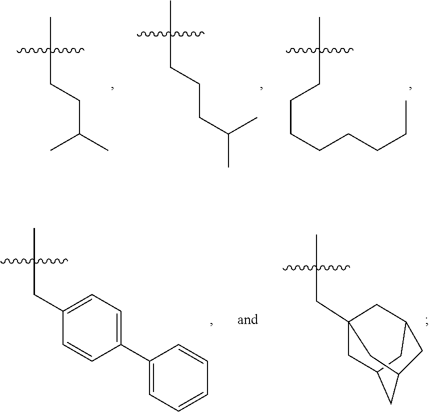

Xₐ is selected from

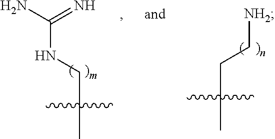

Yₐ is selected from

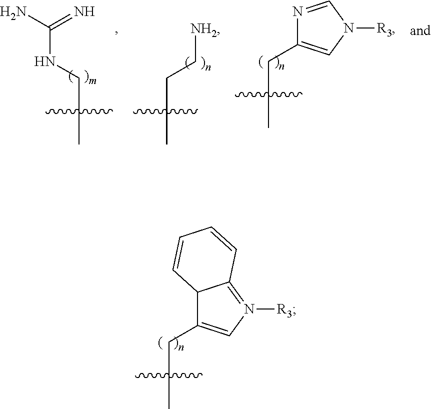

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, this disclosure pertains to a method for treating, preventing, inhibiting, ameliorating or delaying the onset of heart failure (HF). In some embodiments, this disclosure pertains to a method for treating, preventing, inhibiting, ameliorating or delaying the onset of idiopathic cardiomyopathy (ICM). In some embodiments, this disclosure pertains to a method for treating, preventing, inhibiting, ameliorating or delaying the onset of dilated cardiomyopathy (DCM). In some embodiments, this disclosure pertains to a method for treating, preventing, inhibiting, ameliorating or delaying the onset of restrictive cardiomyopathy (RCM).

In some embodiments, this disclosure pertains to a method for treating, preventing, inhibiting, ameliorating or delaying the onset of hypertrophic cardiomyopathy (HCM, including obstructive hypertrophic cardiomyopathy) or hypertensive cardiomyopathy in a mammalian subject in need thereof, comprising administering to the subject a therapeutically effective amount of a peptide of Formula A, or a mixture of peptides of Formula A:

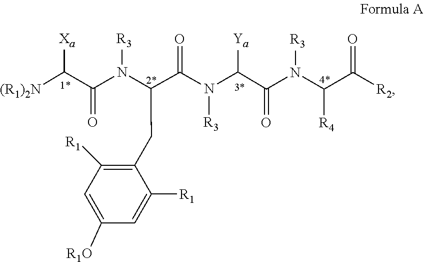

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

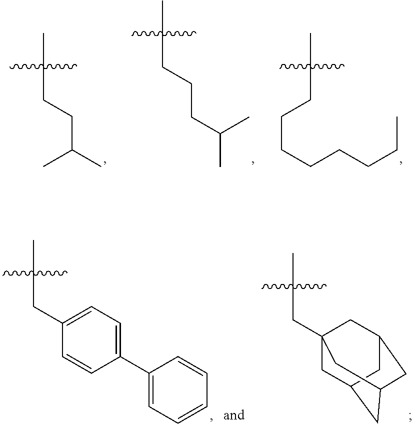

Xₐ is selected from

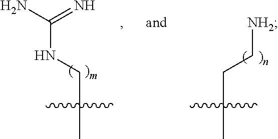

Yₐ is selected from

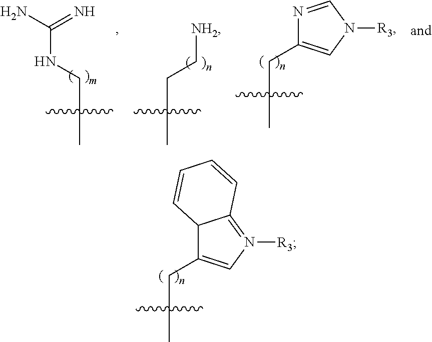

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, this disclosure pertains to a method for treating, preventing, inhibiting, ameliorating or delaying the onset of hypertrophic cardiomyopathy (HCM, including obstructive hypertrophic cardiomyopathy). In some embodiments, this disclosure pertains to a method for treating, preventing, inhibiting, ameliorating or delaying the onset of hypertensive cardiomyopathy (HTM-CM).

In practice of any of the foregoing methods, the peptide or peptides of Formula A is/are a peptide/peptides of Formula A-1 or Formula A-2:

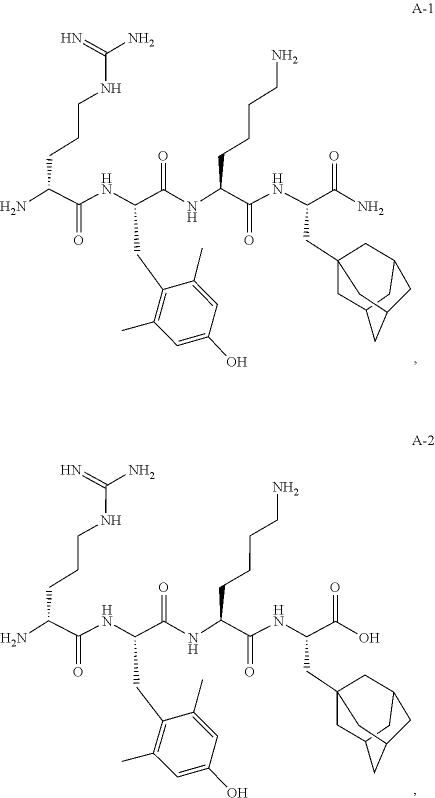 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

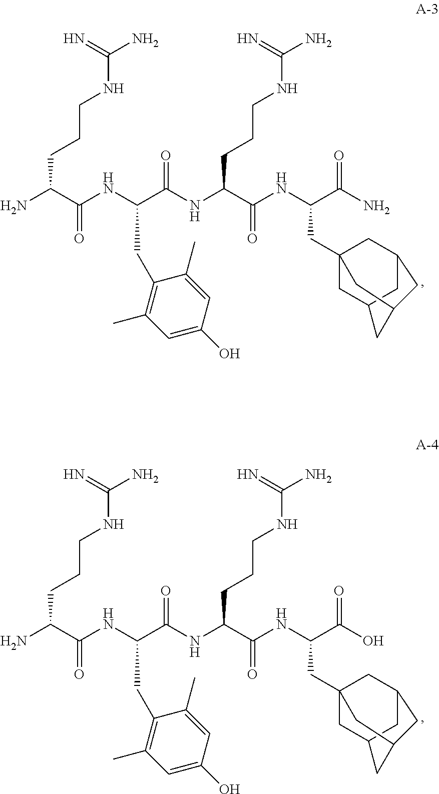 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O]

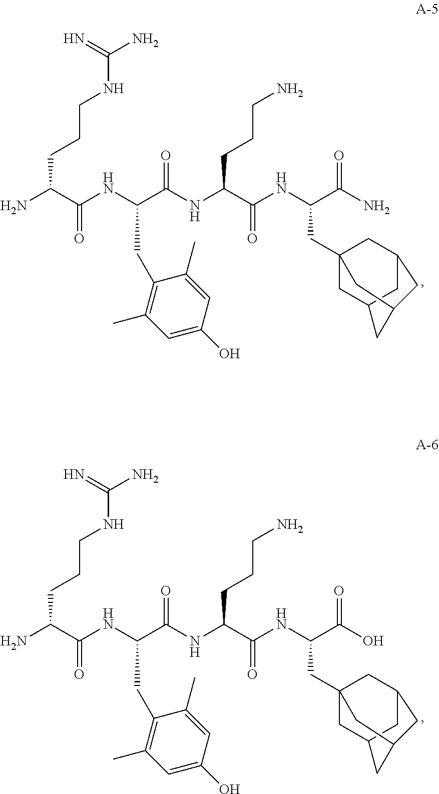 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

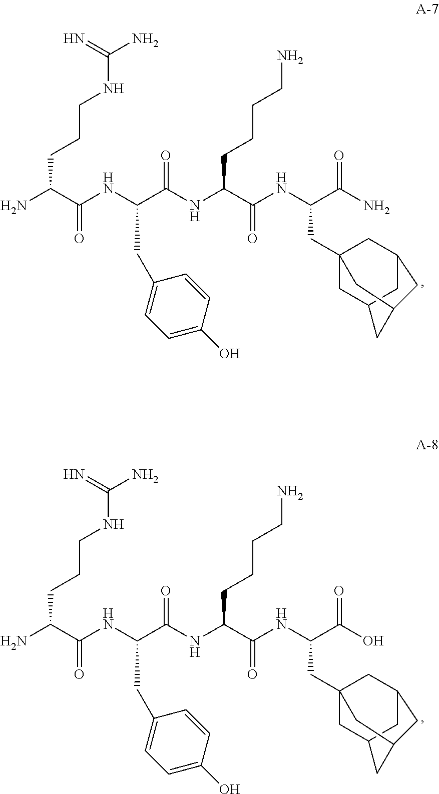 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

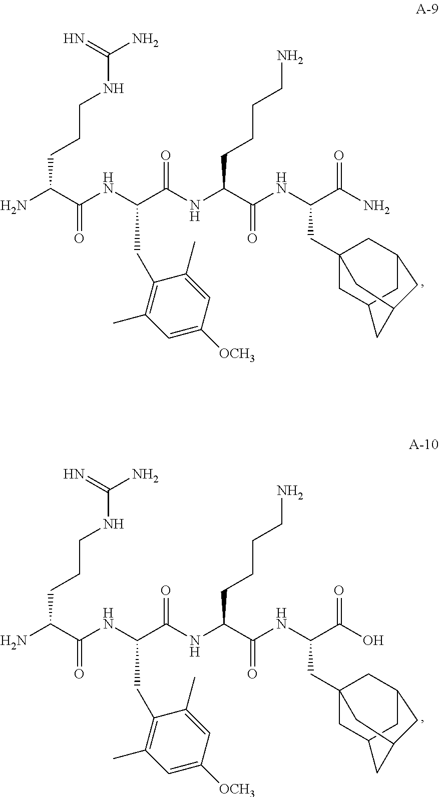 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

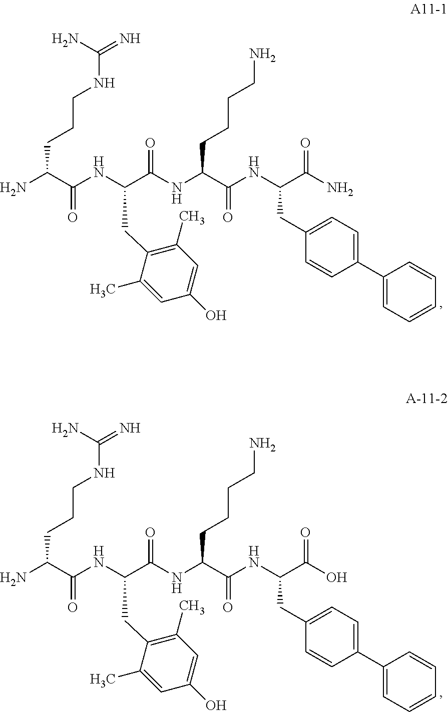 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

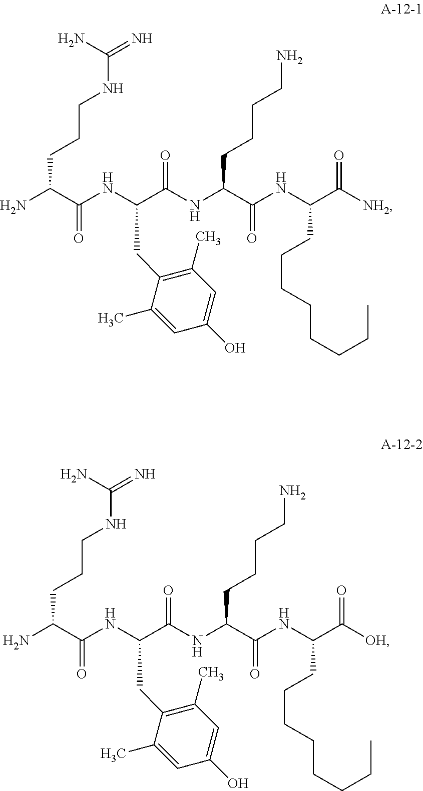 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

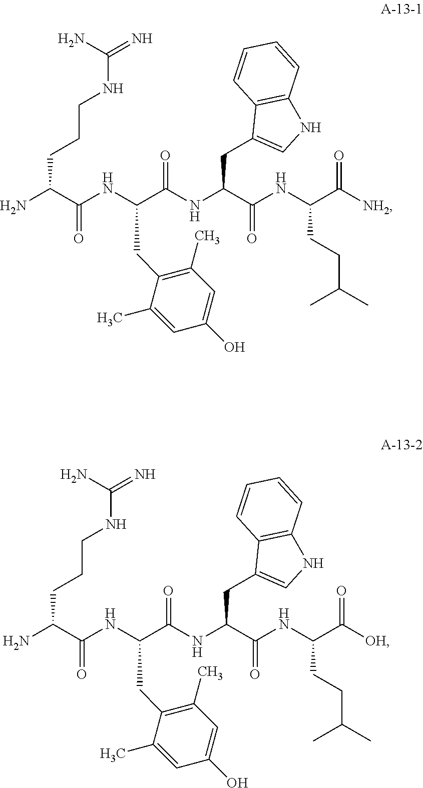 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

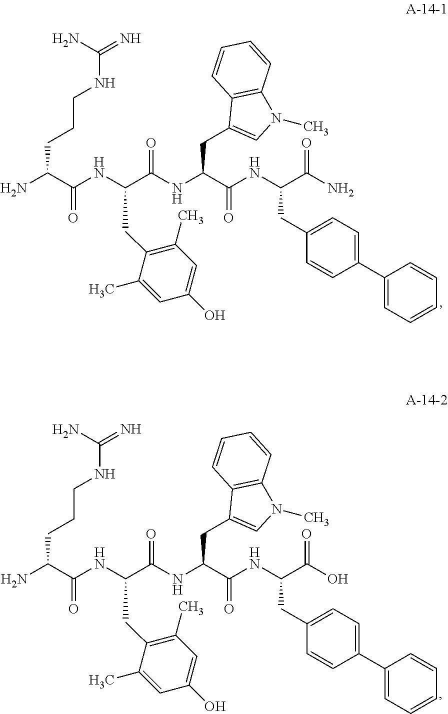 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

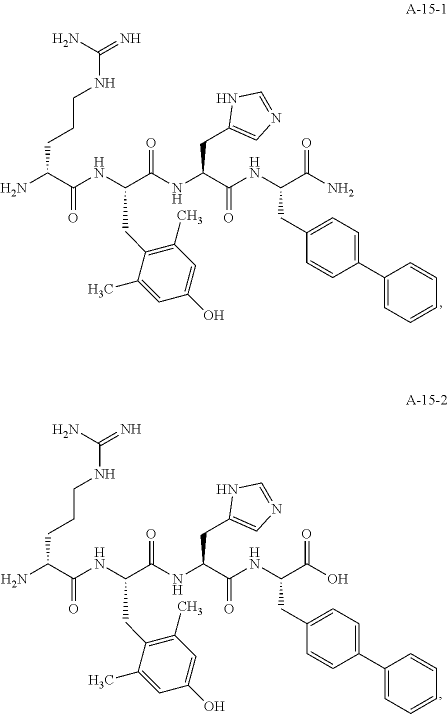 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments of the foregoing methods, one or more hydrogen atoms of the peptide or peptides of Formulas A are optionally substituted with a deuterium or fluorine atom.

In practice of any of the foregoing methods, the heart failure can be: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the method, the heart failure is congestive heart failure (CHF).

In practice of any of the foregoing methods, administration of the peptide or peptides may; (i) ameliorate(s), (ii) delay(s) the onset of, and/or (iii) delay(s) the progression of one or more signs or symptoms of heart failure in the subject as compared with a control subject or group of control subjects to which the peptide or peptides was/were not administered. In some embodiments of the method, the signs or symptoms of heart failure include: (i) shortness of breath, (ii) ankle and/or foot swelling; (iii) leg swelling; (iv) fatigue, and/or (v) labored breathing when lying flat.

In practice of any of the foregoing methods, the peptide can be administered daily for: (i) 12 weeks or more; (ii) 24 weeks or more; (iii) 48 weeks or more; (iv) 72 weeks or more; or (v) 96 weeks or more. In some embodiments of the method, the peptide is administered subcutaneously or intravenously. In some embodiments of the method, the peptide is administered orally, topically, systemically, intraperitoneally, intradermally, transdermally, ophthalmically, intrathecally, intracerebroventricularly, iontophoretically, transmucosally, intravitreally, intranasally, or intramuscularly.

In practice of any of the foregoing methods, the subject can be human.

In practice of any of the foregoing methods, the method may further comprise separately, sequentially, or simultaneously administering an additional therapeutic agent to the subject. In some embodiments, the additional therapeutic agent is an angiotensin-converting enzyme (ACE) inhibitor, an angiotensin receptor blocker (ARB), a beta blocker, a diuretic, a mineralocorticoid receptor antagonist or a calcium channel blocker (CCB). In some embodiments of the method, the angiotensin-converting enzyme (ACE) inhibitor is captopril, enalapril, lisinopril, benazepril, or ramipril. In some embodiments of the method, the angiotensin receptor blocker (ARB) is azilsartan, candesartan, eprosartan, irbesartan, telmisartan, valsartan, losartan, olmesartan, Entresto® (sacubitril/valsartan) or Byvalson™ (nebivolol/valsartan). In some embodiments of the method, the beta blocker is carvedilol, bisoprolol, metoprolol succinate, atenolol, esmolol, nebivolol and propranolol. In some embodiments of the method, the diuretic is bumetanide, furosemide or torsemide. In some embodiments of the method, the mineralocorticoid receptor antagonist is spironolactone or eplerenone. In some embodiments of the method, the calcium channel blocker is amlodipine (Norvasc®), diltiazem (Cardizem), felodipine, isradipine, nicardipine, nifedipine (Procardia®), nisoldipine (Sular®) or verapamil (Verelan®). In some embodiments of the method, the additional therapeutic agent is mavacamten (Camzyos®).

In practice of any of the foregoing methods, the pharmaceutically acceptable salt of the peptide may comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide can be formulated for administration from its tris-HCl salt.

In another aspect, this disclosure relates to a method for normalizing left ventricular ejection fraction, fractional shortening, and/or global longitudinal strain in a mammalian subject in need thereof, comprising administering to the subject a therapeutically effective amount of a peptide of Formula A, or a mixture of peptides of Formula A:

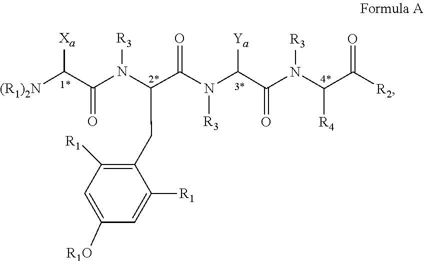

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

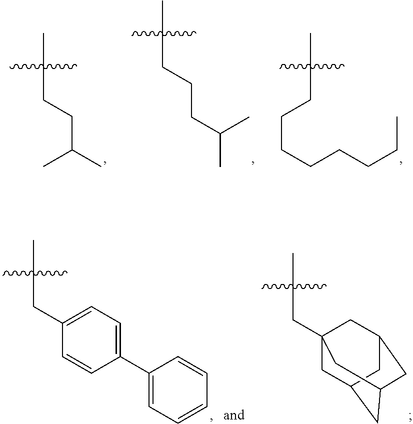

Xₐ is selected from

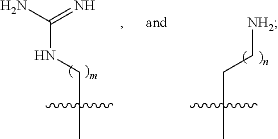

Yₐ is selected from

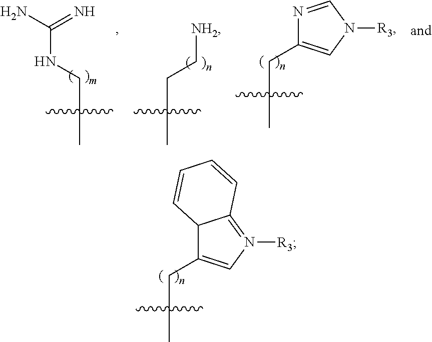

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom, wherein normalization of the left ventricular ejection fraction, fractional shortening, and/or global longitudinal strain occurs in the subject as compared with a control subject to control group of subjects not administered the peptide or peptides of Formula A. In some embodiments of the method, the peptide or peptides of Formula A is/are a peptide/peptides of Formula A-1 or Formula A-2.

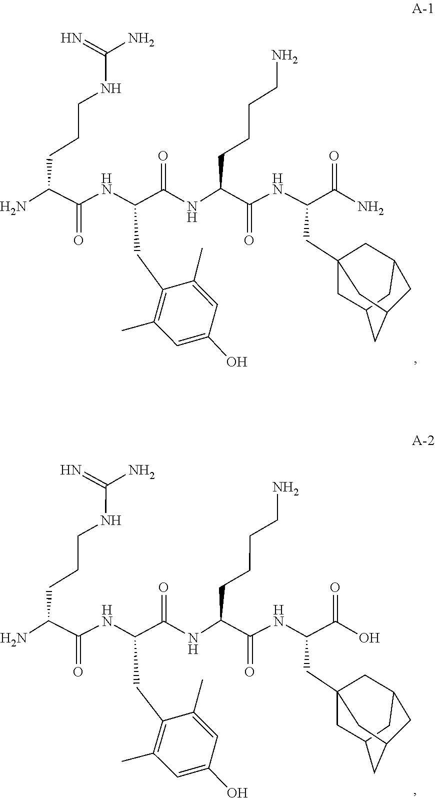 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the method, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

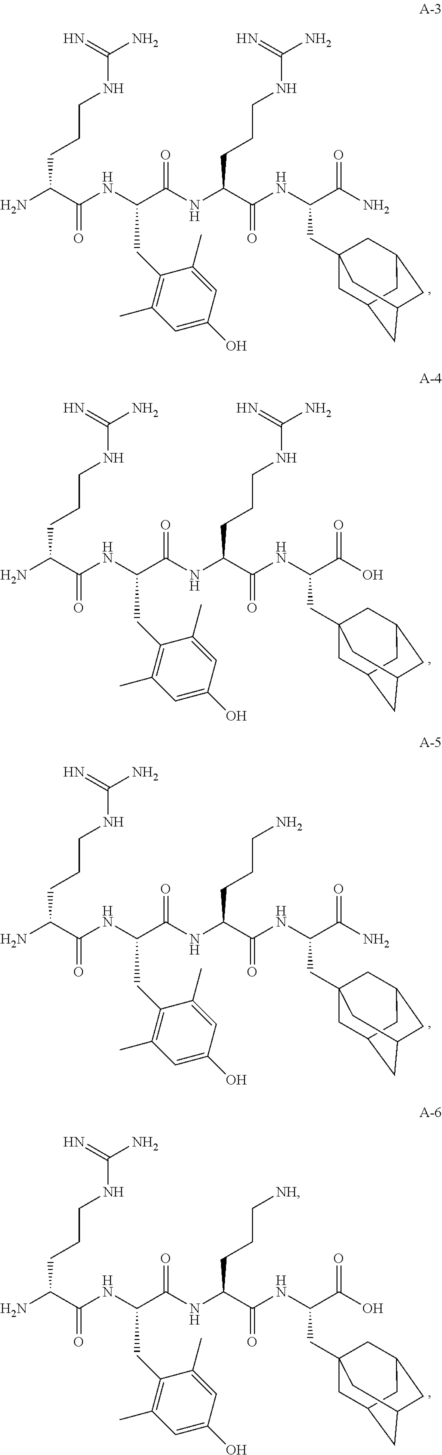

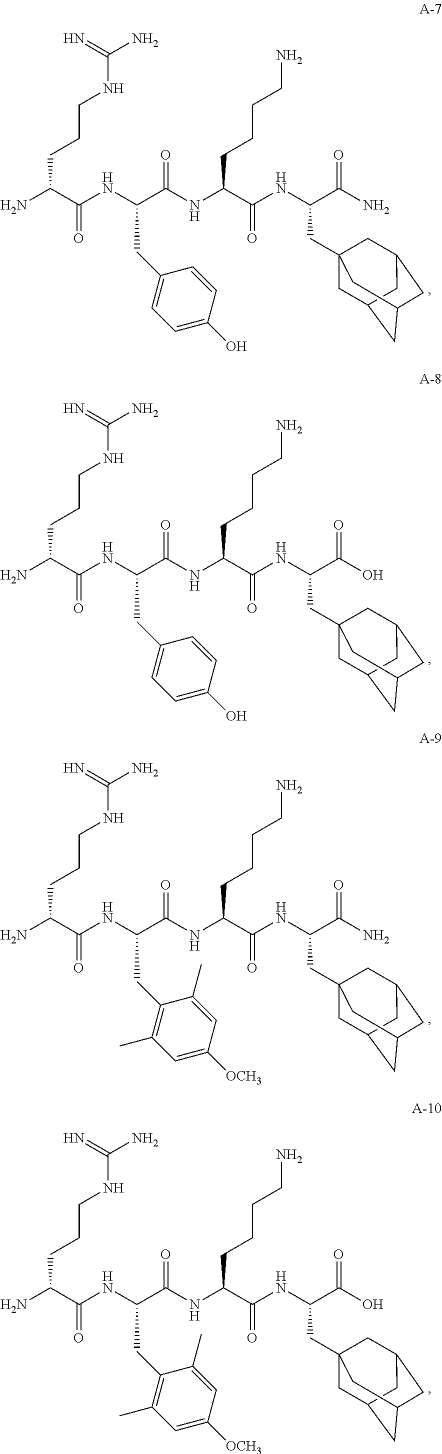 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

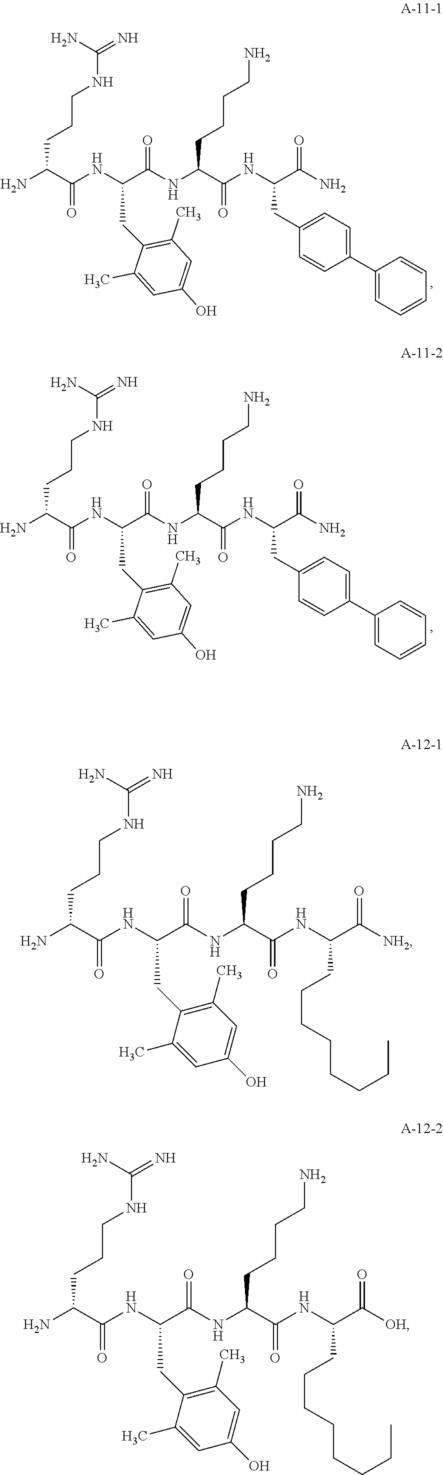 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

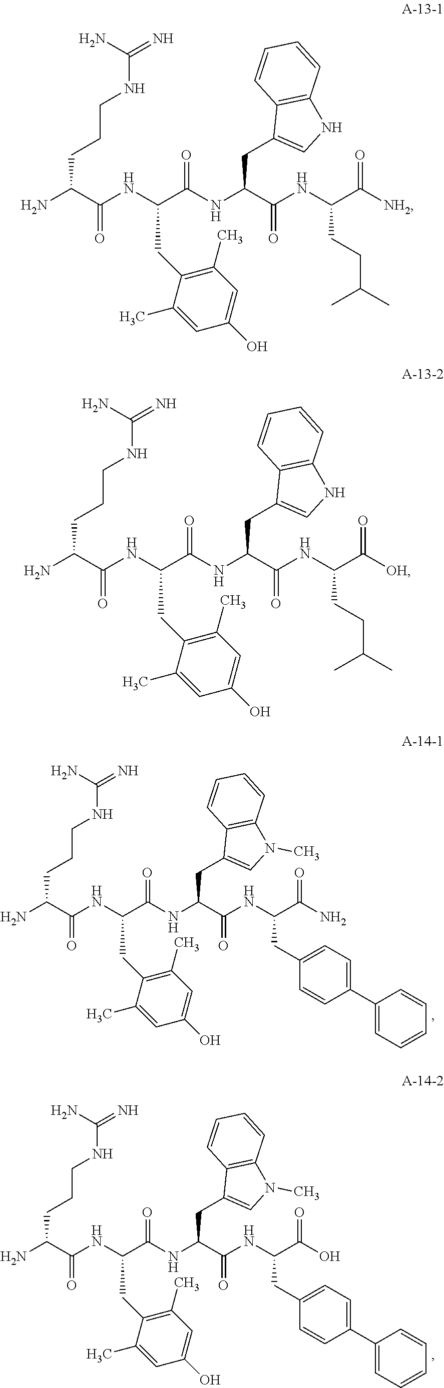 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

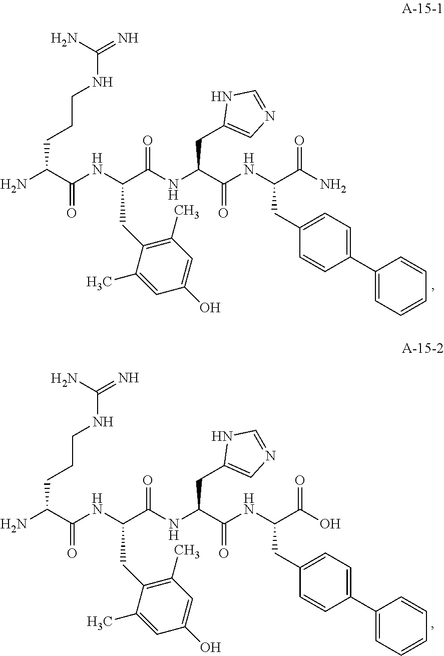 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the method is for normalizing left ventricular ejection fraction. In some embodiments, the method is for normalizing fractional shortening. In some embodiments, the method is for normalizing global longitudinal strain. In some embodiments of the foregoing methods, one or more hydrogen atoms of the peptide or peptides of Formulas A are optionally substituted with a deuterium or fluorine atom.

In some embodiments of the method, the peptide or peptides is/are administered daily for: (i) 12 weeks or more; (ii) 24 weeks or more; (iii) 48 weeks or more; (iv) 72 weeks or more; or (v) 96 weeks or more. In some embodiments of the method, the peptide or peptides is/are administered subcutaneously or intravenously. In some embodiments of the method, the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the method, the subject is human.

In some embodiments, the method further comprises separately, sequentially, or simultaneously administering an additional therapeutic agent to the subject. In some embodiments of the method, the additional therapeutic agent is a beta blocker, a diuretic, a mineralocorticoid receptor antagonist or a calcium channel blocker (CCB). In some embodiments of the method, the beta blocker is carvedilol, bisoprolol, metoprolol succinate, atenolol, esmolol, nebivolol and propranolol. In some embodiments, the diuretic is bumetanide, furosemide or torsemide. In some embodiments of the method, the mineralocorticoid receptor antagonist is spironolactone or eplerenone. In some embodiments of the method, the calcium channel blocker is amlodipine (Norvasc®), diltiazem (Cardizem), felodipine, isradipine, nicardipine, nifedipine (Procardia®), nisoldipine (Sular®) or verapamil (Verelan®). In some embodiments, the additional therapeutic agent is mavacamten (Camzyos®).

In some embodiments of the method, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the method, the peptide or peptides is/are formulated for administration from its/their tris-HCl salt.

### Compositions, Formulations & Medicaments And Their Use

This disclosure further relates to compositions (i.e., formulations or medicaments) that can be used in the disclosed methods wherein the composition comprises at least one peptide of Formula A (e.g., a peptide of Formula: A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 or A-15-2), and optionally may also include one or more of the following additional therapeutic agents such as for example: (i) an angiotensin-converting enzyme (ACE) inhibitor, (ii) an angiotensin receptor blocker (ARB), (iii) a beta blocker, (iv) a diuretic, (v) a mineralocorticoid receptor antagonist or (vi) a calcium channel blocker (CCB). Such a composition can be formed, for example, by dissolving or suspending the selected compound(s)/peptide (or mixture of peptides) in water, buffer, detergent, excipient, organic solvent or a mixture of two or more of the foregoing. In some embodiments, the composition can be prepared by dissolving or suspending the selected compound(s)/peptide(s) in water. In some embodiments, the composition can be prepared by dissolving or suspending the selected compound(s)/peptide(s) in buffer (e.g., saline or phosphate buffered saline). In some embodiments, the composition can be prepared by dissolving or suspending the selected compound(s)/peptide(s) in excipient. In some embodiments, the composition can be prepared by dissolving or suspending the selected compound(s)/peptide(s) in a pharmaceutically acceptable carrier. In some embodiments, the composition is a formulation or is a medicament. Generally, as used herein the terms composition, formulation, and medicament are interchangeable. Other more specific types of formulations and methods for preparation are discussed in more detail below.

The peptide or mixture of peptides and optionally other therapeutic agents/drugs may be administered per se (neat) or in the form of a pharmaceutically acceptable salt. When used in medicine the salts may be pharmaceutically acceptable, but non-pharmaceutically acceptable salts may conveniently be used to prepare pharmaceutically acceptable salts thereof. When compounds/peptides of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts are known in the art and some are disclosed herein. When compounds/peptides of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts are known in the art and some are disclosed herein.

Certain specific compounds of the present disclosure may contain both basic and acidic functionalities that allow the compounds to be converted into either base or acid addition salts (see, e.g., FIG. 2, FIG. 3). These salts may be prepared by methods known to those skilled in the art. Other pharmaceutically acceptable carriers known to those of skill in the art are suitable for use with the present technology.

In some embodiments, the compositions/formulations can be used as medicaments for use in the practice of the methods disclosed herein.

The compositions (and methods of the present disclosure) may be used to treat an individual/subject in need thereof. In certain embodiments, the individual is a mammal such as a human, or a non-human mammal. When administered to an animal, such as a human, the composition or the compound/peptide(s) is/are preferably administered as a pharmaceutical composition comprising, for example, a peptide or mixture of peptides or optionally other therapeutic agent(s) and an excipient or pharmaceutically acceptable carrier. When the peptide or mixture of peptides is used in combination with other therapeutic agent(s), the other therapeutic agent(s) might, in some embodiments, be formulated in the same vehicle as the peptide or mixture of peptides or, in some embodiments, the peptide or mixture of peptides may be formulated independently based on, for example, the nature of those agents and the preferred administration route or timing of administration.

As stated above, an “effective amount” refers to any amount of the active compound/therapeutic agent (e.g., a peptide, mixture of peptides and/or optionally other therapeutic agent(s); alone or as formulated) that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic (i.e., preventative) or therapeutic treatment regimen can be planned which does not cause substantial unwanted toxicity and yet is effective to treat the particular condition or disease of a particular subject. The effective amount for any particular indication can vary depending on such factors as the disease or condition being treated, the particular compound of the present application being administered, the size of the subject, or the severity of the disease or condition. The effective amount may be determined during pre-clinical trials and/or clinical trials by methods familiar to physicians and clinicians. One of ordinary skill in the art can empirically determine the effective amount of a particular peptide or mixture of peptides of the present technology and/or other therapeutic agent(s) without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g., a pump).

For any compound/therapeutic agent/composition (e.g., a peptide or mixture of peptides) described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan. In some embodiments, the daily dose of a peptide of Formula A (e.g., the peptide of Formula A-1) for a subject can be from 0.1 mg/kg to 10 mg/kg (inclusive) of body weight, or be from 1 mg/kg to 10 mg/kg (inclusive) of body weight. In some embodiments, a human daily dose of a peptide of Formula A (e.g., the peptide of Formula A-1) can be from 1 mg to 60 mg administered subcutaneously.

Peptides/compounds (alone or as formulated in a pharmaceutical composition) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing of any of the animal model system known in the art can be used prior to administration to human subjects.

Dosage, toxicity and therapeutic efficacy of any therapeutic peptides, compounds, compositions (e.g., formulations or medicaments), other therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography, optionally coupled with mass spectroscopy detection (e.g. LC/MS).

The effective amount may be determined during pre-clinical trials and clinical trials by methods familiar to physicians and clinicians. An effective amount of the compound(s)/peptide(s) useful in the methods disclosed herein may be administered to a mammal in need thereof by any of a number of well-known methods for administering pharmaceutical compounds. The compound(s)/peptide(s) may be administered systemically or locally.

The skilled artisan will appreciate that certain factors may influence the dosage, mode of administration and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The compound(s)/peptide(s) of a composition (e.g., a formulation or medicament) can be administered by any known or future developed mode of administration. For example, administration can be oral. Administration can be systemic. Administration can be subcutaneous. Administration can be intravenous. Administration can be topical. Administration can be intraperitoneal. Administration can be intradermal. Administration can be transdermal. Administration can be ophthalmical. Administration can be retro-orbital. Administration can be intrathecal. Administration can be intracerebroventricular. Administration can be iontophoretical. Administration can be transmucosal. Administration can be intravitreal Administration can be intranasal Administration can be intramuscular. In some embodiments, peptide, mixture of peptides and/or the other therapeutic agent(s) are separately, sequentially or simultaneously administered. In some embodiments, administration of the peptide or mixture of peptides in combination with other therapeutic agents produces a synergistic effect.

In some embodiments, the peptide or mixture of peptides of a composition and optionally other therapeutic agent(s) is/are administered to the subject for 6 weeks or more. In some embodiments, the peptide, mixture of peptides and/or optionally other therapeutic agent(s) is/are administered to the subject for 12 weeks or more. In some embodiments, the peptide, mixture of peptides and/or optionally other therapeutic agent(s) is/are administered to the subject for 24 weeks or more. In some embodiments, the peptide, mixture of peptides and/or optionally other therapeutic agent(s) is/are administered to the subject for 48 weeks or more. In some embodiments, the peptide mixture of peptides and/or optionally other therapeutic agent(s) is/are administered to the subject for 72 weeks or more. In some embodiments, the peptide, mixture of peptides and/or optionally other therapeutic agent(s) is/are administered to the subject for 96 weeks or more. In some embodiments, the peptide, mixture of peptides and/or optionally other therapeutic agent(s) is/are administered to the subject for 1 year or more. In some embodiments, the peptide, mixture of peptides and/or optionally other therapeutic agent(s) is/are administered to the subject for 2 years or more. In some embodiments, the peptide, mixture of peptides and/or optionally other therapeutic agent(s) is/are administered to the subject for 3 years or more. In some embodiments, the peptide, mixture of peptides and/or optionally other therapeutic agent(s) is/are administered until no continued therapeutic benefit is observed. In some embodiments, the peptide, mixture of peptides and/or optionally other therapeutic agent(s) is/are administered until the end of life of the subject.

The pharmaceutical compositions (e.g., a formulation or medicament) can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 2, 3, 4, 5, 6, 7 days, weeks, months or more of treatment).

Systemic formulations include those designed for administration by injection, e.g., subcutaneous, intravenous, intramuscular, intrathecal or intraperitoneal injection, as well as those designed for transdermal, transmucosal oral or pulmonary administration.

For intravenous and other parenteral routes of administration, a compound (e.g., a peptide or mixture of peptides, and optionally other therapeutic agent(s) of the present technology can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and must be preserved against the contaminating action of microorganisms such as bacteria and fungi.

Sterile injectable solutions (e.g., a formulation or medicament) can be prepared by incorporating the active compound (e.g., a peptide, mixture of peptides, and/or optionally other therapeutic agent(s)) in the required amount in an appropriate solvent with one or a combination of ingredients enumerated above, as required, followed by filtered sterilization. Generally, dispersions are prepared by incorporating the active compound into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof. Sterilization can also be accomplished by exposure of the formulation or medicament to gamma radiation.

The therapeutic compounds (e.g., a peptide, a mixture of peptides, and/or optionally other therapeutic agent(s)) of the composition(s), when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions for parenteral administration include aqueous solutions of the active compounds (e.g., a peptide, a mixture of peptides, or optionally other therapeutic agent(s)) in water-soluble form. Additionally, suspensions of the therapeutic compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the therapeutic compounds to allow for the preparation of highly concentrated solutions.

For oral administration, the compounds (e.g., a peptide, mixture of peptides, or optionally other therapeutic agent(s)) of the composition(s), can be formulated readily by combining the active compound(s) with pharmaceutically acceptable carriers well known in the art. Such carriers enable the compounds of the present application to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above may be chemically modified so that oral delivery of the derivative is efficacious. Generally, the chemical modification contemplated is the attachment of at least one moiety to the therapeutic agent(s), ingredient(s), and/or excipient(s), where said moiety permits: (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of the compound of the present application (or derivative) or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound (e.g., a peptide, mixture of peptides, or optionally other therapeutic agent(s)) or pharmaceutical composition thereof can be included in the formulation as fine multi-particulates in the form of granules or pellets of particle size about 1-2 mm. The formulation of the material for capsule administration could also be as a powder, lightly compressed plugs or even as tablets. The therapeutic compound or pharmaceutical composition could be prepared by compression.

Colorants and flavoring agents may all be included. For example, the compound or pharmaceutical composition of the present application (or derivative) may be formulated and then further contained within an edible product, such as a refrigerated beverage containing colorants and flavoring agents.

One may dilute or increase the volume of the therapeutic compound or pharmaceutical composition with an inert material. These diluents could include carbohydrates, especially mannitol, «-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound or composition into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, fumed silica, pyrogenic silica and hydrated silicoaluminate.

To aid dissolution of the therapeutic compound (e.g., a peptide, mixture of peptides, or optionally other therapeutic agent(s)) or composition thereof into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the present application or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The compounds, peptides, peptide mixtures, or optionally other therapeutic agent(s), and compositions thereof disclosed herein can be included in a formulation as fine multi-particulates in the form of granules or pellets of particle size about 1 mm. The formulation of the material for capsule administration could also be as a powder, lightly compressed plugs or even as tablets. The formulation could be prepared by compression.

For buccal administration, the compositions may take the form of tablets or lozenges formulated in conventional manner.

For topical administration, a peptide, mixture of peptides, or optionally other therapeutic agent(s)) may be formulated as solutions, gels, ointments, creams, suspensions, etc., as are well-known in the art.

For administration by inhalation, a peptide, mixture of peptides, or optionally other therapeutic agent(s) or compositions thereof (e.g. medicament) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament or therapeutic compound can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound and a suitable powder base such as lactose or starch.

Nasal delivery of a therapeutic compound (e.g. a peptide, mixture of peptides, or optionally other therapeutic agent(s)) or pharmaceutical composition of the present application is also contemplated. Nasal delivery allows the passage of a therapeutic compound or pharmaceutical composition of the present application to the blood stream directly after administering the therapeutic compound or pharmaceutical composition to the nose, without the necessity for deposition of the product in the lung. Formulations for nasal delivery include those with dextran or cyclodextran.

For nasal administration, a useful device is a small, hard bottle to which a metered dose sprayer is attached. In some embodiments, the metered dose is delivered by drawing the pharmaceutical composition of the present application solution into a chamber of defined volume, which chamber has an aperture dimensioned to aerosolize and aerosol formulation by forming a spray when a liquid in the chamber is compressed. The chamber is compressed to administer the therapeutic compound or pharmaceutical composition. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound or pharmaceutical composition.

Alternatively, the therapeutic compound (e.g. a peptide, mixture of peptides, or optionally other therapeutic agent(s)) or pharmaceutical composition may be in powder form for constitution with a suitable vehicle, e.g., sterile pyrogen-free water, before use.

Also contemplated herein is pulmonary delivery of the compounds, peptide mixture of peptides, or optionally other therapeutic agents disclosed herein (or salts, hydrates, solvates and/or tautomers thereof). The peptide, mixture of peptides, or optionally other therapeutic agent(s) are delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5):143-146 (1989) (endothelin-1); Hubbard et al., Annal Int Med 3:206-212 (1989) (antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of a peptide, mixture of peptides, or optionally other therapeutic agent(s) disclosed herein. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro. Chemically modified compound may also be prepared in different formulations depending on the type of chemical modification or the type of device employed.

Formulations suitable for use with a nebulizer, either jet or ultrasonic, will typically comprise a peptide, mixture of peptides, or optionally other therapeutic agent(s) disclosed herein dissolved in water at a concentration of about 0.1 to 25 mg of biologically active compound (e.g., a peptide, mixture of peptides, or optionally other therapeutic agent(s)) per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing a peptide, mixture of peptides, or optionally other therapeutic agent(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing a peptide, mixture of peptides, or optionally other therapeutic agent(s) disclosed herein and may also include a bulking agent, such as lactose, sorbitol, sucrose, trehalose, or mannitol in amounts which facilitate dispersal of the powder from the device, e.g., 50 to 90% by weight of the formulation. The compound (or derivative) should advantageously be prepared in particulate form with an average particle size of less than 10 micrometers (μm), most preferably 0.5 to 5 μm, for most effective delivery to the deep lung.

In addition to the formulations described above, a peptide, mixture of peptides, or optionally other therapeutic agent(s) may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The peptide, mixture of peptides, or optionally other therapeutic agent(s) may be provided in particles. Particles as used herein means nanoparticles or microparticles/microspheres (or in some instances larger particles) which can consist in whole or in part of the compound or the other therapeutic agent(s) as described herein. Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt. The particles may contain the therapeutic agent(s) in a core surrounded by a coating, including, but not limited to, an enteric coating. The peptide, mixture of peptides, or optionally other therapeutic agent(s) also may be dispersed throughout the particles. The peptide, mixture of peptides, or optionally other therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the peptide, mixture of peptides, or optionally other therapeutic agent(s), any of those materials routinely used in the art of pharmacy and medicine, including, but not limited to, erodible, nonerodable, biodegradable, or nonbiodegradable material or combinations thereof. The particles may be microcapsules which contain the compound in a solution or in a semi-solid state. The particles may be of virtually any shape.

Both non-biodegradable and biodegradable polymeric materials can be used in the manufacture of particles for delivering the peptide, mixture of peptides, or optionally other therapeutic agent(s). Such polymers may be natural or synthetic polymers. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and polycaprolactone.

A therapeutic compound (e.g., a peptide, mixture of peptides, or optionally other therapeutic agent(s)) or other therapeutic agent or mixtures thereof can be formulated in a carrier system. The carrier can be a colloidal system. The carrier or colloidal system can be a liposome, a phospholipid bilayer vehicle. In one embodiment, therapeutic compound or other therapeutic agent or mixtures thereof can be encapsulated in a liposome while maintaining integrity of the therapeutic compound or other therapeutic agent or mixtures thereof. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, an active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound (e.g., a peptide, mixture of peptides, or optionally other therapeutic agent(s)) or other therapeutic agent or mixtures thereof can be embedded in the polymer matrix, while maintaining integrity of the composition. The polymer can be a microparticle or nanoparticle that encapsulates the therapeutic agent or agents. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or poly lactic/glycolic acid (PLGA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compound (e.g., a peptide, mixture of peptides, or optionally other therapeutic agent(s)) or other therapeutic agent or mixtures thereof are prepared with carriers that will protect the therapeutic compound, other therapeutic agent or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provide the long-term sustained release occurring from the aggregate of the plurality of compositions. “Long-term” release, as used herein, means that the implant or depot formulation is constructed and arranged to deliver therapeutic or prophylactic levels of the active ingredient for at least 2 days. In some embodiments, the implant or depot formulation is constructed and arranged to deliver therapeutic or prophylactic levels of the active ingredient for at least 7 days. In some embodiments, the implant or depot formulation is constructed and arranged to deliver therapeutic or prophylactic levels of the active ingredient for at least 14 days. In some embodiments, the implant or depot formulation is constructed and arranged to deliver therapeutic or prophylactic levels of the active ingredient for at least 30 days. In some embodiments, the implant or depot formulation is constructed and arranged to deliver therapeutic or prophylactic levels of the active ingredient for at least 60 days. In some embodiments, the implant or depot formulation is constructed and arranged to deliver therapeutic or prophylactic levels of the active ingredient for at least 90 days. In some embodiments, the implant or depot formulation is constructed and arranged to deliver therapeutic or prophylactic levels of the active ingredient for at least 180 days. In some embodiments, the implant or depot formulation is constructed and arranged to deliver therapeutic or prophylactic levels of the active ingredient for at least one year. In some embodiments, the implant or depot formulation is constructed and arranged to deliver therapeutic or prophylactic levels of the active ingredient for 15-30 days. In some embodiments, the implant or depot formulation is constructed and arranged to deliver therapeutic or prophylactic levels of the active ingredient for 30-60 days. In some embodiments, the implant or depot formulation is constructed and arranged to deliver therapeutic or prophylactic levels of the active ingredient for 60-90 days. In some embodiments, the implant or depot formulation is constructed and arranged to deliver therapeutic or prophylactic levels of the active ingredient for 90-120 days. In some embodiments, the implant or depot formulation is constructed and arranged to deliver therapeutic or prophylactic levels of the active ingredient for 120-180 days. In some embodiments, the long-term sustained release implants or depot formulation are well-known to those of ordinary skill in the art and include some of the release systems described above. In some embodiments, such implants or depot formulation can be administered surgically. In some embodiments, such implants or depot formulation can be administered topically or by injection.

It will be understood by one of ordinary skill in the relevant arts that other suitable modifications and adaptations to the compositions and methods described herein are readily apparent from the description of the present technology contained herein in view of information known to the ordinarily skilled artisan, and may be made without departing from the scope of the present application or any embodiment thereof.

In view of the foregoing, this disclosure also pertains to a composition or medicament for use in treating, preventing, inhibiting, ameliorating or delaying the onset of heart failure (HF), idiopathic cardiomyopathy (ICM), dilated cardiomyopathy (DCM), or restrictive cardiomyopathy (RCM) in a mammalian subject in need thereof, wherein the composition or medicament comprises a peptide of Formula A, or a mixture of peptides of Formula A:

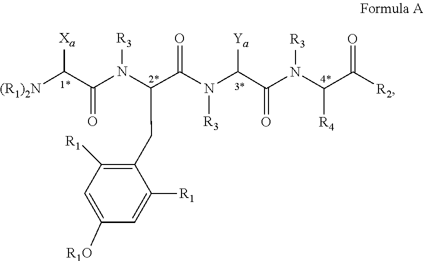

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; R₃ is independently H, or —CH₃; R₄ is selected from:

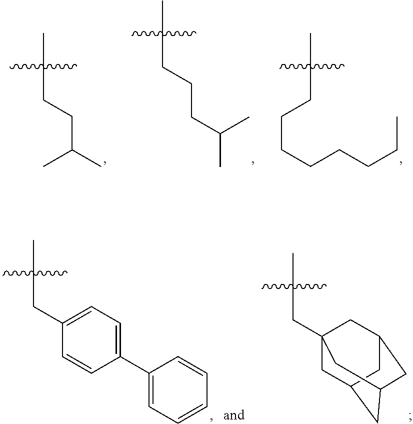

Xₐ is selected from

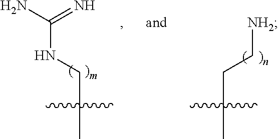

Yₐ is selected from

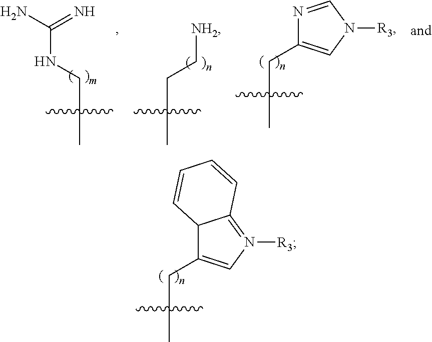

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, the peptide of Formula A is/are a peptide/peptides of Formula A-1 or Formula A-2:

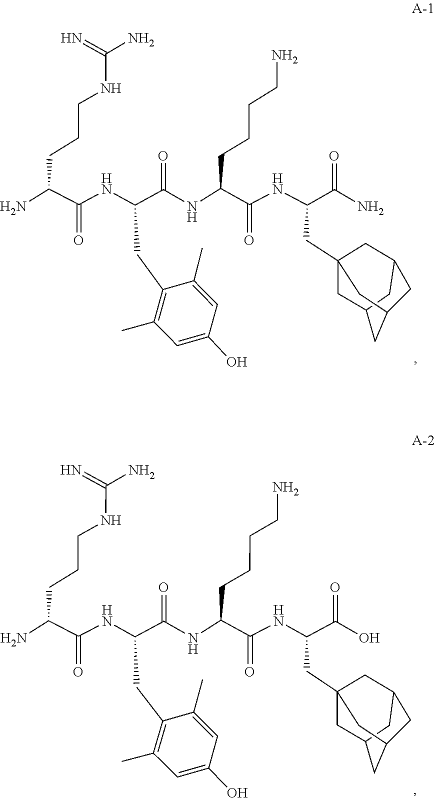 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

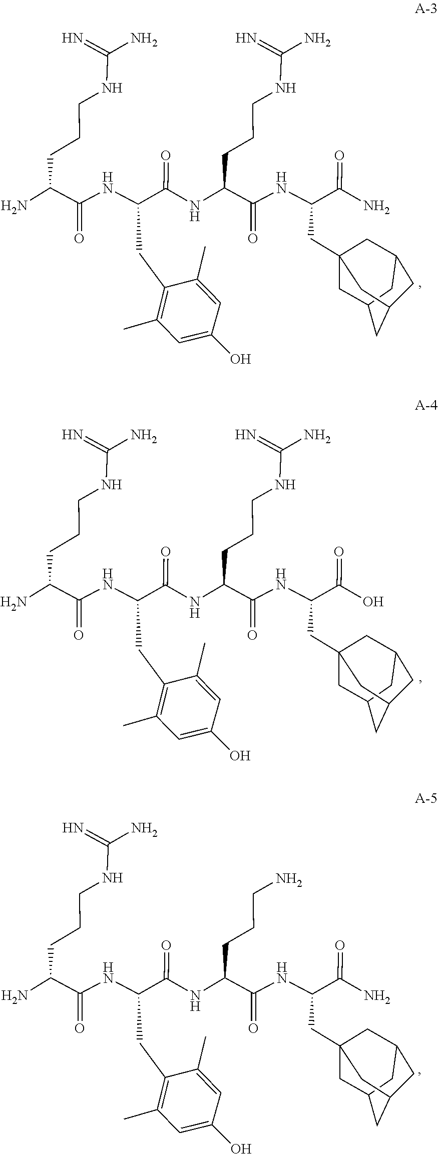 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

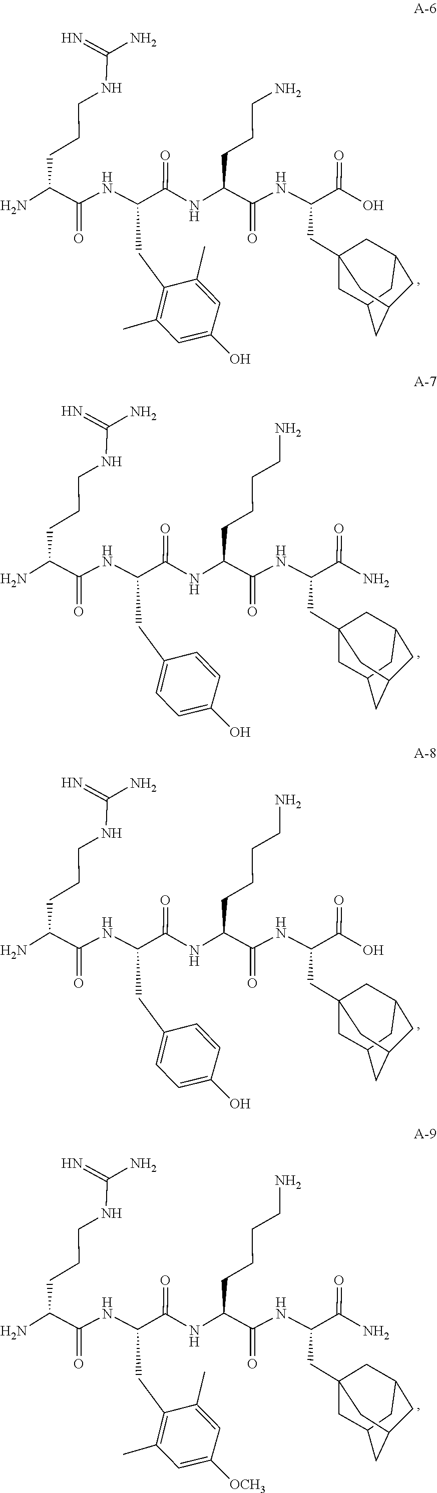 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1]

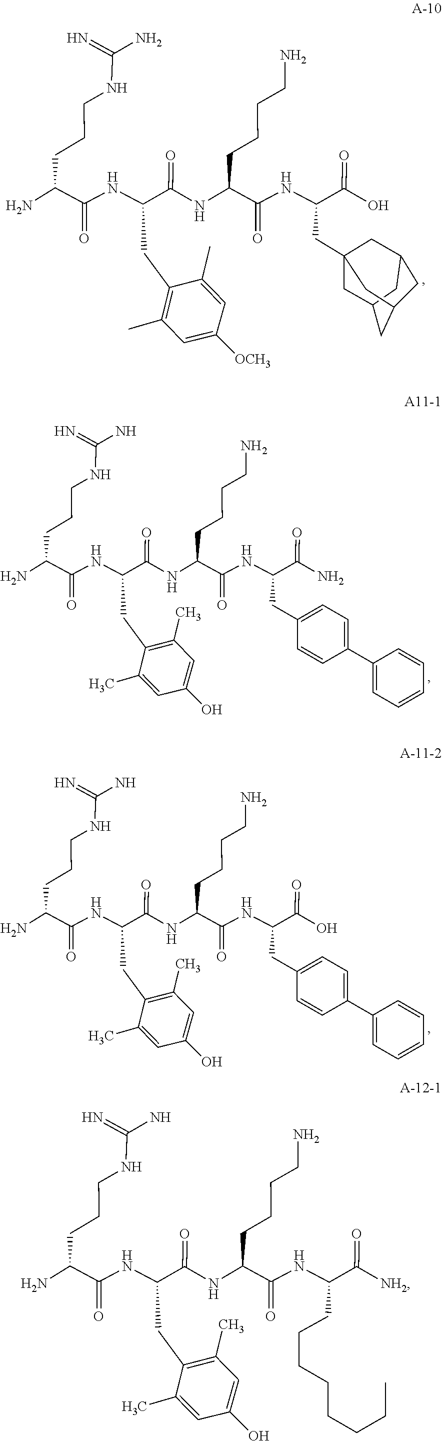 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

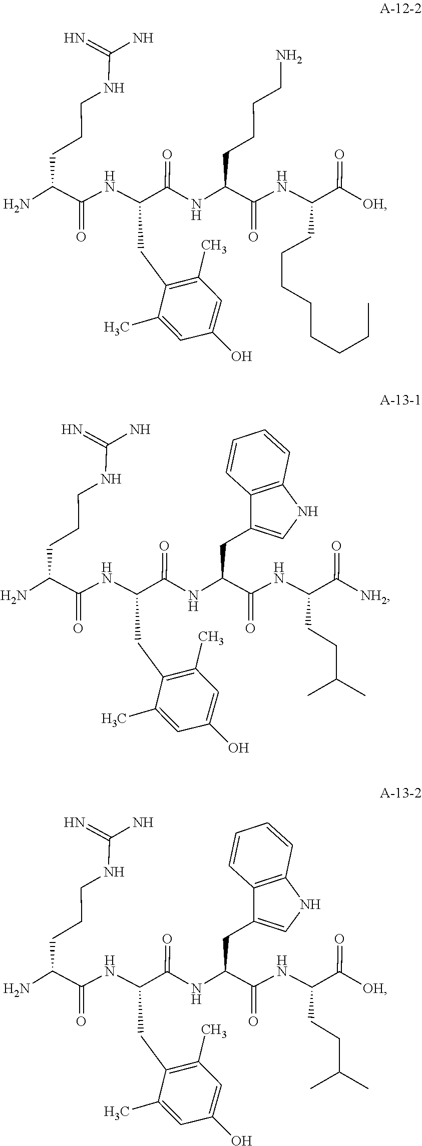 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(=O)O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O]

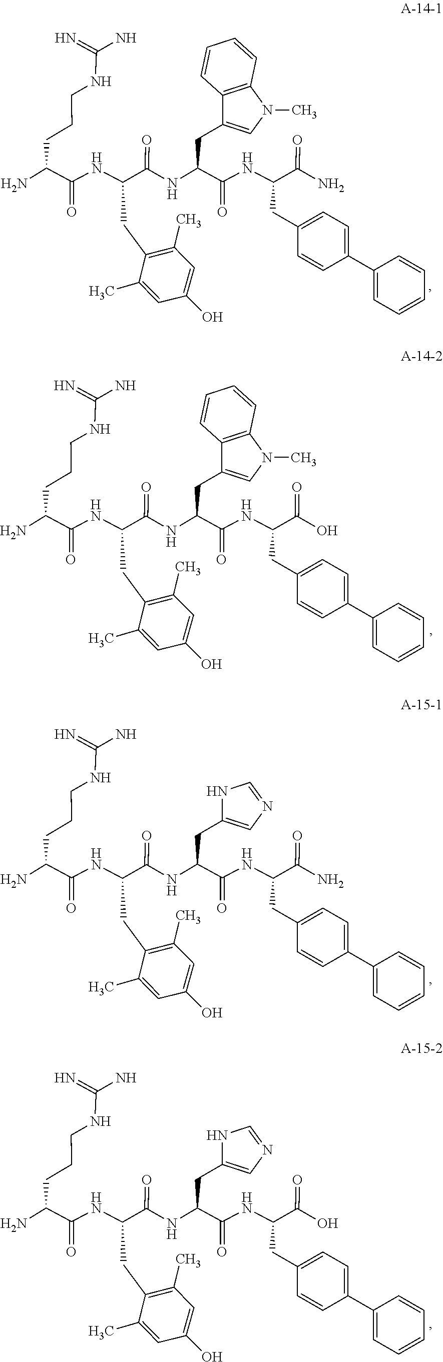 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the composition or medicament provides for treating, preventing, inhibiting, ameliorating or delaying the onset of heart failure (HF). In some embodiments, the composition or medicament provides for treating, preventing, inhibiting, ameliorating or delaying the onset of idiopathic cardiomyopathy (ICM). In some embodiments, the composition or medicament provides for treating, preventing, inhibiting, ameliorating or delaying the onset of dilated cardiomyopathy (DCM). In some embodiments, the composition or medicament provides for treating, preventing, inhibiting, ameliorating or delaying the onset of restrictive cardiomyopathy (RCM). In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the composition or medicament, the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the composition or medicament, the heart failure is congestive heart failure (CHF).

In some embodiments of the composition or medicament, administration of the composition or medicament to the subject; (i) ameliorates, (ii) delays the onset of, and/or (iii) delays the progression of one or more signs or symptoms of heart failure in the subject as compared with a control subject or group of control subjects to which the peptide or peptides was/were not administered. In some embodiments of the composition or medicament, the signs or symptoms of heart failure are: (i) shortness of breath, (ii) ankle and/or foot swelling; (iii) leg swelling; (iv) fatigue, and/or (v) labored breathing when lying flat.

In some embodiments of the composition or medicament, the composition or medicament comprising the peptide is administered daily for: (i) 12 weeks or more; (ii) 24 weeks or more; (iii) 48 weeks or more; (iv) 72 weeks or more; or (v) 96 weeks or more. In some embodiments of the method, the composition or medicament comprising the peptide is administered subcutaneously or intravenously. In some embodiments of the composition or medicament, the composition or medicament comprising the peptide is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide of the composition or medicament is formulated for administration from its tris-HCl salt.

In another aspect, this disclosure pertains to the use of a peptide, mixture of peptides, or a composition or medicament comprising the peptide or mixture of peptides, for treating, preventing, inhibiting, ameliorating or delaying the onset of heart failure (HF), idiopathic cardiomyopathy (ICM), dilated cardiomyopathy (DCM), or restrictive cardiomyopathy (RCM) in a mammalian subject in need thereof, wherein the peptide is a peptide of Formula A, or a mixture of peptides of Formula A:

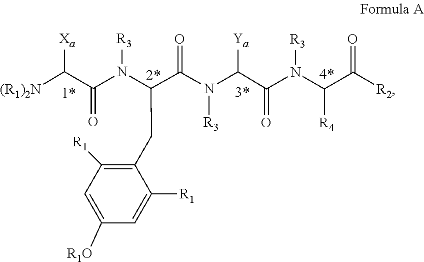

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

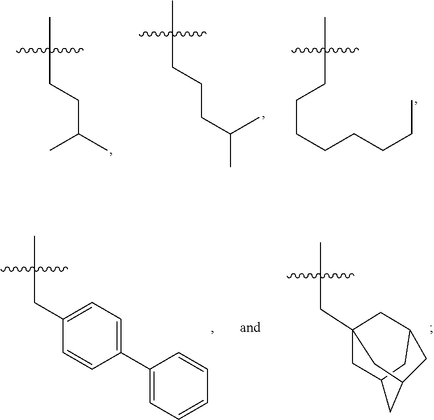

Xₐ is selected from

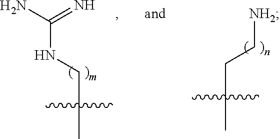

Yₐ is selected from

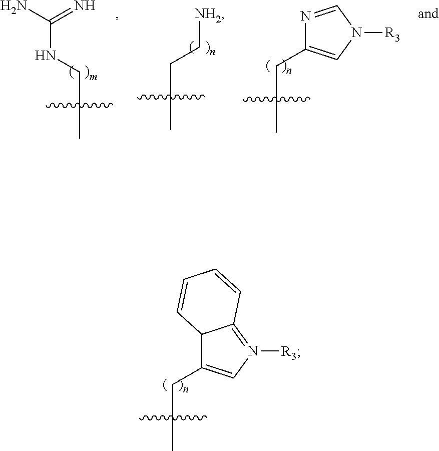

each m is independently 1, 2, 3 or 4; each n is independently 1, 2, or 3; and the absolute stereochemistry at each of stereocenters 1*, 2*, 3*, and 4* is independently R or S, and optionally one or more hydrogen atoms of the peptide of Formula A is substituted with a deuterium or fluorine atom. In some embodiments of the use, the peptide or peptides of Formula A is/are a peptide/peptides of Formula A-1 or Formula A-2:

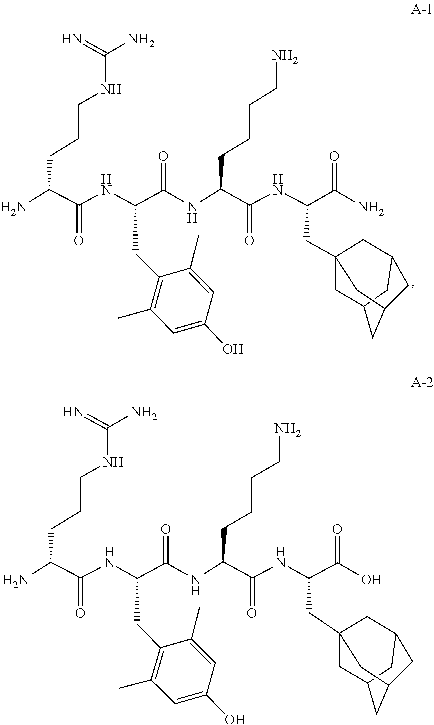 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

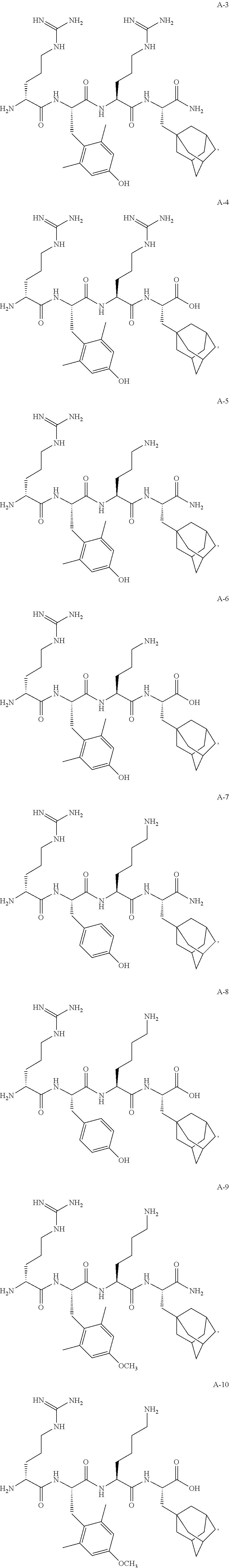

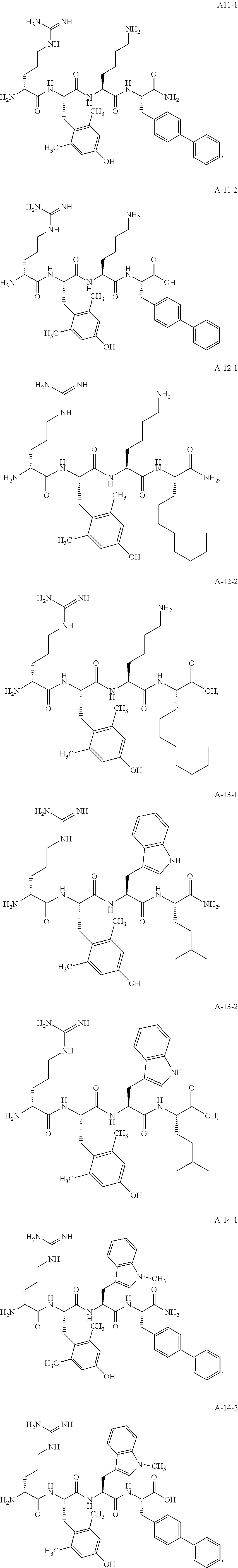

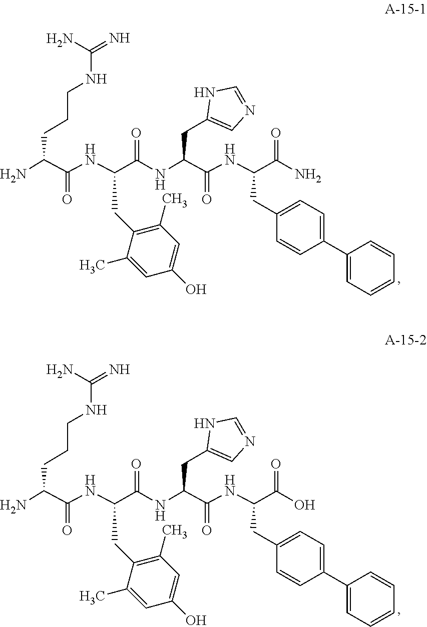 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments of the use, the peptide, mixture of peptides, or the composition or medicament comprising the peptide or mixture of peptides, provides for treating, preventing, inhibiting, ameliorating or delaying the onset of heart failure (HF). In some embodiments of the use, the peptide, mixture of peptides, or the composition or medicament comprising the peptide or mixture of peptides, provides for treating, preventing, inhibiting, ameliorating or delaying the onset of idiopathic cardiomyopathy (ICM). In some embodiments of the use, the peptide, mixture of peptides, or the composition or medicament comprising the peptide or mixture of peptides, provides for treating, preventing, inhibiting, ameliorating or delaying the onset of dilated cardiomyopathy (DCM). In some embodiments of the use, the peptide, mixture of peptides, or the composition or medicament comprising the peptide or mixture of peptides, provides for treating, preventing, inhibiting, ameliorating or delaying the onset of restrictive cardiomyopathy (RCM). In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the use, the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure. In some embodiments of the use, the heart failure is congestive heart failure (CHF).

In some embodiments of the use, administration of the peptide, mixture of peptides, or the composition comprising the peptide or mixture of peptides to the subject; (i) ameliorates, (ii) delays the onset of, and/or (iii) delays the progression of one or more signs or symptoms of heart failure in the subject as compared with a control subject or group of control subjects to which the peptide or peptides was/were not administered. In some embodiments of the use, the signs or symptoms of heart failure are: (i) shortness of breath, (ii) ankle and/or foot swelling; (iii) leg swelling; (iv) fatigue, or (v) labored breathing when lying flat.

In some embodiments of the use, wherein the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides is administered subcutaneously or intravenously. In some embodiments of the use, the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or peptides comprises hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its tris-HCl salt.

In another aspect, this disclosure pertains to a composition or medicament for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, wherein the composition or medicament comprises a peptide of Formula A, or a mixture of peptides of Formula A:

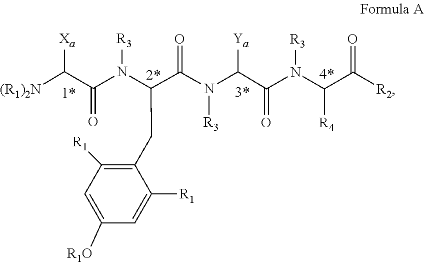

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; R₃ is independently H, or —CH₃; R₄ is selected from:

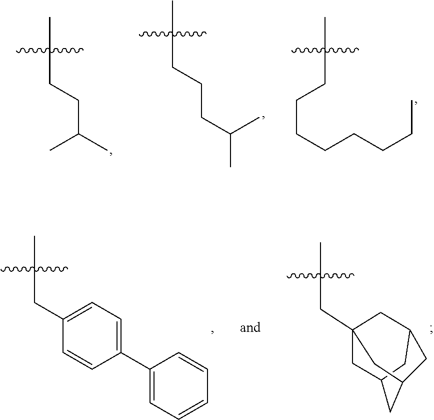

Xₐ is selected from

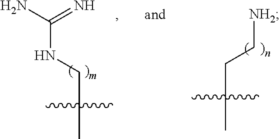

Yₐ is selected from

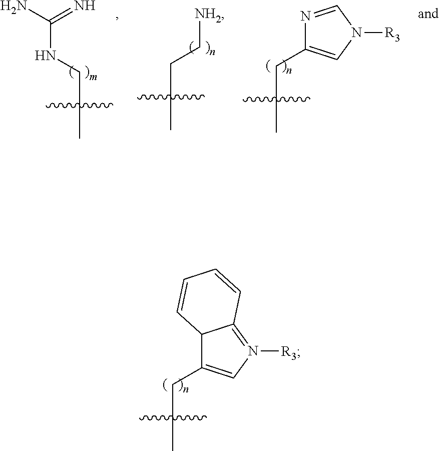

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, the peptide or peptides of Formula A is/are a peptide/peptides of Formula A-1 or Formula A-2:

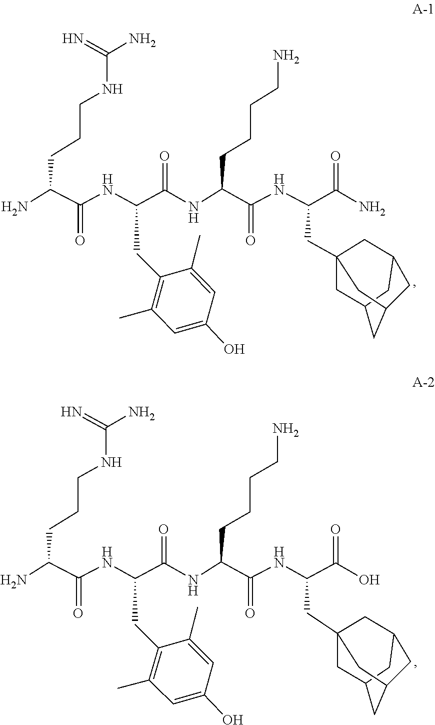 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

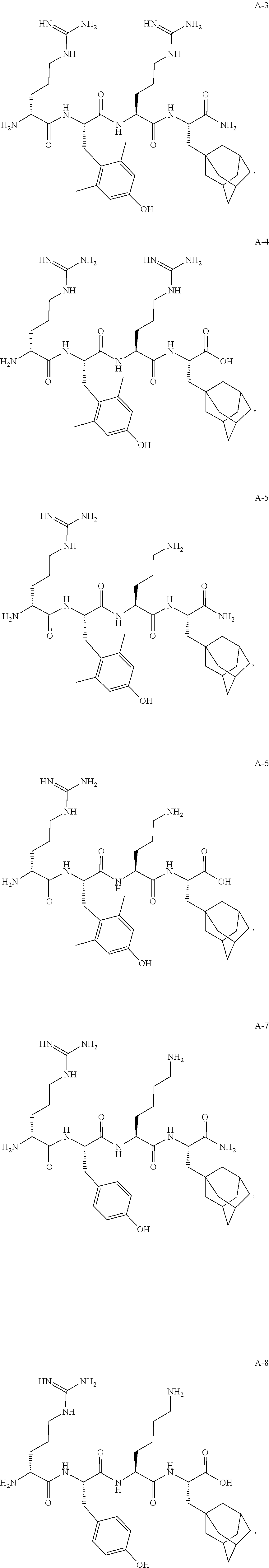

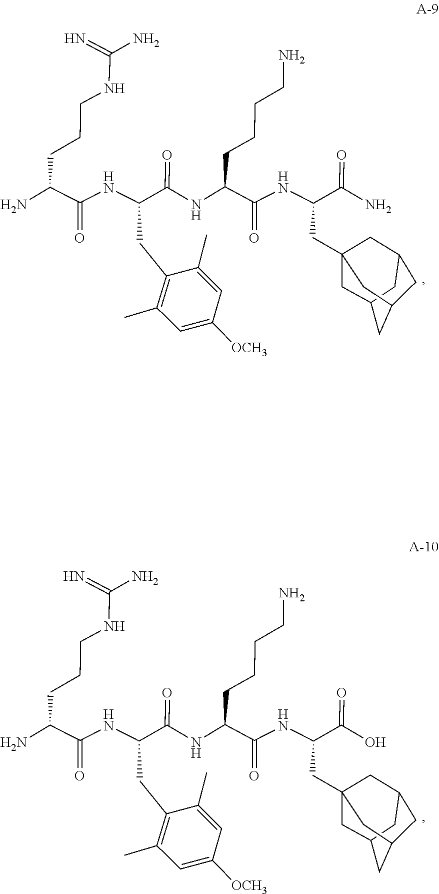 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

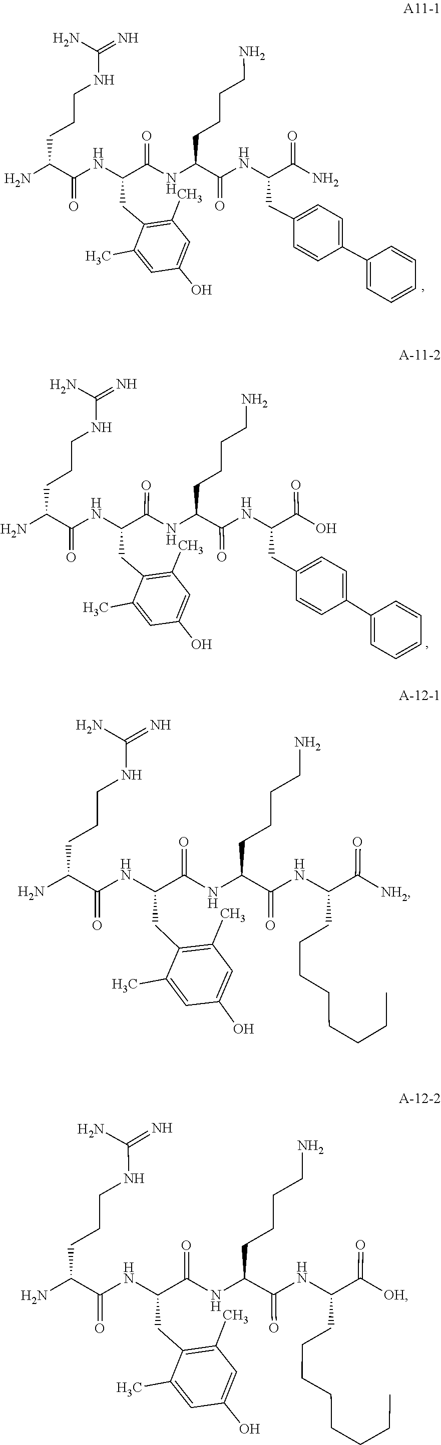 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

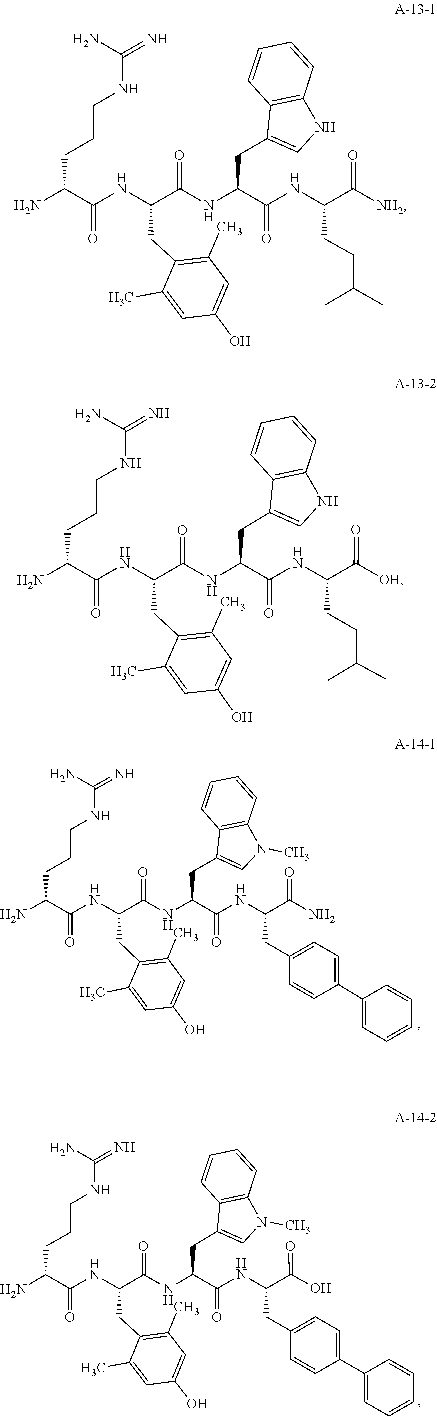 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

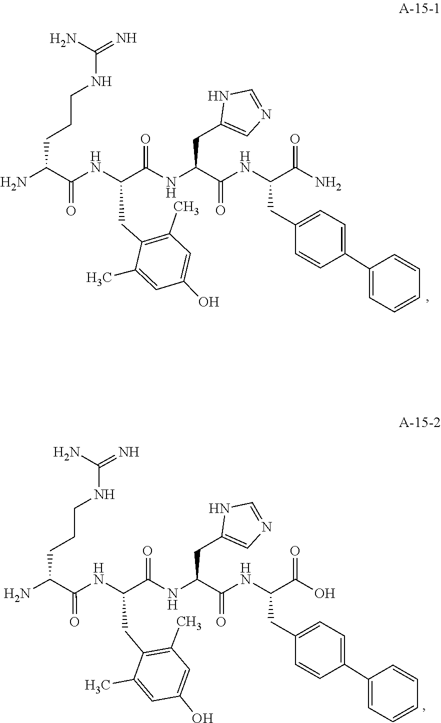 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the composition or medicament provides for treating, preventing, inhibiting, ameliorating or delaying the onset of hypertrophic cardiomyopathy (including obstructive hypertrophic cardiomyopathy). In some embodiments, the composition or medicament provides for treating, preventing, inhibiting, ameliorating or delaying the onset of hypertensive cardiomyopathy. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the composition or medicament, the composition or medicament comprising the peptide is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament comprising the peptide or peptides is administered subcutaneously or intravenously. In some embodiments of the composition or medicament, the composition or medicament comprising the peptide or peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its tris-HCl salt.

In another aspect, this disclosure relates to the use of a peptide, mixture of peptides, or a composition or medicament comprising the peptide or mixture of peptides, for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, wherein the peptide or peptides is/are a peptide of Formula A, or a mixture of peptides of Formula A:

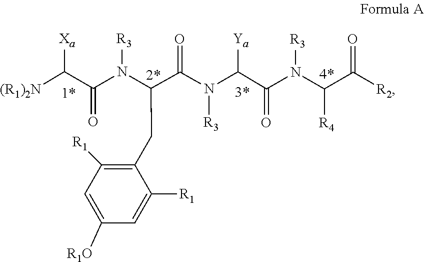

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

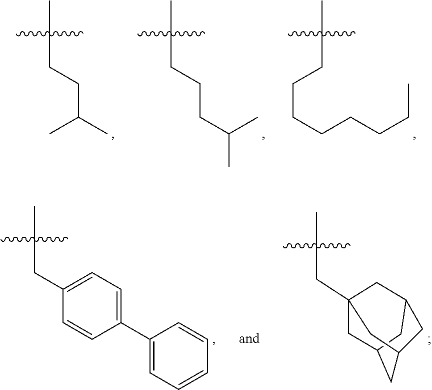

Xₐ is selected from

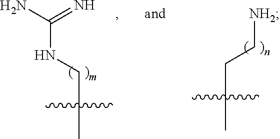

Yₐ is selected from

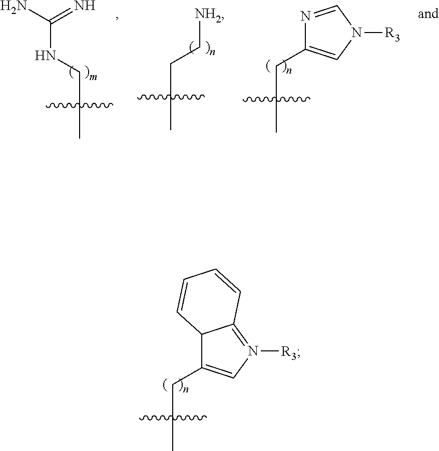

each m is independently 1, 2, 3 or 4; each n is independently 1, 2, or 3; and the absolute stereochemistry at each of stereocenters 1*, 2*, 3*, and 4* is independently R or S, and optionally one or more hydrogen atoms of the peptide of Formula A is substituted with a deuterium or fluorine atom. In some embodiments of the use, the peptide or peptides of Formula A is/are a peptide/peptides of Formula A-1 or Formula A-2:

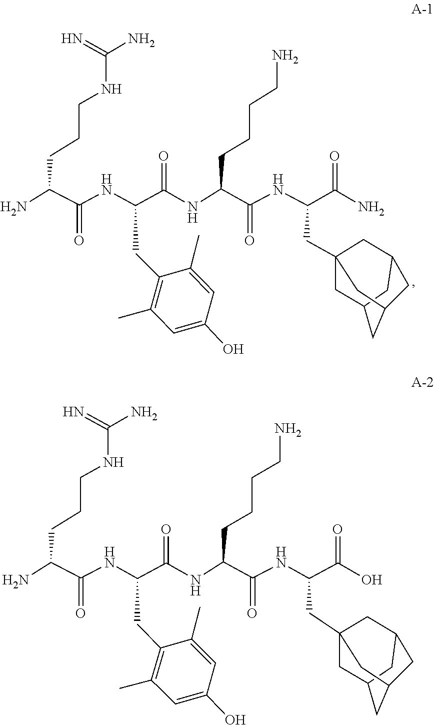 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

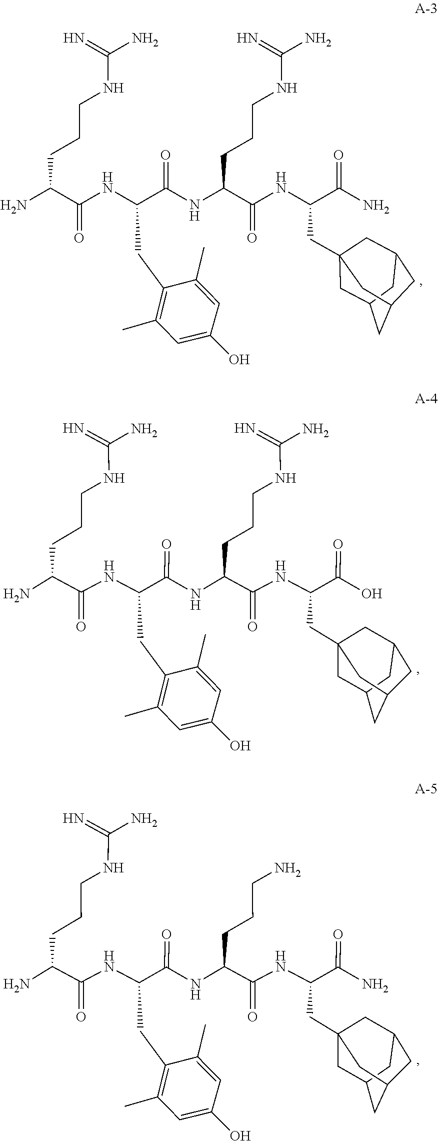 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

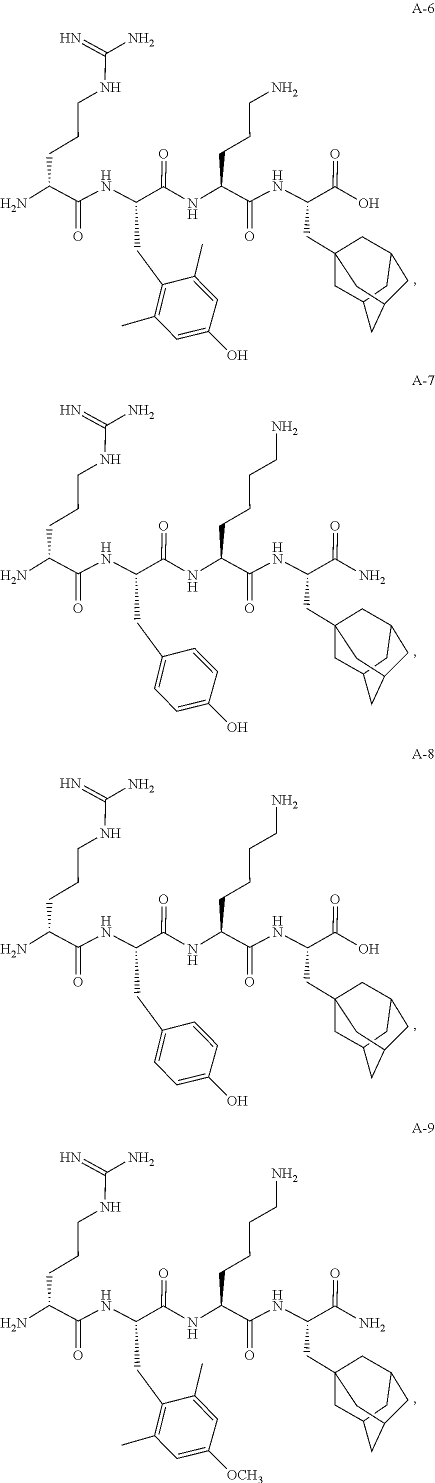 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1]

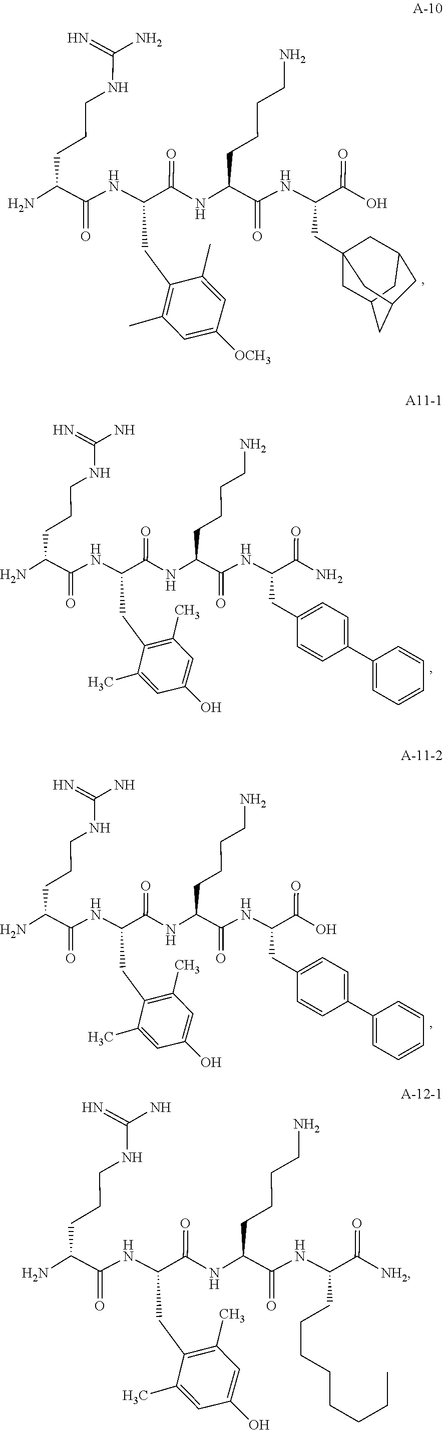 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O]

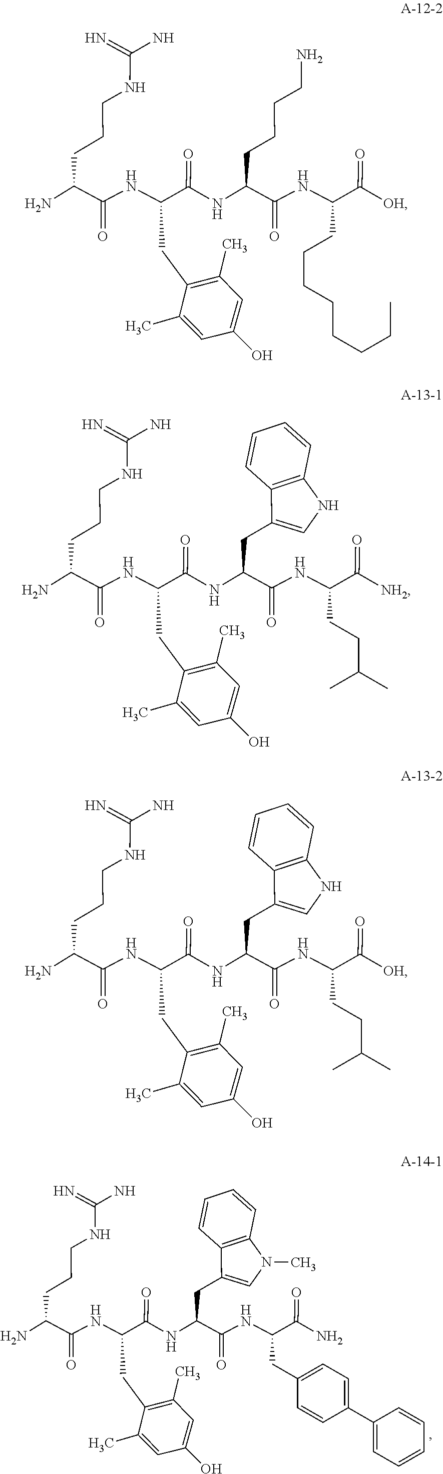 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(=O)O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

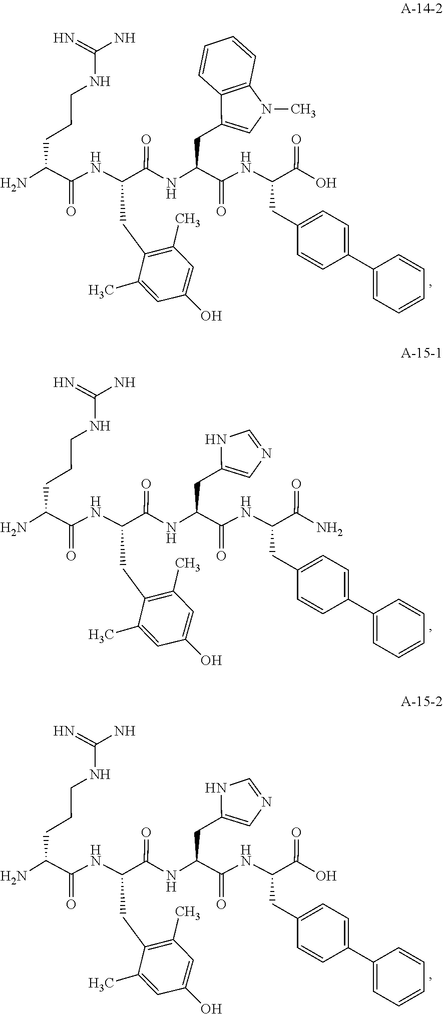 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the peptide, mixture of peptides, or the composition or medicament comprising the peptide or mixture of peptides, provides for treating, preventing, inhibiting, ameliorating or delaying the onset of hypertrophic cardiomyopathy (including obstructive hypertrophic cardiomyopathy). In some embodiments, the peptide, mixture of peptides, or the composition or medicament comprising the peptide or mixture of peptides, provides for treating, preventing, inhibiting, ameliorating or delaying the onset of hypertensive cardiomyopathy. In some embodiments, one or more hydrogen atoms of the peptide or peptides of Formulas A (e.g., peptides of Formulas: A-1, A-2, A-3, A-4, A-5, A-6, A-7, A-8, A-9, A-10, A-11-1, A-11-2, A-12-1, A-12-2, A-13-1, A-13-2, A-14-1, A14-2, A-15-1, A-15-2) are optionally substituted with a deuterium or fluorine atom.

In some embodiments of the use, the peptide, mixture of peptides, or the composition or medicament comprising the peptide or mixture of peptides, is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides, is administered subcutaneously or intravenously. In some embodiments of the use, the peptide mixture of peptides, or the composition or medicament comprising the peptide or mixture of peptides, is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or mixture of peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its tris-HCl salt.

In another aspect, this disclosure relates to a composition or medicament for normalizing left ventricular ejection fraction, fractional shortening, and/or global longitudinal strain in a mammalian subject in need thereof, wherein the composition or medicament comprises a peptide of Formula A, or a mixture of peptides of Formula A:

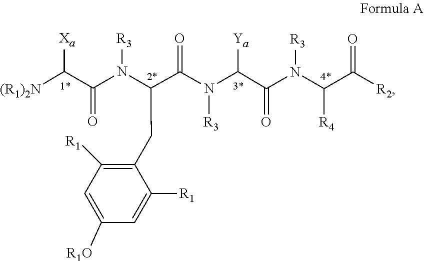

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

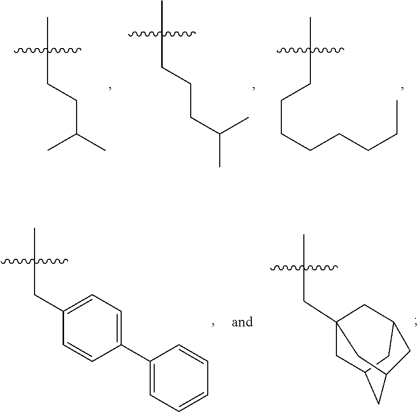

Xₐ is selected from

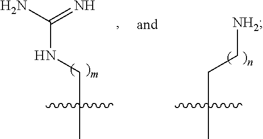

Yₐ is selected from

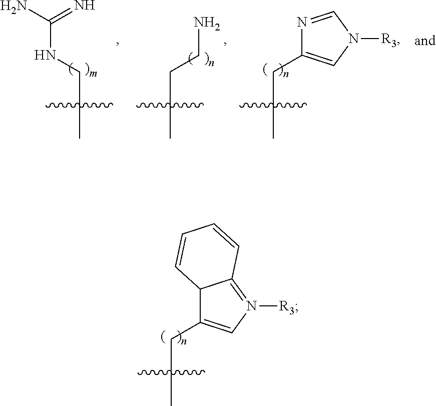

each m is 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom. In some embodiments, the peptide or peptides of Formula A is/are a peptide/peptides of Formula A-1 or Formula A-2:

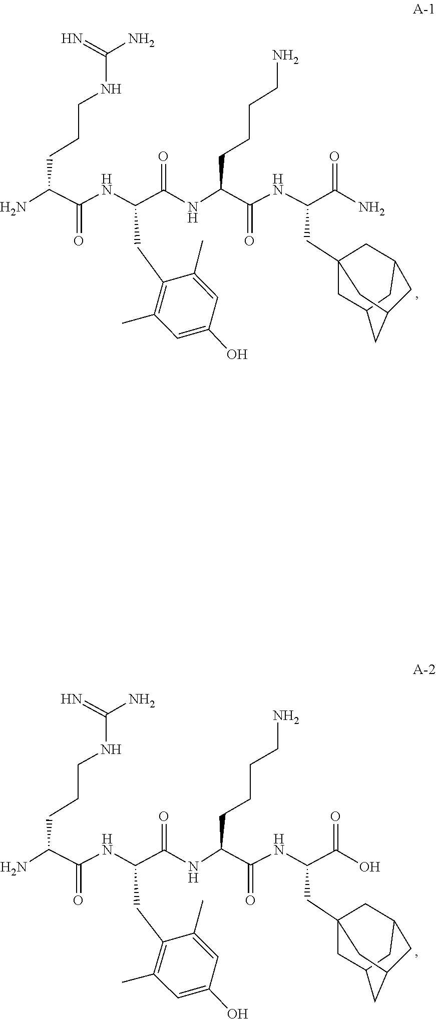 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

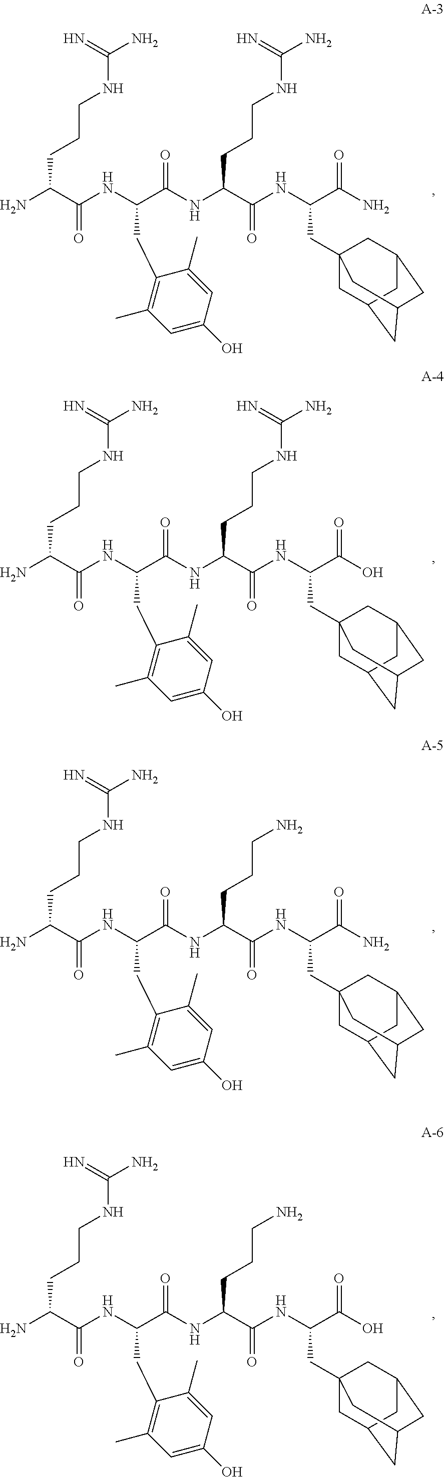 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O]

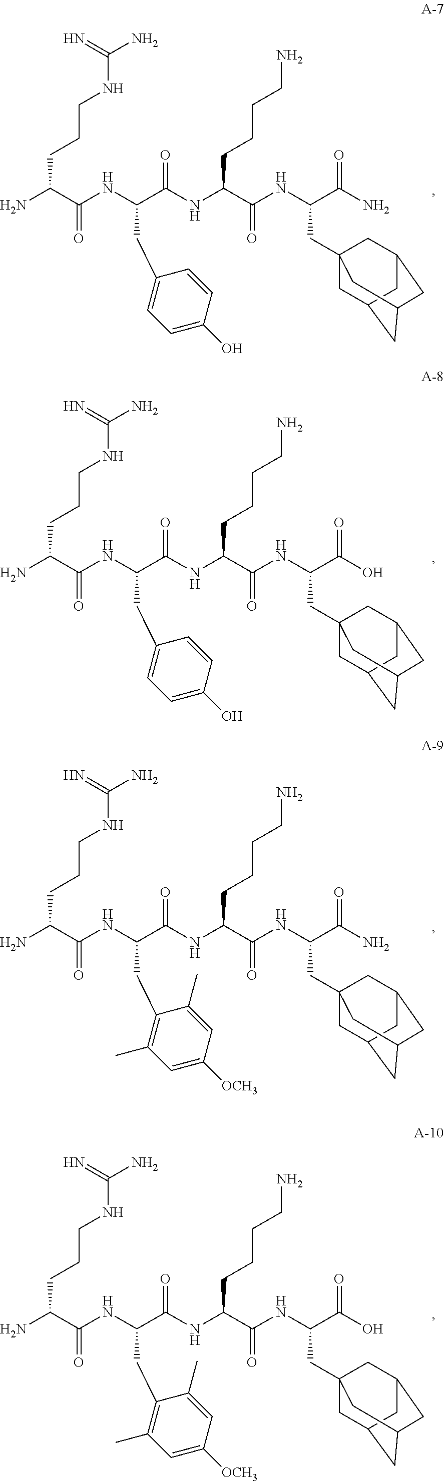 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1]

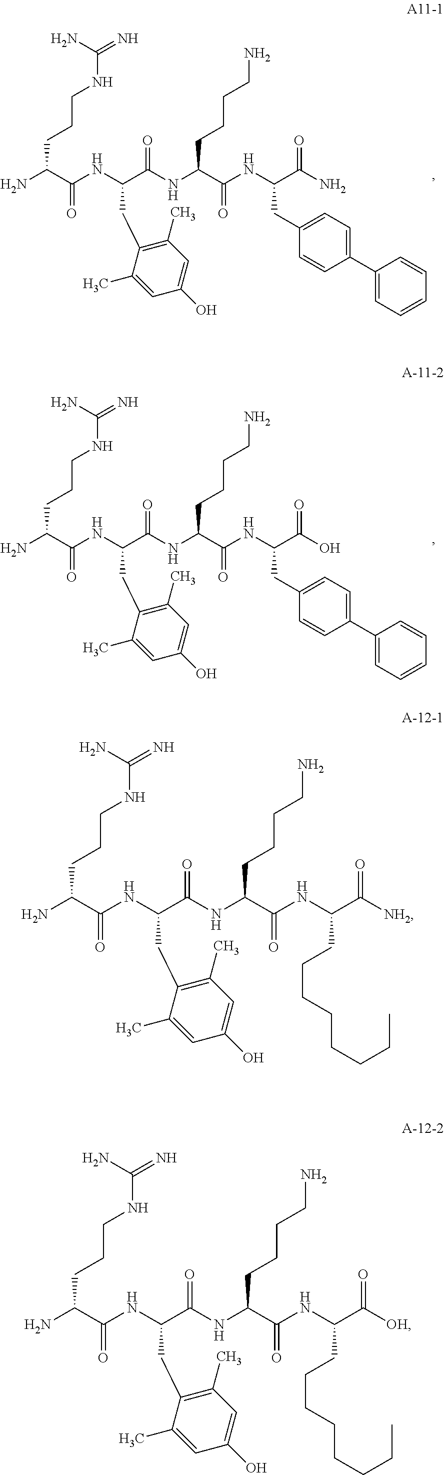 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

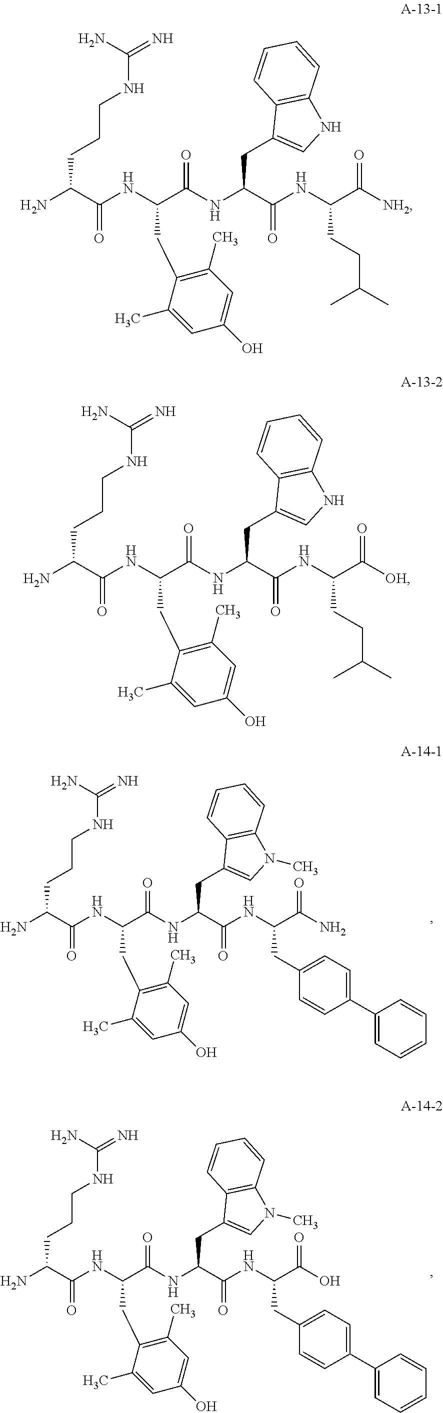 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O]

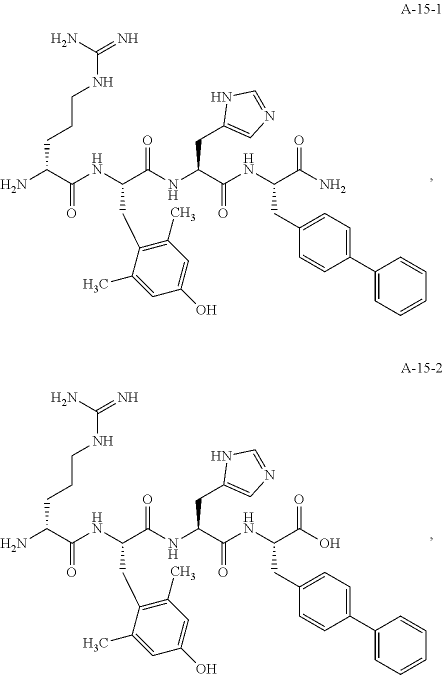 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the composition or medicament provides for normalizing left ventricular ejection fraction. In some embodiments, the composition or medicament provides for normalizing fractional shortening. In some embodiments, the composition or medicament provides for normalizing global longitudinal strain. In some embodiments, one or more hydrogen atoms of the peptide or peptides of Formulas A are optionally substituted with a deuterium or fluorine atom.

In some embodiments of the composition or medicament, the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the composition or medicament, the composition or medicament comprising the peptide or mixture of peptides is administered subcutaneously or intravenously. In some embodiments of the composition or medicament, the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the composition or medicament, the subject is human.

In some embodiments of the composition or medicament, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the composition or medicament, the peptide or peptides of the composition or medicament is/are formulated for administration from its/their tris-HCl salt.

In another aspect, this disclosure relates to the use of a peptide, mixture of peptides or a composition or medicament comprising the peptide or the mixture of peptides for normalizing left ventricular ejection fraction, fractional shortening, and/or global longitudinal strain in a mammalian subject in need thereof, wherein the peptide or peptides of the mixture is/are of Formula A:

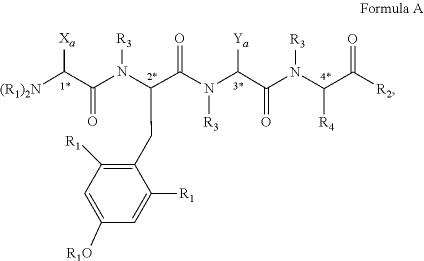

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from:

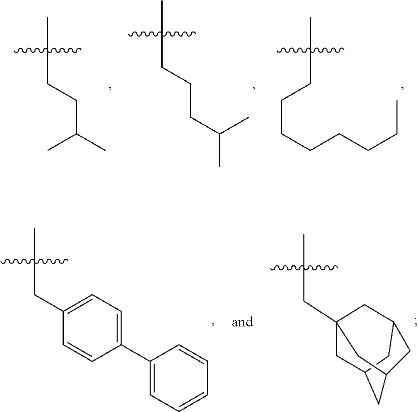

Xₐ is selected from

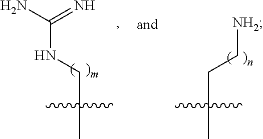

Yₐ is selected from

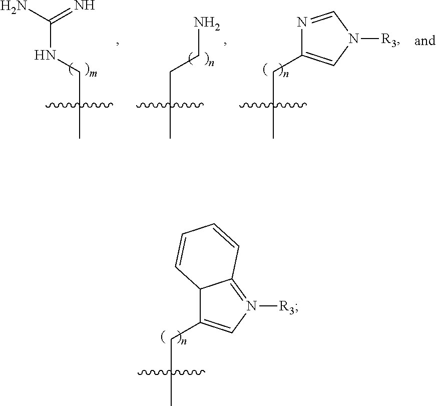

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide of Formula A is substituted with a deuterium or fluorine atom, wherein the normalization of the left ventricular ejection fraction, fractional shortening, and/or global longitudinal strain occurs in the subject as compared with a control subject to control group of subjects not administered the peptide or peptides of Formula A. In some embodiments of the use, the peptide or peptides of Formula A is/are a peptide/peptides of Formula A-1 or Formula A-2:

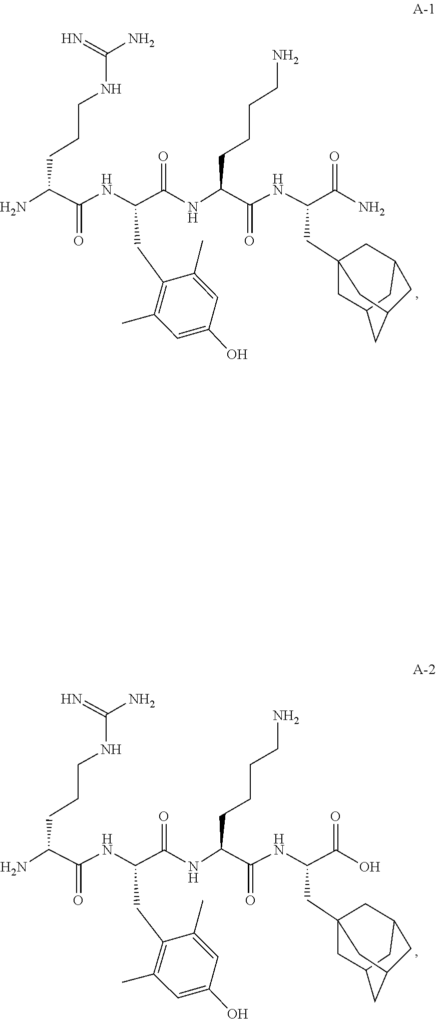 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments of the use, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2:

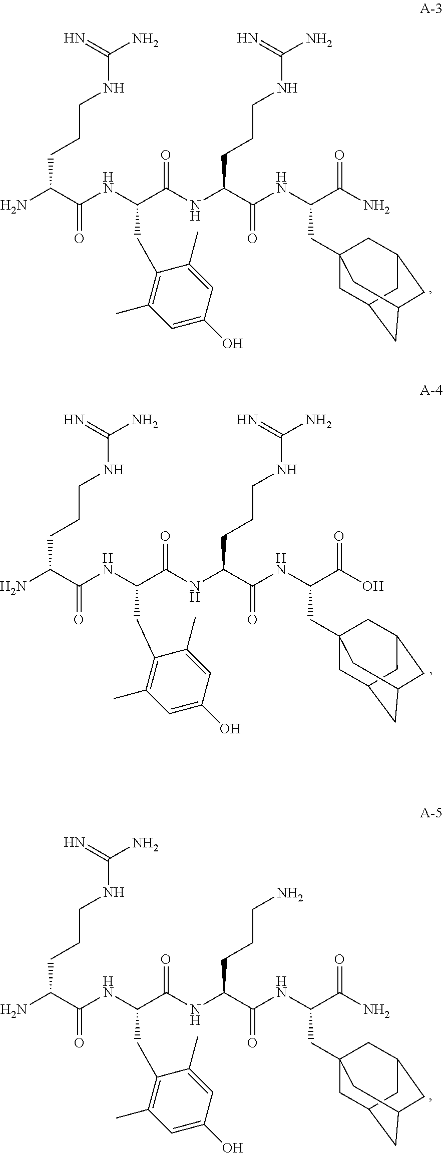 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

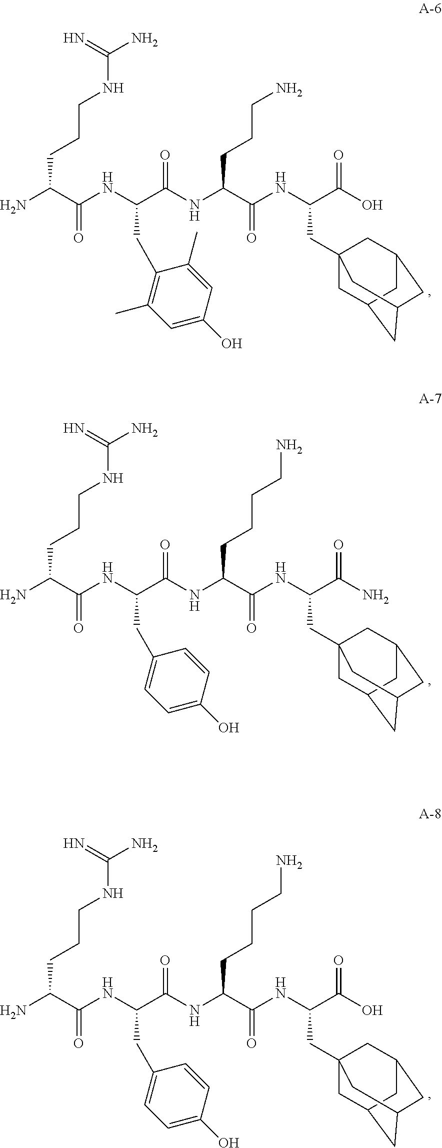 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

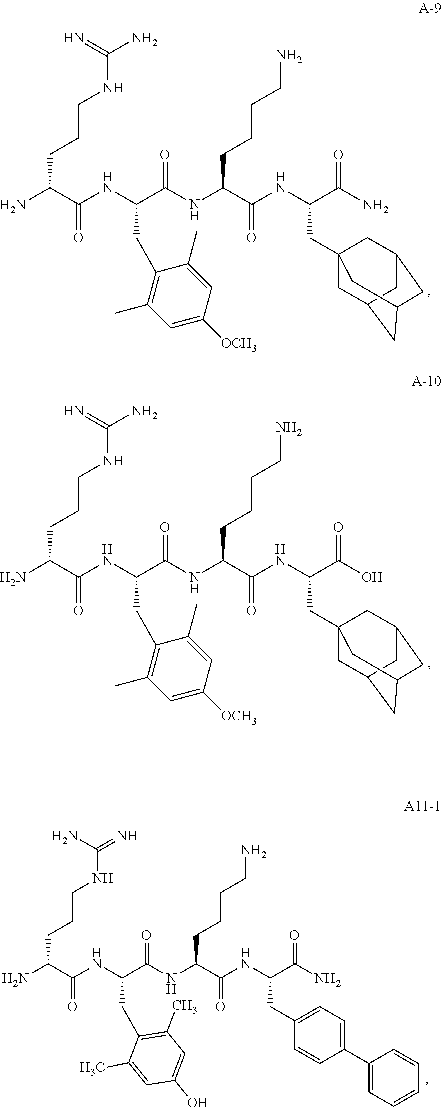 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O]

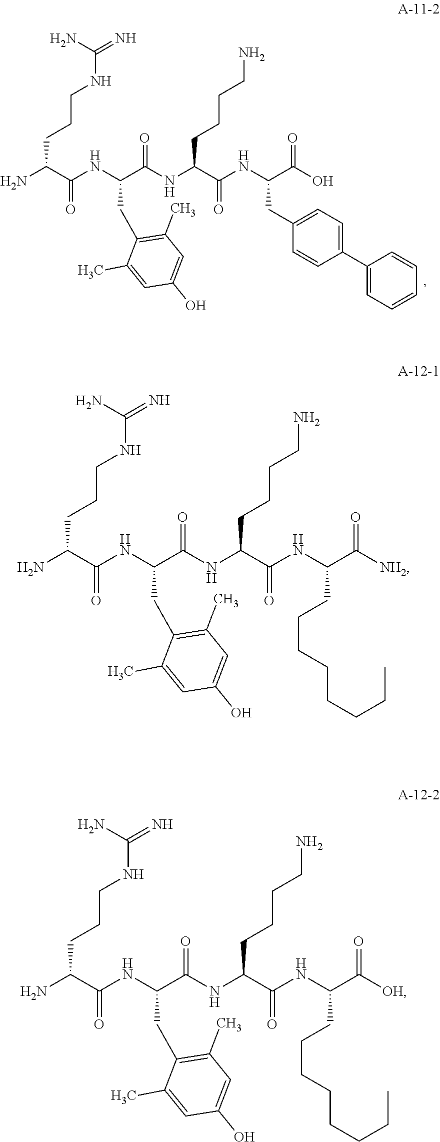 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O]

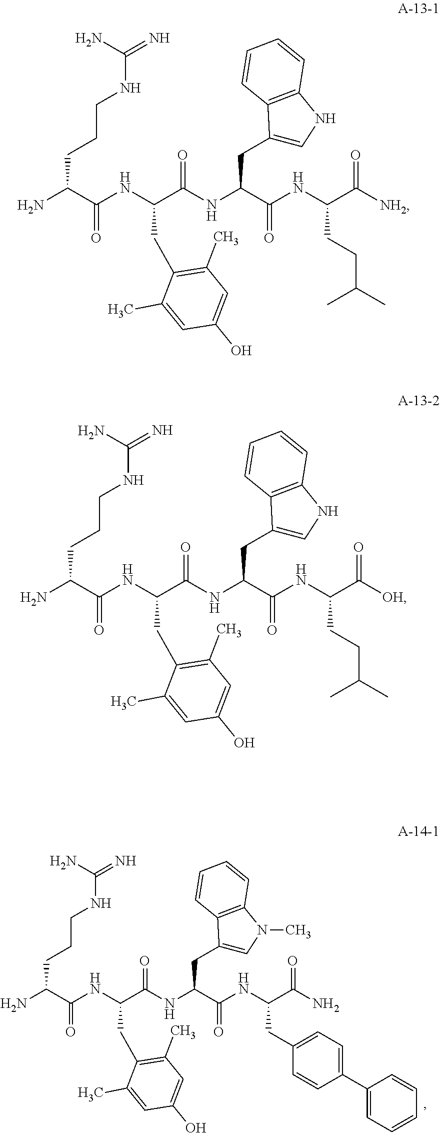 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O]

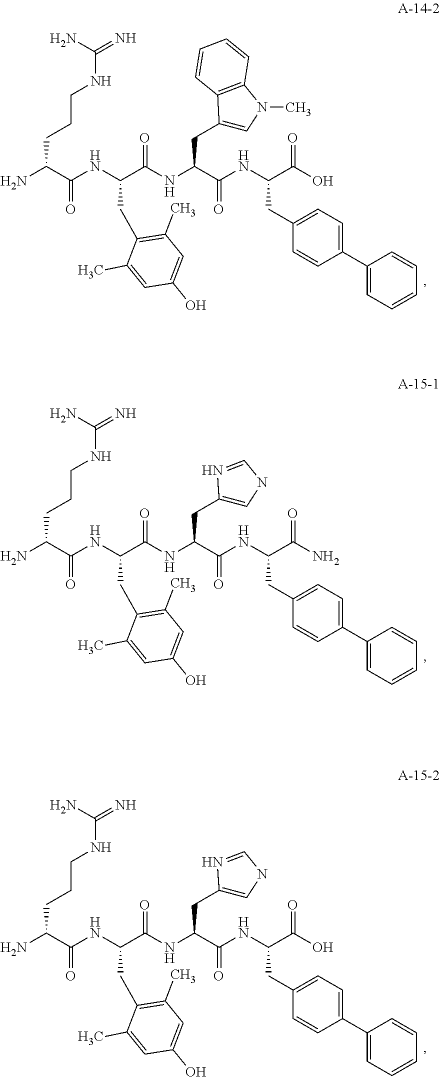 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1c[nH]cn1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1c[nH]cn1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the use of a peptide, mixture of peptides or a composition or medicament comprising the peptide or the mixture of peptides provides for normalizing left ventricular ejection fraction. In some embodiments, the use of a peptide, mixture of peptides or a composition or medicament comprising the peptide or the mixture of peptides provides for normalizing fractional shortening. In some embodiments, the use of a peptide, mixture of peptides or a composition or medicament comprising the peptide or the mixture of peptides provides for normalizing global longitudinal strain. In some embodiments, one or more hydrogen atoms of the peptide or peptides of Formulas A are optionally substituted with a deuterium or fluorine atom.

In some embodiments of the use, the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more. In some embodiments of the use, the peptide, mixture of peptides, or the composition or medicament comprising the peptide or mixture of peptides is administered subcutaneously or intravenously. In some embodiments of the use, the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

In some embodiments of the use, the subject is human.

In some embodiments of the use, the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt. In some embodiments of the use, the peptide or peptides is/are formulated for administration from its/their tris-HCl salt.

### Determination of the Biological Effect of the Peptides or Mixtures of Peptides

In various embodiments, suitable in vitro or in vivo assays are performed to determine the effect of a peptide or mixture of peptides and therefore whether their administration is indicated for treatment. In various embodiments, in vitro assays can be performed with representative animal models, to determine if a given peptide or mixture of peptides exerts the desired effect on the disease. Compounds (e.g., a peptide or mixture of peptides) for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### Combination Therapy

In some embodiments, the peptide or mixtures of peptides disclosed herein (alone or as a composition, formulation or medicament), may be combined with one or more additional therapeutic agents related to the treatment of (including without limitation the inhibition of, prevention of, amelioration of, or delaying the onset of) heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM), including signs or symptoms thereof, in a subject, including a human subject. For example, additional therapeutic agents may include, but are not limited to, (i) corticosteroids (e.g., prednisone), (ii) angiotensin-converting enzyme (ACE) inhibitors, (iii) angiotensin receptor blockers (ARBs), (iv) beta blockers, (v) diuretics, (vi) mineralocorticoid receptor antagonists and/or (vii) calcium channel blockers (CBBs).

In some embodiments, the corticosteroids are selected from the group consisting of prednisone and deflazacort. In some embodiments, the angiotensin receptor blockers (ARB) include: azilsartan, candesartan, eprosartan, irbesartan, telmisartan, valsartan, losartan, olmesartan, Entresto® (sacubitril/valsartan) and Byvalson™ (nebivolol/valsartan). In some embodiments, the beta blockers include: carvedilol, bisoprolol, metoprolol succinate, atenolol, esmolol, nebivolol and propranolol. In some embodiments, the diuretics include: bumetanide, furosemide and torsemide. In some embodiments, the mineralocorticoid receptor antagonists include: spironolactone and eplerenone. In some embodiments, the calcium channel blockers include amlodipine (Norvasc®), diltiazem (Cardizem), felodipine, isradipine, nicardipine, nifedipine (Procardia®), nisoldipine (Sular®) and verapamil (Verelan®).

In some embodiments, when an additional therapeutic agent is administered to a subject in combination with the peptide or mixture of peptides, a synergistic therapeutic effect is produced.

### Kits

The present technology also provides kits for treating heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM). In some embodiments, the kits comprise at least one peptide of Formula A (e.g., a peptide of Formula: A-1, A-2, A-3, A-4, A-5, A-6, A-7, A-8, A-9, A-10, A-11-1, A-11-2, A-12-1, A-12-2, A-13-1, A-13-2, A-14-1, A-14-2, A-15-1 or A-15-2) or mixtures thereof; alone or as formulated into a composition or medicament) and at least one additional therapeutic agent packaged in a suitable container and optionally comprising instructions for its use. Such additional therapeutic agents have been disclosed herein and include: (i) angiotensin-converting enzyme (ACE) inhibitors, (ii) angiotensin receptor blockers (ARBs), (iii) beta blockers, (iv) diuretics, (v) mineralocorticoid receptor antagonists or (vi) calcium channel blockers (CBBs). A non-limiting list of angiotensin-converting enzyme (ACE) inhibitors includes captopril, enalapril, lisinopril, benazepril, and ramipril. A non-limiting list of angiotensin receptor blockers (ARB) includes azilsartan, candesartan, eprosartan, irbesartan, telmisartan, valsartan, losartan, olmesartan, Entresto® (sacubitril/valsartan) and Byvalson™ (nebivolol/valsartan). A non-limiting list of beta blockers includes carvedilol, bisoprolol, metoprolol succinate, atenolol, esmolol, nebivolol and propranolol. A non-limiting list of diuretics includes bumetanide, furosemide and torsemide. A non-limiting list of mineralocorticoid receptor antagonists includes spironolactone and eplerenone. A non-limiting list of calcium channel blockers includes amlodipine (Norvasc®), diltiazem (Cardizem), felodipine, isradipine, nicardipine, nifedipine (Procardia®), nisoldipine (Sular®) and verapamil (Verelan®).

Therefore, in still another aspect pertains to kits for treating, preventing, inhibiting, ameliorating or delaying the onset of heart failure (HF), idiopathic cardiomyopathy (ICM), dilated cardiomyopathy (DCM), restrictive cardiomyopathy (RCM), hypertrophic cardiomyopathy (HCM) or hypertensive cardiomyopathy (HTN-CM) in a mammalian subject in need thereof, wherein the kit comprises a peptide of Formula A, or a mixture of peptides of Formula A (alone or as formulated into a composition or medicament):

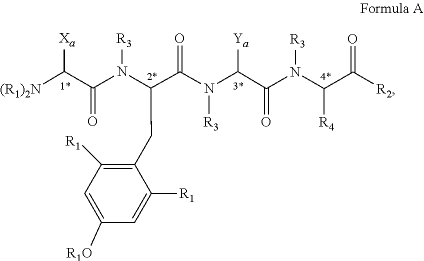

or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; R₃ is independently H, or —CH₃; R₄ is selected from:

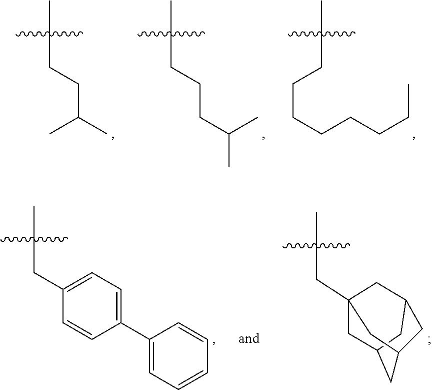

Xₐ is selected from

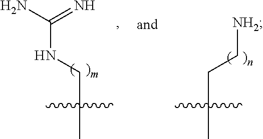

Yₐ is selected from

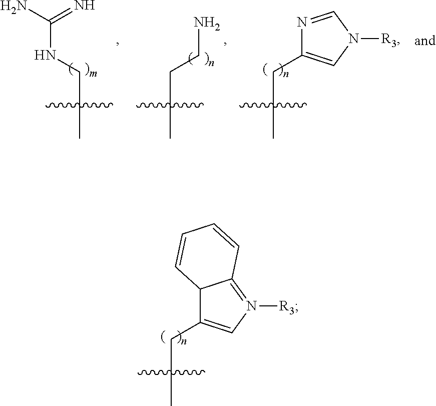

each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom; and an additional therapeutic agent, such as: (i) an angiotensin-converting enzyme (ACE) inhibitor, (ii) an angiotensin receptor blocker (ARB), (iii) a beta blocker, (iv) a diuretic, (v) a mineralocorticoid receptor antagonist or (vi) a calcium channel blocker (CBB). In some embodiments, the peptide of Formula A is a peptide of Formula A-1 or Formula A-2:

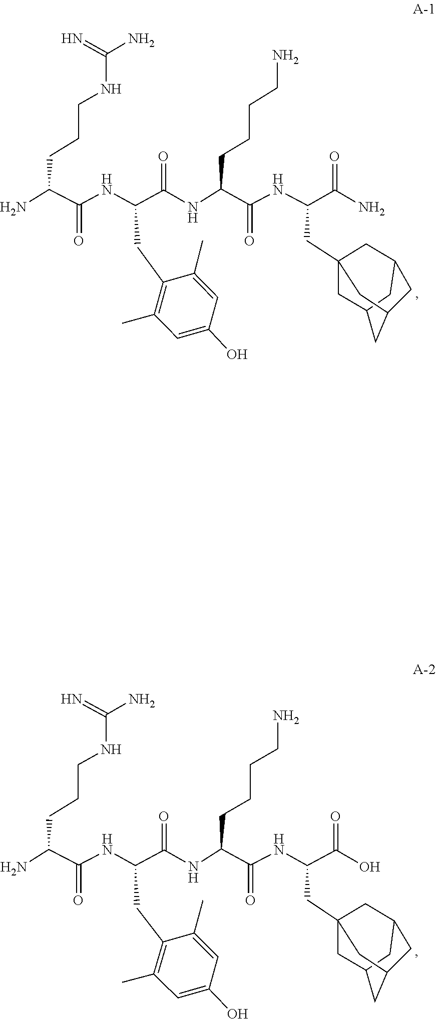 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]

or a pharmaceutically acceptable salt, hydrate, solvate, stereoisomer and/or tautomer thereof. In some embodiments, the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or

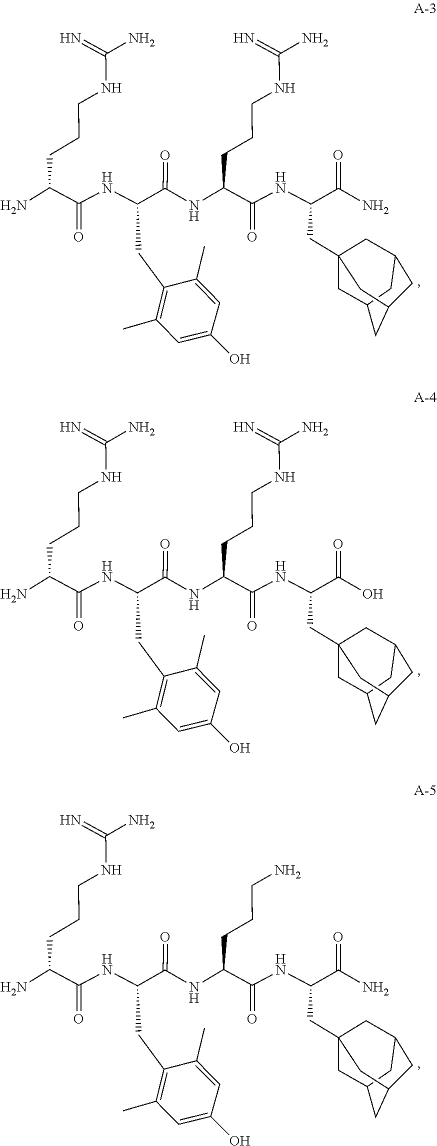 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O]

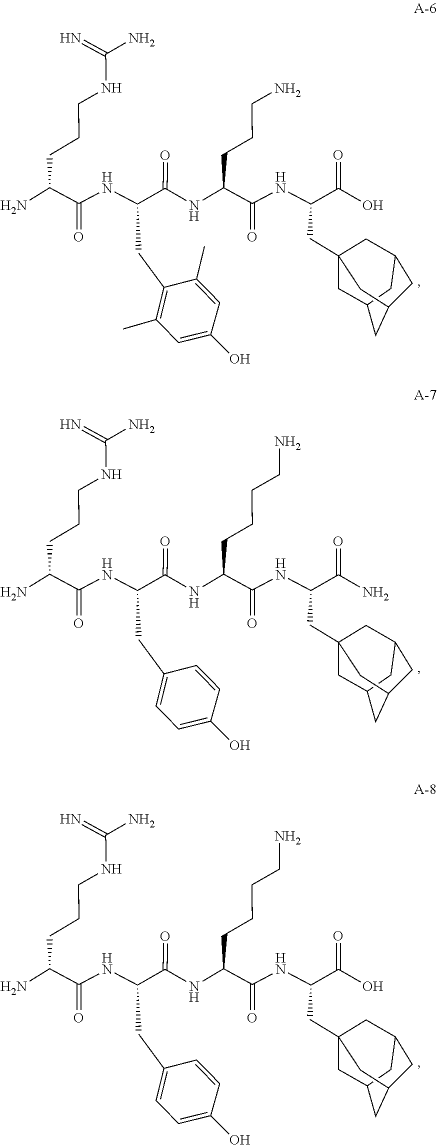 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O]

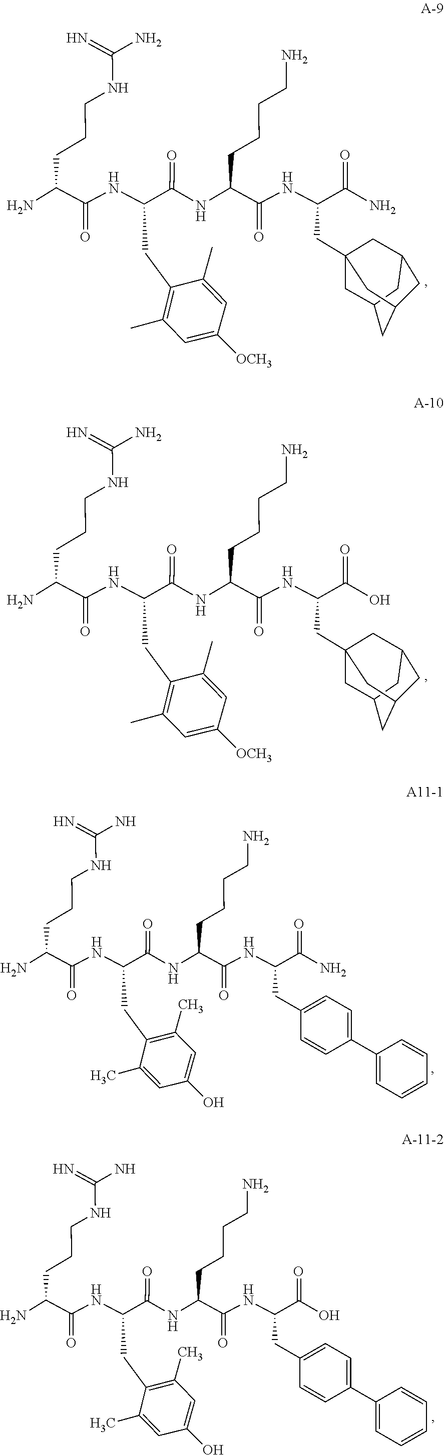 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O]

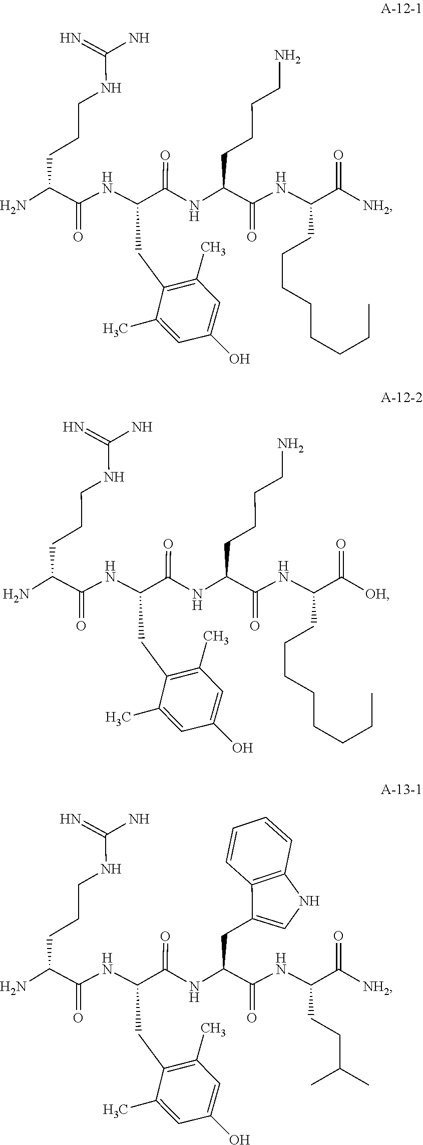 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O]

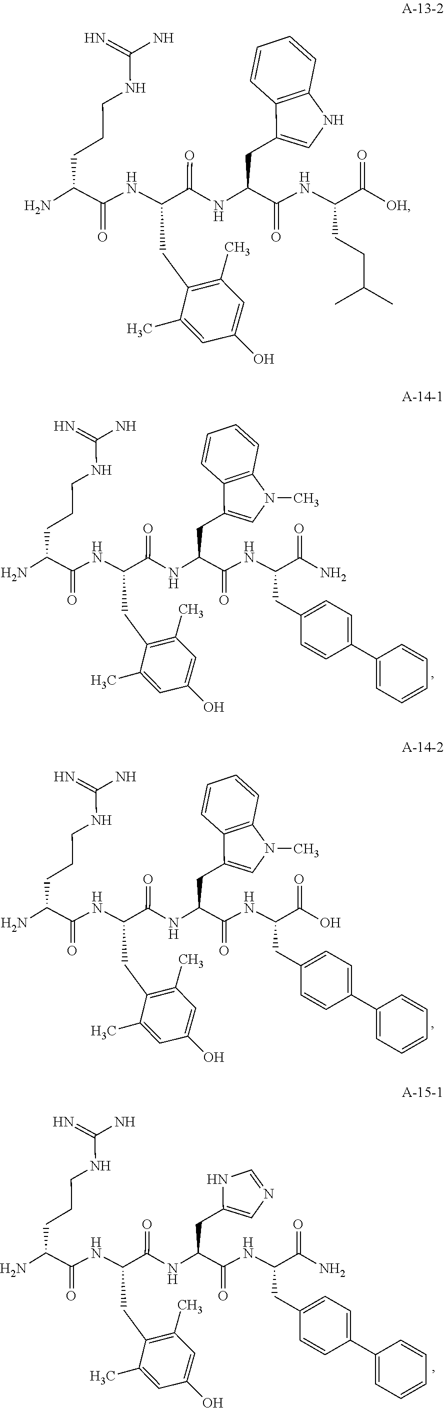 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O]

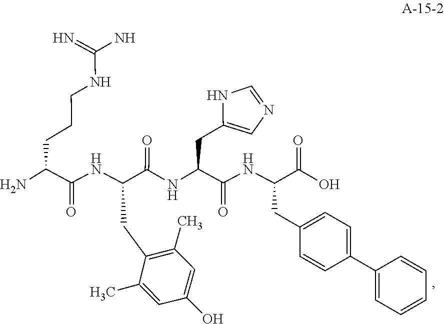 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]

or a pharmaceutically acceptable salt, hydrate, solvate, stereoisomer and/or tautomer thereof. In some embodiments, the kit is provided for treating, preventing, inhibiting, ameliorating or delaying the onset of heart failure (HF). In some embodiments, the kit is provided for treating, preventing, inhibiting, ameliorating or delaying the onset of idiopathic cardiomyopathy (ICM). In some embodiments, the kit is provided for treating, preventing, inhibiting, ameliorating or delaying the onset of dilated cardiomyopathy (DCM). In some embodiments, the kit is provided for treating, preventing, inhibiting, ameliorating or delaying the onset of restrictive cardiomyopathy (RCM). In some embodiments, the kit is provided for treating, preventing, inhibiting, ameliorating or delaying the onset of hypertrophic cardiomyopathy (HCM). In some embodiments, the kit is provided for treating, preventing, inhibiting, ameliorating or delaying the onset of hypertensive cardiomyopathy (HTN-CM). In some embodiments of the foregoing kits, one or more hydrogen atoms of the peptide or peptides of Formulas A are optionally substituted with a deuterium or fluorine atom.

It will be understood by one of ordinary skill in the relevant arts that other suitable modifications and adaptations to the compositions and methods described herein are readily apparent from the description of the technology contained herein in view of information known to the ordinarily skilled artisan, and may be made without departing from the scope of the present technology or any embodiment thereof. Having now described the present technology in detail, the same will be more clearly understood by reference to the following examples, which are included herewith for purposes of illustration only and are not intended to be limiting of the present technology.

### EXAMPLES

The present technology is further illustrated by the following examples, which should not be construed as limiting in any way. For each of the examples below, any peptide of Formula A (e.g., a compound of Formula A-1, Formula A-2, Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, Formula A-15-2 or mixtures thereof) could be used. Regardless, but not by limitation, the peptide used in the following examples below was the peptide of Formula A-1. The peptide of Formula A-1 was prepared by solution-phase methods by first making the protected Boc-D-Arg-DMT-OH dimer (41; See: FIG. 4) and the protected H₂N-Lys (Boc)-ADM-NH₂ dimer (42; See: FIG. 4), followed by condensing the two dimers and deprotecting the tetrapeptide as shown in FIG. 4; Also see: WO2020/131283. All other peptides of Formula A can be prepared using the generic process discussed in the Scheme at FIG. 4 by selecting the appropriate amino acids to form the appropriate peptide dimers.

### Example 1-Assessment of the Pharmacokinetics of Elamipretide and the Peptide of Formula A-1 Following Intraperitoneal Dose Administration to Male C57BL/6 Mice

### a) Dosing Protocol:

Animals: Male/C57Bl/6 mice/−18-25 grams were received from an approved vendor.
An untreated control group of n=3 mice (Group 3) was used to provide control plasma for Group 1 and Group 2, and control tissues for Group 2
Fasting was not required for this study.
Body weights were recorded prior to dose administration. The volume of each dose delivered (mL/kg) was based on each individual animal's body weight.
Doses were administered in accordance with test facility standard operating procedures.
All dose syringes were weighed prior to and following dosing to gravimetrically determine the amount of formulation administered.
All animals were observed at dosing and each scheduled collection. Any abnormalities were recorded.
Elamipretide and the peptide of Formula A-1 were formulated as follows:
   Elamipretide was provided in powder form and formulated for administration by the contract research organization (CRO) vendor. Elamipretide was removed from the refrigerator and the un-opened bottle was allowed to equilibrate at room temperature for a minimum of 2 hours. Then, elamipretide was dissolved in 0.9% saline at a concentration of 1.5 mg/mL, using a correction factor of 83.3% to account for salt content and HPLC purity. The drug formulation was filtered through 0.2 μm polyether sulfone (PES) filter and stored at 2-8° C. until use.
   The peptide of Formula A-1 was provided in powder form and formulated for administration by the CRO vendor. The peptide of Formula A-1 was removed from the refrigerator and the un-opened bottle was allowed to equilibrate at room temperature for a minimum of 2 hours. Then, the peptide of Formula A-1 was dissolved in 0.9% saline at a concentration of 1.5 mg/mL, using a correction factor of 85.3% to account for salt content and HPLC purity. The drug formulation was filtered through 0.2 μm polyether sulfone (PES) filter and stored at 2-8° C. until use.
Study Design is set forth in Table 1, below.

TABLE 1
Study Design

| | Number | | Dose | Dose | Dose | | |
|---|---|---|---|---|---|---|---|
| Group | of | Test | Level | Conc. | Volume | | |
| Number | Animals | Article | (mg/kg) | (mg/mL) | (mL/kg) | Vehicle | Route |
| 1 | 15M | Elam | 5.0 | 1.5 | 3.33 | 0.9% saline | IP |
| 2 | 15M | A-1 | 5.0 | 1.5 | 3.33 | 0.9% saline | IP |
| 3 | 3M | Untreated | N/A | N/A | N/A | N/A | N/A |

15M = 15 male mice;
3M = 3 male mice;
Elam = elamipretide;
A-1 = the peptide of Formula A-1;
N/A = not applicable;
IP = intraperitoneal

### b) Sample Collection-Post Dosing:

Terminal blood samples were collected via cardiac puncture following inhalation of anesthesia in accordance with test facility standard operating procedures. Group 1 animals were sacrificed following cardiac puncture.
Blood samples were collected into tubes with appropriate anticoagulant as specified in Table 2. The tubes were stored on wet ice until processed to plasma by centrifugation (3500 rpm at 5° C. for 10 minutes) within 20 minutes of collection. Sample aliquots of 100 μL were transferred into individual uniquely labeled matrix tubes and stored at nominal −80° C. until transferred to analytical chemistry for analysis by LC/MS/MS.
Immediately following each terminal blood collection for Groups 2 and 3, animals were sacrificed and perfused with cold PBS. The following tissues were collected: heart and muscle in accordance with Table 2.
Plasma and tissue samples were processed for analysis by LC/MS/MS to determine the amounts/concentrations reported in FIG. 5A and FIG. 5B.

TABLE 2
Sample Collection Design

| | Terminal Blood | Tissue Collections |
|---|---|---|
| Group | Collections Post-Dose | (n = 3 per |
| Number | (N = 3 per dose timepoint) | dose timepoint) |
| 1 | 0.5, 1, 2, 8, and 24 hours | N/A |
| | (n = 3 per time point) | |
| 2 | 0.5, 1, 2, 8, and 24 hours | Saline perfusion |
| | | followed by collection |
| | (n = 3 per time point) | of heart and muscle |
| 3 | 0 hours | Saline perfusion |
| | | followed by collection |
| | | of heart and muscle |
| Anticoagulant | K2 EDTA | N/A |
| Volume/ | Max obtainable (terminal) | N/A |
| Timepoint | | |

### Results:

The results are presented in graphic form in FIG. 5A and FIG. 5B. With reference to FIG. 5A, the plasma exposure of the peptide of Formula A-1 is compared with the plasma exposure of elamipretide. The data indicates that when both are administered in the same manner and at the same dose, the peptide of Formula A-1 is roughly 3-4 times more concentrated in the plasma at 30 minutes to one hour as compared with elamipretide and is still present in the plasma at greater than 100 ng/mL after 4 hours whereas elamipretide is present at 100 ng/ml at 2 hours and generally dropped below the 1 ng/ml lower limit of quantification at 4 hours.

With reference to FIG. 5B, data is presented for the concentration of the peptide of Formula A-1 in plasma, heart, and muscle. The data demonstrates that the there is good uptake of the peptide of Formula A-1 in heart and muscle, without any significant difference observed in uptake between the two tissues (i.e., muscle and heart). Whilst tissue concentrations from 30 minutes to 2 hours were about 10 times lower than the plasma concentrations (assuming a tissue density of 1 g/mL), the peptide of Formula A-1 provides reasonable penetration into muscle and heart tissue sufficient to potentially produce an efficacious drug effect. By 8 hours, the concentration of the peptide of Formula A-1 present in the tissues (heart and muscle) is higher than that in the plasma. It is also worth noting that administration of the peptides (elamipretide and Formula A-1) was well tolerated by the animals.

### Example 2—Assessment of the Pharmacokinetics of Elamipretide and Dose Escalation of the Peptide of Formula A-1 Following Intraperitoneal Administration to Male C57BL/6 Mice

### a) Dosing Protocol:

Animals: Male/C57BL/6 mice/−18-25 grams were received from an approved vendor and allowed to acclimate to the test facility for 2 days prior to the start of the study. Group 4 animals were monitored on days 1 and 2 for 2 hours post dosing.
Fasting was not required for this study.
Body weights were recorded prior to dose administration. The volume of each dose delivered (mL/kg) was based on each individual animal's body weight.
Doses were administered in accordance with test facility standard operating procedures.
This was a multiple dose terminal study dosed once per day over 5 days. Mice were taken down following their 5th and final dose.
All dose syringes were weighed prior to and following dosing to gravimetrically determine the amount of formulation administered.
All animals were observed at dosing and each scheduled collection. Any abnormalities were recorded.
Elamipretide and the peptide of Formula A-1 were formulated as follows:
   Elamipretide was provided in powder form. Elamipretide was removed from the refrigerator and the un-opened bottle was allowed to equilibrate at room temperature for a minimum of 2 hours. Then, elamipretide was dissolved in 0.9% saline at a concentration of 1.5 mg/mL, using a correction factor of 83.3% to account for slat content and HPLC purity. The drug formulation was filtered through 0.2 μm polyether sulfone (PES) filter and stored at 2-8° C. until use.
   The peptide of Formula A-1 was provided in powder form. The peptide of Formula A-1 was removed from the refrigerator and the un-opened bottle was allowed to equilibrate at room temperature for a minimum of 2 hours. Then, the peptide of Formula A-1 was dissolved in 0.9% saline at a concentration of 0.3 mg/mL (Group 2) 1.5 mg/mL (Group 3) and 4.5 mg/mL (Group 4), in each case using a correction factor of 85.3% to account for salt content and HPLC purity. The drug formulation was filtered through 0.2 μm PES filter and stored at 2-8° C. until use.
Study Design is set forth in Table 3, below.

TABLE 3
Study Design

| | Number | | Dose | Dose | Dose | | | |
|---|---|---|---|---|---|---|---|---|
| Group | of | Test | Level | Conc. | Volume | | | |
| Number | Animals | Article | (mg/kg) | (mg/mL) | (mL/kg) | Dose Schedule | Vehicle | Route |
| 1 | 3M | Elam | 5.0 | 1.5 | 3.33 | 1x daily for 5 days | 0.9% saline | IP |
| 2 | 3M | A-1 | 1.0 | 0.3 | 3.33 | 1x daily for 5 days | 0.9% saline | IP |
| 3 | 3M | A-1 | 5.0 | 1.5 | 3.33 | 1x daily for 5 days | 0.9% saline | IP |
| 4 | 3M | A-1 | 15.0 | 4.5 | 3.33 | 1x daily for 5 days | 0.9% saline | IP |

3M—3 male mice;
Elam = elamipretide;
A-1—the peptide of Formula A-1;
IP = intraperitoneal

b) Sample collection-Post Dosing:
Terminal blood samples were collected via cardiac puncture following inhalation of anesthesia in accordance with test facility standard operating procedures.
Immediately following each terminal blood collection for all groups, animals were sacrificed and perfused with cold PBS. The following tissues were collected: heart, gastrocnemius muscle, and liver in accordance with Table 4.
Plasma and tissue samples were processed for analysis by LC/MS/MS to determine the amounts/concentrations reported in the FIG. 6.

TABLE 4
Sample Collection Design

| | Terminal Blood | |
|---|---|---|
| | Collections Post-Dose | |
| Group | (N = 3 per | |
| Number | dose timepoint) | Tissue Collections |
| 1-4 | 2 hours following 5^{th} | Cold PBS perfusion followed |
| | | by collection of heart, |
| | and final dose | gastrocnemius muscle, and liver |
| Anticoagulant | K2 EDTA | N/A |
| Volume/ | Max obtainable | N/A |
| Timepoint | (terminal) | |

### Results:

With reference to FIG. 6, the concentration (measured in ng test article/gram of tissue) of test articles (elamipretide and the peptide of Formula A-1) is presented in a bar graph. The graph compares the concentration in muscle (gastrocnemius), heart, and liver of elamipretide dosed at 5 mg/kg IP as compared with the peptide of Formula A-1, dosed at 1 mg/kg, 5 mg/kg and 15 mg/kg, each by IP. With reference to FIG. 6, the tissue concentrations of elamipretide administered at 5 mg/kg was roughly equivalent to the tissue concentrations of the peptide of Formula A-1 administered at a fivefold lower amount (i.e., 1 mg/kg). By 8 hours, the concentration of the peptide of Formula A-1 present in the tissues (heart and muscle) is higher than that in the plasma. The data further demonstrates that the peptide of Formula A-1 appears to penetrate all tissues, therefore suggesting that the compound of Formula A-1 can be delivered to tissues in an amount to produce an efficacious drug effect. It is also worth noting that administration of the peptides (elamipretide and the peptide of Formula A-1) was well tolerated by the animals.

### Example 3—Assessment of Tissues/Mitochondria from Failing Human Hearts Treated with Elamipretide and with the Peptide of Formula A-1

This example was performed to examine and compare the protective effects of elampretide and the peptide of Formula A-1 on the heart tissue/mitochondria from (explanted) failing human hearts. As these hearts are significantly impaired when explanted (as part of a heart transplant operation) this example tests the applicability of the test articles (i.e., elamipretide and the peptide of Formula A-1) to improve cardiac function in the heart tissues/mitochondria.

### Methods:

Respiration of human heart mitochondria was measured by high resolution respirometry (Oxygraph, Oroboros Instruments, Innsbruck, Austria) using a stepwise protocol to evaluate various components of the electron transport system. Ventricular heart tissue was cut into pea-sized pieces and placed in BIOPS (10 mM Ca-EGTA buffer, 0.1 μM free calcium, 20 mM imidazole, 20 mM taurine, 50 mM (2-morpholinoethanesulfonic acid) potassium (K-MES), 0.5 mM dithiothreitol, (DTT), 6.56 mM magnesium chloride (MgCl₂), 5.77 mM adenosine triphosphate (ATP), 15 mM phosphocreatine, pH 7.1.) immediately following explanation and kept at 4° C. Not more than 12 hours later, the explanted heart tissue was incubated in BIOPS containing 100 μM elamipretide or 0.1 μM of the peptide of Formula A-1 or BIOPS alone (vehicle control) at 4° C. for 2 hours. Following incubation, 10 mg tissue was placed in 1 mL of KME solution (100 mM potassium chloride (KCl), 50 mM 3-morpholinopropane-1-sulfonic acid (MOPS) and 0.5 mM ethylene glycol tetraacetic acid (EGTA), pH 7.4). Tissue was homogenized using the IKA®T25 ULTRA-TURRAX® homogenizer, speed 4, 2 sec on, 2 sec off for total of 8 s (4 bursts). 25 μL of this homogenate was then placed in the chambers of the Oroboros O₂K apparatus at 37° C. containing respiration medium and either vehicle (water), elamipretide or peptide of Formula A-1 depending on the treatment. Standard protocols were followed for calibration of the chambers and followed by stepwise addition of 0.2 mM palmitoylcarnitine, 1 mM malate, 4 mM adenosine diphosphate (ADP), 0.4 mM octonylcarnitine, 5 mM pyruvate, 10 mM glutamate, 10 mM succinate, 0.5 μM steps of CCCP (until maximum rate reached) followed by 2 μM rotenone and 5 μM antimycin A. Oxygen flux rates were normalized using the amount of protein added in μg after a protein assay was performed on the homogenate.

Mitochondrial superoxide in human heart was measured by EPR spectroscopy using a mitochondrial targeted probe, 1-hydroxy-4-[2-triphenylphosphonio)-acetamido]-2,2,6,6-tetramethylpiperidine, 1-Hydroxy-2,2,6,6-tetramethyl-4-[2-(triphenylphosphonio) acetamido] piperidinium dichloride (mito-TEMPO-H) (Enzo Life Sciences, Farmingdale, NY). This compound is a cyclic hydroxylamine that is oxidized upon the reaction with superoxide to generate a more stable radical called nitroxide. The concentration of nitroxide radical (mito-TEMPO.) following oxidation of (mito-TEMPO-H) was measured in the human heart tissue/mitochondria. Tissue was treated with elamipretide or the peptide of Formula A-1 and homogenized as detailed for respirometry studies. Following homogenization, 120 μL of tissue homogenate was incubated with 0.2 mM mito-TEMPO-H in Krebs-Henseleit KHB containing 100 μM in 200 μL of total volume for 1 h at 37° C. 150 μL of the reaction mixture were loaded in PTFE tubing flash froze in liquid nitrogen. EPR measurements were performed at 77° K using the Bruker EMXnano X-band spectrometer. EPR acquisition parameters were as follows: microwave frequency=9.65 GHz; center field=3438 G; modulation amplitude=6.0 G; sweep width=150 G; microwave power=0.316 mW; total number of scans=3; sweep time=60 s; and time constant=1.28 ms. mito-TEMPO nitroxide radical concentration was obtained by double integration followed by Spin Count module (Bruker) and expressed as the nitroxide radical per gram of protein.

### Results:

With reference to FIG. 7A, O₂ flux of failing human heart mitochondria is presented in a line graph. The graph compares O₂ flux of vehicle (water)-treated, elamipretide-treated, or peptide of Formula A-1-treated mitochondria after the stepwise addition of 0.2 mM palmitoylcarnitine (PC), 1 mM malate (Mal), 4 mM ADP, 0.4 mM octonylcarnitine (OC), 5 mM pyruvate (Pyr), 10 mM glutamate (Glu), 10 mM succinate (Succ), 0.5 μM steps of CCCP (until maximum rate reached) followed by 2 μM rotenone (Rot) and 5 μM antimycin A (AMA). With reference to FIG. 7A, treatment with the peptide of Formula A-1 improved O₂ flux of failing human heart mitochondria to a greater degree than treatment with elamipretide. Treatment with either elamipretide or the peptide of Formula A-1 resulted in improved O₂ flux of failing human heart mitochondria in all cases as compared with the control. In this experiment, the peptide of Formula A-1 outperformed elamipretide at an identical concentration.

FIG. 7B and FIG. 7C are bar graphs of data collected for superoxide production by mitochondria of failing (explanted) human hearts. These graphs compare superoxide production by failing human hearts treated with vehicle control (i.e., water), elamipretide (100 μM-FIG. 7B), or the peptide of Formula A-1 (100 nM-FIG. 7C). With reference to FIG. 7B, treatment with elamipretide (100 μM) reduced superoxide production relative to vehicle-treated control. By comparison, with reference to FIG. 7C, treatment with peptide of Formula A-1 at 100 nM (i.e., 3 orders of magnitude lower than elamipretide) reduced superoxide production relative to vehicle-treated control to a similar level as was seen with the significantly higher concentration of elamipretide. This data suggests that administration of the peptide of Formula A-1 is superior to administration of elamipretide with respect to suppression of superoxide production in the mitochondria of the failing human hearts.

With reference to FIG. 7D and FIG. 7E, data is presented in line graph form for O₂ flux of mitochondria from failing (explanted) human heart tissues. The graphs compare complex I+II respiration (FIG. 7D) and maximal respiration (FIG. 7E) of mitochondria from failing (explanted) human heart tissue treated with vehicle (untreated) or treated with 10 nM of the peptide of Formula A-1. With reference to FIG. 7D, treatment with the peptide of Formula A-1 significantly improved complex I+II respiration relative to untreated control (p=0.018). Similarly, with reference to FIG. 7E, treatment of the sample with the peptide of Formula A-1 significantly improved maximal respiration of failing human heart mitochondria relative to untreated control (p=0.03). These data suggest that treatment of the failing human heart tissues with the peptide of Formula A-1 will improve mitochondrial function.

### Example 4—Assessment of Mitochondria from Mouse Heart Treated with Elamipretide and the Peptide of Formula A-1

### Methods:

Murine hearts were collected immediately following sacrifice of C57BL/6 mice and transferred on ice in isolation buffer (180 mM KCl, 10 mM Tris HCl, and 1 mM EGTA (pH 7.7)) to a site for further analysis. When received, the heart tissue was transferred to an ice-cold petri dish, cut into small pieces with dissection scissors, and finally homogenized with 10-12 strokes of a Dounce homogenizer. The slurry was centrifuged at 1000×g for five minutes at 4° C. in the Sorvall XPro Series centrifuge. To collect a mitochondrial rich fraction, the supernatant was harvested and again centrifuged at 10,000×g for ten minutes at 4° C. (Sorvall Legend Micro 21R Centrifuge). The remaining pellet was resuspended in 150 μL cold MiR05 buffer (Oroboros) and stored on ice. Protein concentration was determined by Bradford Assay.

Isolated murine heart mitochondria were incubated with the peptide of Formula A-1 (100 nM or 1 μM), 10 μM elamipretide, or vehicle control (dH₂O) for thirty minutes on ice and subsequently subjected to repeated freeze/thaw cycles to induce mitochondrial damage. Specifically, the mitochondria were flash frozen on dry ice for ninety seconds and immediately thawed in a 37° C. water bath for 45 seconds; this cycle was repeated three times. Following the final thaw, the mitochondria were mixed well and added to each chamber of a high-resolution respirometer, the O2K (Oroboros). This repeated freeze-thaw cycling was used to damage/impair the mitochondria isolated from mouse hearts, effectively uncoupling electron transport chain activity (oxygen consumption) from ATP synthesis.

Once added to the O2K, basal levels of mitochondrial respiration were achieved, and each chamber was supplemented with 10 mM glutamate, 2 mM malate, and 4 mM adenosine diphosphate (ADP). Oxygen concentration and flux were recorded in DatLab files. DatLab files were exported to GraphPad Prism for data plotting and analysis. State 3 respiration was determined as the measurement by which all conditions were compared.

### Results:

Data for the oxygen consumption rate (OCR) of the freeze/thaw treated mouse heart mitochondria treated with vehicle control, elamipretide (10 μM), or peptide of Formula A-1 (100 nM or 1 μM) is presented in a bar graph format in FIG. 7F. With reference to FIG. 7F, treatment with the peptide of Formula A-1 (100 nM or 1 μM) significantly improved mitochondrial oxygen consumption relative to vehicle-treated control. By comparison, in this assay, treatment with elamipretide (10 μM) had no apparent effect. Again, these data suggest that the peptide of Formula A-1 is superior to elamipretide in protecting or improving mitochondrial function of damaged/impaired heart tissue.

### Example 5—Assessment of Heart Function in Aged-Mice Treated with the Peptide of Formula A-1

### Methods:

24-26-month-old C57BL/6J (i.e., aged-) mice were treated with the peptide of the Formula A-1 (3 mg/kg-0.3 and 1 mg/kg were also used but that data are omitted as it was not significant) by osmotic minipumps (Alzet #1004) for 8 weeks. Briefly, minipumps were loaded with isotonic saline or the peptide of the Formula A-1 in isotonic saline. Alzet osmotic pumps were primed to deliver vehicle (saline) or 3 mg/kg/day of the peptide of Formula A-1 by continuous administration over the 24-hour period. Animals were induced for anesthesia using 4% isoflurane in 1 L/min O₂ and maintained during surgery at 1.5-2% isoflurane on a circulating water pad at 37° C. An approximate 1 cm incision was made along the midline of the lower back, pumps were implanted subcutaneously, and the incision was stapled shut using two 7 mm wound clips and dropped with Vetbond tissue glue (3M). Four weeks after initial surgery the animals were anesthetized as before, pumps were removed and replaced with freshly filled and primed Alzet osmotic pumps.

In vivo echocardiography was measured before and after pump installation. 2D echocardiography of the left ventricle was performed using the Vevo 3100 Preclinical Imaging System, Vevo Imaging Station, and MS400 probe from VisualSonics (Toronto, Canada). Mice are anesthetized with 4% isoflurane and fur is removed from their chest with Nair. The mice are placed in a supine position and heart rate, respiration rate, and core body temperature are monitored using the Vevo Animal Monitoring System SM200 and Vevo Monitor App software from VisualSonics. The SM200 also provides heat support to the mice while they are maintained on 1-3% isoflurane anesthesia. Parasternal long axis view (PLAX) B-mode and parasternal short-axis view (PSAX) B-mode and M-mode images were acquired. Resting cardiac workload images were acquired while maintaining the heart rate of the mouse between 400 and 500 beats per minute (bpm). Echocardiography was analyzed using the Vevo LAB software with Vevo Strain package. Strain analysis, including global longitudinal strain (GLS), was analyzed from 5 consecutive cardiac cycles without a respiration from the PLAX B-mode images using Vevo Strain package. The results were exported to Microsoft Excel and statistical analysis and graphing was performed using GraphPad Prism software and are presented in FIGS. 8A-8C.

### Results:

With reference to FIG. 8A, there was a statistically significant improvement in the percent change in fractional shortening of the hearts of the treated (3 mg/kg peptide of Formula A-1) mice as compared with untreated mice. With reference to FIG. 8B, there was a statistically significant improvement in the percent change in ejection fraction of the hearts of the treated (3 mg/kg peptide of Formula A-1) mice as compared with untreated mice. With reference to FIG. 8C, there was an improvement/normalization (albeit not statistically significant) in global longitudinal strain of the hearts of the treated (3 mg/kg peptide of Formula A-1) mice as compared with untreated mice. Collectively, these data suggest that treatment with peptide of Formula A-1 for 8 weeks significantly normalized fractional shortening (FS) and ejection fraction (EF) in the treated aged-mice and trended towards normalization of global longitudinal strain (GS) in aged mice, and is useful in methods for treating, preventing, ameliorating, or delaying the onset of heart failure and/or cardiomyopathy.

### Example 6—Langendorff Study

### Ischemia-Reperfusion Injury-Langendorff Heart Preparation Protocol:

An infarction study was performed according to the Langendorff technique as described in Kuka J, Vilskersts R, Cirule H, Makrecka M, Pugovics O, Kalvinsh I, et al. The cardioprotective effect of mildronate is diminished after co-treatment with L-carnitine. J Cardiovasc Pharmacol Ther. 2012; 17:215-222, with some modifications. Rats were anaesthetized with sodium pentobarbital (60 mg/kg) and heparin was administered intraperitoneally. For the infarction studies, the hearts were perfused with oxygenated (95% 02-5% CO2) Krebs-Henseleit (KH) buffer solution (118 mmol/L NaCl, 4.7 mmol/L KCl, 1.24 mmol/L CaCl2, 1.64 mmol/L MgCl₂, 24.88 mmol/L NaHCO₃, 1.18 mmol/L KH₂PO₄, and 0.05 mmol/L EDTA; pH 7.3-7.5; 36.8-37.0° C.) supplemented with 10 mM glucose at a constant perfusion pressure of 60 mmHg. A water-ethanol mixture (1:1)-filled balloon connected to a physiological pressure transducer (ADInstruments) was inserted into the left ventricle, and the baseline end-diastolic pressure set at 5-10 mmHg. The heart rate (HR), flow, left-ventricle developed pressure (LVDP), contractility (+dp/dt) were continuously recorded using a PowerLab 8/35 system from ADInstruments. The isolated rat hearts were adapted for 20 min and the left anterior descending coronary artery (LAD) was subsequently occluded for 30 min followed by 120 min of reperfusion. KH perfusion solution with or without added compound of interest (vehicle or 1 mM concentration of elamipretide or of peptides of Formula A (e.g., peptides of Formula A-1, A-11, A-12, A-13, A-14, or A-15) was used for the whole time of isolated heart perfusion.

### Compounds of Interest:

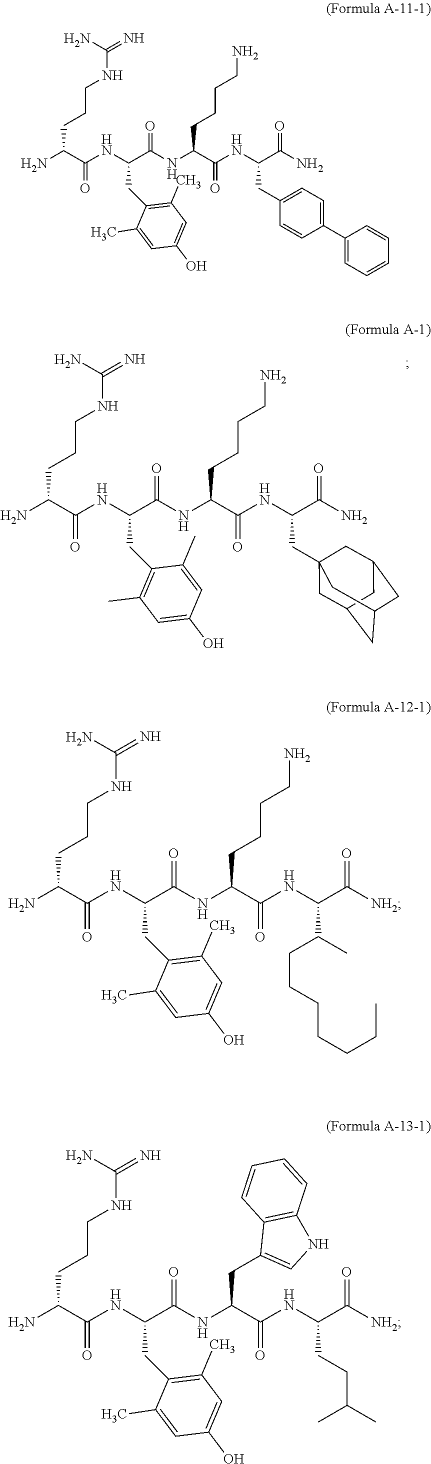 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; CCCCCCCC(C)[C@H](NC(=O)[C@H](CCCCN)NC(=O)[C@H](Cc1c(C)cc(O)cc1C)NC(=O)[C@H](N)CCCNC(=N)N)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O]

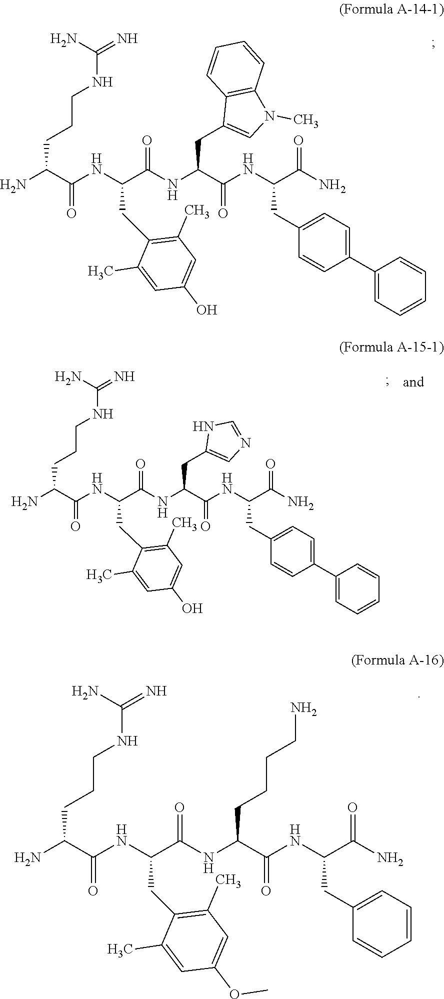 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; COc1cc(C)c(C[C@H](NC(=O)[C@H](N)CCCNC(=N)N)C(=O)N[C@@H](CCCCN)C(=O)N[C@@H](Cc2ccccc2)C(N)=O)c(C)c1]

Occlusion was confirmed by ~40% drop in coronary flow. The infarct size was determined as described in Kuka J, Vilskersts R, Cirule H, Makrecka M, Pugovics O, Kalvinsh I, Dambrova M, Liepinsh E. The cardioprotective effect of mildronate is diminished after co-treatment with L-carnitine. J Cardiovasc Pharmacol Ther. 2012 June; 17(2):215-22. doi:10.1177/1074248411419502; Liepinsh E, Kuka J, Dambrova M. Troubleshooting digital macro photography for image acquisition and the analysis of biological samples. J Pharmacol Toxicol Methods. 2013 March-April; 67(2):98-106. doi:10.1016/j.vascn.2012.11.00L and Herr, D. J., Aune, S. E., Menick, D. R. Induction and Assessment of Ischemia-reperfusion Injury in Langendorff-perfused Rat Hearts. J. Vis. Exp. (101), e52908, doi:10.3791/52908 (2015). Briefly, at the end of the reperfusion, the LAD was re-occluded, and the heart was perfused with 0.1% methylene blue dissolved in KH buffer solution. Afterwards, hearts were sectioned transversely from the apex to the base in 6 slices (5 if smaller heart) of 2 mm thickness and incubated in 1% triphenyl-tetrazolium chloride in phosphate buffer (pH 7.4, 37° C.) for 10 min to stain viable tissue red and necrotic tissue white. The planimetric analysis of cross-sectional images was performed using Image-Pro Plus v6.3 software to determine the area at risk (AR) and area of necrosis (AN), each expressed as a percentage of cross-sectional slice area. The obtained values were then used to calculate the infarct size (IS) as a percentage of the risk area according to the formula:

$$IS ( \% ) = AN / AR \times 100 \%$$

Area of necrosis was determined by combining areas of the white necrotic and pink tissue.

### Study Outline:

20 min. adaptation+30 min. ischemia (LAD ligation)+120 min. reperfusion (vehicle or compound 1 mM)
Endpoints: HR, flow, LVDP, ±dP/dt, infarct size-area of necrosis
CTRL (vehicle)+up to 4 test compounds (n=8 per treatment) tested per set

Results are shown in FIGS. 9A-9G. These results demonstrate that the peptides of the present technology are cardioprotective, all being roughly equivalent or superior to elamipretide in this ex-vivo assay.

### EQUIVALENTS

The present technology is not to be limited in terms of the particular embodiments described herein, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Various embodiments are set forth within the following claims.

What is claimed is:

1. A method for treating, preventing, inhibiting, ameliorating or delaying the onset of heart failure, idiopathic cardiomyopathy, dilated cardiomyopathy, or restrictive cardiomyopathy in a mammalian subject in need thereof, comprising administering to the subject a therapeutically effective amount of a peptide of Formula A, or a mixture of peptides of Formula A: 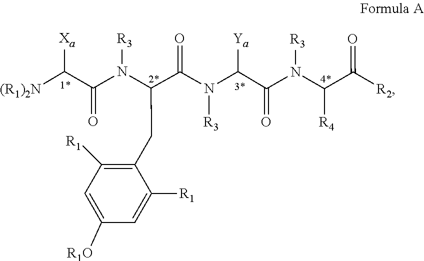
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 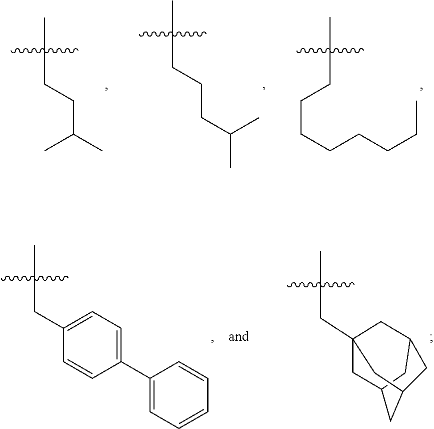
Xₐ is selected from 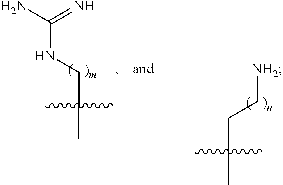
Yₐ is selected from 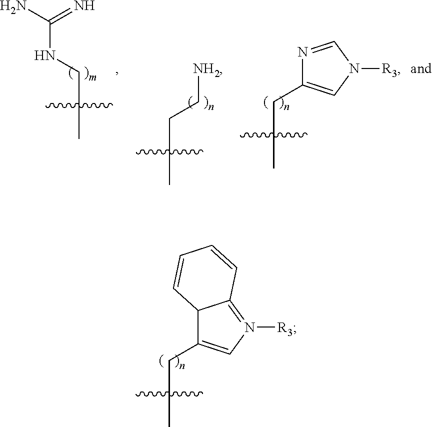
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

2. The method of claim 1, wherein the peptide or peptides of Formula A is/are a peptide/peptides of Formula A-1 or Formula A-2: 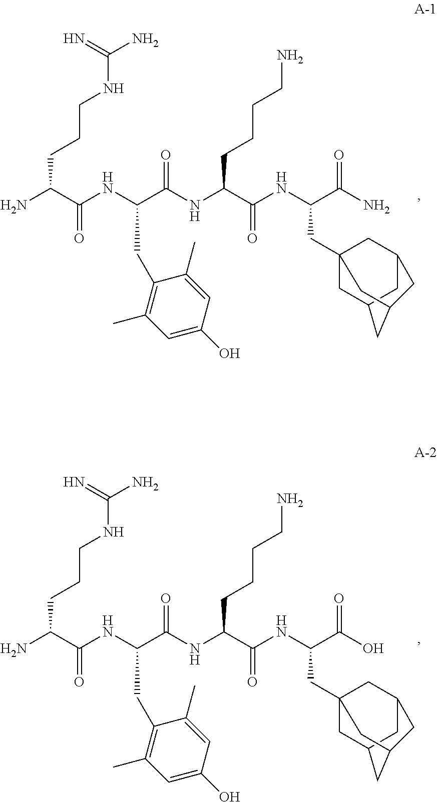 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

3. The method of claim 1, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 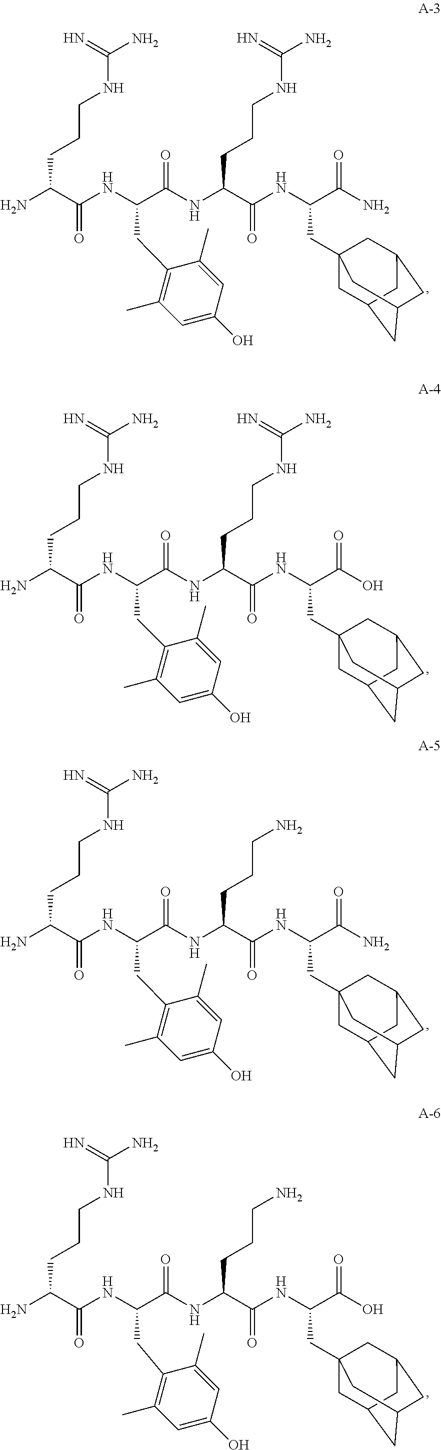 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 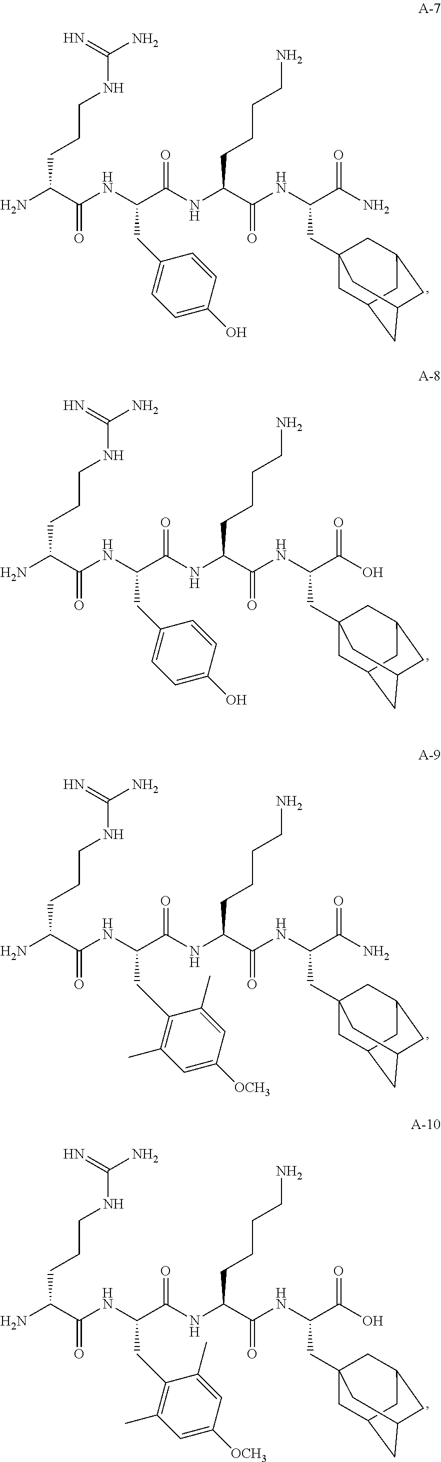 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 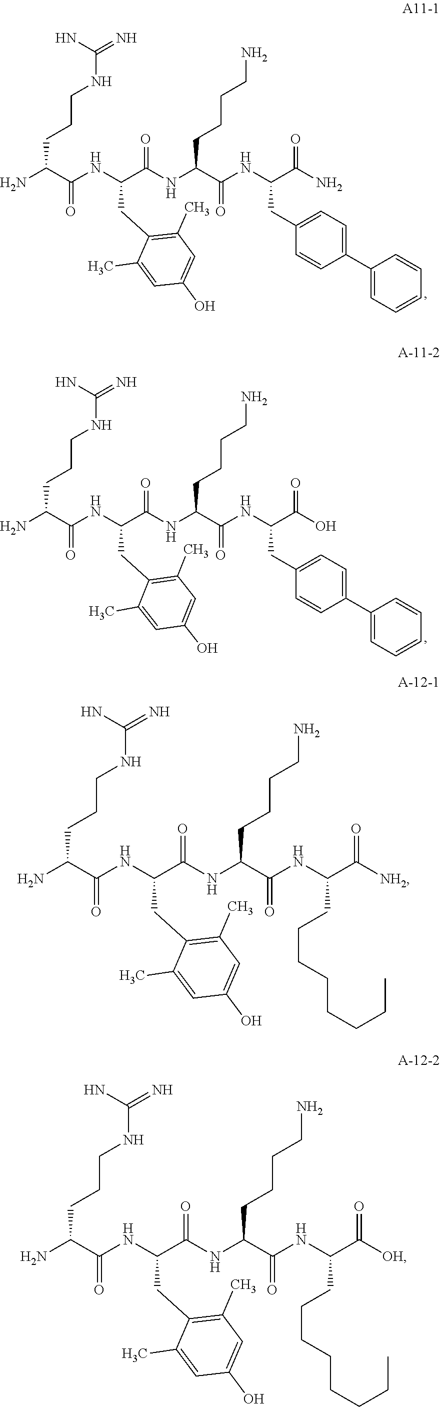 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 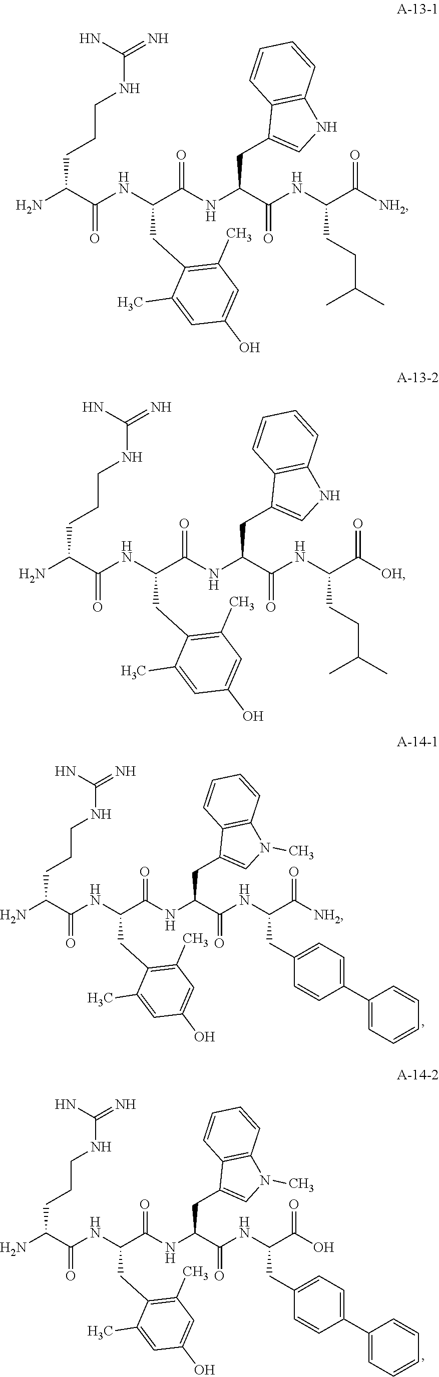 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 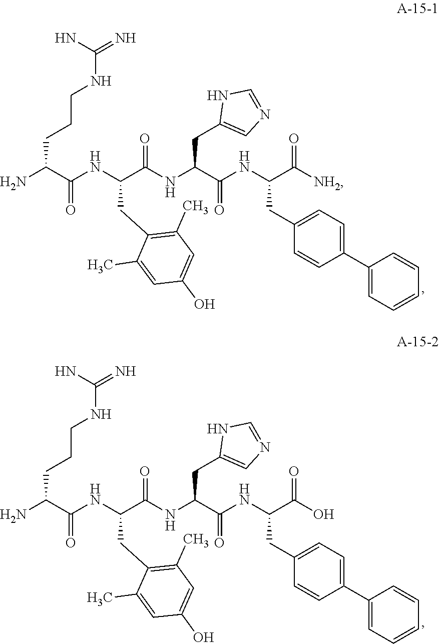 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

4. The method of any one of claims 1 to 3, wherein the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure.

5. The method of any one of claims 1 to 3, wherein the heart failure is congestive heart failure (CHF).

6. The method of any one of claims 1 to 5, wherein administration of the peptide or peptides, (i) ameliorate(s), (ii) delay(s) the onset of, and/or (iii) delays the progression of one or more signs or symptoms of heart failure in the subject as compared with a control subject or group of control subjects to which the peptide or peptides was/were not administered.

7. The method of claim 6, wherein the signs or symptoms of heart failure are: (i) shortness of breath, (ii) ankle and/or foot swelling; (iii) leg swelling; (iv) fatigue, and/or (v) labored breathing when lying flat.

8. The method of any one of claims 1 to 7, wherein the peptide or peptides is/are administered daily for: (i) 12 weeks or more; (ii) 24 weeks or more; (iii) 48 weeks or more; (iv) 72 weeks or more; or (v) 96 weeks or more.

9. The method of any one of claims 1 to 8, wherein the peptide or peptides is/are administered subcutaneously or intravenously.

10. The method of any one of claims 1 to 8, wherein the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

11. The method of any one of claims 1 to 10, wherein the subject is human.

12. The method of any one of claims 1 to 11, further comprising separately, sequentially, or simultaneously administering an additional therapeutic agent to the subject.

13. The method of claim 12, wherein the additional therapeutic agent is an angiotensin-converting enzyme (ACE) inhibitor, an angiotensin receptor blocker (ARB), a beta blocker, a diuretic, a mineralocorticoid receptor antagonist, or a calcium channel blocker (CBB).

14. The method of claim 13, wherein the angiotensin-converting enzyme (ACE) inhibitor is captopril, enalapril, lisinopril, benazepril, or ramipril.

15. The method of claim 13, wherein the angiotensin receptor blocker (ARB) is azilsartan, candesartan, eprosartan, irbesartan, telmisartan, valsartan, losartan, olmesartan, Entresto® (sacubitril/valsartan), or Byvalson™ (nebivolol/valsartan).

16. The method of claim 13, wherein the beta blocker is carvedilol, bisoprolol, metoprolol succinate, atenolol, esmolol, nebivolol, or propranolol.

17. The method of claim 13, wherein the diuretic is bumetanide, furosemide, or torsemide.

18. The method of claim 13, wherein the mineralocorticoid receptor antagonist is spironolactone or eplerenone.

19. The method of claim 13, wherein the calcium channel blocker is amlodipine (Norvasc®), diltiazem (Cardizem), felodipine, isradipine, nicardipine, nifedipine (Procardia®), nisoldipine (Sular®), or verapamil (Verelan®).

20. The method of claim 12, wherein the additional therapeutic agent is mavacamten (Camzyos®).

21. The method of any one of claims 1 to 20, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

22. The method of any one of claims 1 to 21, wherein the peptide or peptides is/are formulated for administration from its/their tris-HCl salt.

23. A composition or medicament for use in treating, preventing, inhibiting, ameliorating or delaying the onset of heart failure, idiopathic cardiomyopathy, dilated cardiomyopathy, or restrictive cardiomyopathy in a mammalian subject in need thereof, wherein the composition or medicament comprises a peptide of Formula A, or a mixture of peptides of Formula A: 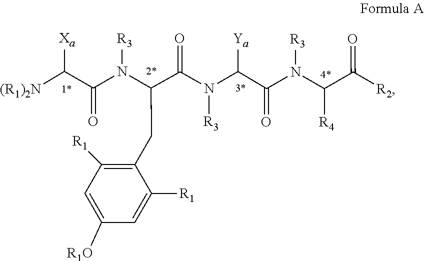
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 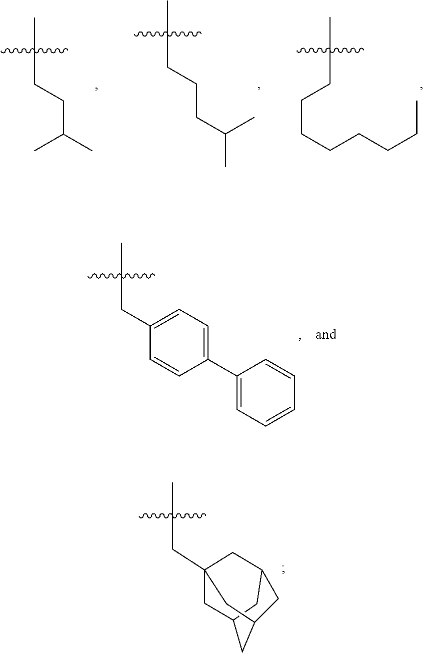
Xₐ is selected from 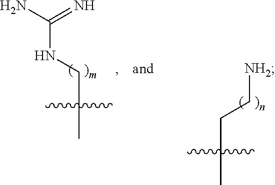
Yₐ is selected from 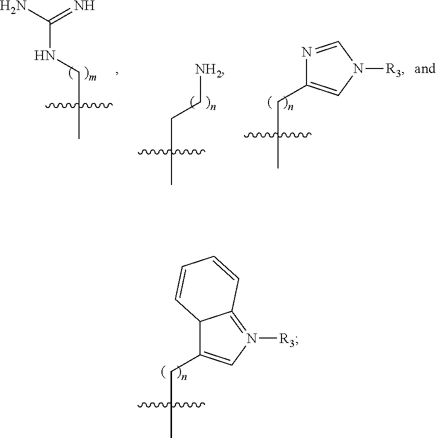
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

24. The composition or medicament of claim 23, wherein the peptide or peptides of Formula A is/are a peptide/peptides of Formula A-1 or Formula A-2: 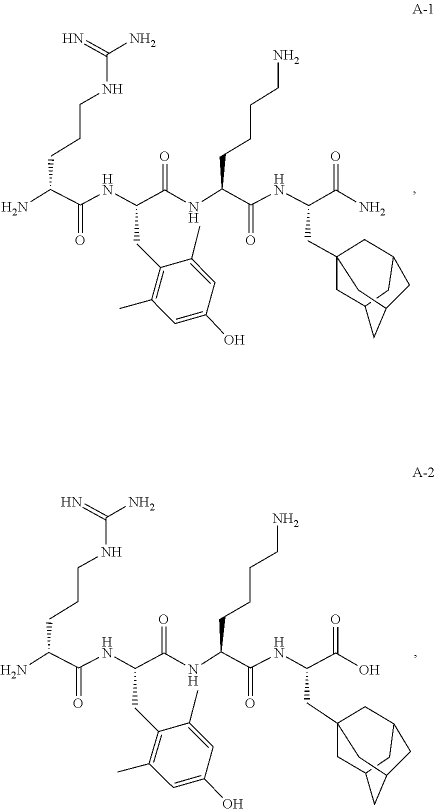 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

25. The composition or medicament of claim 23, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 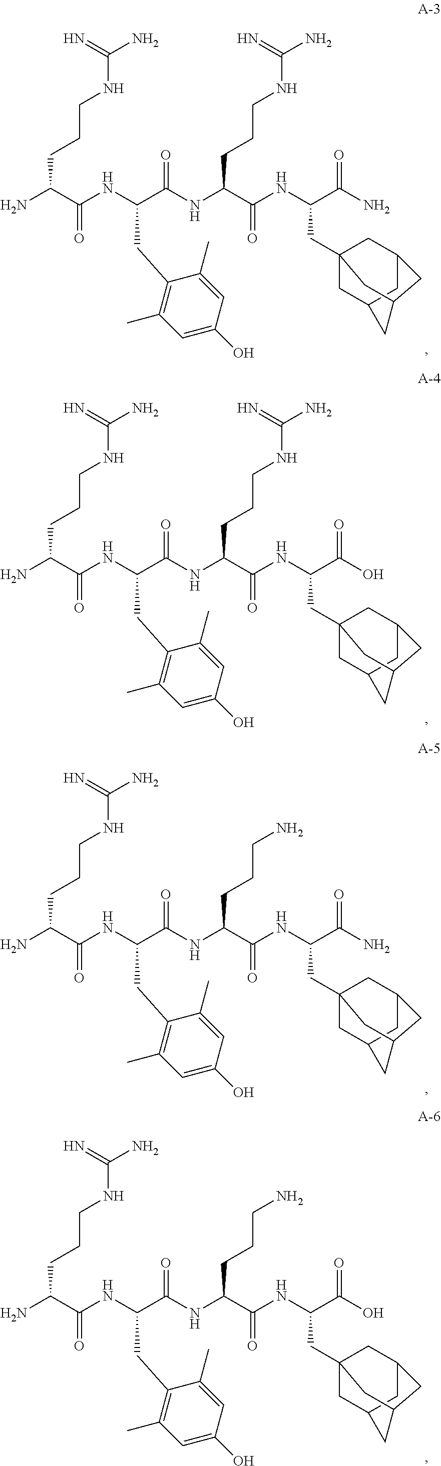 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 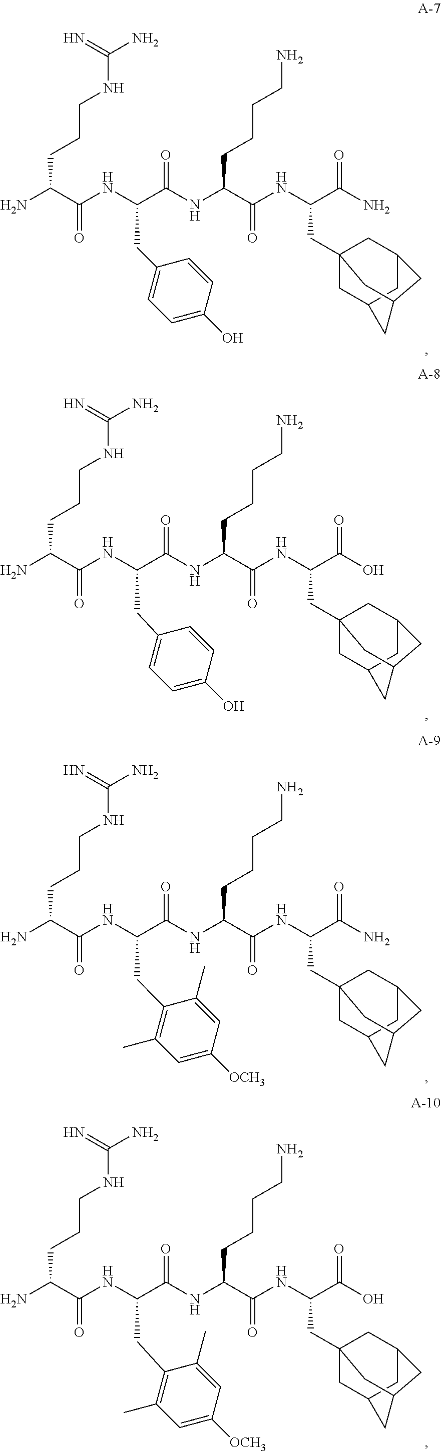 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 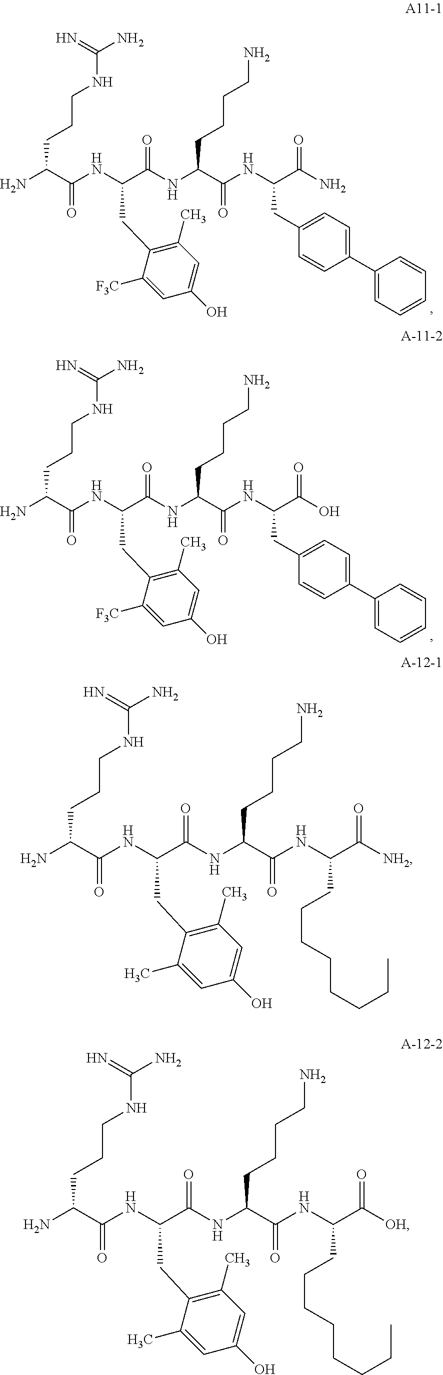 [SMILES: Cc1cc(O)cc(C(F)(F)F)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C(F)(F)F)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 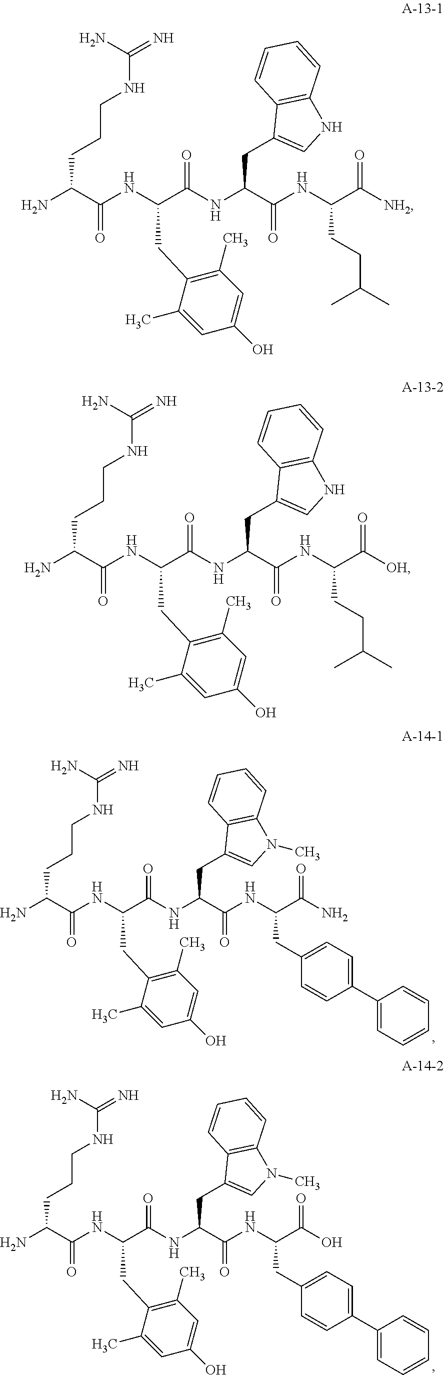 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 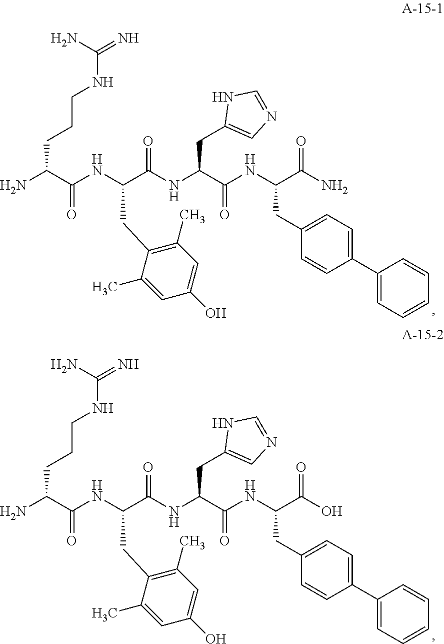 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

26. The composition or medicament of any one of claims 23 to 25, wherein the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure.

27. The composition or medicament of any one of claims 23 to 25, wherein the heart failure is congestive heart failure (CHF).

28. The composition or medicament of any one of claims 23 to 27, wherein administration of the composition or medicament to the subject, (i) ameliorates, (ii) delays the onset of, and/or (iii) delays the progression of one or more signs or symptoms of heart failure in the subject as compared with a control subject or group of control subjects to which the peptide or peptides was/were not administered.

29. The composition or medicament of claim 28, wherein the signs or symptoms of heart failure are: (i) shortness of breath, (ii) ankle and/or foot swelling; (iii) leg swelling; (iv) fatigue, or (v) labored breathing when lying flat.

30. The composition or medicament of any one of claims 23 to 29, wherein the composition or medicament is administered daily for: (i) 12 weeks or more; (ii) 24 weeks or more; (iii) 48 weeks or more; (iv) 72 weeks or more; or (v) 96 weeks or more.

31. The composition or medicament of any one of claims 23 to 30, wherein the composition or medicament is administered subcutaneously or intravenously.

32. The composition or medicament of any one of claims 23 to 30, wherein the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

33. The composition or medicament of any one of claims 23 to 32, wherein the subject is human.

34. The composition or medicament of any one of claims 23 to 33, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

35. The composition or medicament of any one of claims 23 to 33, wherein the peptide or peptides of the composition or medicament is/are formulated for administration from its/their tris-HCl salt.

36. Use of a peptide, mixture of peptides, or a composition or medicament comprising the peptide or mixture of peptides for treating, preventing, inhibiting, ameliorating or delaying the onset of heart failure, idiopathic cardiomyopathy, dilated cardiomyopathy, or restrictive cardiomyopathy in a mammalian subject in need thereof, wherein the peptide or peptides of the mixture is/are of Formula A: 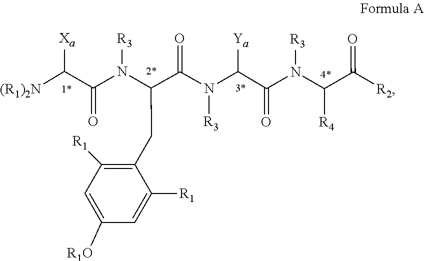
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 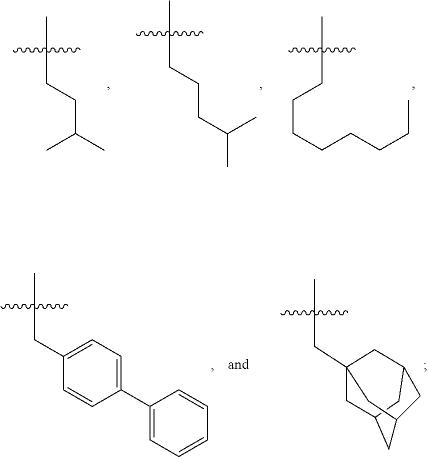
Xₐ is selected from 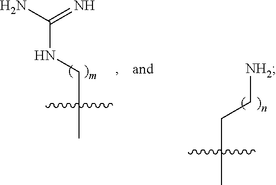
Yₐ is selected from 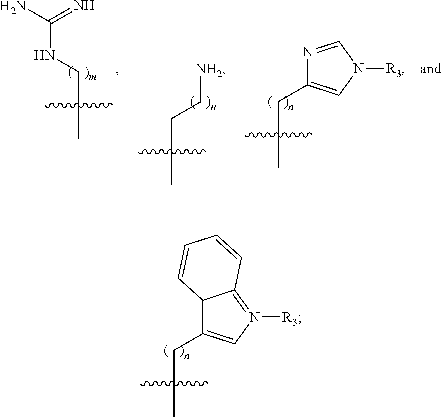
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

37. The use of claim 36, wherein the peptide or peptides of Formula A is/are a peptide/peptides of Formula A-1 or Formula A-2: 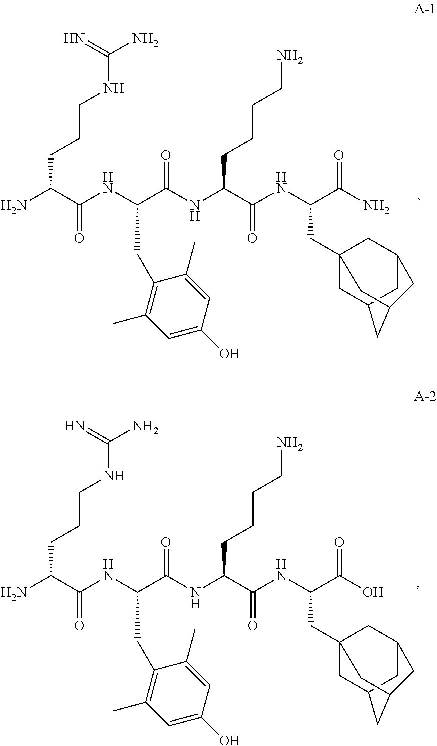 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

38. The use of claim 36, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 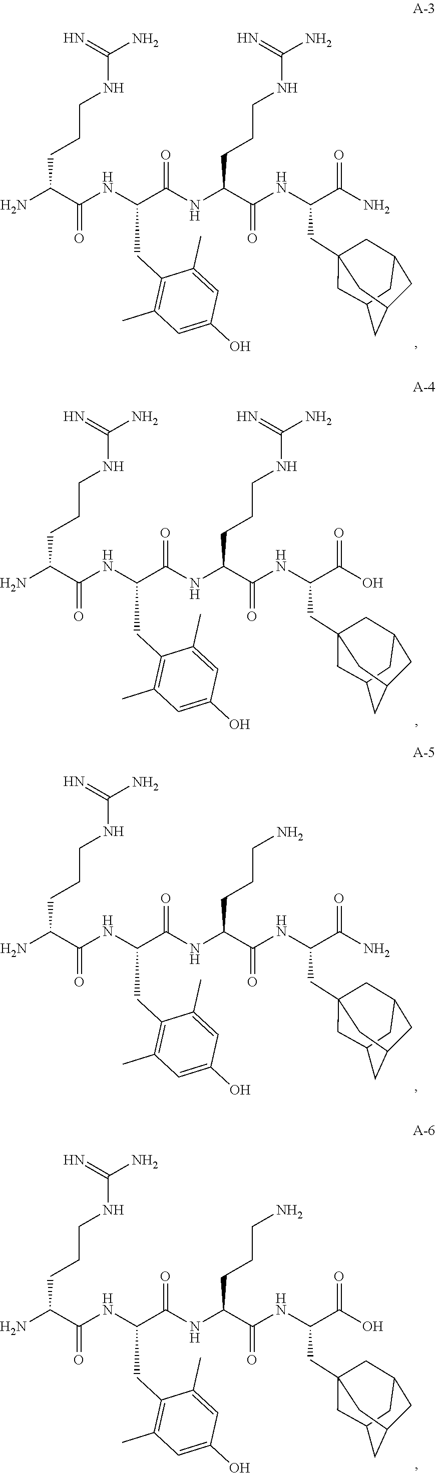 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 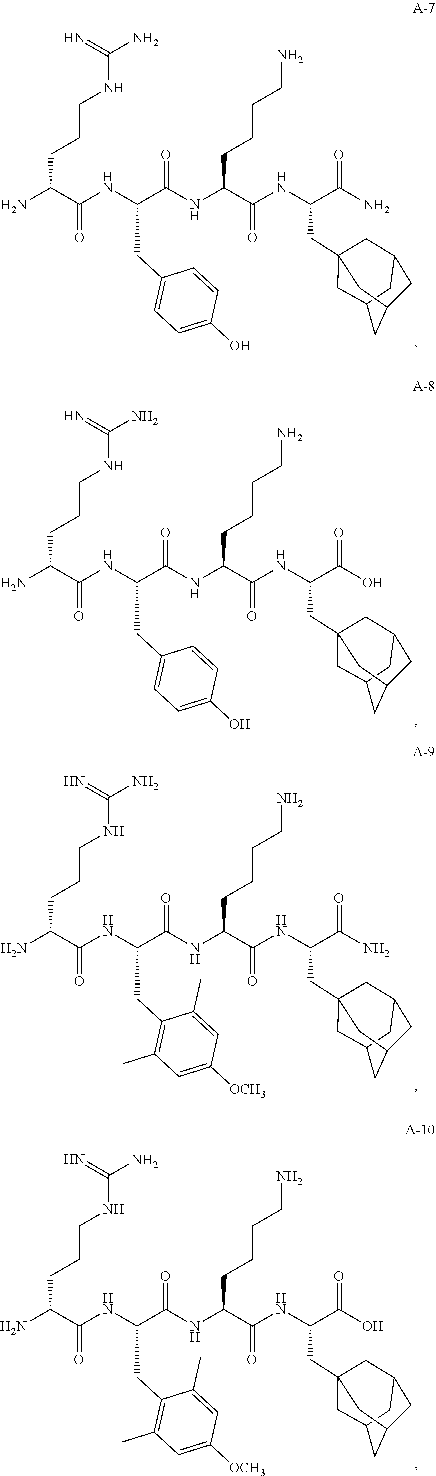 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 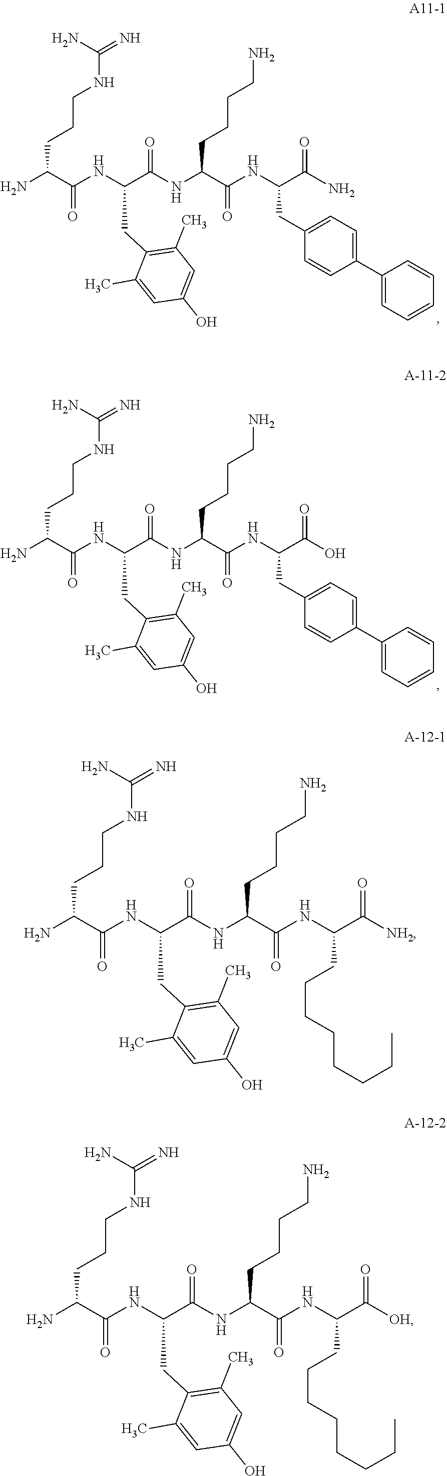 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 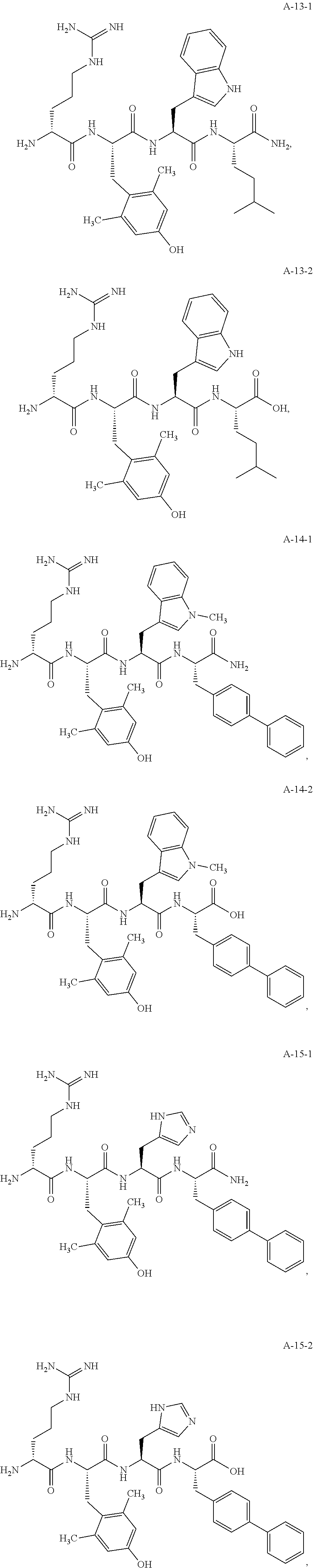
or a pharmaceutically acceptable salt, hydrate, solvate, stereoisomer and/or tautomer thereof.

39. The use of any one of claims 36 to 38, wherein the heart failure is: (i) heart failure with reduced ejection fraction (HFrEF), (ii) heart failure with preserved ejection fraction (HFpEF), (iii) right-sided heart failure, (iv) left-sided heart failure, (v) biventricular heart failure, or (vi) high-output heart failure.

40. The use of any one of claims 36 to 38, wherein the heart failure is congestive heart failure (CHF).

41. The use of any one of claims 36 to 40, wherein administration of the peptide, mixture of peptides or the composition comprising the peptide or mixture of peptides to the subject, (i) ameliorates, (ii) delays the onset of, and/or (iii) delays the progression of one or more signs or symptoms of heart failure in the subject as compared with a control subject or group of control subjects to which the peptide or peptides was/were not administered.

42. The use of claim 41, wherein the signs or symptoms of heart failure are: (i) shortness of breath, (ii) ankle and/or foot swelling; (iii) leg swelling; (iv) fatigue, or (v) labored breathing when lying flat.

43. The use of any one of claims 36 to 42, wherein the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more.

44. The use of any one of claims 36 to 43, wherein the peptide, mixture of peptides, or the composition or medicament comprising the peptide or mixture of peptides is administered subcutaneously or intravenously.

45. The use of any one of claims 36 to 43, wherein the peptide, mixture of peptides or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

46. The use of any one of claims 36 to 45, wherein the subject is human.

47. The use of any one of claims 36 to 46, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

48. The use of any one of claims 36 to 46, wherein the peptide or peptides is/are formulated for administration from its/their tris-HCl salt.

49. A method for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, comprising administering to the subject a therapeutically effective amount of a peptide of Formula A, or a mixture of peptides of Formula A: 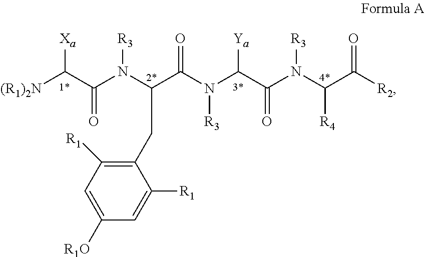
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 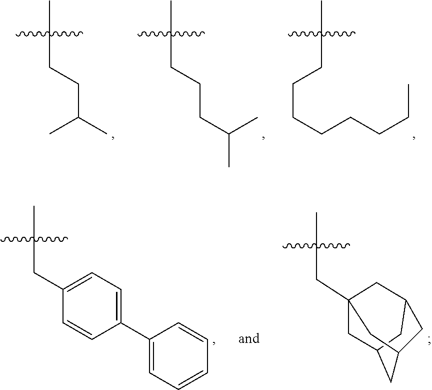
Xₐ is selected from 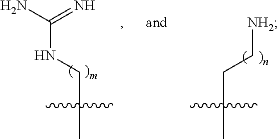
Yₐ is selected from 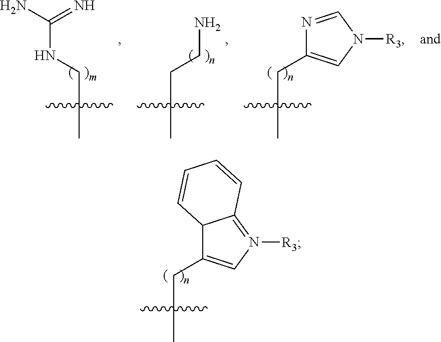
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

50. The method of claim 49, wherein the peptide or peptides of Formula A is/are a peptide/peptides of Formula A-1 or Formula A-2: 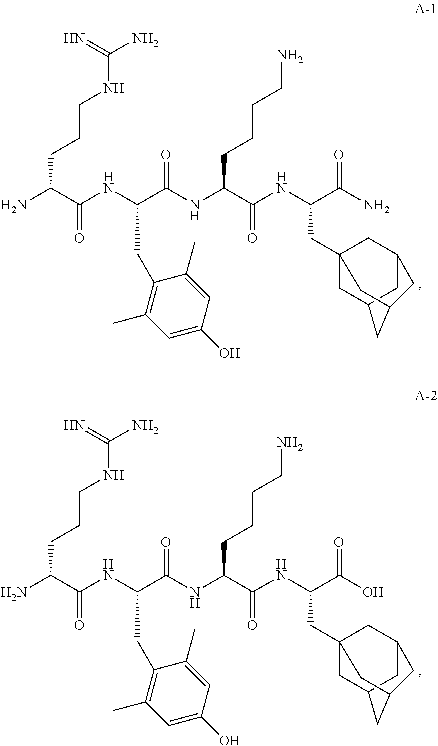 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

51. The method of claim 49, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 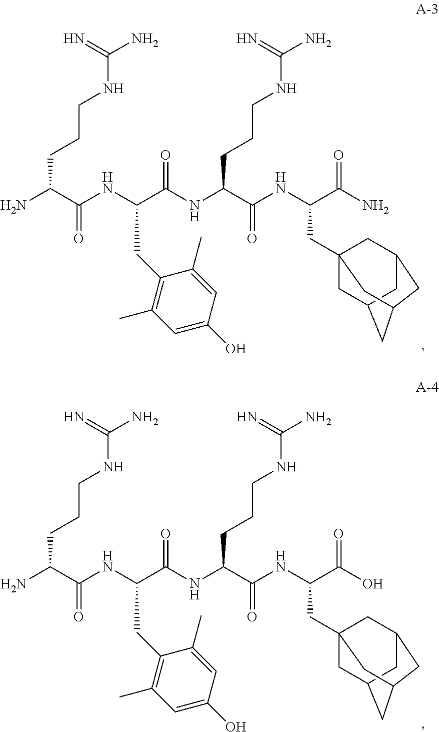 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O] 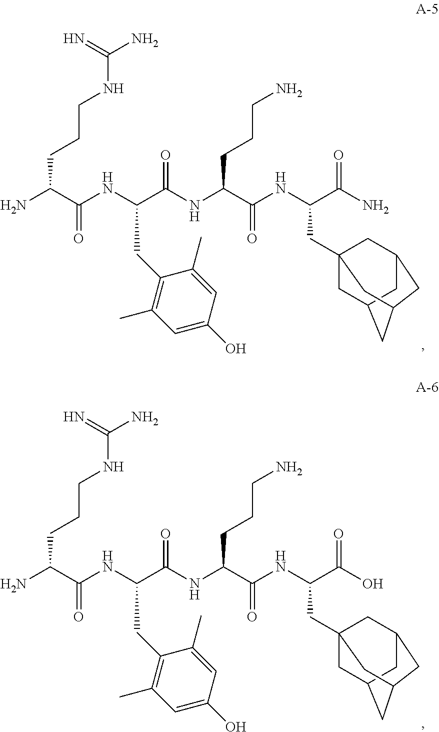 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 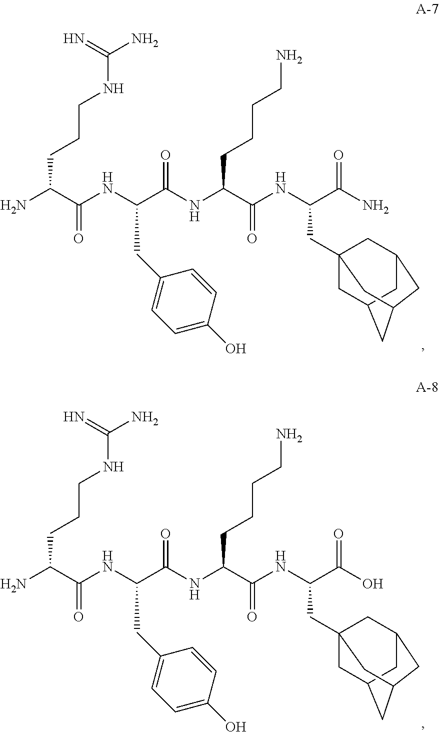 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 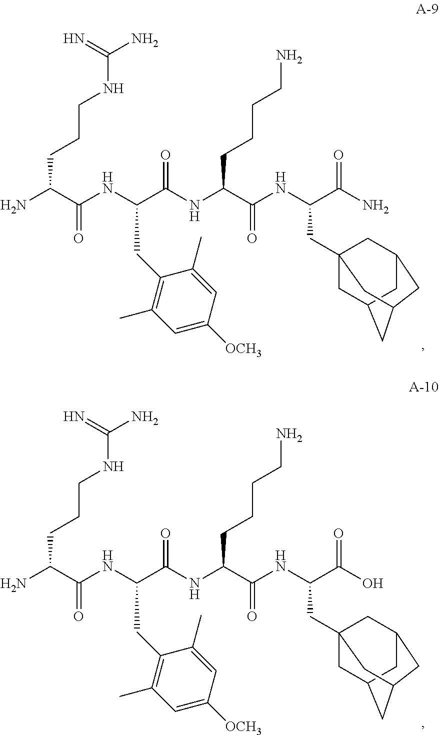 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 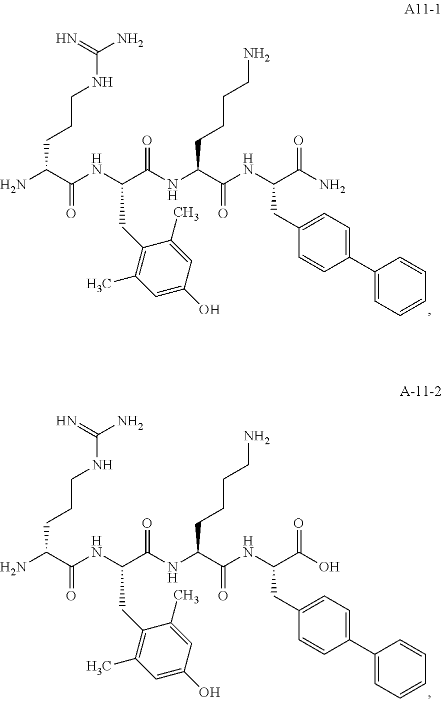 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O] 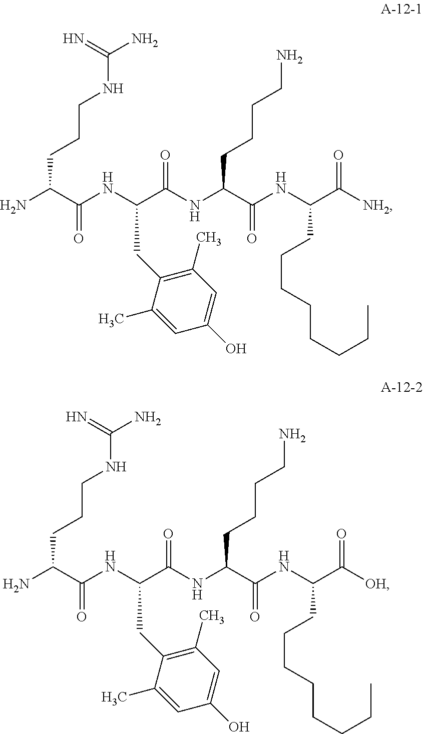 [SMILES: CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 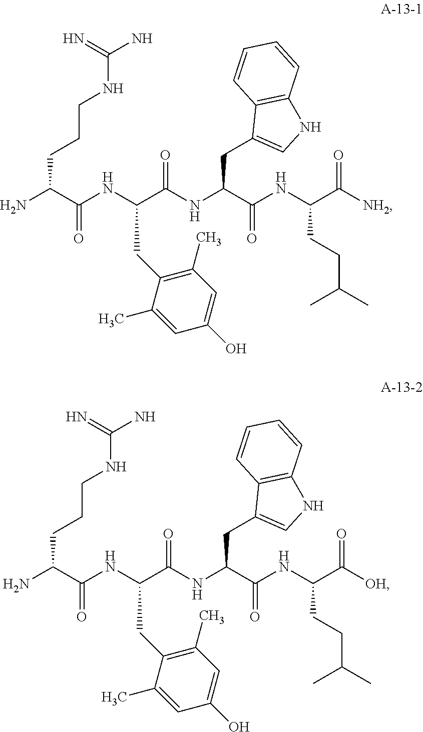 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O] 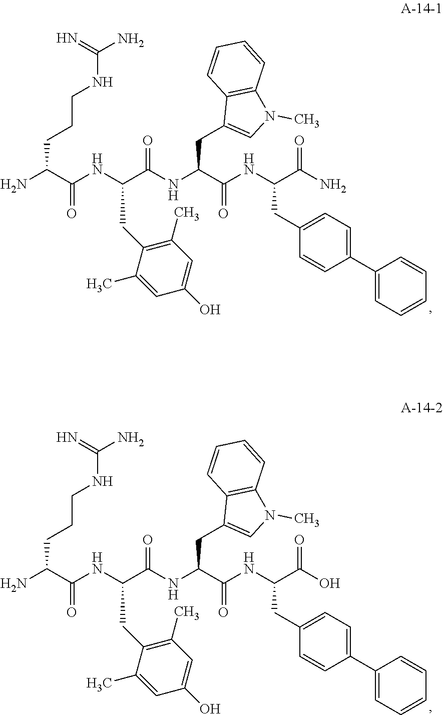 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O] 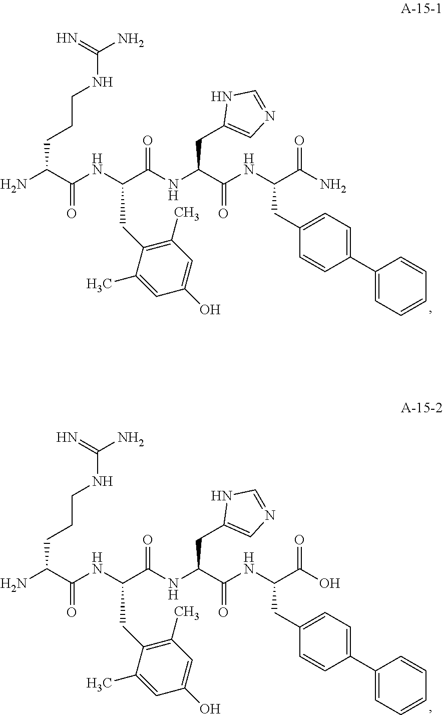 [SMILES: Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

52. The method of any one of claims 49 to 51, wherein the peptide or peptides is/are administered daily for: (i) 12 weeks or more; (ii) 24 weeks or more; (iii) 48 weeks or more; (iv) 72 weeks or more; or (v) 96 weeks or more.

53. The method of any one of claims 49 to 52, wherein the peptide or peptides is/are administered subcutaneously or intravenously.

54. The method of any one of claims 49 to 52, wherein the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

55. The method of any one of claims 49 to 54, wherein the subject is human.

56. The method of any one of claims 49 to 55, further comprising separately, sequentially, or simultaneously administering an additional therapeutic agent to the subject.

57. The method of claim 56, wherein the additional therapeutic agent is a beta blocker, a diuretic, a mineralocorticoid receptor antagonist or a calcium channel blocker (CCB).

58. The method of claim 57, wherein the beta blocker is carvedilol, bisoprolol, metoprolol succinate, atenolol, esmolol, nebivolol, or propranolol.

59. The method of claim 57, wherein the diuretic is bumetanide, furosemide, or torsemide.

60. The method of claim 57, wherein the mineralocorticoid receptor antagonist is spironolactone or eplerenone.

61. The method of claim 57, wherein the calcium channel blocker is amlodipine (Norvasc®), diltiazem (Cardizem), felodipine, isradipine, nicardipine, nifedipine (Procardia®), nisoldipine (Sular®), or verapamil (Verelan®).

62. The method of claim 56, wherein the additional therapeutic agent is mavacamten (Camzyos®).

63. The method of any one of claims 49 to 62, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

64. The method of any one of claims 49 to 62, wherein the peptide or peptides is/are formulated for administration from its/their tris-HCl salt.

65. A composition or medicament for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, wherein the composition or medicament comprises a peptide of Formula A, or a mixture of peptides of Formula A: 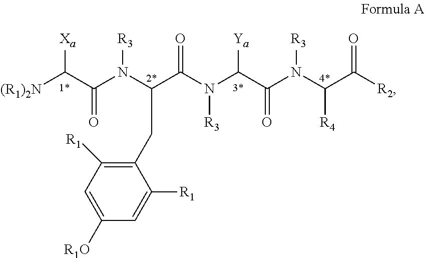
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 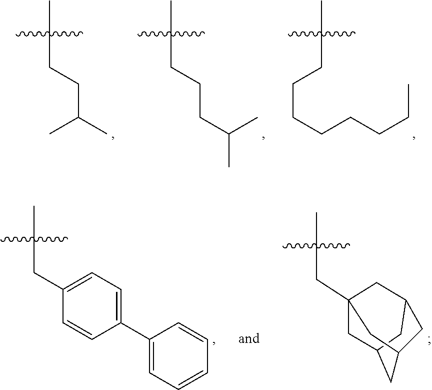
Xₐ is selected from 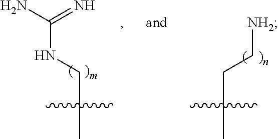
Yₐ is selected from 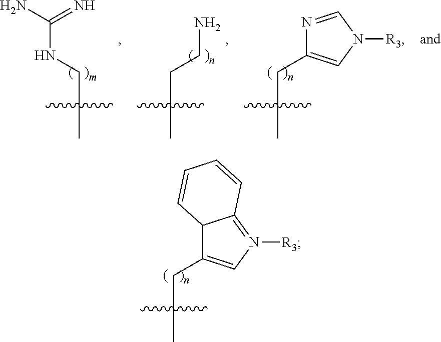
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide or peptides of Formula A is/are substituted with a deuterium or fluorine atom.

66. The composition or medicament of claim 65, wherein the peptide or peptides of Formula A is/are a peptide/peptides of Formula A-1 or Formula A-2: 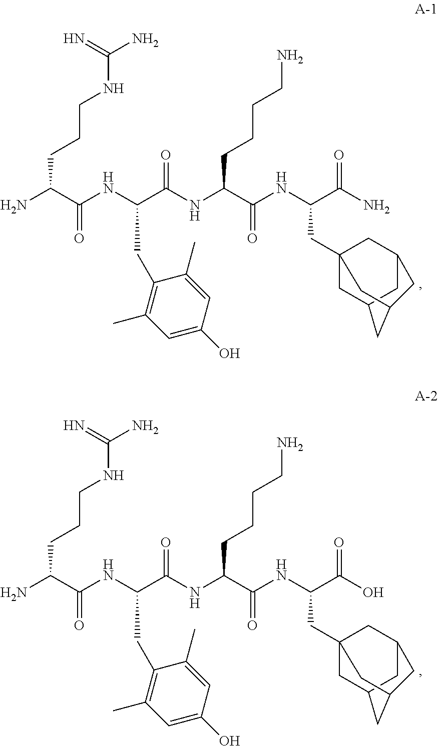 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

67. The composition or medicament of claim 65, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 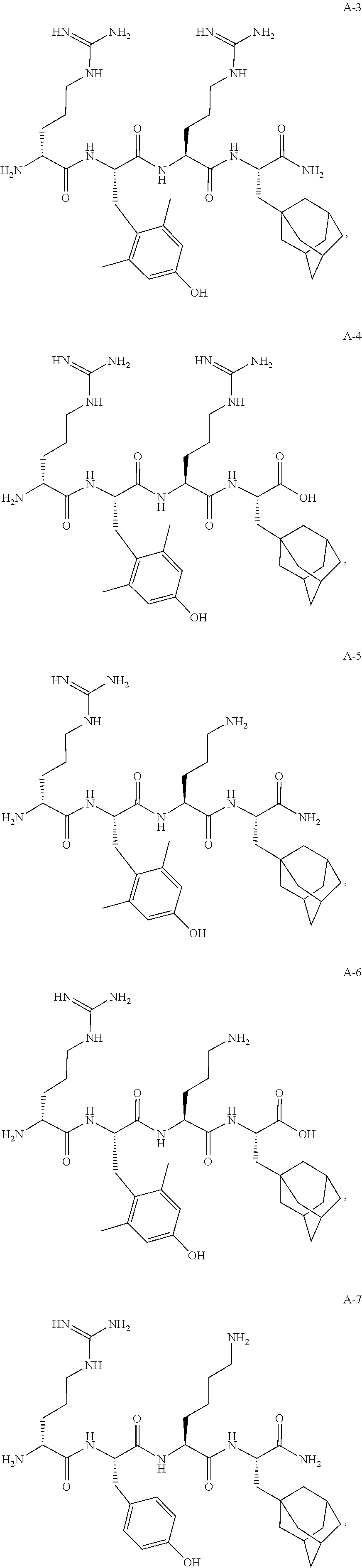 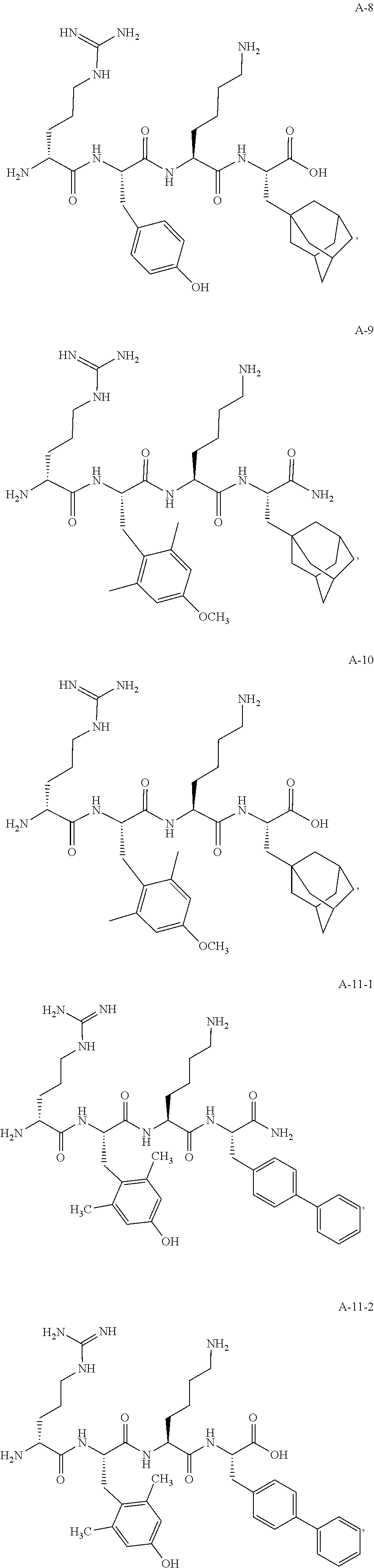 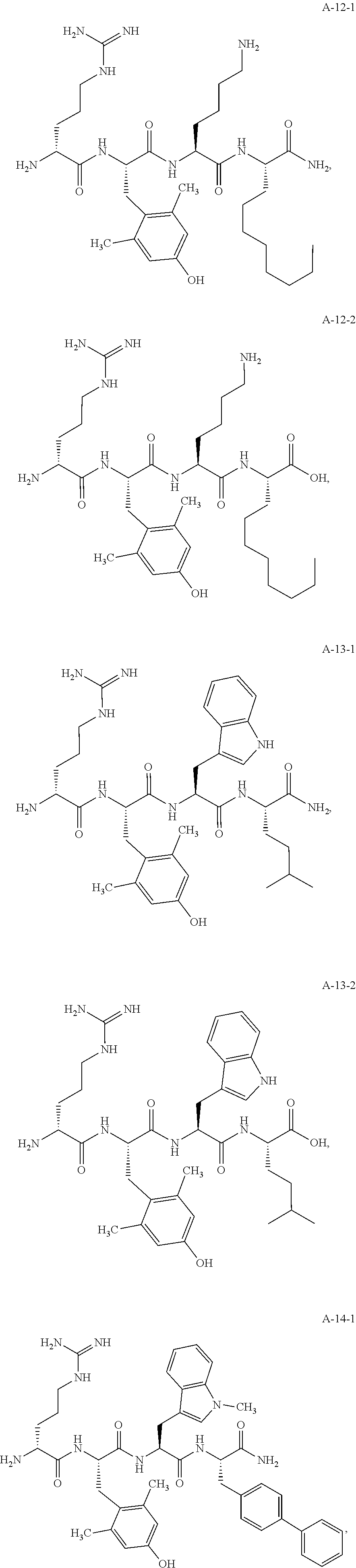 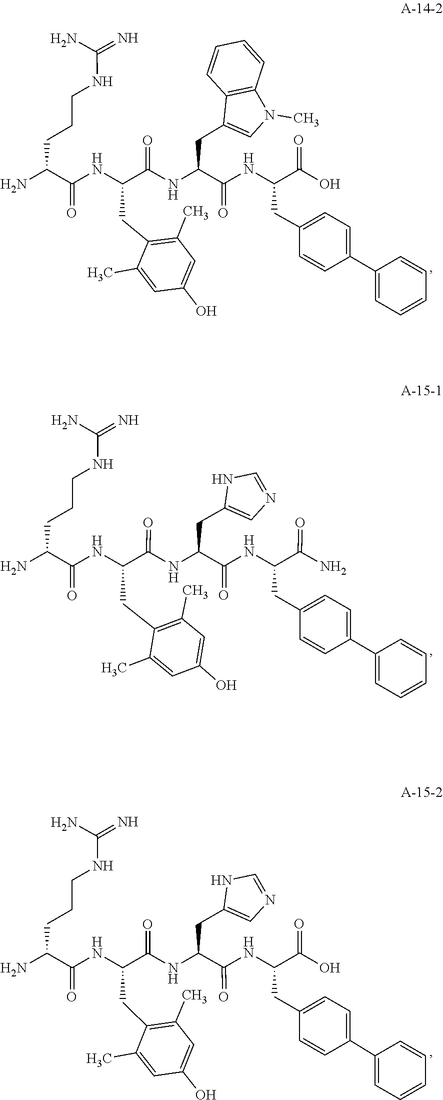 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

68. The composition or medicament of any one of claims 65 to 67, wherein the composition or medicament is administered daily for: (i) 12 weeks or more; (ii) 24 weeks or more; (iii) 48 weeks or more; (iv) 72 weeks or more; or (v) 96 weeks or more.

69. The composition or medicament of any one of claims 65 to 68, wherein the composition or medicament is administered subcutaneously or intravenously.

70. The composition or medicament of any one of claims 65 to 68, wherein the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

71. The composition or medicament of any one of claims 65 to 70, wherein the subject is human.

72. The composition or medicament of any one of claims 65 to 71, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

73. The composition or medicament of any one of claims 65 to 71, wherein the peptide or peptides of the composition or medicament is/are formulated for administration from its/their tris-HCl salt.

74. Use of a peptide, mixture of peptides, or a composition or medicament comprising the peptide or mixture of peptides for treating, preventing, inhibiting, ameliorating or delaying the onset of hypertrophic cardiomyopathy (including obstructive hypertrophic cardiomyopathy) or hypertensive cardiomyopathy in a mammalian subject in need thereof, wherein the peptide or peptides is/are of Formula A: 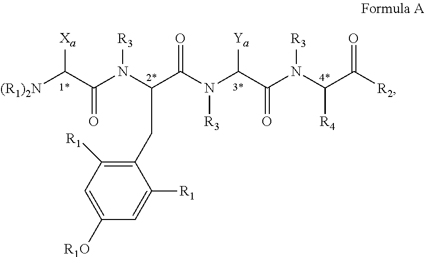
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 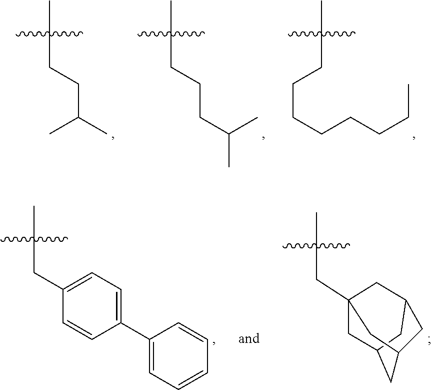
Xₐ is selected from 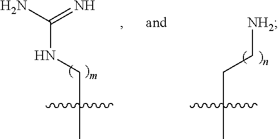
Yₐ is selected from 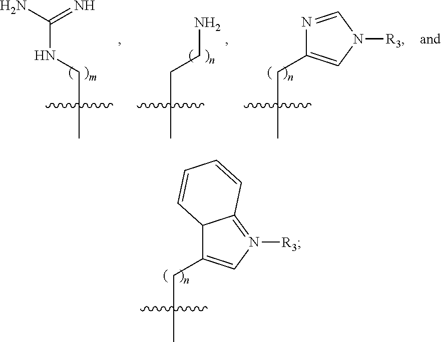
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide or peptides of Formula A is/are substituted with a deuterium or fluorine atom.

75. The use of claim 74, wherein the peptide or peptides of Formula A is/are a peptide/peptides of Formula A-1 or Formula A-2: 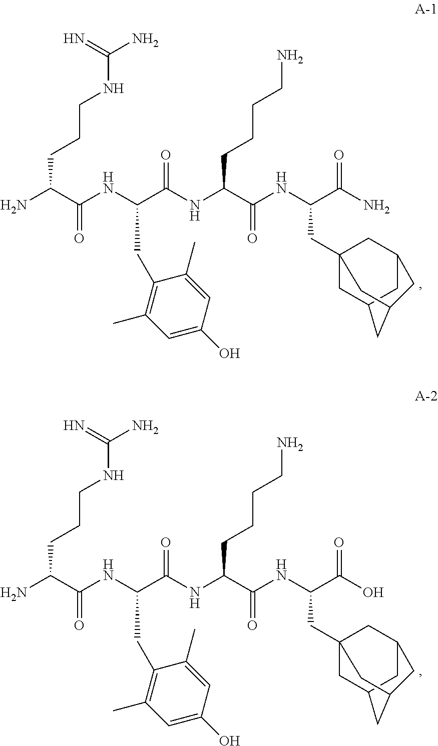 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

76. The use of claim 74, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 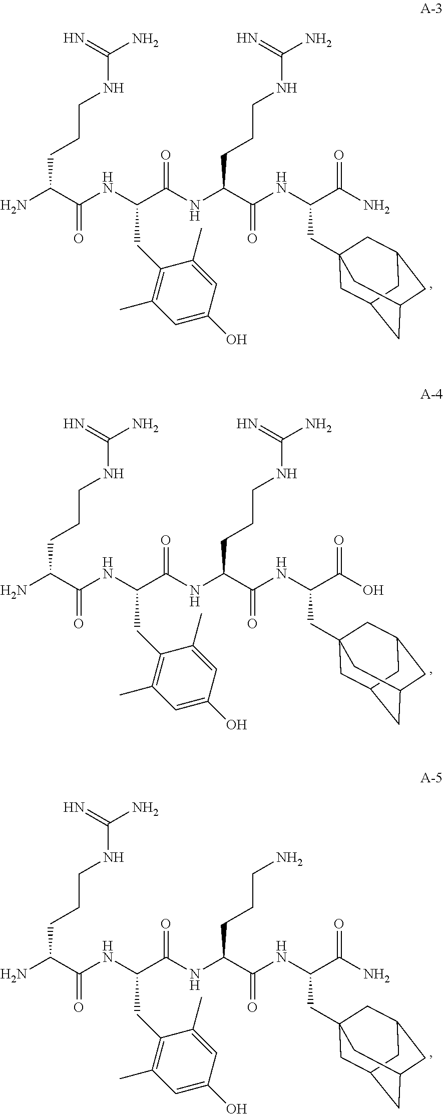 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O] 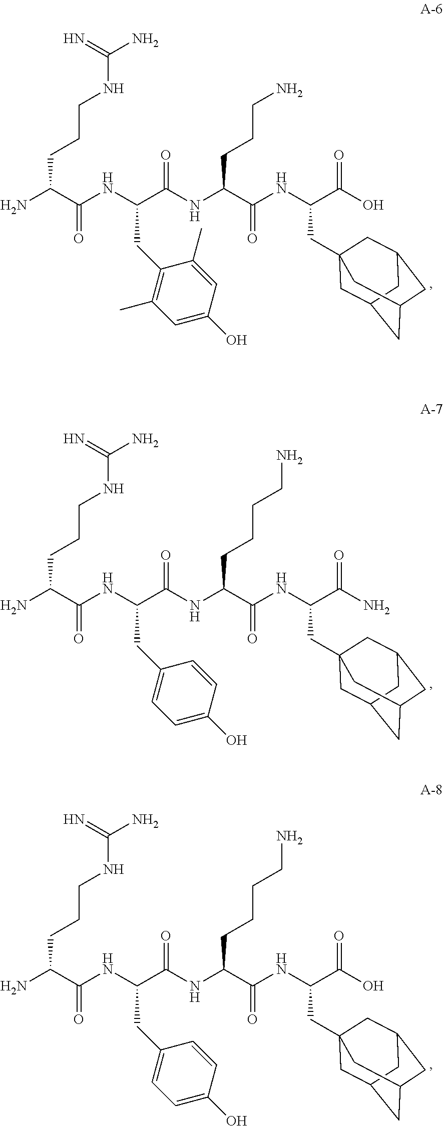 [SMILES: Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O] 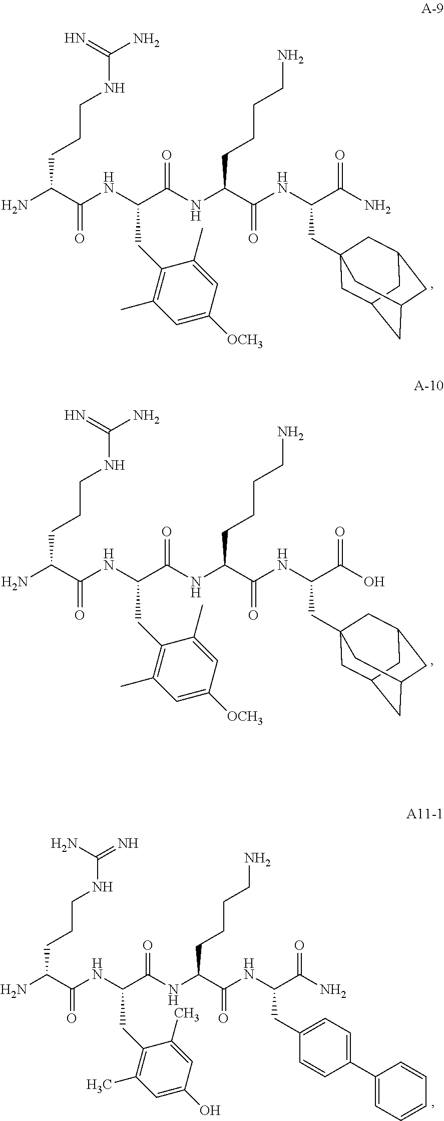 [SMILES: COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1 ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O] 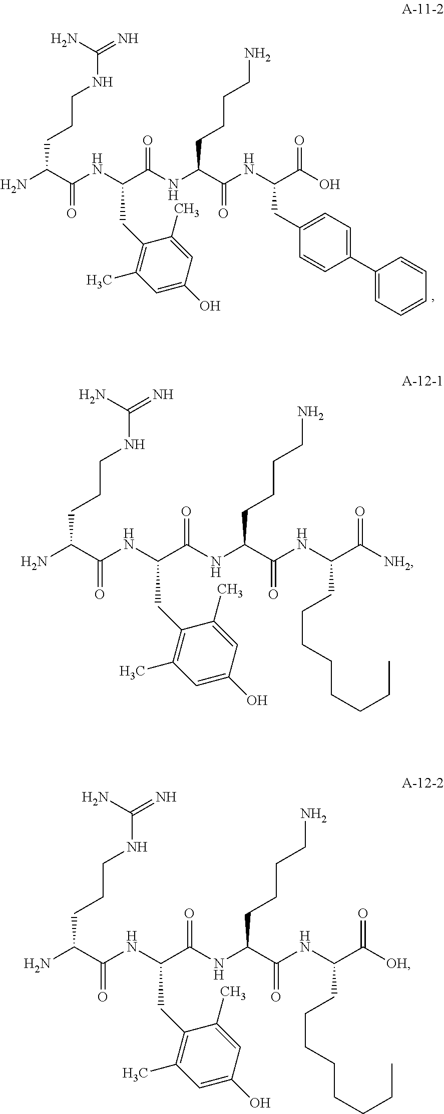 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 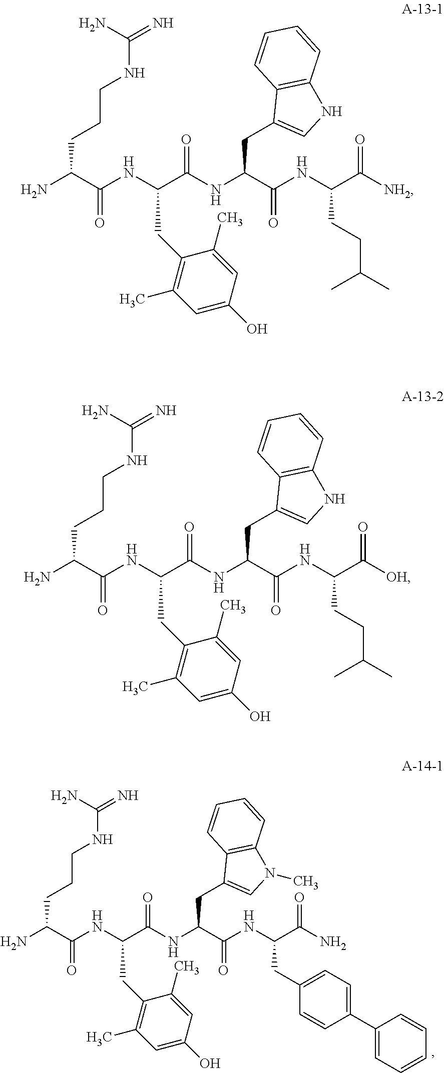 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O] 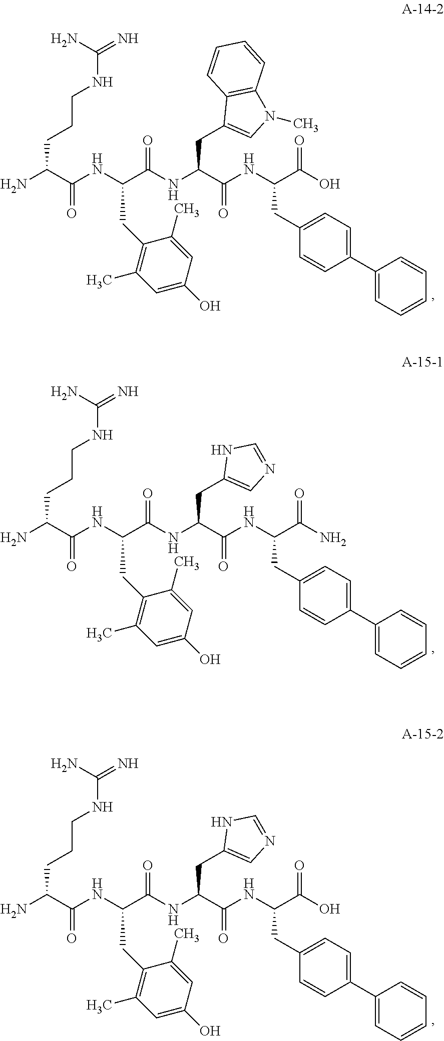 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

77. The use of any one of claims 74 to 76, wherein the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more.

78. The use of any one of claims 74 to 77, wherein the peptide, mixture of peptides, or the composition or medicament comprising the peptide or mixture of peptides is administered subcutaneously or intravenously.

79. The use of any one of claims 74 to 77, wherein the peptide, mixture of peptides, or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

80. The use of any one of claims 74 to 79, wherein the subject is human.

81. The use of any one of claims 74 to 80, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

82. The use of any one of claims 74 to 80, wherein the peptide or peptides is/are formulated for administration from its/their tris-HCl salt.

83. A method for normalizing left ventricular ejection fraction, fractional shortening, and/or global longitudinal strain in a mammalian subject in need thereof, comprising administering to the subject a therapeutically effective amount of a peptide of Formula A, or a mixture of peptides of Formula A: 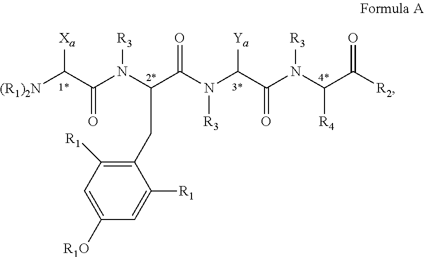
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 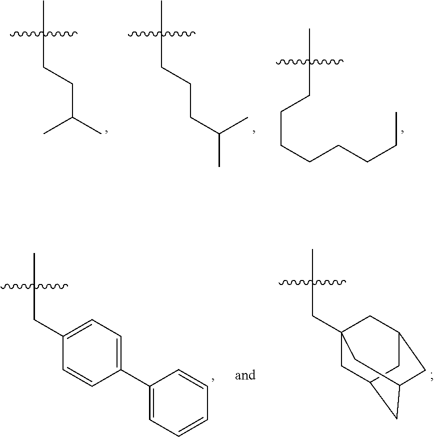
Xₐ is selected from 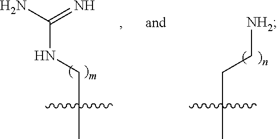
Yₐ is selected from 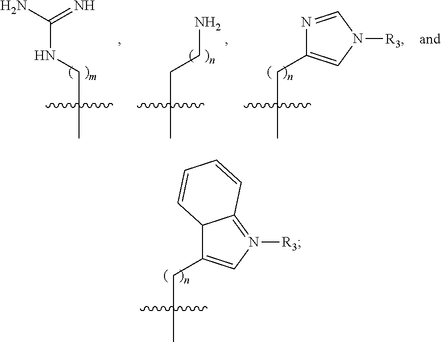
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom, wherein normalization or the left ventricular ejection fraction, fractional shortening, and/or global longitudinal strain occurs in the subject as compared with a control subject to control group of subjects not administered the peptide or peptides of Formula A.

84. The method of claim 83, wherein the peptide or peptides of Formula A is/are a peptide/peptides of Formula A-1 or Formula A-2: 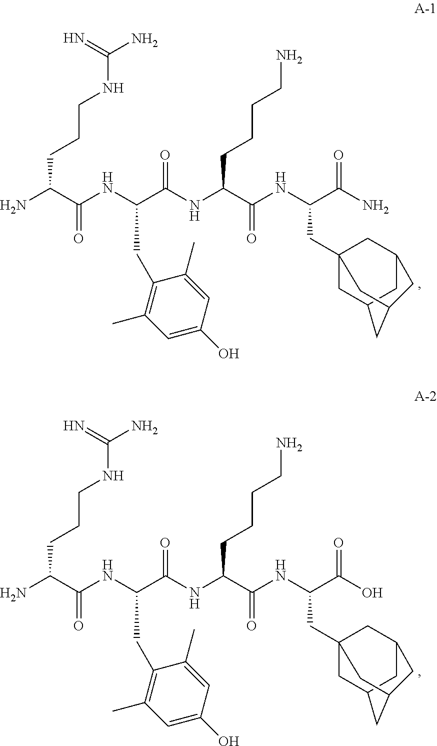 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

85. The method of claim 83, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 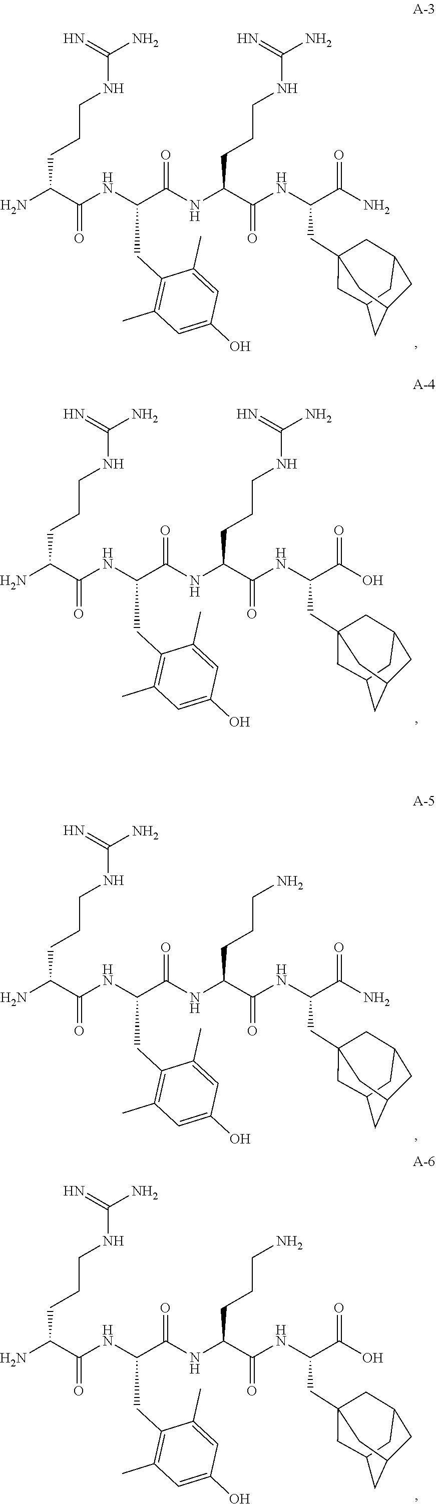 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 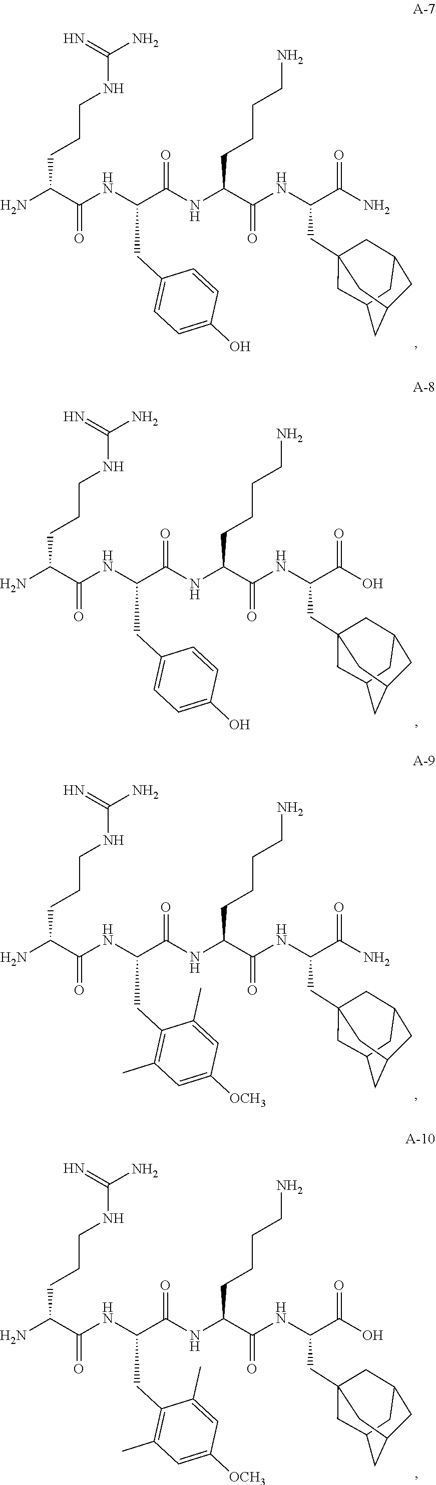 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 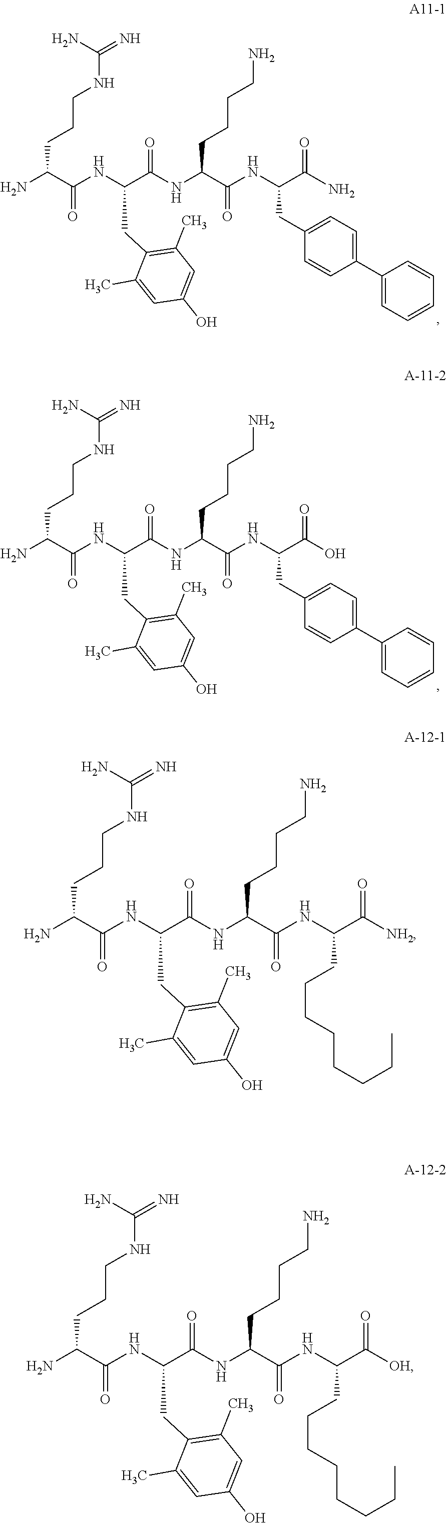 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 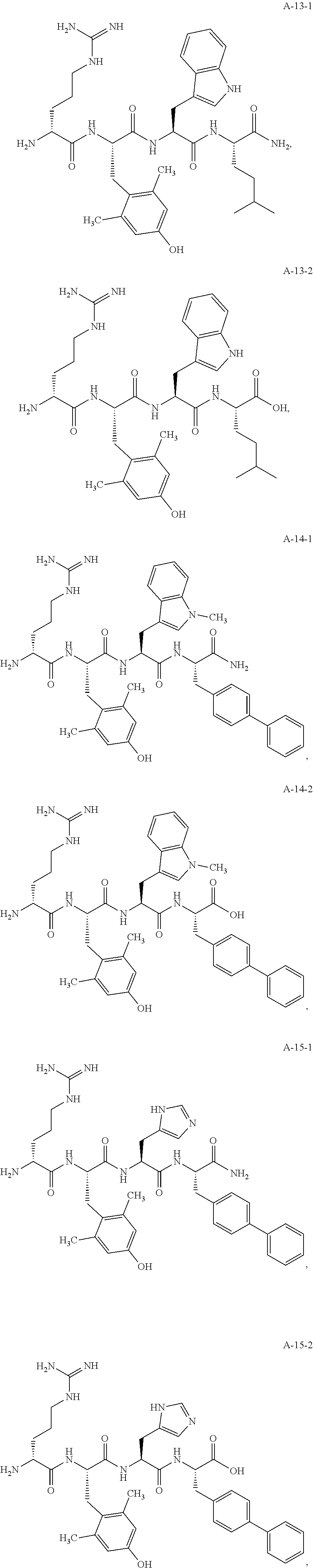
or a pharmaceutically acceptable salt, hydrate, solvate, stereoisomer and/or tautomer thereof.

86. The method of any one of claims 83 to 85, wherein the peptide or peptides is/are administered daily for: (i) 12 weeks or more; (ii) 24 weeks or more; (iii) 48 weeks or more; (iv) 72 weeks or more; or (v) 96 weeks or more.

87. The method of any one of claims 83 to 86, wherein the peptide or peptides is/are administered subcutaneously or intravenously.

88. The method of any one of claims 83 to 86, wherein the peptide or peptides is/are administered orally, topically, systemically, intraperitoneally, intradermally, transdermally, ophthalmically, intrathecally, intracerebroventricularly, iontophoretically, transmucosally, intravitreally, intranasally, or intramuscularly.

89. The method of any one of claims 83 to 88, wherein the subject is human.

90. The method of any one of claims 83 to 89, further comprising separately, sequentially, or simultaneously administering an additional therapeutic agent to the subject.

91. The method of claim 90, wherein the additional therapeutic agent is a beta blocker, a diuretic, a mineralocorticoid receptor antagonist or a calcium channel blocker (CCB).

92. The method of claim 91, wherein the beta blocker is carvedilol, bisoprolol, metoprolol succinate, atenolol, esmolol, nebivolol, or propranolol.

93. The method of claim 91, wherein the diuretic is bumetanide, furosemide, or torsemide.

94. The method of claim 91, wherein the mineralocorticoid receptor antagonist is spironolactone or eplerenone.

95. The method of claim 91, wherein the calcium channel blocker is amlodipine (Norvasc®), diltiazem (Cardizem), felodipine, isradipine, nicardipine, nifedipine (Procardia®), nisoldipine (Sular®), or verapamil (Verelan®).

96. The method of claim 90, wherein the additional therapeutic agent is mavacamten (Camzyos®).

97. The method of any one of claims 83 to 96, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

98. The method of any one of claims 83 to 96, wherein the peptide or peptides is/are formulated for administration from its/their tris-HCl salt.

99. A composition or medicament for normalizing left ventricular ejection fraction, fractional shortening, and/or global longitudinal strain in a mammalian subject in need thereof, wherein the composition or medicament comprises a peptide of Formula A, or a mixture of peptides of Formula A: 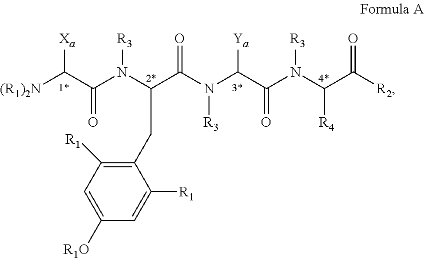
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 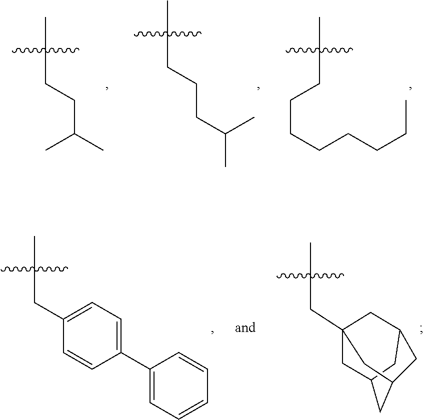
Xₐ is selected from 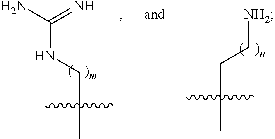
Yₐ is selected from 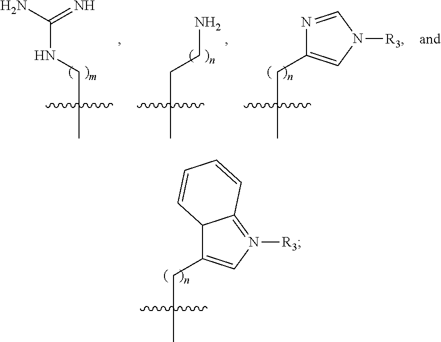
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms in the peptide of Formula A is substituted with a deuterium or fluorine atom, wherein normalization or the left ventricular ejection fraction, fractional shortening, and/or global longitudinal strain occurs in the subject as compared with a control subject to control group of subjects not administered the peptide or peptides of Formula A.

100. The composition or medicament of claim 99, wherein the peptide or peptides of Formula A is/are a peptide/peptides of Formula A-1 or Formula A-2: 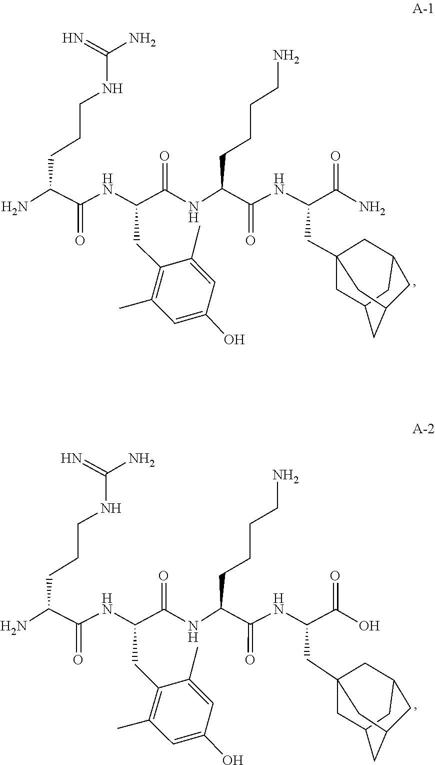 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

101. The composition or medicament of claim 99, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 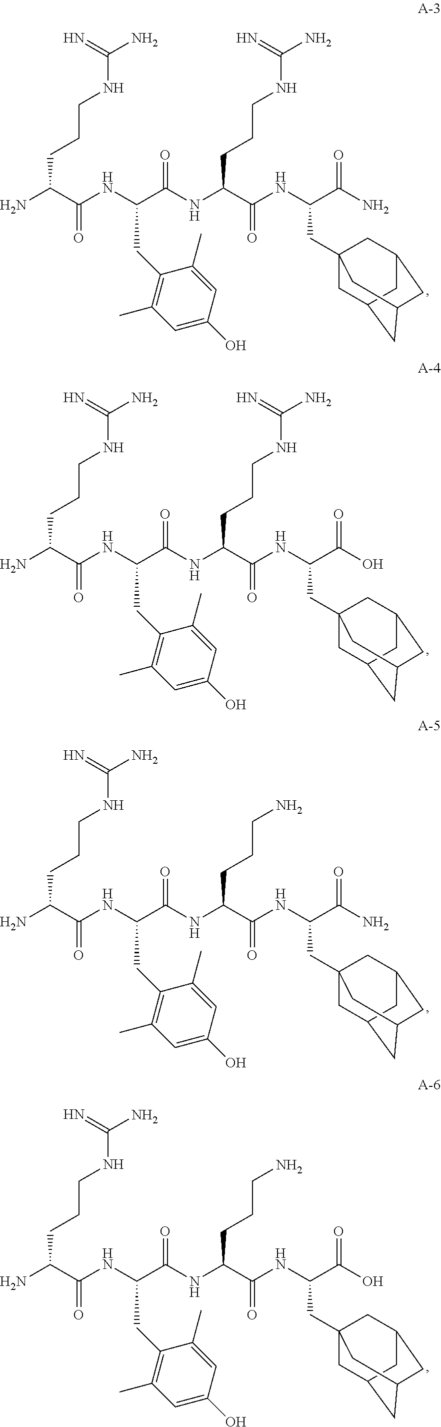 [SMILES: Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCNC(=N)N)C(=O)N[C@@H](CC12CC3CC(CC(C3)C1)C2)C(=O)O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCN)C(=O)N[C@@H](CC12CC3CC(CC(C3)C1)C2)C(=O)O] 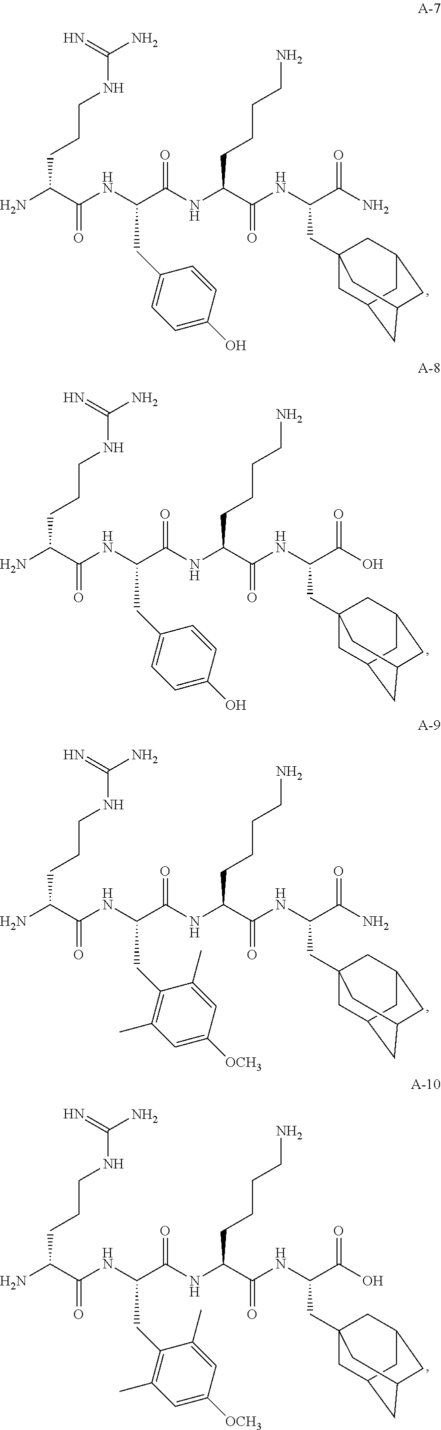 [SMILES: N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(N)=O ; N=C(N)NCCC[C@@H](N)C(=O)N[C@@H](Cc1ccc(O)cc1)C(=O)N[C@@H](CCCCN)C(=O)N[C@@H](CC12CC3CC(CC(C3)C1)C2)C(=O)O ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(N)=O)c(C)c1 ; COc1cc(C)c(C[C@H](NC(=O)[C@H](N)CCCNC(=N)N)C(=O)N[C@@H](CCCCN)C(=O)N[C@@H](CC23CC4CC(CC(C4)C2)C3)C(=O)O)c(C)c1] 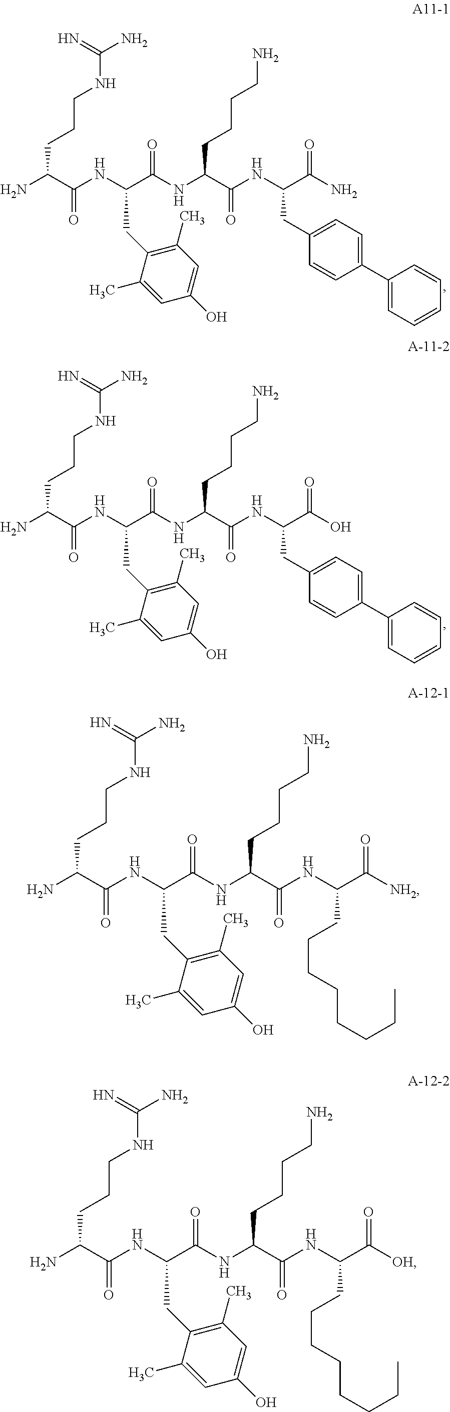 [SMILES: Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](CCCCN)C(=O)N[C@@H](Cc1ccc(-c2ccccc2)cc1)C(=O)O ; CCCCCCCC[C@H](NC(=O)[C@H](CCCCN)NC(=O)[C@H](Cc1c(C)cc(O)cc1C)NC(=O)[C@H](N)CCCNC(=N)N)C(N)=O ; CCCCCCCC[C@H](NC(=O)[C@H](CCCCN)NC(=O)[C@H](Cc1c(C)cc(O)cc1C)NC(=O)[C@H](N)CCCNC(=N)N)C(=O)O] 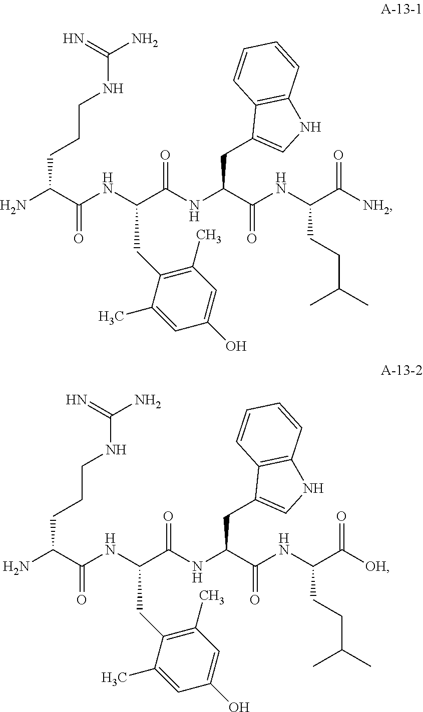 [SMILES: Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(N)=O ; Cc1cc(O)cc(C)c1C[C@H](NC(=O)[C@H](N)CCCNC(=N)N)C(=O)N[C@@H](Cc1c[nH]c2ccccc12)C(=O)N[C@@H](CCC(C)C)C(=O)O] 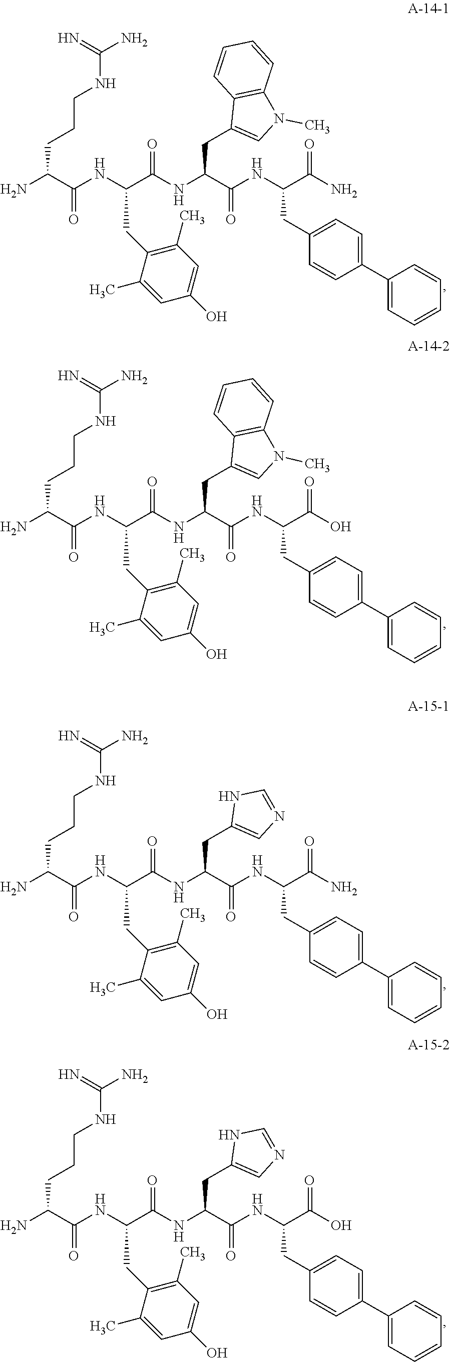 [SMILES: Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cn(C)c2ccccc12)C(=O)N[C@@H](Cc1ccc(-c2ccccc2)cc1)C(=O)O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(N)=O ; Cc1cc(O)cc(C)c1C[C@H](NC(=O)[C@H](N)CCCNC(=N)N)C(=O)N[C@@H](Cc1cnc[nH]1)C(=O)N[C@@H](Cc1ccc(-c2ccccc2)cc1)C(=O)O]
or a pharmaceutically acceptable salt, hydrate, solvate, stereoisomer and/or tautomer thereof.

102. The composition or medicament of any one of claims 99 to 101, wherein the composition or medicament is administered daily for: (i) 12 weeks or more; (ii) 24 weeks or more; (iii) 48 weeks or more; (iv) 72 weeks or more; or (v) 96 weeks or more.

103. The composition or medicament of any one of claims 99 to 102, wherein the composition or medicament is administered subcutaneously or intravenously.

104. The composition or medicament of any one of claims 99 to 102, wherein the composition or medicament is administered orally, topically, systemically, intraperitoneally, intradermally, transdermally, ophthalmically, intrathecally, intracerebroventricularly, iontophoretically, transmucosally, intravitreally, intranasally, or intramuscularly.

105. The composition or medicament of any one of claims 99 to 104, wherein the subject is human.

106. The composition or medicament of any one of claims 99 to 105, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

107. The composition or medicament of any one of claims 99 to 105, wherein the peptide or peptides of the composition or medicament is/are formulated for administration from its/their tris-HCl salt.

108. Use of a peptide, mixture of peptides or a composition or medicament comprising the peptide or the mixture of peptides for normalizing left ventricular ejection fraction, fractional shortening, and/or global longitudinal strain in a mammalian subject in need thereof, wherein the peptide or peptides of the mixture is/are of Formula A: 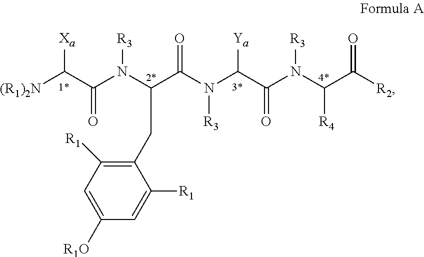
or a pharmaceutically acceptable salt, hydrate, solvate, and/or tautomer thereof, wherein, each R₁ is independently H, —CH₃ or —CH₂CH₃; R₂ is —OH, —NH₂, —NHR₁ or —N(R₁)₂; each R₃ is independently H, or —CH₃; R₄ is selected from: 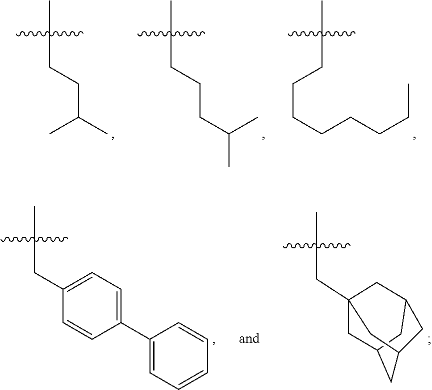
Xₐ is selected from 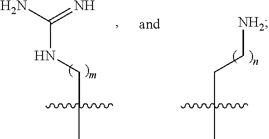
Yₐ is selected from 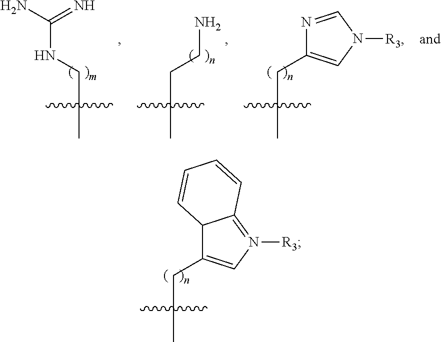
each m is independently 1, 2, 3 or 4; each n is independently 1, 2, or 3; the absolute stereochemistry at each of stereocenters 1*, 2*, 3*, and 4* is independently R or S, and optionally one or more hydrogen atoms of the peptide of Formula A is substituted with a deuterium or fluorine atom, wherein normalization or the left ventricular ejection fraction, fractional shortening, and/or global longitudinal strain occurs in the subject as compared with a control subject to control group of subjects not administered the peptide or peptides of Formula A.

109. The use of claim 108, wherein the peptide or peptides of Formula A is/are a peptide/peptides of Formula A-1 or Formula A-2: 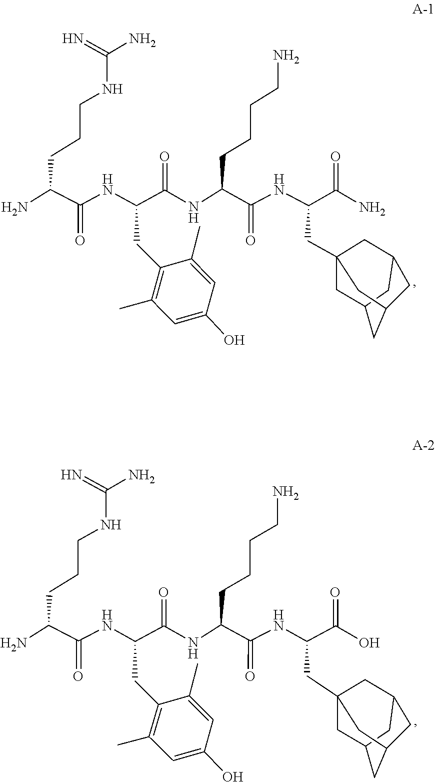 [SMILES: Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(N)=O ; Cc1cc(O)cc(C)c1C[C@H](NC(=O)[C@H](N)CCCNC(=N)N)C(=O)N[C@@H](CCCCN)C(=O)N[C@@H](CC12CC3CC(CC(C3)C1)C2)C(=O)O]
or a pharmaceutically acceptable salt, hydrate, solvate, stereoisomer and/or tautomer thereof.

110. The use of claim 108, wherein the peptide or peptides of Formula A is/are a peptide/peptides of Formula A-3, Formula A-4, Formula A-5, Formula A-6, Formula A-7, Formula A-8, Formula A-9, Formula A-10, Formula A-11-1, Formula A-11-2, Formula A-12-1, Formula A-12-2, Formula A-13-1, Formula A-13-2, Formula A-14-1, Formula A-14-2, Formula A-15-1, or Formula A-15-2: 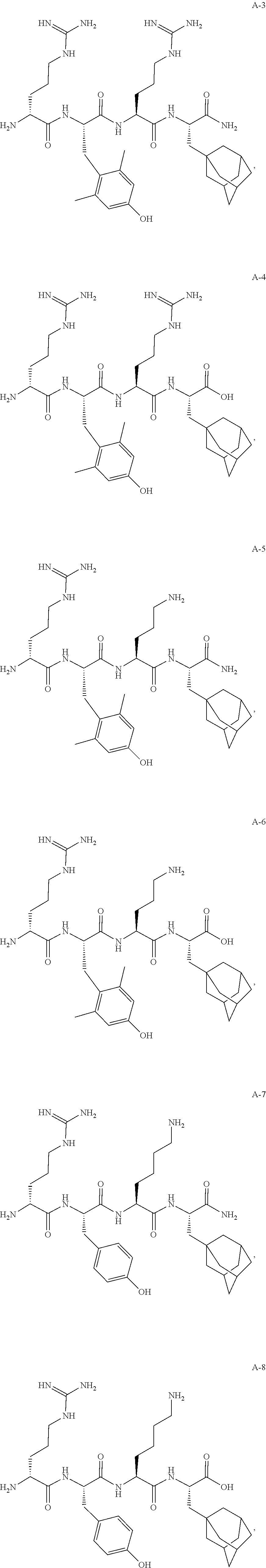 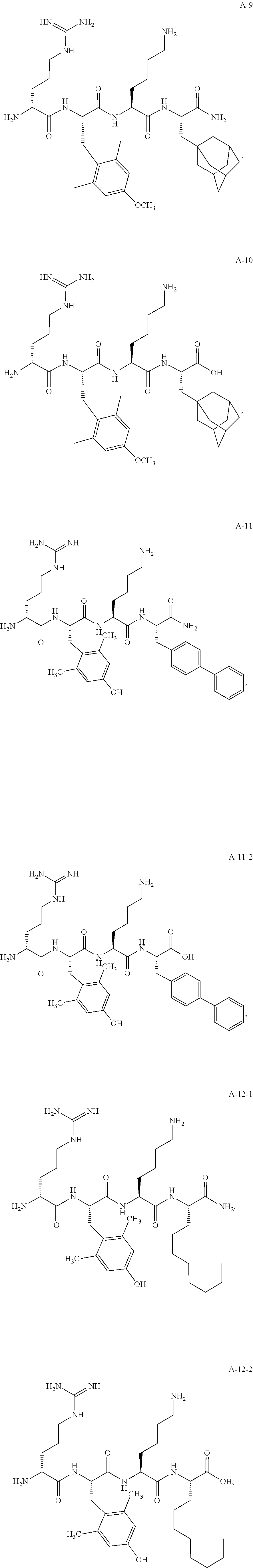 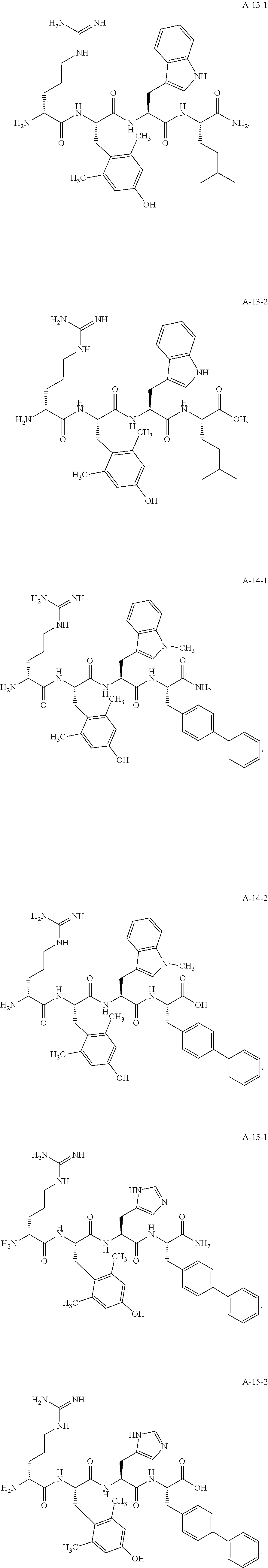
or a pharmaceutically acceptable salt, hydrate, solvate, stereoisomer and/or tautomer thereof.

111. The use of any one of claims 108 to 110, wherein the peptide, mixture of peptides, or the composition or medicament comprising the peptide or mixture of peptides is administered daily for: (i) 12 weeks or more; (ii) 24 weeks or more; (iii) 48 weeks or more; (iv) 72 weeks or more; or (v) 96 weeks or more.

112. The use of any one of claims 108 to 111, wherein the peptide, mixture of peptides or the composition or medicament comprising the peptide or mixture of peptides is administered subcutaneously or intravenously.

113. The use of any one of claims 108 to 111, wherein the peptide, mixture of peptides or the composition or medicament comprising the peptide or mixture of peptides is administered orally, topically, systemically, intraperitoneally, intradermally, transdermally, ophthalmically, intrathecally, intracerebroventricularly, iontophoretically, transmucosally, intravitreally, intranasally, or intramuscularly.

114. The use of any one of claims 108 to 113, wherein the subject is human.

115. The use of any one of claims 108 to 114, wherein the pharmaceutically acceptable salt of the peptide or peptides comprises/comprise hydrochloride, hydrobromide, acetate, citrate, benzoate, succinate, suberate, fumarate, lactate, oxalate, phthalate, methanesulfonate, benzenesulfonate, p-toluenesulfonate, tartrate, maleate, or trifluoroacetate salt.

116. The use of any one of claims 108 to 114, wherein the peptide or peptides is/are formulated for administration from its/their tris-HCl salt.
